SEQUENCE LISTING (107 sequences)

SEQ ID NO: 1 - protein, 10 aa, Artificial Sequence
  Complementarity determining region (CDR)
AFYDDYDYNV

SEQ ID NO: 2 - protein, 9 aa, Artificial Sequence
  Complementarity determining region (CDR)
NSGDAQFNI

SEQ ID NO: 3 - protein, 9 aa, Artificial Sequence
  Complementarity determining region (CDR)
STTDAQFNI

SEQ ID NO: 4 - protein, 7 aa, Artificial Sequence
  Complementarity determining region (CDR)
EIPYFNV

SEQ ID NO: 5 - protein, 16 aa, Artificial Sequence
  Complementarity determining region (CDR)
AGYVGAVYVTLTRLDL

SEQ ID NO: 6 - protein, 12 aa, Artificial Sequence
  Complementarity determining region (CDR)
ARAFYDDYDYNV

SEQ ID NO: 7 - protein, 11 aa, Artificial Sequence
  Complementarity determining region (CDR)
ARNSGDAQFNI

SEQ ID NO: 8 - protein, 11 aa, Artificial Sequence
  Complementarity determining region (CDR)
ARSTTDAQFNI

SEQ ID NO: 9 - protein, 9 aa, Artificial Sequence
  Complementarity determining region (CDR)
ATEIPYFNV

SEQ ID NO: 10 - protein, 18 aa, Artificial Sequence
  Complementarity determining region (CDR)
ARAGYVGAVYVTLTRLDL

SEQ ID NO: 11 - protein, 8 aa, Artificial Sequence
  Complementarity determining region (CDR)
IISGGTIG

SEQ ID NO: 12 - protein, 9 aa, Artificial Sequence
  Complementarity determining region (CDR)
SFYADSGTT

SEQ ID NO: 13 - protein, 9 aa, Artificial Sequence
  Complementarity determining region (CDR)
IIYPDTGTT

SEQ ID NO: 14 - protein, 9 aa, Artificial Sequence
  Complementarity determining region (CDR)
IGIGGSGST

SEQ ID NO: 15 - protein, 9 aa, Artificial Sequence
  Complementarity determining region (CDR)
IYVGSSGVS

SEQ ID NO: 16 - protein, 16 aa, Artificial Sequence
  Complementarity determining region (CDR)
IISGGTIGHYASWAKG

SEQ ID NO: 17 - protein, 17 aa, Artificial Sequence
  Complementarity determining region (CDR)
SFYADSGTTWYATWVKG

SEQ ID NO: 18 - protein, 17 aa, Artificial Sequence
  Complementarity determining region (CDR)
IIYPDTGTTWYASWVKG

SEQ ID NO: 19 - protein, 18 aa, Artificial Sequence
  Complementarity determining region (CDR)
CIGIGGSGSTYYAGWAKG

SEQ ID NO: 20 - protein, 18 aa, Artificial Sequence
  Complementarity determining region (CDR)
CIYVGSSGVSYYATWAKG

SEQ ID NO: 21 - protein, 4 aa, Artificial Sequence
  Complementarity determining region (CDR)
DTYW

SEQ ID NO: 22 - protein, 4 aa, Artificial Sequence
  Complementarity determining region (CDR)
SVYY

SEQ ID NO: 23 - protein, 5 aa, Artificial Sequence
  Complementarity determining region (CDR)
SYNMG

SEQ ID NO: 24 - protein, 5 aa, Artificial Sequence
  Complementarity determining region (CDR)
RYYIS

SEQ ID NO: 25 - protein, 5 aa, Artificial Sequence
  Complementarity determining region (CDR)
RYYMT

SEQ ID NO: 26 - protein, 6 aa, Artificial Sequence
  Complementarity determining region (CDR)
DTYWIC

SEQ ID NO: 27 - protein, 6 aa, Artificial Sequence
  Complementarity determining region (CDR)
SVYYMC

SEQ ID NO: 28 - protein, 8 aa, Artificial Sequence
  Complementarity determining region (CDR)
GFSLYSYN

SEQ ID NO: 29 - protein, 8 aa, Artificial Sequence
  Complementarity determining region (CDR)
GFSLSRYY

SEQ ID NO: 30 - protein, 8 aa, Artificial Sequence
  Complementarity determining region (CDR)
GFSLSRYY

SEQ ID NO: 31 - protein, 9 aa, Artificial Sequence
  Complementarity determining region (CDR)
GIDFSDTYW

SEQ ID NO: 32 - protein, 9 aa, Artificial Sequence
  Complementarity determining region (CDR)
GIDFSSVYY

SEQ ID NO: 33 - protein, 11 aa, Artificial Sequence
  Complementarity determining region (CDR)
AGGYSSSSDTT

SEQ ID NO: 34 - protein, 11 aa, Artificial Sequence
  Complementarity determining region (CDR)
AGGYSVTSDTT

SEQ ID NO: 35 - protein, 11 aa, Artificial Sequence
  Complementarity determining region (CDR)
AGGYSTTSDTT

SEQ ID NO: 36 - protein, 12 aa, Artificial Sequence
  Complementarity determining region (CDR)
QGTYDVNGWLVA

SEQ ID NO: 37 - protein, 11 aa, Artificial Sequence
  Complementarity determining region (CDR)
LGGYDDDADNA

SEQ ID NO: 38 - protein, 7 aa, Artificial Sequence
  Complementarity determining region (CDR)
QASKLET

SEQ ID NO: 39 - protein, 7 aa, Artificial Sequence
  Complementarity determining region (CDR)
DASTLAS

SEQ ID NO: 40 - protein, 7 aa, Artificial Sequence
  Complementarity determining region (CDR)
DASKLTS

SEQ ID NO: 41 - protein, 7 aa, Artificial Sequence
  Complementarity determining region (CDR)
DASKLAS

SEQ ID NO: 42 - protein, 8 aa, Artificial Sequence
  Complementarity determining region (CDR)
QSVYGNNQ

SEQ ID NO: 43 - protein, 8 aa, Artificial Sequence
  Complementarity determining region (CDR)
ESVYNKNQ

SEQ ID NO: 44 - protein, 8 aa, Artificial Sequence
  Complementarity determining region (CDR)
ENVYTDNQ

SEQ ID NO: 45 - protein, 8 aa, Artificial Sequence
  Complementarity determining region (CDR)
QSVYNKNW

SEQ ID NO: 46 - protein, 8 aa, Artificial Sequence
  Complementarity determining region (CDR)
QSIYTNND

SEQ ID NO: 47 - protein, 13 aa, Artificial Sequence
  Complementarity determining region (CDR)
QSSQSVYGNNQLS

SEQ ID NO: 48 - protein, 13 aa, Artificial Sequence
  Complementarity determining region (CDR)
QSSESVYNKNQLC

SEQ ID NO: 49 - protein, 13 aa, Artificial Sequence
  Complementarity determining region (CDR)
QSSENVYTDNQLC

SEQ ID NO: 50 - protein, 13 aa, Artificial Sequence
  Complementarity determining region (CDR)
QSSQSVYNKNWLA

SEQ ID NO: 51 - protein, 13 aa, Artificial Sequence
  Complementarity determining region (CDR)
QSSQSIYTNNDLA

SEQ ID NO: 52 - protein, 116 aa, Artificial Sequence
  Heavy chain variable region (VH) (MAB-19-0202)
QSVEESGGRLVTPGTPLTLTCTVSGFSLYSYNMGWVRQAPGKGLEYIGIISGGTIGHYASWAKGRFTISKTSSTTVDLKMTSLTTEDTATYFCARAFYDDYDYNVWGPGTLVTVSS

SEQ ID NO: 53 - protein, 116 aa, Artificial Sequence
  Heavy chain variable region (VH)(MAB-19-0208)
QSVEESGGRLVTPGTPLTLTCTVSGFSLSRYYISWVRQAPGKGLEWIGSFYADSGTTWYATWVKGRFTFSTASSTTVDLKMTSPTTEDTATYFCARNSGDAQFNIWGPGTLVTVSS

SEQ ID NO: 54 - protein, 116 aa, Artificial Sequence
  Heavy chain variable region (VH) (MAB-19-0217)
QSVEESGGRLVTPGTPLTLTCTVSGFSLSRYYMTWVRQAPGKGLEWIGIIYPDTGTTWYASWVKGRFTFSKTSSTTVDLKMTSPTTEDTATYFCARSTTDAQFNIWGPGTLVTVSS

SEQ ID NO: 55 - protein, 117 aa, Artificial Sequence
  Heavy chain variable region (VH) (MAB-19-0223)
QEHLVESGGGLVQPEGSLTLTCKASGIDFSDTYWICWVRQPPGKGLEWIGCIGIGGSGSTYYAGWAKGRFTISKTSSTTVTLQMTTLTDADTATYFCATEIPYFNVWGPGTLVTVSS

SEQ ID NO: 56 - protein, 125 aa, Artificial Sequence
  Heavy chain variable region (VH) (MAB-19-0233)
QSLEESGGDLVKPGASLTLTCKASGIDFSSVYYMCWVRQAPGKGLEWIACIYVGSSGVSYYATWAKGRFTISKTSSTTVTLQMTSLTAADTATYFCARAGYVGAVYVTLTRLDLWGQGTLVTVSS

SEQ ID NO: 57 - protein, 111 aa, Artificial Sequence
  Light chain variable region (VL) (MAB-19-0202)
AAVLTQTPSPVSAAVGGTVTISCQSSQSVYGNNQLSWYQQKPGQPPKLLIYQASKLETGVPSRFKGSGSGTQFTLTISDLESDDAATYYCAGGYSSSSDTTFGGGTEVVVK

SEQ ID NO: 58 - protein, 111 aa, Artificial Sequence
  Light chain variable region (VL) (MAB-19-0208)
AAVLTQTPSPVSAAVGGTVSISCQSSESVYNKNQLCWYQQKPGQRPKLLIYDASTLASGVPSRFSGSGSGTQFTLTISDVQSDAAATYYCAGGYSVTSDTTFGGGTEVVVR

SEQ ID NO: 59 - protein, 111 aa, Artificial Sequence
  Light chain variable region (VL) (MAB-19-0217)
AAVLTQTPSPVSAAVGGTVSISCQSSENVYTDNQLCWYQQKPGQRPKLLIYDASTLASGVPSRFSGSGSGTQFTLTISGVQSDDAATYYCAGGYSTTSDTTFGGGTEVVVK

SEQ ID NO: 60 - protein, 112 aa, Artificial Sequence
  Light chain variable region (VL) (MAB-19-0223)
AQVLTQTPSSVSAAVGGTVTINCQSSQSVYNKNWLAWYQQKPGQPPKLLIYDASKLTSGVPSRFKGSGSGTQFTLTISGVQSDDAATYYCQGTYDVNGWLVAFGGGAEVVVK

SEQ ID NO: 61 - protein, 111 aa, Artificial Sequence
  Light chain variable region (VL) (MAB-19-0233)
AAVLTQTPSPVSAAVGGTVTISCQSSQSIYTNNDLAWYQQKPGQPPKLLIYDASKLASGVPSRFSGSGSGTQFTLTISGVQSDDAATYYCLGGYDDDADNAFGGGTEVVVK

SEQ ID NO: 62 - protein, 118 aa, Artificial Sequence
  Heavy chain variable region (H5) (derived from MAB-19-0202)
QVQLVESGGGLVQPGTSLRLSCSVSGFSLYSYNMGWVRQAPGKGLEYIGIISGGTIGHYASWAKGRFTISRDTSKTTLYLQMNSLTTEDTATYFCARAFYDDYDYNVWGPGTLVTVSS

SEQ ID NO: 63 - protein, 127 aa, Artificial Sequence
  Heavy chain variable region (H5) (derived from MAB-19-0233)
QVQLVESGGDVVKPGRSLRLSCKASGIDFSSVYYMCWVRQAPGKGLEWIACIYVGSSGVSYYATWAKGRFTISRDTSTSTLFLQMNSLRAGDTATYYCARAGYVGAVYVTLTRLDLWGQGTLVTVSS

SEQ ID NO: 64 - protein, 127 aa, Artificial Sequence
  Heavy chain variable region (H1) (derived from MAB-19-0233)
EVQLEESGGGLVKPGGSLRLSCAASGIDFSSVYYMCWVRQAPGKGLEWVSCIYVGSSGVSYYATWAKGRFTISRDNSKNTLYLQMNSLRAEDTAVYYCARAGYVGAVYVTLTRLDLWGRGTLVTVSS

SEQ ID NO: 65 - protein, 111 aa, Artificial Sequence
  Light chain variable region (L1) (derived from MAB-19-0202)
DIVMTQSPSSLSASVGDRVTITCQSSQSVYGNNQLSWYQQKPGKAPKLLIYQASKLETGVPSRFSGSGSGTDFTLTISSLQPEDFATYYCAGGYSSSSDTTFGGGTKVVIK

SEQ ID NO: 66 - protein, 111 aa, Artificial Sequence
  Light chain variable region (L2) (derived from MAB-19-0202)
DIQMTQSPSTLSASVGDRVTITCQSSQSVYGNNQLSWYQQKPGKAPKLLIYQASKLETGVPSRFSGSGSGTQFTLTISSLQPDDFASYYCAGGYSSSSDTTFGQGTKVEIK

SEQ ID NO: 67 - protein, 111 aa, Artificial Sequence
  Light chain variable region (L3) (derived from MAB-19-0202)
DIQMTQSPSSLSASVGDRVTITCQSSQSVYGNNQLSWYQKKPGQAPKLLIYQASKLETGVPSRFSGSGSGTDFTLTISSLQPEDFATYYCAGGYSSSSDTTFGPGTKVDIK

SEQ ID NO: 68 - protein, 111 aa, Artificial Sequence
  Light chain variable region (L4) (derived from MAB-19-0202)
AIQLTQSPSSLSASVGGTVTITCQSSQSVYGNNQLSWYQQKPGQPPKLLIYQASKLETGVPSRFRGSGSGTQFTLTISSLQSEDFATYYCAGGYSSSSDTTFGGGTEVVVK

SEQ ID NO: 69 - protein, 111 aa, Artificial Sequence
  Light chain variable region (L1) (derived from MAB-19-0233)
DVVMTQSPSTVSASVGDRVTLTCQSSQSIYTNNDLAWYQQKPGQPPKLLIYDASKLASGVPDRFSGSGSGTDFTLTISSLQADDFATYYCLGGYDDDADNAFGQGTKVEIK

SEQ ID NO: 70 - protein, 111 aa, Artificial Sequence
  Light chain variable region (L4) (derived from MAB-19-0233)
DIQMTQSPSSLSASVGGTVTITCQSSQSIYTNNDLAWYQQKPGQPPKLLIYDASKLASGVPSRFSGSGSGTQFTLTISSLQSEDAATYYCLGGYDDDADNAFGGGTEVVVK

SEQ ID NO: 71 - protein, 288 aa, Homo sapiens
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 72 - protein, 147 aa, Homo sapiens
FLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLV

SEQ ID NO: 73 - DNA, 2115 bp, Homo sapiens
agtttcccttccgctcacctccgcctgagcagtggagaaggcggcactctggtggggctgctccaggcatgcagatcccacaggcgccctggccagtcgtctgggcggtgctacaactgggctggcggccaggatggttcttagactccccagacaggccctggaacccccccaccttctccccagccctgctcgtggtgaccgaaggggacaacgccaccttcacctgcagcttctccaacacatcggagagcttcgtgctaaactggtaccgcatgagccccagcaaccagacggacaagctggccgccttccccgaggaccgcagccagcccggccaggactgccgcttccgtgtcacacaactgcccaacgggcgtgacttccacatgagcgtggtcagggcccggcgcaatgacagcggcacctacctctgtggggccatctccctggcccccaaggcgcagatcaaagagagcctgcgggcagagctcagggtgacagagagaagggcagaagtgcccacagcccaccccagcccctcacccaggccagccggccagttccaaaccctggtggttggtgtcgtgggcggcctgctgggcagcctggtgctgctagtctgggtcctggccgtcatctgctcccgggccgcacgagggacaataggagccaggcgcaccggccagcccctgaaggaggacccctcagccgtgcctgtgttctctgtggactatggggagctggatttccagtggcgagagaagaccccggagccccccgtgccctgtgtccctgagcagacggagtatgccaccattgtctttcctagcggaatgggcacctcatcccccgcccgcaggggctcagctgacggccctcggagtgcccagccactgaggcctgaggatggacactgctcttggcccctctgaccggcttccttggccaccagtgttctgcagaccctccaccatgagcccgggtcagcgcatttcctcaggagaagcaggcagggtgcaggccattgcaggccgtccaggggctgagctgcctgggggcgaccggggctccagcctgcacctgcaccaggcacagccccaccacaggactcatgtctcaatgcccacagtgagcccaggcagcaggtgtcaccgtcccctacagggagggccagatgcagtcactgcttcaggtcctgccagcacagagctgcctgcgtccagctccctgaatctctgctgctgctgctgctgctgctgctgctgcctgcggcccggggctgaaggcgccgtggccctgcctgacgccccggagcctcctgcctgaacttgggggctggttggagatggccttggagcagccaaggtgcccctggcagtggcatcccgaaacgccctggacgcagggcccaagactgggcacaggagtgggaggtacatggggctggggactccccaggagttatctgctccctgcaggcctagagaagtttcagggaaggtcagaagagctcctggctgtggtgggcagggcaggaaacccctccacctttacacatgcccaggcagcacctcaggccctttgtggggcagggaagctgaggcagtaagcgggcaggcagagctggaggcctttcaggcccagccagcactctggcctcctgccgccgcattccaccccagcccctcacaccactcgggagagggacatcctacggtcccaaggtcaggagggcagggctggggttgactcaggcccctcccagctgtggccacctgggtgttgggagggcagaagtgcaggcacctagggccccccatgtgcccaccctgggagctctccttggaacccattcctgaaattatttaaaggggttggccgggctcccaccagggcctgggtgggaaggtacaggcgttcccccggggcctagtacccccgccgtggcctatccactcctcacatccacacactgcacccccactcctggggcagggccaccagcatccaggcggccagcaggcacctgagtggctgggacaagggatcccccttccctgtggttctattatattataattataattaaatatgagagcatgctaaggaaaa

SEQ ID NO: 74 - DNA, 348 bp, Artificial Sequence
  Heavy chain variable region (VH) (MAB-19-0202)
cagagcgtggaagaatctggcggcagactggtcacacctggcacacctctgacactgacctgtaccgtgtccggcttcagcctgtacagctacaacatgggctgggtccgacaggcccctggaaagggactcgagtacatcggcatcatcagcggcggcacaatcggccactatgcctcttgggccaagggcagattcaccatcagcaagaccagcagcaccaccgtggacctgaagatgaccagcctgaccaccgaggacaccgccacctacttttgcgccagagccttctacgacgactacgactacaacgtgtggggcccaggcacactcgtgacagtctcctct

SEQ ID NO: 75 - DNA, 348 bp, Artificial Sequence
  Heavy chain variable region (VH) (MAB-19-0208)
cagagcgtggaagaatctggcggcagactggtcacacctggcacacctctgacactgacctgtaccgtgtccggcttcagcctgagccggtactacatcagctgggtccgacaggcccctggcaaaggactggaatggatcggcagcttctacgccgatagcggcacaacttggtacgccacctgggtcaagggcagattcacctttagcaccgccagcagcaccaccgtggacctgaagatgacaagccccaccaccgaggacaccgccacctacttttgcgccagaaacagcggcgacgcccagttcaatatctggggccctggaacactggtcaccgtgtcatct

SEQ ID NO: 76 - DNA, 348 bp, Artificial Sequence
  Heavy chain variable region (VH) (MAB-19-0217)
cagagcgtggaagaatctggcggcagactggtcacacctggcacacctctgacactgacctgtaccgtgtccggcttcagcctgagccggtactacatgacctgggtccgacaggcccctggcaaaggactggaatggatcggcatcatctaccccgacaccggcacaacttggtacgcctcttgggtcaagggcagattcaccttcagcaagaccagcagcaccaccgtggacctgaagatgacaagccccaccaccgaggacaccgccacctacttttgtgccagaagcaccacagacgcccagttcaacatctggggccctggaacactggtcaccgtgtcatct

SEQ ID NO: 77 - DNA, 351 bp, Artificial Sequence
  Heavy chain variable region (VH) (MAB-19-0223)
caagagcacctggtggaatctggcggaggactggttcagcctgagggctctctgaccctgacctgtaaagccagcggcatcgacttcagcgacacctactggatctgctgggtccgacagcctcctggcaaaggcctggaatggatcggctgtatcggcatcggcggcagcggcagcacatattatgccggatgggccaagggcagattcaccatcagcaagaccagcagcaccaccgtgacactgcagatgaccacactgaccgacgccgacaccgccacctatttctgtgccaccgagattccctacttcaacgtgtggggccctggcacactggtcacagtctcttct

SEQ ID NO: 78 - DNA, 375 bp, Artificial Sequence
  Heavy chain variable region (VH) (MAB-19-0233)
cagagcctggaagaatctggcggcgatcttgtgaaacctggcgcctctctgaccctgacatgtaaagccagcggcatcgacttcagcagcgtgtactacatgtgttgggtccgacaggcccctggcaaaggcctggaatggatcgcctgtatctacgtgggcagcagcggcgtgtcctactatgccacatgggccaagggcagattcaccatcagcaagaccagcagcaccaccgtgacactgcagatgacatctctgacagccgccgacaccgccacctacttttgtgccagagccggatatgtgggcgccgtgtatgtgacactgaccagactggatctgtggggccagggcacactggtcacagtctcctct

SEQ ID NO: 79 - DNA, 333 bp, Artificial Sequence
  Light chain variable region (VL) (MAB-19-0202)
gctgctgtgctgacccagacaccttctccagtgtctgccgccgttggcggcacagtgacaatcagctgtcagagcagccagagcgtgtacggcaacaaccagctgtcctggtatcagcagaagcccggccagcctcctaagctgctgatctaccaggccagcaagctggaaacaggcgtgcccagcagattcaaaggcagcggctctggcacccagttcaccctgacaatctccgacctggaaagcgacgatgccgccacctactattgtgccggcggatacagcagcagctccgacacaacatttggcggcggaacagaggtggtggtcaag

SEQ ID NO: 80 - DNA, 333 bp, Artificial Sequence
  Light chain variable region (VL) (MAB-19-0208)
gctgctgtgctgacccagacaccttctccagtgtctgccgctgttggcggcacagtgtctatcagctgtcagagcagcgagagcgtgtacaacaagaaccagctgtgctggtatcagcagaagcccggccagaggcctaagctgctgatctacgatgccagcacactggccagcggagtgcctagcagattttctggcagcggctctggcacccagttcaccctgacaatctccgacgtgcagtctgatgccgccgctacctactattgtgccggcggatacagcgtgaccagcgacacaacatttggcggcggaacagaggtggtcgtcaga

SEQ ID NO: 81 - DNA, 333 bp, Artificial Sequence
  Light chain variable region (VL) (MAB-19-0217)
gctgctgtgctgacccagacaccttctccagtgtctgccgctgttggcggcacagtgtctatcagctgtcagagcagcgagaacgtgtacaccgacaaccagctgtgctggtatcagcagaagcctggccagaggcctaagctgctgatctacgatgccagcacactggccagcggagtgcctagcagattttctggcagcggctctggcacccagttcaccctgacaattagcggcgtgcagtccgatgatgccgccacctattattgcgctggcggctacagcaccaccagcgatacaacatttggcggcggaaccgaggtggtggtcaaa

SEQ ID NO: 82 - DNA, 336 bp, Artificial Sequence
  Light chain variable region (VL) (MAB-19-0223)
gctcaggtgctgacacagacacctagctctgtgtctgccgccgttggcggcaccgtgaccatcaattgtcagagcagccagagcgtgtacaacaagaactggctggcctggtatcagcagaagcctggccagcctcctaagctgctgatctacgatgccagcaagctgaccagcggcgtgccctctagattcaaaggctctggcagcggcacccagttcaccctgacaatttctggcgtgcagagcgacgacgccgccacctattattgccaaggcacctacgacgtgaacggctggctggttgcttttggaggcggagccgaagtggtggtcaaa

SEQ ID NO: 83 - DNA, 333 bp, Artificial Sequence
  Light chain variable region (VL) (MAB-19-0233)
gctgctgtgctgacccagacaccttctccagtgtctgccgccgttggcggcacagtgacaatcagctgtcagagcagccagagcatctacaccaacaacgacctggcctggtatcagcagaagcctggccagcctcctaagctgctgatctacgatgccagcaagctggcctctggcgtgccaagcagattttctggcagcggctctggcacccagttcaccctgacaattagcggcgtgcagtccgatgatgccgccacctattattgcctcggcggctacgatgacgacgccgacaatgcttttggcggcggaacagaggtggtggtcaaa

SEQ ID NO: 84 - DNA, 354 bp, Artificial Sequence
  Heavy chain variable region (H5) (derived from MAB-19-0202)
caggtgcagctggttgaatctggcggaggactggtgcagcctggcacatctctgagactgagctgtagcgtgtccggcttcagcctgtacagctacaacatgggctgggtccgacaggcccctggaaagggactcgagtacatcggcatcatcagcggcggcacaatcggccactatgcctcttgggccaagggcagattcaccatcagccgggacaccagcaagaccacactgtacctgcagatgaacagcctgaccaccgaggacaccgccacctacttttgcgccagagccttctacgacgactacgactacaacgtgtggggccctggcacactggtcacagtctcttct

SEQ ID NO: 85 - DNA, 381 bp, Artificial Sequence
  Heavy chain variable region (H5) (derived from MAB-19-0233)
caggtgcagctggttgagtctggcggagatgtggtcaagcctggcagaagcctgagactgagctgtaaagccagcggcatcgacttcagcagcgtgtactacatgtgctgggtccgacaggcccctggcaaaggactggaatggatcgcctgtatctacgtgggcagcagcggcgtgtcctactatgccacatgggccaagggcagattcaccatcagccgggacacctctaccagcacactgtttctgcagatgaacagcctgagagccggcgacacagccacctactattgtgccagagccggctatgtgggcgccgtgtatgtgaccctgaccagactggatctgtggggccagggaacactggtcacagtgtcatct

SEQ ID NO: 86 - DNA, 381 bp, Artificial Sequence
  Heavy chain variable region (H1) (derived from MAB-19-0233)
gaggtgcagctggaagaatctggcggcggacttgtgaagcctggcggatctctgagactgagctgtgccgcctctggcatcgatttcagcagcgtgtactacatgtgctgggtccgacaggcccctggcaaaggacttgaatgggtgtcctgcatctacgtgggcagcagcggcgtgtcctactatgccacatgggccaagggcagattcaccatcagccgggacaacagcaagaacaccctgtacctgcagatgaacagcctgagagccgaggacaccgccgtgtactattgtgccagagccggatatgtgggcgccgtgtatgtgaccctgaccagactggatctgtggggcagaggcacactggtcacagtgtcatct

SEQ ID NO: 87 - DNA, 333 bp, Artificial Sequence
  Light chain variable region (L1) (derived from MAB-19-0202)
gacatcgtgatgacacagagccctagcagcctgtctgccagcgtgggagacagagtgaccatcacctgtcagagcagccagagcgtgtacggcaacaaccagctgtcctggtatcagcagaagcccggcaaggcccctaagctgctgatctaccaggccagcaagctggaaacaggcgtgcccagcagattttctggcagcggctctggcaccgacttcaccctgaccatatctagcctgcagcctgaggacttcgccacctactattgtgccggcggatacagcagcagctccgacacaacatttggcggaggcaccaaggtggtcatcaag

SEQ ID NO: 88 - DNA, 333 bp, Artificial Sequence
  Light chain variable region (L2) (derived from MAB-19-0202)
gacatccagatgacacagagccccagcacactgtctgccagcgtgggagacagagtgaccatcacctgtcagagcagccagagcgtgtacggcaacaaccagctgtcctggtatcagcagaagcccggcaaggcccctaagctgctgatctaccaggccagcaagctggaaacaggcgtgcccagcagattttctggcagcggctctggcacccagttcaccctgaccatcagtagcctgcagcctgacgacttcgccagctactattgtgccggcggatacagcagcagctccgataccacatttggccagggcaccaaggtggaaatcaag

SEQ ID NO: 89 - DNA, 333 bp, Artificial Sequence
  Light chain variable region (L3) (derived from MAB-19-0202)
gacatccagatgacacagagccctagcagcctgtctgccagcgtgggagacagagtgaccatcacctgtcagagcagccagagcgtgtacggcaacaaccagctgtcctggtatcagaagaagcccggacaggcccctaagctgctgatctaccaggccagcaagctggaaacaggcgtgcccagcagattttctggcagcggctctggcaccgacttcaccctgaccatatctagcctgcagcctgaggacttcgccacctactattgtgccggcggatacagcagcagctccgacacaacatttggccctggcaccaaggtggacatcaag

SEQ ID NO: 90 - DNA, 333 bp, Artificial Sequence
  Light chain variable region (L4) (derived from MAB-19-0202)
gccattcagctgacacagagcccttctagcctgagcgcctctgttggcggcaccgtgacaatcacctgtcagagcagccagagcgtgtacggcaacaaccagctgtcctggtatcagcagaagcccggccagcctcctaagctgctgatctaccaggccagcaagctggaaacaggcgtgccctctagattcagaggcagcggctctggcacccagttcacactgacaatcagcagcctgcagagcgaggacttcgccacctactattgtgccggcggatacagcagcagctccgacacaacatttggcggcggaacagaggtggtggtcaag

SEQ ID NO: 91 - DNA, 333 bp, Artificial Sequence
  Light chain variable region (L1) (derived from MAB-19-0233)
gacgtggtcatgacacagagccctagcacagtgtctgccagcgtgggcgatagagtgaccctgacctgtcagagcagccagagcatctacaccaacaacgacctggcctggtatcagcagaagcctggccagcctcctaagctgctgatctacgatgccagcaagctggcctctggcgtgcccgatagattttctggcagcggctctggcaccgacttcaccctgacaattagctccctgcaggccgacgacttcgccacctattattgtctcggcggctacgacgacgacgccgataatgcttttggccagggcaccaaggtggaaatcaag

SEQ ID NO: 92 - DNA, 333 bp, Artificial Sequence
  Light chain variable region (L4) (derived from MAB-19-0233)
gacatccagatgacacagagccctagcagcctgtctgcctctgttggcggcaccgtgacaatcacctgtcagagcagccagagcatctacaccaacaacgacctggcctggtatcagcagaagcctggccagcctcctaagctgctgatctacgatgccagcaagctggcctctggcgtgccaagcagattttctggcagcggctctggcacccagttcaccctgacaatctctagcctgcagagcgaggatgccgccacctactattgtctcggcggctacgacgacgacgccgacaatgcttttggcggcggaacagaggtggtggtcaaa

SEQ ID NO: 93 - DNA, 1886 bp, Artificial Sequence
  pST4-vector (HC; RiboMab-19-0202)
attcttctggtccccacagactcagagagaacccgccaccatgagagtgatggcccccagaaccctgatcctgctgctgtctggcgccctggccctgacagagacatgggccggaagccagagcgtggaagaatctggcggcagactggtcacacctggcacacctctgacactgacctgtaccgtgtccggcttcagcctgtacagctacaacatgggctgggtccgacaggcccctggaaagggactcgagtacatcggcatcatcagcggcggcacaatcggccactatgcctcttgggccaagggcagattcaccatcagcaagaccagcagcaccaccgtggacctgaagatgaccagcctgaccaccgaggacaccgccacctacttttgcgccagagccttctacgacgactacgactacaacgtgtggggcccaggcacactcgtgacagtctcctctgcctctacaaagggccctagcgtgttccctctggctcctagcagcaagtctacaagcggaggaacagccgctctgggctgcctggtcaaggattactttcccgagcctgtgaccgtgtcctggaattctggcgctctgacaagcggcgtgcacacctttccagctgtgctgcaaagcagcggcctgtactctctgagcagcgtggtcacagtgccaagctctagcctgggcacccagacctacatctgcaatgtgaaccacaagcctagcaacaccaaggtggacaagaaggtggaacccaagagctgcgacaagacccacacctgtcctccatgtcctgctccagaagctgctggcggcccttccgtgtttctgttccctccaaagcctaaggacaccctgatgatcagcagaacccctgaagtgacctgcgtggtggtggatgtgtcccacgaggatcccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctgggcgctcccatcgagaaaaccatctctaaggccaagggacagccccgcgaacctcaggtttacacactgcctccaagccgggaagaaatgaccaagaaccaggtgtccctgacctgcctcgtgaagggcttctacccttccgatatcgccgtggaatgggagagcaatggccagcctgagaacaactacaagacaacccctcctgtgctggactccgatggctcattcttcctgtacagcaagctgacagtggacaagtccagatggcagcagggcaacgtgttcagctgcagcgtgatgcacgaggccctgcacaaccactacacccagaagtccctgagcctgtctcctggatgatgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccgagacctggtccagagtcgctagccgcgtcgctaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 94 - DNA, 34 bp, Artificial Sequence
  5′-UTR
attcttctggtccccacagactcagagagaaccc

SEQ ID NO: 95 - DNA, 6 bp, Artificial Sequence
  Kozac sequence
gccacc

SEQ ID NO: 96 - DNA, 78 bp, Artificial Sequence
  signal peptide sequence
atgagagtgatggcccccagaaccctgatcctgctgctgtctggcgccctggccctgacagagacatgggccggaagc

SEQ ID NO: 97 - DNA, 294 bp, Artificial Sequence
  constant domain CH1
gcctctacaaagggccctagcgtgttccctctggctcctagcagcaagtctacaagcggaggaacagccgctctgggctgcctggtcaaggattactttcccgagcctgtgaccgtgtcctggaattctggcgctctgacaagcggcgtgcacacctttccagctgtgctgcaaagcagcggcctgtactctctgagcagcgtggtcacagtgccaagctctagcctgggcacccagacctacatctgcaatgtgaaccacaagcctagcaacaccaaggtggacaagaaggtg

SEQ ID NO: 98 - DNA, 45 bp, Artificial Sequence
  hinge region
gaacccaagagctgcgacaagacccacacctgtcctccatgtcct

SEQ ID NO: 99 - DNA, 330 bp, Artificial Sequence
  constant domain CH2
gctccagaagctgctggcggcccttccgtgtttctgttccctccaaagcctaaggacaccctgatgatcagcagaacccctgaagtgacctgcgtggtggtggatgtgtcccacgaggatcccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctgggcgctcccatcgagaaaaccatctctaaggccaag

SEQ ID NO: 100 - DNA, 318 bp, Artificial Sequence
  constant domain CH3
ggacagccccgcgaacctcaggtttacacactgcctccaagccgggaagaaatgaccaagaaccaggtgtccctgacctgcctcgtgaagggcttctacccttccgatatcgccgtggaatgggagagcaatggccagcctgagaacaactacaagacaacccctcctgtgctggactccgatggctcattcttcctgtacagcaagctgacagtggacaagtccagatggcagcagggcaacgtgttcagctgcagcgtgatgcacgaggccctgcacaaccactacacccagaagtccctgagcctgtctcctgga

SEQ ID NO: 101 - DNA, 136 bp, Artificial Sequence
  F-element
ctggtactgcatgcacgcaatgctagctgcccctttcccgtcctgggtaccccgagtctcccccgacctcgggtcccaggtatgctcccacctccacctgccccactcaccacctctgctagttccagacacctcc

SEQ ID NO: 102 - DNA, 142 bp, Artificial Sequence
  I-element
caagcacgcagcaatgcagctcaaaacgcttagcctagccacacccccacgggaaacagcagtgattaacctttagcaataaacgaaagtttaactaagctatactaaccccagggttggtcaatttcgtgccagccacacc

SEQ ID NO: 103 - DNA, 110 bp, Artificial Sequence
  poly(A) tail
aaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 104 - DNA, 1205 bp, Artificial Sequence
  pST4-vector (LC; RiboMab-19-0202)
attcttctggtccccacagactcagagagaacccgccaccatgagagtgatggcccccagaaccctgatcctgctgctgtctggcgccctggccctgacagagacatgggccggaagcgctgctgtgctgacccagacaccttctccagtgtctgccgccgttggcggcacagtgacaatcagctgtcagagcagccagagcgtgtacggcaacaaccagctgtcctggtatcagcagaagcccggccagcctcctaagctgctgatctaccaggccagcaagctggaaacaggcgtgcccagcagattcaaaggcagcggctctggcacccagttcaccctgacaatctccgacctggaaagcgacgatgccgccacctactattgtgccggcggatacagcagcagctccgacacaacatttggcggcggaacagaggtggtggtcaagcgtacggtggccgctcctagcgtgttcatcttcccaccttccgacgagcagctgaagtctggcacagccagcgttgtgtgcctgctgaacaacttctaccccagagaagccaaggtgcagtggaaggtggacaacgccctgcagagcggcaatagccaagagagcgtgaccgagcaggacagcaaggactctacctacagcctgagcagcaccctgacactgagcaaggccgactacgagaagcacaaagtgtacgcctgcgaagtgacccaccagggcctttctagccctgtgaccaagagcttcaaccggggcgaatgttgatgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccgagacctggtccagagtcgctagccgcgtcgctaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 105 - DNA, 321 bp, Artificial Sequence
  constant domain CL kappa
cgtacggtggccgctcctagcgtgttcatcttcccaccttccgacgagcagctgaagtctggcacagccagcgttgtgtgcctgctgaacaacttctaccccagagaagccaaggtgcagtggaaggtggacaacgccctgcagagcggcaatagccaagagagcgtgaccgagcaggacagcaaggactctacctacagcctgagcagcaccctgacactgagcaaggccgactacgagaagcacaaagtgtacgcctgcgaagtgacccaccagggcctttctagccctgtgaccaagagcttcaaccggggcgaatgt

SEQ ID NO: 106 - DNA, 1913 bp, Artificial Sequence
  pST4-vector (HC; RiboMab-19-0233)
attcttctggtccccacagactcagagagaacccgccaccatgagagtgatggcccccagaaccctgatcctgctgctgtctggcgccctggccctgacagagacatgggccggaagccagagcctggaagaatctggcggcgatcttgtgaaacctggcgcctctctgaccctgacatgtaaagccagcggcatcgacttcagcagcgtgtactacatgtgttgggtccgacaggcccctggcaaaggcctggaatggatcgcctgtatctacgtgggcagcagcggcgtgtcctactatgccacatgggccaagggcagattcaccatcagcaagaccagcagcaccaccgtgacactgcagatgacatctctgacagccgccgacaccgccacctacttttgtgccagagccggatatgtgggcgccgtgtatgtgacactgaccagactggatctgtggggccagggcacactggtcacagtctcctctgcctctacaaagggccctagcgtgttccctctggctcctagcagcaagtctacaagcggaggaacagccgctctgggctgcctggtcaaggattactttcccgagcctgtgaccgtgtcctggaattctggcgctctgacaagcggcgtgcacacctttccagctgtgctgcaaagcagcggcctgtactctctgagcagcgtggtcacagtgccaagctctagcctgggcacccagacctacatctgcaatgtgaaccacaagcctagcaacaccaaggtggacaagaaggtggaacccaagagctgcgacaagacccacacctgtcctccatgtcctgctccagaagctgctggcggcccttccgtgtttctgttccctccaaagcctaaggacaccctgatgatcagcagaacccctgaagtgacctgcgtggtggtggatgtgtcccacgaggatcccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctgggcgctcccatcgagaaaaccatctctaaggccaagggacagccccgcgaacctcaggtttacacactgcctccaagccgggaagaaatgaccaagaaccaggtgtccctgacctgcctcgtgaagggcttctacccttccgatatcgccgtggaatgggagagcaatggccagcctgagaacaactacaagacaacccctcctgtgctggactccgatggctcattcttcctgtacagcaagctgacagtggacaagtccagatggcagcagggcaacgtgttcagctgcagcgtgatgcacgaggccctgcacaaccactacacccagaagtccctgagcctgtctcctggatgatgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccgagacctggtccagagtcgctagccgcgtcgctaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 107 - DNA, 1205 bp, Artificial Sequence
  pST4-vector (LC; RiboMab-19-0233)
attcttctggtccccacagactcagagagaacccgccaccatgagagtgatggcccccagaaccctgatcctgctgctgtctggcgccctggccctgacagagacatgggccggaagcgctgctgtgctgacccagacaccttctccagtgtctgccgccgttggcggcacagtgacaatcagctgtcagagcagccagagcatctacaccaacaacgacctggcctggtatcagcagaagcctggccagcctcctaagctgctgatctacgatgccagcaagctggcctctggcgtgccaagcagattttctggcagcggctctggcacccagttcaccctgacaattagcggcgtgcagtccgatgatgccgccacctattattgcctcggcggctacgatgacgacgccgacaatgcttttggcggcggaacagaggtggtggtcaaacgtacggtggccgctcctagcgtgttcatcttcccaccttccgacgagcagctgaagtctggcacagccagcgttgtgtgcctgctgaacaacttctaccccagagaagccaaggtgcagtggaaggtggacaacgccctgcagagcggcaatagccaagagagcgtgaccgagcaggacagcaaggactctacctacagcctgagcagcaccctgacactgagcaaggccgactacgagaagcacaaagtgtacgcctgcgaagtgacccaccagggcctttctagccctgtgaccaagagcttcaaccggggcgaatgttgatgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccgagacctggtccagagtcgctagccgcgtcgctaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

(19) United States
(12) Patent Application Publication
(10) Pub. No.: US 2023/0399403 A1
(43) Pub. Date: Dec. 14, 2023

(54) MONOCLONAL ANTIBODIES DIRECTED AGAINST PROGRAMMED DEATH-1 PROTEIN AND THEIR USE IN MEDICINE

(71) Applicant: BioNTech SE, Mainz (DE)

(72) Inventors: Ugur SAHIN, Mainz (DE); Karsten BECKMANN, Mainz (DE); Claudia PAULMANN, Mainz (DE); Sina FELLERMEIER-KOPF, Mainz (DE); Friederike GIESEKE, Mainz (DE); Alexander MUIK, Mainz (DE); Ivan KUZMANOV, Mainz (DE)

(21) Appl. No.: 18/035,172

(22) PCT Filed: Nov. 11, 2021
(86) PCT No.: PCT/EP2021/081417

§ 371 Date: May 3, 2023

(30) Foreign Application Priority Data
Nov. 11, 2020 (WO) PCT/EP2020/081746

(51) Int. Cl. C07K 16/28; G01N 33/574

(52) U.S. Cl. CPC C07K 16/2818; G01N 33/57492; C07K 2317/565; G01N 2333/70521; C07K 2317/92; C07K 2317/76; C07K 2317/24

(57) ABSTRACT

The present disclosure relates to antibodies having the ability of binding to the immune checkpoint protein programmed death-1 (PD-1), such as human PD-1, or nucleic acids encoding such antibodies. The present disclosure also relates to compositions or kits comprising said antibodies or nucleic acids, as well as to the use of these antibodies or nucleic acids or compositions in the field of medicine, preferably in the field of immunotherapy, e.g., for the treatment of cancers. The present invention further relates to methods for inducing an immune response in a subject comprising providing to the subject an antibody having the ability of binding to the immune checkpoint protein PD-1, such as human PD-1, or a nucleic acid encoding such an antibody, or a composition comprising said antibody or nucleic acid.

### TECHNICAL FIELD OF THE INVENTION

The present invention relates to antibodies having the ability of binding to the immune checkpoint protein programmed death-1 (PD-1), such as human PD-1, or nucleic acids encoding such antibodies. The present invention also relates to compositions or kits comprising said antibodies or nucleic acids, as well as to the use of these antibodies or nucleic acids or compositions in the field of medicine, preferably in the field of immunotherapy for the treatment of cancers. The present invention further relates to methods for inducing an immune response in a subject comprising providing to the subject an antibody having the ability of binding to the immune checkpoint protein PD-1, such as human PD-1, or a nucleic acid encoding such an antibody or a composition comprising said antibody or nucleic acid.

### BACKGROUND OF THE INVENTION

Immunotherapy aims to enhance or induce specific immune responses in patients to control infectious or malignant diseases. The identification of a growing number of pathogen- and tumor-associated antigens (TAA) led to a broad collection of suitable targets for immunotherapy. Cells presenting immunogenic peptides (epitopes) derived from these antigens can be specifically targeted by either active or passive immunization strategies. Active immunization tends to induce and expand antigen-specific T cells in the patient, which are able to specifically recognize and kill diseased cells. In contrast passive immunization may rely on the adoptive transfer of T cells, which were expanded and optional genetically engineered in vitro (adoptive T cell therapy).

In vertebrates, the evolution of the immune system resulted in a highly effective network based on two types of defense: the innate and the adoptive immunity. In contrast to the evolutionary ancient innate immune system that relies on invariant receptors recognizing common molecular patterns associated with pathogens, the adoptive immunity is based on highly specific antigen receptors on B cells (B lymphocytes) and T cells (T lymphocytes) and clonal selection. The immune system plays a crucial role during cancer development, progression and therapy. CD8⁺ T cells and NK cells can directly lyse tumor cells and high tumor-infiltration of these cells is generally regarded as favorable for the outcome of various tumor diseases. CD4⁺ T cells contribute to the anti-tumor immune response by secretion of IFNγ or licensing of antigen-presenting dendritic cells (DCs), which in turn prime and activate CD8⁺ T cells (Kreiter S. et al. Nature 520, 692-6 (2015)). The recognition and elimination of tumor cells by CD8⁺ T cells depends on antigen presentation via the Major Histocompatibility Complex (MHC) class I. Antigen-specific T cell responses can be elicited by vaccination. Vaccination can be achieved by administering vaccine RNA, i.e., RNA encoding an antigen or epitope against which an immune response is to be induced.

Not only stimulation through antigen receptors (TCR), but also an additional stimulative inducement through conjugated stimulative molecular groups (for example, CD28) could by necessary for activation of T cells. Cancer cells can avoid and suppress immune responses through upregulation of inhibitory immune checkpoint proteins, such as PD-1, and CTLA-4 on T cells or PD-L1 on tumor cells, tumor stroma or other cells within the tumor microenvironment. CTLA4 and PD-1 are known to transmit signals that suppresses T-cell activation. Blocking the activities of these proteins with monoclonal antibodies, and thus restoring T cell function, has delivered breakthrough therapies against cancer.

PD-1 (also known as CD279) is an immunoregulatory receptor expressed on the surface of activated T cells, B cells, and monocytes. The protein PD-1 has two naturally occurring ligands, which are known as PD-L1 (also referred to as CD274) and PD-L2 (also known as CD273). A wide variety of cancers express PD-L 1, including melanoma, lung, renal, bladder, esophageal, gastric and other cancers. Thus, in cancer, the PD-1/PD-L1 system can upon the interaction of PD-L1 with PD-1 inhibit the proliferation of T lymphocytes, release of cytokines, and cytotoxicity, thereby providing cancer cells the opportunity to avoid a T cell mediated immune response.

Monoclonal antibodies suitable for regulating the activity of the PD-1/PD-L1 axis are known. The PD-1/PD-L1 interaction can be inhibited by pembrolizumab (also named MK-3475, lambrolizumab or Keytruda). Another monoclonal antibody suitable for this purpose is nivolumab (also named ONO-4538, BMS-936558 or Opdivo).

Antibody-based therapies for cancer have the potential of higher specificity and a lower side effect profile as compared to conventional drugs and may therefore be advantageous to conventional therapies. But by activating the immune system, immune checkpoint inhibitors may also cause autoimmune side effects in some patients. Other patients may fail to respond to the treatment.

Furthermore, anti-PD-1 antibodies have the potential to mitigate autoimmune diseases without the collateral suppression of normal immunity. E.g., an anti-PD-1 binding fragment coupled to an immunotoxin was able to delay disease onset in autoimmune diabetes, and ameliorates symptoms in an autoimmune encephalomyelitis model in mice (Zhao P. et al. Nat Biomed Eng. 3(4): 292-305 (2019)).

Thus, despite impressive benefits associated with immune checkpoint inhibitor therapy, there is still an unmet need for the development of improved antibodies targeting these checkpoints and to provide further benefits for immunotherapy, in particular cancer immunotherapy.

### SUMMARY OF THE INVENTION

The present invention generally provides antibodies useful as therapeutics for treating and/or preventing diseases, such as cancers or infectious diseases. The treatment aims in activating the immune system and/or inducing an immune response.

The antibodies of the present invention show binding characteristics to PD-1, preferably to human-PD-1, and the ability to blockade a PD-1/PD-L1 interaction, so that they are capable of inducing an immune response.

The antibodies of the invention may have one or more of the following properties: The antibodies of the present invention (i) bind, preferably specifically bind, to PD-1; (ii) may have binding properties to PD-1 on immune cells; (iii) may have binding properties to PD-1 epitopes; (iv) may have binding properties to a non-human PD-1 variant, particularly to PD-1 variants from mice, rats, rabbits and primates; (v) may prevent or reduce the induction of inhibitory signals by PD-1; (vi) may inhibit the interaction/binding of ligands of PD-1 with PD-1, preferably of the ligand PD-L1 thereby blocking the inhibitory PD-1/PD-L1 axis, for example, they may inhibit the binding of human PD-L1 to human PD-1; (vii) may inhibit the immunosuppressive signal of PD-L1 or PD-L2; (viii) may enhance or initiate the immune function, preferably by enhancing or initiating a T-cell mediated immune response, preferably by inducing CD8⁺ cell proliferation; (ix) may inhibit cancer proliferation; (x) may deplete tumor cells and/or suppress cancer metastasis; and/or (xi) may deplete immune cells and/or ameliorates autoimmune disease.

In the following reference is given to sequences and SEQ ID NOs which are shown inter alia in the sequence listing. Also, reference is given to specific examples of antibodies of the invention described herein, but without limiting the present invention thereto: MAB-19-0202, MAB-19-0208, MAB-19-0217, MAB-19-0223, MAB-19-0233, MAB-19-0603, MAB-19-0608, MAB-19-0613, MAB-19-0618, MAB-19-0583, MAB-19-0594, and MAB-19-0598. These examplatory, but not limiting antibodies of the invention are designated herein by referring to the designation of the antibody.

In one aspect, the invention relates to an antibody having the ability of binding to PD-1 and thereby preferably inhibiting the immunosuppressive signal of PD-1.

In another aspect of the invention, the antibody depletes activate immune cells and thereby ameliorates autoimmune diseases.

An antibody of the invention comprises a heavy chain variable region (VH) comprising a complementarity-determining region 3 (HCDR3) having or comprising a sequence as set forth in any one of SEQ ID NO: 1, SEQ ID NO: 2, SEQ ID NO: 3, SEQ ID NO: 4 or SEQ ID NO: 5. In one embodiment, the HCDR3 of the heavy chain variable region has or comprises a sequence as set forth in any one of SEQ ID NO: 6, SEQ ID NO: 7, SEQ ID NO: 8, SEQ ID NO: 9 or SEQ ID NO: 10.

In one embodiment, the heavy chain variable region (VH) of the said antibody comprises a complementarity-determining region 2 (HCDR2) having or comprising a sequence as set forth in any one of SEQ ID NO: 11, SEQ ID NO: 12, SEQ ID NO: 13, SEQ ID NO: 14 or SEQ ID NO: 15. In one embodiment, the HCDR2 has or comprises a sequence as set forth in any one of SEQ ID NO: 16, SEQ ID NO: 17, SEQ ID NO: 18, SEQ ID NO: 19 or SEQ ID NO: 20.

In one embodiment, the heavy chain variable region (VH) of the said antibody comprises a complementarity-determining region 1 (HCDR1) having or comprising a sequence selected from SYN, RYY, as set forth in SEQ ID NO: 21 or SEQ ID NO: 22. In one embodiment, the HCDR1 has or comprises a sequence as set forth in any one of SEQ ID NO: 23, SEQ ID NO: 24, SEQ ID NO: 25, SEQ ID NO: 26 or SEQ ID NO: 27. In one embodiment, the HCDR1 has or comprises a sequence as set forth in any one of SEQ ID NO: 28, SEQ ID NO: 29, SEQ ID NO: 30, SEQ ID NO: 31 or SEQ ID NO: 32.

In one embodiment of the said antibody, the antibody comprises a heavy chain variable region (VH) comprising a HCDR1, HCDR2 and HCDR3 sequence, wherein the HCDR1 sequence is selected from a sequence having or comprising SYN, SEQ ID NO: 23 or SEQ ID NO: 28, the HCDR2 sequence is selected from a sequence having or comprising SEQ ID NO: 11 or SEQ ID NO: 16, and the HCDR3 sequence is selected from a sequence having or comprising SEQ ID NO: 1 or SEQ ID NO: 6. In one embodiment of the said antibody, the antibody comprises a heavy chain variable region (VH) comprising a HCDR1, HCDR2 and HCDR3 sequence, wherein the HCDR1 sequence is selected from a sequence having or comprising RYY, SEQ ID NO: 24 or SEQ ID NO: 29, the HCDR2 sequence is selected from a sequence having or comprising SEQ ID NO: 12 or SEQ ID NO: 17, and the HCDR3 sequence is selected from a sequence having or comprising SEQ ID NO: 2 or SEQ ID NO: 7. In one embodiment of the said antibody, the antibody comprises a heavy chain variable region (VH) comprising a HCDR1, HCDR2 and HCDR3 sequence, wherein the HCDR1 sequence is selected from a sequence having or comprising RYY, SEQ ID NO: 25 or SEQ ID NO: 30, the HCDR2 sequence is selected from a sequence having or comprising SEQ ID NO: 13 or SEQ ID NO: 18, and the HCDR3 sequence is selected from a sequence having or comprising SEQ ID NO: 3 or SEQ ID NO: 8. In one embodiment of the said antibody, the antibody comprises a heavy chain variable region (VH) comprising a HCDR1, HCDR2 and HCDR3 sequence, wherein the HCDR1 sequence is selected from a sequence having or comprising SEQ ID NO: 21, SEQ ID NO: 26 or SEQ ID NO: 31, the HCDR2 sequence is selected from a sequence having or comprising SEQ ID NO: 14 or SEQ ID NO: 19, and the HCDR3 sequence is selected from a sequence having or comprising SEQ ID NO: 4 or SEQ ID NO: 9. In one embodiment of the said antibody, the antibody comprises a heavy chain variable region (VH) comprising a HCDR1, HCDR2 and HCDR3 sequence, wherein the HCDR1 sequence is selected from a sequence having or comprising SEQ ID NO: 22, SEQ ID NO: 27 or SEQ ID NO: 32, the HCDR2 sequence is selected from a sequence having or comprising SEQ ID NO: 15 or SEQ ID NO: 20, and the HCDR3 sequence is selected from a sequence having or comprising SEQ ID NO: 5 or SEQ ID NO: 10.

In one embodiment of the said antibody, the antibody comprises a heavy chain variable region (VH) comprising a HCDR1, HCDR2, and HCDR3 sequence, wherein the HCDR1, HCDR2 and HCDR3 sequence is or comprises SYN, SEQ ID NO: 11 and SEQ ID NO: 1, respectively. In one embodiment of the said antibody, the antibody comprises a heavy chain variable region (VH) comprising a HCDR1, HCDR2, and HCDR3 sequence, wherein the HCDR1, HCDR2 and HCDR3 sequence is or comprises RYY, SEQ ID NO: 12 and SEQ ID NO: 2, respectively. In one embodiment of the said antibody, the antibody comprises a heavy chain variable region (VH) comprising a HCDR1, HCDR2, and HCDR3 sequence, wherein the HCDR1, HCDR2 and HCDR3 sequence is or comprises RYY, SEQ ID NO: 13 and SEQ ID NO: 3, respectively. In one embodiment of the said antibody, the antibody comprises a heavy chain variable region (VH) comprising a HCDR1, HCDR2, and HCDR3 sequence, wherein the HCDR1, HCDR2 and HCDR3 sequence is or comprises SEQ ID NO: 21, SEQ ID NO: 14 and SEQ ID NO: 4, respectively. In one embodiment of the said antibody, the antibody comprises a heavy chain variable region (VH) comprising a HCDR1, HCDR2, and HCDR3 sequence, wherein the HCDR1, HCDR2 and HCDR3 sequence is or comprises SEQ ID NO: 22, SEQ ID NO: 15 and SEQ ID NO: 5, respectively.

In one embodiment, the antibody comprises a heavy chain variable region (VH) comprising a HCDR1, HCDR2, and HCDR3 sequence, wherein the HCDR1, HCDR2 and HCDR3 sequence is or comprises SEQ ID NO: 23, SEQ ID NO: 16 and SEQ ID NO: 1, respectively. In one embodiment, the antibody comprises a heavy chain variable region (VH) comprising a HCDR1, HCDR2, and HCDR3 sequence, wherein the HCDR1, HCDR2 and HCDR3 sequence is or comprises SEQ ID NO: 24, SEQ ID NO: 17 and SEQ ID NO: 2, respectively. In one embodiment, the antibody comprises a heavy chain variable region (VH) comprising a HCDR1, HCDR2, and HCDR3 sequence, wherein the HCDR1, HCDR2 and HCDR3 sequence is or comprises SEQ ID NO: 25, SEQ ID NO: 18 and SEQ ID NO: 3, respectively. In one embodiment, the antibody comprises a heavy chain variable region (VH) comprising a HCDR1, HCDR2, and HCDR3 sequence, wherein the HCDR1, HCDR2 and HCDR3 sequence is or comprises SEQ ID NO: 26, SEQ ID NO: 19 and SEQ ID NO: 4, respectively. In one embodiment, the antibody comprises a heavy chain variable region (VH) comprising a HCDR1, HCDR2, and HCDR3 sequence, wherein the HCDR1, HCDR2 and HCDR3 sequence is or comprises SEQ ID NO: 27, SEQ ID NO: 20 and SEQ ID NO: 5, respectively.

In one embodiment, the antibody comprises a heavy chain variable region (VH) comprising a HCDR1, HCDR2, and HCDR3 sequence, wherein the HCDR1, HCDR2 and HCDR3 sequence is or comprises SEQ ID NO: 28, SEQ ID NO: 11 and SEQ ID NO: 6, respectively. In one embodiment, the antibody comprises a heavy chain variable region (VH) comprising a HCDR1, HCDR2, and HCDR3 sequence, wherein the HCDR1, HCDR2 and HCDR3 sequence is or comprises SEQ ID NO: 29, SEQ ID NO: 12 and SEQ ID NO: 7, respectively. In one embodiment, the antibody comprises a heavy chain variable region (VH) comprising a HCDR1, HCDR2, and HCDR3 sequence, wherein the HCDR1, HCDR2 and HCDR3 sequence is or comprises SEQ ID NO: 30, SEQ ID NO: 13 and SEQ ID NO: 8, respectively. In one embodiment, the antibody comprises a heavy chain variable region (VH) comprising a HCDR1, HCDR2, and HCDR3 sequence, wherein the HCDR1, HCDR2 and HCDR3 sequence is or comprises SEQ ID NO: 31, SEQ ID NO: 14 and SEQ ID NO: 9, respectively. In one embodiment, the antibody comprises a heavy chain variable region (VH) comprising a HCDR1, HCDR2, and HCDR3 sequence, wherein the HCDR1, HCDR2 and HCDR3 sequence is or comprises SEQ ID NO: 32, SEQ ID NO: 15 and SEQ ID NO: 10, respectively.

In one embodiment of the above aspect and in another aspect, the invention relates to an antibody having the ability of binding to PD-1 and thereby preferably inhibiting the immunosuppressive signal of PD-1. The antibody comprises a light chain variable region (VL) comprising a complementarity-determining region 3 (LCDR3) having or comprising a sequence as set forth in any one of SEQ ID NO: 33, SEQ ID NO: 34, SEQ ID NO: 35, SEQ ID NO: 36 or SEQ ID NO: 37.

In one embodiment, the light chain variable region (VL) of the said antibody comprises a complementarity-determining region 2 (LCDR2) having or comprising a sequence selected from QAS or DAS. In one embodiment, the light chain variable region (VL) comprises a complementarity-determining region 2 (LCDR2) having or comprising a sequence as set forth in any one of SEQ ID NO: 38, SEQ ID NO: 39, SEQ ID NO: 40 or SEQ ID NO: 41.

In one embodiment, the light chain variable region (VL) of the said antibody comprises a complementarity-determining region 1 (LCDR1) having or comprising a sequence as set forth in any one of SEQ ID NO: 42, SEQ ID NO: 43, SEQ ID NO: 44, SEQ ID NO: 45 or SEQ ID NO: 46. In one embodiment, the light chain variable region (VL) comprises a complementarity-determining region 1 (LCDR1) having or comprising a sequence as set forth in any one of SEQ ID NO: 47, SEQ ID NO: 48, SEQ ID NO: 49, SEQ ID NO: 50 or SEQ ID NO: 51.

In one embodiment, the antibody comprises a light chain variable region (VL) comprising a LCDR1, LCDR2, and LCDR3 sequence, wherein the LCDR1 sequence is selected from a sequence having or comprising SEQ ID NO: 42 or SEQ ID NO: 47, the LCDR2 sequence is selected from a sequence having or comprising QAS or SEQ ID NO: 38, and the LCDR3 sequence is a sequence having or comprising SEQ ID NO: 33. In one embodiment, the antibody comprises a light chain variable region (VL) comprising a LCDR1, LCDR2, and LCDR3 sequence, wherein the LCDR1 sequence is selected from a sequence having or comprising SEQ ID NO: 43 or SEQ ID NO: 48, the LCDR2 sequence is selected from a sequence having or comprising DAS or SEQ ID NO: 39, and the LCDR3 sequence is a sequence having or comprising SEQ ID NO: 34. In one embodiment, the antibody comprises a light chain variable region (VL) comprising a LCDR1, LCDR2, and LCDR3 sequence, wherein the LCDR1 sequence is selected from a sequence having or comprising SEQ ID NO: 44 or SEQ ID NO: 49, the LCDR2 sequence is selected from a sequence having or comprising DAS or SEQ ID NO: 39, and the LCDR3 sequence is a sequence having or comprising SEQ ID NO: 35. In one embodiment, the antibody comprises a light chain variable region (VL) comprising a LCDR1, LCDR2, and LCDR3 sequence, wherein the LCDR1 sequence is selected from a sequence having or comprising SEQ ID NO: 45 or SEQ ID NO: 50, the LCDR2 sequence is selected from a sequence having or comprising DAS or SEQ ID NO: 40, and the LCDR3 sequence is a sequence having or comprising SEQ ID NO: 36. In one embodiment, the antibody comprises a light chain variable region (VL) comprising a LCDR1, LCDR2, and LCDR3 sequence, wherein the LCDR1 sequence is selected from a sequence having or comprising SEQ ID NO: 46 or SEQ ID NO: 51, the LCDR2 sequence is selected from a sequence having or comprising DAS or SEQ ID NO: 41, and the LCDR3 sequence is a sequence having or comprising SEQ ID NO: 37.

In one embodiment, the antibody comprises a light chain variable region (VL) comprising a LCDR1, LCDR2, and LCDR3 sequence, wherein the LCDR1, LCDR2 and LCDR3 sequence is or comprises SEQ ID NO: 42, QAS, and SEQ ID NO: 33, respectively. In one embodiment, the antibody comprises a light chain variable region (VL) comprising a LCDR1, LCDR2, and LCDR3 sequence, wherein the LCDR1, LCDR2 and LCDR3 sequence is or comprises SEQ ID NO: 43, DAS, and SEQ ID NO: 34, respectively. In one embodiment, the antibody comprises a light chain variable region (VL) comprising a LCDR1, LCDR2, and LCDR3 sequence, wherein the LCDR1, LCDR2 and LCDR3 sequence is or comprises SEQ ID NO: 44, DAS, and SEQ ID NO: 35, respectively. In one embodiment, the antibody comprises a light chain variable region (VL) comprising a LCDR1, LCDR2, and LCDR3 sequence, wherein the LCDR1, LCDR2 and LCDR3 sequence is or comprises SEQ ID NO: 45, DAS, and SEQ ID NO: 36, respectively. In one embodiment, the antibody comprises a light chain variable region (VL) comprising a LCDR1, LCDR2, and LCDR3 sequence, wherein the LCDR1, LCDR2 and LCDR3 sequence is or comprises SEQ ID NO: 46, DAS, and SEQ ID NO: 37, respectively.

In one embodiment, the antibody comprises a light chain variable region (VL) comprising a LCDR1, LCDR2, and LCDR3 sequence, wherein the LCDR1, LCDR2 and LCDR3 sequence is or comprises SEQ ID NO: 47, SEQ ID NO: 38, and SEQ ID NO: 33, respectively. In one embodiment, the antibody comprises a light chain variable region (VL) comprising a LCDR1, LCDR2, and LCDR3 sequence, wherein the LCDR1, LCDR2 and LCDR3 sequence is or comprises SEQ ID NO: 48, SEQ ID NO: 39, and SEQ ID NO: 34, respectively. In one embodiment, the antibody comprises a light chain variable region (VL) comprising a LCDR1, LCDR2, and LCDR3 sequence, wherein the LCDR1, LCDR2 and LCDR3 sequence is or comprises SEQ ID NO: 49, SEQ ID NO: 39, and SEQ ID NO: 35, respectively. In one embodiment, the antibody comprises a light chain variable region (VL) comprising a LCDR1, LCDR2, and LCDR3 sequence, wherein the LCDR1, LCDR2 and LCDR3 sequence is or comprises SEQ ID NO: 50, SEQ ID NO: 40, and SEQ ID NO: 36, respectively. In one embodiment, the antibody comprises a light chain variable region (VL) comprising a LCDR1, LCDR2, and LCDR3 sequence, wherein the LCDR1, LCDR2 and LCDR3 sequence is or comprises SEQ ID NO: 51, SEQ ID NO: 41, and SEQ ID NO: 37, respectively.

In another aspect, the invention relates to an antibody having the ability of binding to PD-1, wherein the antibody comprises a heavy chain variable region (VH) of the above first aspect of the invention and/or a light chain variable region (VL) of the above second aspect of the invention.

In one embodiment of the above aspects, the antibody comprises a heavy chain variable region (VH) comprising a HCDR1, HCDR2, and HCDR3 sequence and a light chain variable region (VL) comprising a LCDR1, LCDR2, and LCDR3 sequence, wherein the HCDR1, HCDR2 and HCDR3 sequence comprises or has the sequence SYN, as set forth in SEQ ID NO: 11 and SEQ ID NO: 1, respectively, and the LCDR1, LCDR2 and LCDR3 sequence comprises or has the sequence as set forth in SEQ ID NO: 42, QAS, and SEQ ID NO: 33, respectively. A specific, but not limiting example of such an antibody is MAB-19-0202.

In one embodiment of the above aspects, the antibody comprises a heavy chain variable region (VH) comprising a HCDR1, HCDR2, and HCDR3 sequence and a light chain variable region (VL) comprising a LCDR1, LCDR2, and LCDR3 sequence, wherein the HCDR1, HCDR2 and HCDR3 sequence comprises or has the sequence as set forth in SEQ ID NO: 23, SEQ ID NO: 16, and SEQ ID NO: 1, respectively, and the LCDR1, LCDR2 and LCDR3 sequence comprises or has the sequence as set forth in SEQ ID NO: 47, SEQ ID NO: 38, and SEQ ID NO: 33, respectively. A specific, but not limiting example of such an antibody is MAB-19-0202.

In one embodiment of the above aspects, the antibody comprises a heavy chain variable region (VH) comprising a HCDR1, HCDR2, and HCDR3 sequence and a light chain variable region (VL) comprising a LCDR1, LCDR2, and LCDR3 sequence, wherein the HCDR1, HCDR2 and HCDR3 sequence comprises or has the sequence as set forth in SEQ ID NO: 28, SEQ ID NO: 11, and SEQ ID NO: 6, respectively, and the LCDR1, LCDR2 and LCDR3 sequence comprises or has the sequence as set forth in SEQ ID NO: 42, QAS, and SEQ ID NO: 33, respectively. A specific, but not limiting example of such an antibody is MAB-19-0202.

In one embodiment of the above aspects, the antibody comprises a heavy chain variable region (VH) comprising a HCDR1, HCDR2, and HCDR3 sequence and a light chain variable region (VL) comprising a LCDR1, LCDR2, and LCDR3 sequence, wherein the HCDR1, HCDR2 and HCDR3 sequence comprises or has the sequence RYY, as set forth in SEQ ID NO: 12 and SEQ ID NO: 2, respectively, and the LCDR1, LCDR2 and LCDR3 sequence comprises or has the sequence as set forth in SEQ ID NO: 43, DAS, and SEQ ID NO: 34, respectively. A specific, but not limiting example of such an antibody is MAB-19-0208.

In one embodiment of the above aspects, the antibody comprises a heavy chain variable region (VH) comprising a HCDR1, HCDR2, and HCDR3 sequence and a light chain variable region (VL) comprising a LCDR1, LCDR2, and LCDR3 sequence, wherein the HCDR1, HCDR2 and HCDR3 sequence comprises or has the sequence as set forth in SEQ ID NO: 24, SEQ ID NO: 17, and SEQ ID NO: 2, respectively, and the LCDR1, LCDR2 and LCDR3 sequence comprises or has the sequence as set forth in SEQ ID NO: 48, SEQ ID NO: 39, and SEQ ID NO: 34, respectively. A specific, but not limiting example of such an antibody is MAB-19-0208.

In one embodiment of the above aspects, the antibody comprises a heavy chain variable region (VH) comprising a HCDR1, HCDR2, and HCDR3 sequence and a light chain variable region (VL) comprising a LCDR1, LCDR2, and LCDR3 sequence, wherein the HCDR1, HCDR2 and HCDR3 sequence comprises or has the sequence as set forth in SEQ ID NO: 29, SEQ ID NO: 12, and SEQ ID NO: 7, respectively, and the LCDR1, LCDR2 and LCDR3 sequence comprises or has the sequence as set forth in SEQ ID NO: 43, DAS, and SEQ ID NO: 34, respectively. A specific, but not limiting example of such an antibody is MAB-19-0208.

In one embodiment of the above aspects, the antibody comprises a heavy chain variable region (VH) comprising a HCDR1, HCDR2, and HCDR3 sequence and a light chain variable region (VL) comprising a LCDR1, LCDR2, and LCDR3 sequence, wherein the HCDR1, HCDR2 and HCDR3 sequence comprises or has the sequence RYY, as set forth in SEQ ID NO: 13 and SEQ ID NO: 3, respectively, and the LCDR1, LCDR2 and LCDR3 sequence comprises or has the sequence as set forth in SEQ ID NO: 44, DAS, and SEQ ID NO: 35, respectively. A specific, but not limiting example of such an antibody is MAB-19-0217.

In one embodiment of the above aspects, the antibody comprises a heavy chain variable region (VH) comprising a HCDR1, HCDR2, and HCDR3 sequence and a light chain variable region (VL) comprising a LCDR1, LCDR2, and LCDR3 sequence, wherein the HCDR1, HCDR2 and HCDR3 sequence comprises or has the sequence as set forth in SEQ ID NO: 25, SEQ ID NO: 18, and SEQ ID NO: 3, respectively, and the LCDR1, LCDR2 and LCDR3 sequence comprises or has the sequence as set forth in SEQ ID NO: 49, SEQ ID NO: 39, and SEQ ID NO: 35, respectively. A specific, but not limiting example of such an antibody is MAB-19-0217.

In one embodiment of the above aspects, the antibody comprises a heavy chain variable region (VH) comprising a HCDR1, HCDR2, and HCDR3 sequence and a light chain variable region (VL) comprising a LCDR1, LCDR2, and LCDR3 sequence, wherein the HCDR1, HCDR2 and HCDR3 sequence comprises or has the sequence as set forth in SEQ ID NO: 30, SEQ ID NO: 13, and SEQ ID NO: 8, respectively, and the LCDR1, LCDR2 and LCDR3 sequence comprises or has the sequence as set forth in SEQ ID NO: 44, DAS, and SEQ ID NO: 35, respectively. A specific, but not limiting example of such an antibody is MAB-19-0217.

In one embodiment of the above aspects, the antibody comprises a heavy chain variable region (VH) comprising a HCDR1, HCDR2, and HCDR3 sequence and a light chain variable region (VL) comprising a LCDR1, LCDR2, and LCDR3 sequence, wherein the HCDR1, HCDR2 and HCDR3 sequence comprises or has the sequence as set forth in SEQ ID NO: 21, SEQ ID NO: 14 and SEQ ID NO: 4, respectively, and the LCDR1, LCDR2 and LCDR3 sequence comprises or has the sequence as set forth in SEQ ID NO: 45, DAS, and SEQ ID NO: 36, respectively. A specific, but not limiting example of such an antibody is MAB-19-0223.

In one embodiment of the above aspects, the antibody comprises a heavy chain variable region (VH) comprising a HCDR1, HCDR2, and HCDR3 sequence and a light chain variable region (VL) comprising a LCDR1, LCDR2, and LCDR3 sequence, wherein the HCDR1, HCDR2 and HCDR3 sequence comprises or has the sequence as set forth in SEQ ID NO: 26, SEQ ID NO: 19, and SEQ ID NO: 4, respectively, and the LCDR1, LCDR2 and LCDR3 sequence comprises or has the sequence as set forth in SEQ ID NO: 50, SEQ ID NO: 40, and SEQ ID NO: 36, respectively. A specific, but not limiting example of such an antibody is MAB-19-0223.

In one embodiment of the above aspects, the antibody comprises a heavy chain variable region (VH) comprising a HCDR1, HCDR2, and HCDR3 sequence and a light chain variable region (VL) comprising a LCDR1, LCDR2, and LCDR3 sequence, wherein the HCDR1, HCDR2 and HCDR3 sequence comprises or has the sequence as set forth in SEQ ID NO: 31, SEQ ID NO: 14, and SEQ ID NO: 9, respectively, and the LCDR1, LCDR2 and LCDR3 sequence comprises or has the sequence as set forth in SEQ ID NO: 45, DAS, and SEQ ID NO: 36, respectively. A specific, but not limiting example of such an antibody is MAB-19-0223.

In one embodiment of the above aspects, the antibody comprises a heavy chain variable region (VH) comprising a HCDR1, HCDR2, and HCDR3 sequence and a light chain variable region (VL) comprising a LCDR1, LCDR2, and LCDR3 sequence, wherein the HCDR1, HCDR2 and HCDR3 sequence comprises or has the sequence as set forth in SEQ ID NO: 22, SEQ ID NO: 15 and SEQ ID NO: 5, respectively, and the LCDR1, LCDR2 and LCDR3 sequence comprises or has the sequence as set forth in SEQ ID NO: 46, DAS, and SEQ ID NO: 37, respectively. A specific, but not limiting example of such an antibody is MAB-19-0233.

In one embodiment of the above aspects, the antibody comprises a heavy chain variable region (VH) comprising a HCDR1, HCDR2, and HCDR3 sequence and a light chain variable region (VL) comprising a LCDR1, LCDR2, and LCDR3 sequence, wherein the HCDR1, HCDR2 and HCDR3 sequence comprises or has the sequence as set forth in SEQ ID NO: 27, SEQ ID NO: 20, and SEQ ID NO: 5, respectively, and the LCDR1, LCDR2 and LCDR3 sequence comprises or has the sequence as set forth in SEQ ID NO: 51, SEQ ID NO: 41, and SEQ ID NO: 37, respectively. A specific, but not limiting example of such an antibody is MAB-19-0233.

In one embodiment of the above aspects, the antibody comprises a heavy chain variable region (VH) comprising a HCDR1, HCDR2, and HCDR3 sequence and a light chain variable region (VL) comprising a LCDR1, LCDR2, and LCDR3 sequence, wherein the HCDR1, HCDR2 and HCDR3 sequence comprises or has the sequence as set forth in SEQ ID NO: 32, SEQ ID NO: 15, and SEQ ID NO: 10, respectively, and the LCDR1, LCDR2 and LCDR3 sequence comprises or has the sequence as set forth in SEQ ID NO: 46, DAS, and SEQ ID NO: 37, respectively. A specific, but not limiting example of such an antibody is MAB-19-0233.

In one embodiment of the above aspects, an antibody of the invention comprising one or more CDRs, a set of CDRs or a combination of sets of CDRs as described herein comprises said CDRs together with their intervening framework regions (also referred to as framing region or FR herein) or with portions of said framework regions. Preferably, the portion will include at least about 50% of either or both of the first and fourth framework regions, the 50% being the C-terminal 50% of the first framework region and the N-terminal 50% of the fourth framework region. Construction of antibodies of the present invention made by recombinant DNA techniques may result in the introduction of residues N- or C-terminal to the variable regions encoded by linkers introduced to facilitate cloning or other manipulation steps, including the introduction of linkers to join variable regions of the invention to further protein sequences including immunoglobulin heavy chains, other variable domains (for example in the production of diabodies) or protein labels.

In one embodiment of the above aspects, the antibody comprises a heavy chain variable region (VH) comprising a sequence having at least 70%, at least 75%, at least 80%, at least 85%, at least 90%, at least 95%, at least 97%, at least 99%, or 100% identity to the amino acid sequence of the VH sequence as set forth in any one of SEQ ID NO: 52 to SEQ ID NO: 56. In one embodiment of the above aspects, the antibody comprises a heavy chain variable region (VH), wherein the VH comprises the sequence as set forth in any one of SEQ ID NO: 52 to SEQ ID NO: 56.

In one embodiment of the above aspects, the antibody comprises a light chain variable region (VL) comprising a sequence having at least 70%, at least 75%, at least 80%, at least 85%, at least 90%, at least 95%, at least 97%, at least 99%, or 100% identity to the amino acid sequence of the VL sequence as set forth in any one of SEQ ID NO: 57 to SEQ ID NO: 61. In one embodiment of the above aspects, the antibody comprises a light chain variable region (VL), wherein the VL comprises the sequence as set forth in any one of SEQ ID NO: 57 to SEQ ID NO: 61.

In one embodiment of the above aspects, the antibody comprises a heavy chain variable region (VH) and a light chain variable region (VL), wherein the VH comprises or has the sequence as set forth in SEQ ID NO: 52 and the VL comprises or has the sequence as set forth in SEQ ID NO: 57. A specific, but not limiting example of such an antibody is MAB-19-0202. In one embodiment of the above aspects, the antibody comprises a heavy chain variable region (VH) and a light chain variable region (VL), wherein the VH comprises or has the sequence as set forth in SEQ ID NO: 53 and the VL comprises or has the sequence as set forth in SEQ ID NO: 58. A specific, but not limiting example of such an antibody is MAB-19-0208. In one embodiment of the above aspects, the antibody comprises a heavy chain variable region (VH) and a light chain variable region (VL), wherein the VH comprises or has the sequence as set forth in SEQ ID NO: 54 and the VL comprises or has the sequence as set forth in SEQ ID NO: 59. A specific, but not limiting example of such an antibody is MAB-19-0217. In one embodiment of the above aspects, the antibody comprises a heavy chain variable region (VH) and a light chain variable region (VL), wherein the VH comprises or has the sequence as set forth in SEQ ID NO: 55 and the VL comprises or has the sequence as set forth in SEQ ID NO: 60. A specific, but not limiting example of such an antibody is MAB-19-0223. In one embodiment of the above aspects, the antibody comprises a heavy chain variable region (VH) and a light chain variable region (VL), wherein the VH comprises or has the sequence as set forth in SEQ ID NO: 56 and the VL comprises or has the sequence as set forth in SEQ ID NO: 61. A specific, but not limiting example of such an antibody is MAB-19-0233. Also encompassed by the present invention are variants of the said heavy chain variable regions (VH) and the said light chain variable regions (VL) and the respective combinations of these variant VHs and VLs.

Antibodies of the invention may be derived from different species, including but not limited to rabbit, mouse, rat, guinea pig and human. The antibodies can be polyclonal or monoclonal. In one embodiment or a preferred embodiment, the antibodies of the present invention are monoclonal. Antibodies of the present invention may, in one embodiment, include chimeric molecules in which an antibody constant region derived from one species, preferably human, is combined with the antigen binding site derived from another species. In one embodiment, the antibodies are monoclonal chimeric antibodies, wherein the constant region is preferably a human immunoglobin constant part, for example a human IgG1/κ constant part. Moreover, in one embodiment, antibodies of the invention include humanized molecules, preferably monoclonal humanized molecules, in which the antigen binding sites of an antibody derived from a non-human species are combined with constant and framework regions of human origin. In one embodiment, an antibody of the invention comprises one or more CDRs, a set of CDRs or a combination of sets of CDRs as described herein comprises said CDRs in a human antibody framework. In one or a preferred embodiment, the antibody of the present invention is a monoclonal humanized antibody, wherein the constant region is preferably a human immunoglobin constant part, for example a human IgG1/κ constant part.

In one embodiment of the above aspects, the antibody comprises a heavy chain variable region (VH) comprising a sequence having at least 70%, at least 75%, at least 80%, at least 85%, at least 90%, at least 95%, at least 97%, at least 99%, or 100% identity to the amino acid sequence of the VH sequence as set forth in any one of SEQ ID NO: 62 to SEQ ID NO: 64. In one embodiment of the above aspects, the antibody comprises a heavy chain variable region (VH), wherein the VH comprises the sequence as set forth in any one of SEQ ID NO: 62 to SEQ ID NO: 64. In one embodiment of the above aspects, the antibody comprises a light chain variable region (VL) comprising a sequence having at least 70%, at least 75%, at least 80%, at least 85%, at least 90%, at least 95%, at least 97%, at least 99%, or 100% identity to the amino acid sequence of the VL sequence as set forth in any one of SEQ ID NO: 65 to SEQ ID NO: 70. In one embodiment of the above aspects, the antibody comprises a light chain variable region (VL), wherein the VL comprises the sequence as set forth in any one of SEQ ID NO: 65 to SEQ ID NO: 70.

The presentation invention encompasses all possible combinations of these preferred heavy chain variable regions as set forth in SEQ ID Nos: 62 to 64 of the sequence listing and these preferred light chain variable regions as set forth in SEQ ID Nos: 65 to 70 of the sequence listing, or respective variants of these sequences.

In one embodiment, the antibody comprises a heavy chain variable region (VH) and a light chain variable region (VL), wherein the VH comprises or has the sequence as set forth in SEQ ID NO: 62 and the VL comprises or has the sequence as set forth in SEQ ID NO: 65 or SEQ ID NO: 66 or SEQ ID NO: 67 or SEQ ID NO: 68, or respective variants of these sequences. For example, an antibody of the present invention may comprise a VH comprising or having the sequence as set forth in SEQ ID NO: 62, or a variant thereof, and a VL comprising or having the sequence as set forth in SEQ ID NO: 65, or a variant thereof. A specific, but not limiting example of such an antibody is MAB-19-0603. Another example of an antibody of the present invention may comprise a VH comprising or having the sequence as set forth in SEQ ID NO: 62, or a variant thereof, and a VL comprising or having the sequence as set forth in SEQ ID NO: 66, or a variant thereof. A specific, but not limiting example of such an antibody is MAB-19-0608. Another example of an antibody of the present invention may comprise a VH comprising or having the sequence as set forth in SEQ ID NO: 62, or a variant thereof, and a VL comprising or having the sequence as set forth in SEQ ID NO: 67, or a variant thereof. A specific, but not limiting example of such an antibody is MAB-19-0613.

Another example of an antibody of the present invention may comprise a VH comprising or having the sequence as set forth in SEQ ID NO: 62, or a variant thereof, and a VL comprising or having the sequence as set forth in SEQ ID NO: 68, or a variant thereof. A specific, but not limiting example of such an antibody is MAB-19-0618. The antibodies MAB-19-0603, MAB-19-0608, MAB-19-0613 and MAB-19-0618 have been derived from MAB-19-0202. Also encompassed by the present invention are variants of the said heavy chain variable regions (VH) and the said light chain variable regions (VL) and the respective combinations of these variant VHs and VLs.

In one embodiment, the antibody comprises a heavy chain variable region (VH) and a light chain variable region (VL), wherein the VH comprises or has the sequence as set forth in SEQ ID NO: 63 or a variant thereof, and the VL comprises or has the sequence as set forth in SEQ ID NO: 69 or SEQ ID NO: 70 or respective variants thereof, or wherein the VH comprises or has the sequence as set forth in SEQ ID NO: 64 or a variant thereof and the VL comprises or has the sequence as set forth in SEQ ID NO: 70 or a variant thereof. For example, an antibody of the present invention may comprise a VH comprising or having the sequence as set forth in SEQ ID NO: 63, or a variant thereof, and a VL comprising or having the sequence as set forth in SEQ ID NO: 69, or a variant thereof. A specific, but not limiting example of such an antibody is MAB-19-0583. Another example of an antibody of the present invention may comprise a VH comprising or having the sequence as set forth in SEQ ID NO: 64, or a variant thereof, and a VL comprising or having the sequence as set forth in SEQ ID NO: 70, or a variant thereof. A specific, but not limiting example of such an antibody is MAB-19-0594. Another example of an antibody of the present invention may comprise a VH comprising or having the sequence as set forth in SEQ ID NO: 63, or a variant thereof, and a VL comprising or having the sequence as set forth in SEQ ID NO: 70, or a variant thereof. A specific, but not limiting example of such an antibody is MAB-19-0598. The antibodies MAB-19-0583, MAB-19-0594 and MAB-19-0598 have been derived from MAB-19-0233. Also encompassed by the present invention are variants of the said heavy chain variable regions (VH) and the said light chain variable regions (VL) and the respective combinations of these variant VHs and VLs.

In all aspects of the present invention, antibodies of the present invention can include IgG1, IgG2, IgG3, IgG4, IgM, IgA1, IgA4, secretory IgA, IgD, and IgE antibodies and combinations thereof, wherein the heavy chains are of different isotypes and/or subclasses. In various embodiments, the antibody is an IgG1 antibody, more particularly an IgG1, kappa or IgG1, lambda isotype (i.e., IgG1, κ, λ), an IgG2a antibody (e.g., IgG2a, κ, λ), an IgG2b antibody (e.g., IgG2b, κ, λ), an IgG3 antibody (e.g., IgG3, κ, λ) or an IgG4 antibody (e.g., IgG4, κ, λ). For example or in a preferred embodiment, an antibody, preferably a monoclonal antibody, of the present invention is a IgG1, κ isotype or λ isotype, preferably comprising human IgG1/κ or human IgG1/A constant parts, or the antibody, preferably the monoclonal antibody, is derived from a IgG1, λ (lambda) or IgG1, κ (kappa) antibody, preferably from a human IgG1, λ (lambda) or a human IgG1, κ (kappa) antibody.

In one embodiment of the invention, the binding agent is a full-length IgG1 antibody. In one embodiment of the invention, the binding agent is a full-length human IgG1 antibody. In one embodiment of the invention, the binding agent is a full-length human IgG1 antibody with one or more mutations in the constant region.

In one embodiment of the invention, the antibody comprises at least one heavy chain constant region, wherein in at least one of said constant regions one or more amino acids in the positions corresponding to positions L234, L235, G237, D265, D270, K322, P329, and P331 in a human IgG1 heavy chain according to EU numbering, are not L, L, G, D, D, K, P, and P, respectively. For example, the amino acid corresponding to position 234 in a human IgG1 heavy chain according to EU numbering is not L, but preferably selected from F or A, and the amino acid corresponding to position 235 in a human IgG1 heavy chain according to EU numbering is not L, but preferably selected from E or A. In one embodiment of the invention, the positions corresponding to positions L234, L235, and D265 in a human IgG1 heavy chain according to EU numbering have been substituted. In one embodiment of the invention, the positions corresponding to positions L234, L235, and P331 in a human IgG1 heavy chain according to EU numbering have been substituted. In one embodiment of the invention, the positions corresponding to positions L234, L235, and P329 in a human IgG1 heavy chain according to EU numbering have been substituted.

In one embodiment, the at least one heavy chain constant region has been modified so that binding of C1q to said antibody is reduced compared to a wild-type antibody, preferably reduced by at least 70%, at least 80%, at least 90%, at least 95%, at least 97%, or 100%, wherein C1q binding is preferably determined by ELISA.

In one embodiment of the above aspects, the antibody is a monoclonal, chimeric or a monoclonal, humanized antibody or a fragment of such an antibody. The antibodies can be whole antibodies or antigen-binding fragments thereof including, for example, Fab, F(ab′)₂, Fv, single chain Fv fragments or bispecific antibodies. Furthermore, the antigen-binding fragments can include binding-domain immunoglobulin fusion proteins comprising (i) a binding domain polypeptide (such as a heavy chain variable region or a light chain variable region) that is fused to an immunoglobulin hinge region polypeptide, (ii) an immunoglobulin heavy chain CH2 constant region fused to the hinge region, and (iii) an immunoglobulin heavy chain CH3 constant region fused to the CH2 constant region. Such binding-domain immunoglobulin fusion proteins are further disclosed in US 2003/0118592 and US 2003/0133939.

In one embodiment of the above aspects, the antibody is a Fab fragment, F(ab′)₂ fragment, Fv fragment, or a single-chain (scFv) antibody. A single-chain variable fragment (scFv) can be a fusion protein of the variable regions of the heavy (VH) and light chains (VL) of immunoglobulins, connected with a short linker peptide, preferably of ten to about 25 amino acids. The linker can be rich in glycine for flexibility, as well as serine or threonine for solubility, and can either connect the N-terminus of the VH with the C-terminus of the V_{L}, or vice versa. This protein usually retains the specificity of the original immunoglobulin, despite removal of the constant regions and the introduction of the linker.

The antibodies of the present invention may or may not be capable of inducing at least one of complement dependent cytotoxicity (CDC) mediated lysis, antibody dependent cellular cytotoxicity (ADCC) mediated lysis, apotosis, homotypic adhesion and/or phagocytosis. In one embodiment, antibodies of the invention induce complement dependent cytotoxicity (CDC), e.g., at least about 20-40% CDC mediated lysis, preferably about 40-50% CDC mediated lysis, and more preferably more than 50% CDC mediated lysis of cells expressing PD-1. In one embodiment, antibodies of the invention do not induce complement dependent cytotoxicity (CDC). Alternatively or in addition, to inducing or not inducing CDC, antibodies of the invention may induce antibody dependent cellular cytotoxicity (ADCC) of cells expressing PD-1 in the presence of effector cells (e.g., monocytes, mononuclear cells, NK cells and PMNs). In one embodiment, antibodies of the invention do not induce antibody dependent cellular cytotoxicity (ADCC). Antibodies of the invention may have or may not have the ability to induce apoptosis, induce homotypic adhesion of cells and/or induce phagocytosis in the presence of macrophages. The antibodies of the invention may have one or more of the above described functional properties. Preferably, antibodies of the invention do not induce CDC mediated lysis and ADCC mediated lysis of cells expressing PD-1 and/or do not induce ADCC mediated lysis of cells expressing PD-1.

In one embodiment of all the above aspects, the PD-1 to which the antibody is able to bind is human PD-1. In one embodiment, the PD-1 has or comprises the amino acid sequence as set forth in SEQ ID NO: 71 or SEQ ID NO: 72, or the amino acid sequence of PD-1 has at least 70%, at least 75%, at least 80%, at least 85%, at least 90%, at least 95%, at least 97%, at least 99%, or 100% identity to the amino acid sequence as set forth in SEQ ID NO: 71 or SEQ ID NO: 72, or is an immunogenic fragment thereof. In one embodiment, the antibody has the ability of binding to a native epitope of PD-1 present on the surface of living cells.

In one embodiment of the above aspects, the antibodies of the present invention can be derivatized, linked to or co-expressed to other binding specificities. In another embodiment, the antibodies of the invention can be derivatized, linked to or co-expressed with another functional molecule, e.g., another peptide or protein (e.g., a Fab′ fragment). For example, an antibody of the invention can be functionally linked (e.g., by chemical coupling, genetic fusion, noncovalent association or otherwise) to one or more other molecular entities, such as another antibody (e.g., to produce a bispecific or a multispecific antibody).

In one embodiment of the above aspects, the antibody is a multispecific antibody comprising a first antigen-binding region binding to PD-1 and at least one further antigen-binding region binding to another antigen. In one embodiment, the antibody is a bispecific antibody comprising a first antigen-binding region binding to PD-1 and a second antigen-binding region binding to another antigen.

In one embodiment, the first and second binding arms are derived from full-length antibodies, such as from full-length IgG1, λ (lambda) or IgG1, κ (kappa) antibodies as mentioned above. In one embodiment, the first and second binding arms are derived from monoclonal antibodies.

For example or in a preferred embodiment, the first and/or second binding arm is derived from a IgG1, κ isotype or λ isotype, preferably comprising human IgG1/κ or human IgG1/λ constant parts. The first and/or second binding arms can comprise one or more mutations in the constant region, for example one or more amino acids in the positions corresponding to positions L234, L235, G237, D265, D270, K322, P329, and P331 in a human IgG1 heavy chain according to EU numbering, are not L, L, G, D, D, K, P, and P, respectively.

In this regard, in one embodiment, the invention provides a bispecific or multispecific molecule comprising at least one first binding specificity for PD-1 (e.g., an anti-PD-1 antibody or mimetic thereof), and a second or further binding specificity for another immune checkpoint, in order to either inhibit or activate/stimulate the respective other checkpoint. Other checkpoint inhibitors which may be targeted include, but are not limited to CTLA4, PD-L1, TIM-3, KIR or LAG-3. Checkpoint activators which may be targeted by the second binding specificity include, but are not limited to CD27, CD28, CD40, CD122, CD137, OX40, GITR, or ICOS. Preferred combinations of binding specificities in a bispecific or multispecific antibody or molecule include, for example, anti-PD1 and anti-PD-L1 or anti-PD-1 and anti-CTLA4.

In one embodiment, the invention provides a bispecific or multispecific molecule comprising at least one first binding specificity for PD-1 (e.g., an anti-PD-1 antibody or mimetic thereof), and a second or further binding specificity for, alternatively or in addition to the above, providing an antiangiogenesis activity. Thus, the second or further binding specifity can be capable of targeting vascular endothelial growth factor (VEGF) or its receptor VEGFR, for example VEGFR1, 2, 3. Alternatively or in addition, the second binding specifity may be capable of targeting PDGFR, c-Kit, Raf and/or RET.

In one embodiment, the invention provides a bispecific or multispecific molecule comprising at least one first binding specificity for PD-1 (e.g., an anti-PD-1 antibody or mimetic thereof), and a second or further binding specificity targeting a tumor antigen, which enables a specificity of the antibody of the present invention for cancer cells. In one embodiment of the present invention, the cancer cells can be selected from the group consisting of melanoma, lung cancer, renal cell carcinoma, bladder cancer, breast cancer, gastric and gastroesophageal junction cancers, pancreatic adenocarcinoma, ovarian cancer and lymphomas.

In one embodiment, in addition to a tumor antigen specificity and an anti-PD-1 binding specificity, a multispecific antibody of the present invention can comprise a third binding specificity. In one embodiment, the third binding specificity is directed to an Fc receptor, e.g., human Fc-gammaRI (CD64) or a human Fc-alpha receptor (CD89). Therefore, the invention includes multispecific molecules capable of binding to PD-1, to Fc-gammaR, Fc-alphaR or Fc-epsilonR expressing effector cells (e.g., monocytes, macrophagesor polymorphonuclear cells (PMNs)), and to target cancer cells expressing a tumor antigen.

The said first antigen-binding region binding to PD-1 of the multispecific or bispecific antibody of the present invention may comprise heavy and light chain variable regions of an antibody which competes for PD-1 binding with PD-L1 and/or PD-L2. In one embodiment of the multispecific or bispecific antibody, the first antigen-binding region binding to PD-1 comprises the heavy chain variable region (VH) and/or the light chain variable region (VL) as set forth herein.

In one embodiment of the above aspects, the antibody is obtainable by a method comprising the step of immunizing an animal with a protein or peptide having an amino acid sequence as set forth in SEQ ID NO: 71 or SEQ ID NO: 72, or an immunogenic fragment thereof, or a nucleic acid or host cell or virus expressing said protein or peptide, or an immunogenic fragment thereof. Preferably, the thus obtained antibody is specific for the afore mentioned protein, peptides or immunogenic fragments thereof. The nucleic acid or host cell or virus may be a nucleic acid or a host cell or a virus as disclosed herein.

The invention also provides isolated B cells from a non-human animal as described above. The isolated B cells can then be immortalized by fusion to an immortalized cell to provide a source (e.g., a hybridoma) of antibodies of the invention. Such hybridomas (i.e., which produce antibodies of the invention) are also included within the scope of the invention.

Thus, in a further aspect, the invention provides a hybridoma capable of producing the antibody of all of the above aspects. As exemplified herein, antibodies of the invention can be obtained directly from hybridomas which express the antibody, or can be cloned and recombinantly expressed in a host cell (e.g., a CHO cell, or a lymphocytic cell). Further examples of host cells are microorganisms, such as E. coli, and fungi, such as yeast. Alternatively, they can be produced recombinantly in a transgenic non-human animal or plant. Preferred antibodies of the invention are those produced by and obtainable from the above-described hybridomas, host cells or viruses, and the chimerized and humanized forms thereof. In a further aspect, the invention provides a conjugate comprising an antibody of the present invention coupled to a moiety or agent. In one embodiment of this aspect, the moiety or agent is selected from the group consisting of a radioisotope, an enzyme, a dye, a drug, a toxin and a cytotoxic agent. The dye can, for example, be a fluorescence dye or fluorescent tag. In one embodiment, the moiety or agent is capable of achieving immune cell activation. For example, the moiety or agent can be CD80 which interacts with CD28 on T cells.

The antibodies of the invention can be coupled to or functionally linked (e.g., by chemical coupling, genetic fusion, noncovalent association or otherwise) to one or more other molecular entities, such as another antibody having a binding specificity to PD-1. The one or more other antibodies are preferably antibodies of the present invention.

Thus, in a further aspect, the present invention provides a multimer, comprising at least two antibodies of the present invention or at least two conjugates of the present invention or a mixture of one or more antibodies of the present invention and one or more conjugates of the present invention. In one embodiment, the multimer comprising 4 to 8 antibodies of the present invention or 4 to 8 conjugates of the present invention. The antibodies or conjugates of the multimer of the invention may be linked to each other by peptides. Multimers of the present invention are characterized by an increased number of antigen binding sites to PD-1.

Accordingly, the present invention encompasses a large variety of antibody conjugates, bispecific and multispecific molecules, and fusion proteins, all of which bind to PD-1 expressing cells and which can be used to target other molecules to such cells.

In a further aspect, the present invention also relates to nucleic acids comprising genes or nucleic acid sequences encoding an antibody of the present invention or a fragment thereof. The encoded antibody chain may be a chain as described herein.

The nucleic acids may be comprised in a vector, e.g., a plasmid, cosmid, virus, bacteriophage or another vector used e.g., conventionally in genetic engineering. The vector may comprise further genes such as marker genes which allow for the selection of the vector in a suitable host cell and under suitable conditions. Furthermore, the vector may comprise expression control elements allowing proper expression of the coding regions in suitable hosts. Such control elements are known to the artisan and may include a promoter, a splice cassette, and a translation initiation codon. Preferably, the nucleic acid of the invention is operatively attached to the above expression control sequences allowing expression in eukaryotic or prokaryotic cells. Control elements ensuring expression in eukaryotic or prokaryotic cells are well known to those skilled in the art. Methods for construction of nucleic acid molecules according to the present invention, for construction of vectors comprising the above nucleic acid molecules, for introduction of the vectors into appropriately chosen host cells, for causing or achieving the expression are well-known in the art.

In one embodiment, the nucleic acid is RNA.

In one embodiment, the nucleic acid is associated with at least one agent having a stabilizing effect on the nucleic acid. The stabilizing effect can comprise protection from RNA degradation. In one embodiment of the present invention, the at least one agent forms a complex with and/or encloses said RNA. In one embodiment the at least one agent comprises at least one agent selected from the group consisting of an RNA-complexing lipid, an RNA complexing polymer and an RNA-complexing peptide or protein. For example, the at least one agent selected from at least one out of the group consisting of polyethyleneimine, protamine, a poly-L-lysine, a poly-L-arginine and a histone.

In a further aspect, the invention provides a vector comprising the nucleic acid of the present invention. In one embodiment, the vector is a multilamellar vesicle, an unilamellar vesicle, or a mixture thereof. In one embodiment, the vector is a liposome, preferably a cationic liposome. The liposome can comprise a phospholipid such as phosphatidylcholine and/or a sterol such as cholesterol. In one embodiment, the liposome has a particle diameter in the range of from about 50 nm to about 200 nm. In one embodiment, the vector as described herein further comprising a ligand for site specific targeting. The said ligand is for example an antibody. In one embodiment, the ligand, e.g., the antibody is capable of binding to a cancer cell, in particular a cancer cell as described herein. In one embodiment, the vector releases the RNA at a tumor cell and/or enters a tumor cell. In one embodiment, the ligand, e.g., the antibody binds to a protein associated with the surface of a diseased cell such as a tumor cell. For example, the ligand or antibody may bind to an extracellular portion of the disease-associated antigen.

A further aspect of the present invention relates to a host cell comprising a nucleic acid of the present invention or comprising a vector of the present invention. The host cell can be prokaryotic and/or eukaryotic host cells. Into these host cells, an exogenous nucleic acid and/or a vector can be introduced. In one embodiment, the host cell is an eukaryotic host cell, preferably a mammalian host cell. In one embodiment, the mammalian host cell is a CHO (Chinese hamster ovary) cell, a derivate of the CHO cell line, such as CHO-K1 and CHO pro-3, or a lymphocytic cell. In one embodiment, the mammalian host cell is selected from mouse myeloma cells, such as NS0 and Sp2/0, HEK293 (human embryonic kidney) cells or derivates thereof, such as HEK293T, HEK293T/17 and/or HEK293F, COS and Vero cells (both green African monkey kidney), and/or HeLa (human cervical cancer) cells. In one embodiment, the mammalian host cell is selected from HEK293, HEK293T and/or HEK293T/17 cells. Further examples of host cells are microorganisms, such as E. coli, and fungi, such as yeast, e.g., Saccharomyces cerevisiae or filamentous fungi, such as Neurospora and Aspergillus hosts.

In a further aspect, the invention provides a virus comprising a nucleic acid of the present invention or comprising a vector of the present invention.

In a further aspect, the invention provides a composition, preferably a pharmaceutical composition, comprising an active agent and a pharmaceutically acceptable carrier, wherein the active agent is at least one selected from:
(i) an antibody of the present invention;
(ii) a conjugate of the present invention;
(iii) a multimer of the present invention;
(iv) a nucleic acid of the present invention;
(v) a vector of the present invention;
(vi) a host cell of the present invention; and/or
(vii) a virus of the present invention.

In one embodiment, the pharmaceutical composition is formulated for parenteral administration, preferably for cardiovascular, in particular intravenous or intraarterial administration.

A further aspect of the present invention relates to the pharmaceutical composition of the present invention for use in a prophylactic and/or therapeutic treatment of a disease. In one embodiment of the medical use, the disease is cancer growth and/or cancer metastasis. In one embodiment of the medical use, the disease is characterized by comprising diseased cells or cancer cells which are characterized by expressing PD-L1 and/or being characterized by association of PD-L1 with their surface. In one embodiment of the medical use, the pharmaceutical composition is for use in a method of preventing or treating cancer. In one embodiment of the medical use, the cancer is selected from the group consisting of melanoma, lung cancer, renal cell carcinoma, bladder cancer, breast cancer, gastric and gastroesophageal junction cancers, pancreatic adenocarcinoma, ovarian cancer, kidney tumor, glioblastoma and lymphomas, preferably Hodgkin's lymphomas.

In one embodiment of the medical use, the pharmaceutical composition is to be specifically delivered to, accumulated in and/or are retained in a target organ or tissue. In one embodiment of the medical use, the target organ or tissue is a cancer tissue, in particular a cancer tissue as specified herein. For example, the diseased organ or tissue can be characterized by cells expressing a disease-associated antigen and/or being characterized by association of a disease-associated antigen with their surface. The disease-associated antigen can be a tumor-associated antigen. The disease-associated antigen can be associated with the surface of a diseased cell such as a tumor cell. In one embodiment of the medical use, the vector or the virus releases the nucleic acid at the target organ or tissue and/or enters cells at the target organ or tissue. In one embodiment of the medical use, the antibody is to be expressed in cells of the target organ or tissue.

In one embodiment of the medical use, the treatment is a monotherapy or a combination therapy. Preferably, the combinatorial treatment is at least one treatment selected from the group consisting chemotherapy, molecular-targeted therapy, radiation therapy, and other forms of immune therapy. Other forms of immune therapy may target other checkpoint inhibitors, thereby either inhibiting (antagonists) or activating/stimulating (agonists) the respective other checkpoint. Other checkpoint inhibitors which may be targeted include, but are not limited to CTLA4, PD-L1, TIM-3, KIR or LAG-3, checkpoint activators which may be targeted by the second binding specificity include, but are not limited to CD27, CD28, CD40, CD122, CD137, OX40, GITR, or ICOS. For example: Preferred combinations of binding specificities include anti-PD1 and anti-PD-L1 or anti-PD-1 and anti-CTLA4. Alternatively or in addition, the immune therapy can provide an antiangiogenesis activity. For example, by targeting the vascular endothelial growth factor (VEGF) or its receptor VEGFR (for example VEGFR1, 2, 3). Alternatively or in addition, it may be capable of targeting PDGFR, c-Kit, Raf and/or RET.

The antibodies of the present invention can also be used in combination with one or more vaccines, wherein the vaccines are for stimulating the immune system against an antigen expressed by diseased cells such as tumor cells. For example, the antigen can be one or more of the tumor antigens as specified herein. The vaccination can be achieved by administering vaccine RNA, i.e., RNA encoding an antigen or epitope against which an immune response is to be induced. Alternatively, a peptide or protein comprising an epitope for inducing an immune response against an antigen can be administered.

Accordingly, the present invention also provides a composition, preferably a pharmaceutical composition, comprising (i) peptide or protein comprising an epitope for inducing an immune response against an antigen in a subject, or a polynucleotide encoding the peptide or protein; and (ii) at least one selected from an antibody of the present invention, a conjugate of the present invention, a multimer of the present invention, a nucleic acid of the present invention, a vector of the present invention, a host cell of the present invention, and/or a virus of the present invention.

In one embodiment, the composition comprises RNA encoding the peptide or protein comprising an epitope for inducing an immune response against an antigen in a subject.

In one embodiment of the medical use, the subject is a human.

In a further aspect, the invention provides a method of treating or preventing a disease in a subject comprising administering to a subject at least one active agent, wherein the active agent is at least one selected from:
(i) an antibody of the present invention;
(ii) a conjugate of the present invention;
(iii) a multimer of the present invention;
(iv) a nucleic acid of the present invention;
(v) a vector of the present invention;
(vi) a host cell of the present invention; and/or
(vii) a virus of the present invention.

In one embodiment of the method, a pharmaceutical composition of the present invention is administered to the subject. In one embodiment of the method, the subject has a diseased organ or tissue characterized by cells expressing PD-L1 and/or being characterized by association of PD-L1 with their surface. In one embodiment of the method, the disease is cancer growth and/or cancer metastasis. In one embodiment of the method, the method is for treating or preventing cancer growth and/or cancer metastasis in a subject that has or is at risk of developing cancers and/or cancer metastases. In one embodiment of the method, an effective amount of the active agent is provided. Preferably, the antibody is provided at a dose in the range of 0.1 to 20 mg/kg, more preferably in a range of 0.3 to 10 mg/kg, in one or multiple doses. The said dose may be provided for example every 1 to 4 weeks, still more preferably every 2 to 3 weeks, for example very 2 or 3 weeks.

In one embodiment of the method, the cancer is selected from the group consisting of melanoma, lung cancer, renal cell carcinoma, bladder cancer, breast cancer, gastric and gastroesophageal junction cancers, pancreatic adenocarcinoma, ovarian cancer, kidney tumor, glioblastoma and lymphomas, preferably Hodgkin's lymphomas.

In one embodiment of the method, the active agent or the pharmaceutical composition is administered into the cardiovascular system, preferably the active agent or the pharmaceutical composition is administered by intravenous or intraarterial administration such as administration into a peripheral vein. In one embodiment of the method, the active agent or the pharmaceutical composition are specifically delivered to, accumulate in and/or are retained in a target organ or tissue. In one embodiment of the method, the target organ or tissue is a cancer tissue, in particular a cancer tissue as specified herein. For example, the diseased organ or tissue can be characterized by cells expressing a disease-associated antigen and/or being characterized by association of a disease-associated antigen with their surface. The disease-associated antigen can be a tumor-associated antigen. The disease-associated antigen can be associated with the surface of a diseased cell such as a tumor cell. In one embodiment of the method, the vector, the host cell or the virus releases the nucleic acid at the target organ or tissue and/or enters cells at the target organ or tissue, preferably, wherein the antibody is expressed in cells of the target organ or tissue.

In one embodiment of the method, the treatment is a monotherapy or a combination therapy. Preferably, the combinatorial treatment is at least one treatment selected from the group consisting of chemotherapy, molecular-targeted therapy, radiation therapy, and other forms of immune therapy. Other forms of immune therapy include vaccination e.g., RNA vaccination and/or may target other checkpoint inhibitors, thereby either inhibiting (antagonists) or activating/stimulating (agonists) the respective other checkpoint. Other checkpoint inhibitors which may be targeted include, but are not limited to CTLA4, PD-L1, TIM-3, KIR or LAG-3. Checkpoint activators which may be targeted by the second binding specificity include, but are not limited to CD27, CD28, CD40, CD122, CD137, OX40, GITR, or ICOS. For example: Preferred combinations of binding specificities include anti-PD1 and anti-PD-L1 or anti-PD-1 and anti-CTLA4. Alternatively or in addition, the immune therapy can provide an antiangiogenesis activity. For example, by targeting the vascular endothelial growth factor (VEGF) or its receptor VEGFR (for example VEGFR1, 2, 3). Alternatively or in addition, it may be capable of targeting PDGFR, c-Kit, Raf and/or RET.

In a preferred embodiment of the method, the treatment is a combination therapy, wherein the treatment comprises administering to the subject:
(i) peptide or protein comprising an epitope for inducing an immune response against an antigen in the subject, or a polynucleotide encoding the peptide or protein; and
(ii) at least one selected from an antibody of the present invention, a conjugate of the present invention, a multimer of the present invention, a nucleic acid of the present invention, a vector of the present invention, a host cell of the present invention, and/or a virus of the present invention.

In one embodiment, the peptide or protein comprising an epitope for inducing an immune response against an antigen in the subject or the polynucleotide encoding the peptide or protein and the at least one active compound as specified in (ii) are administered sequentially. In one embodiment, the at least one active compound as specified in (ii) is administered following administration of the peptide or protein comprising an epitope for inducing an immune response against an antigen in the subject or the polynucleotide encoding the peptide or protein. In one embodiment, the at least one active compound as specified in (ii) is administered 6 hours or later, 12 hours or later or 24 hours or later following administration of the peptide or protein comprising an epitope for inducing an immune response against an antigen in the subject or the polynucleotide encoding the peptide or protein. In one embodiment, the at least one active compound as specified in (ii) is administered between 12 hours and 48 hours following administration of the peptide or protein comprising an epitope for inducing an immune response against an antigen in the subject or the polynucleotide encoding the peptide or protein.

In one embodiment, the method of the invention comprises administering to the subject an RNA encoding the peptide or protein comprising an epitope for inducing an immune response against an antigen in the subject. In one embodiment of the method, the subject is a human.

In a further aspect, the invention provides a kit for qualitative or quantitative detection of PD-1 in a sample, wherein the kit comprises an antibody of the present invention or a conjugate of the present invention or a multimer of the present invention.

In a still further aspect, the invention provides the use of an antibody of the present invention or of a conjugate of the present invention or of a multimer of the present invention or of a kit of the present invention in a method of determining the presence or quantity of PD-1 expressed in a sample, the method comprising the steps of:
(i) contacting a sample with the antibody or the conjugate or the multimer, and
(ii) detecting the formation of and/or determining the quantity of a complex between the antibody or the conjugate or the multimer and PD-1.

In one embodiment, the kit or method allows quantitative and/or qualitative evaluations, e.g., absolute and/or relative measurements of PD-1.

Other features and advantages of the instant invention will be apparent from the following detailed description and claims.

### BRIEF DESCRIPTION OF THE DRAWINGS

FIG. 1 shows the binding of chimeric anti-PD-1 antibodies MAB-19-0202, MAB-19-0208, MAB-19-0217, MAB-19-0223, and MAB-19-0233 to recombinant human-PD-1 extracellular domain. The binding ability was determined by ELISA. Chimeric anti-PD-1 antibodies were tested in serial dilution ranging from 0.06 ng/mL to 1 μg/mL. As reference antibodies, anti-hPD-1-Ni-hIgG4 (features the variable region of Nivolumab) and anti-hPD1-Pem-hIgG4 (features the variable region of Pembrolizumab) were used. Data was fitted with a 4-parameter logistic model.

FIG. 2 shows the binding of chimeric anti-PD-1 antibodies MAB-19-0202, MAB-19-0208, MAB-19-0217, MAB-19-0223, and MAB-19-0233 to HEK-293-hPD-1. The binding was assessed using a CellInsight CX5 high content imager device. Chimeric anti-PD-1 antibodies were tested in serial dilution ranging from 0.07 ng/mL to 1 μg/mL. As reference antibodies, anti-hPD-1-Ni-hIgG4 (features the variable region of Nivolumab) and anti-hPD1-Pem-hIgG4 (features the variable region of Pembrolizumab) were used. RFU is Relative fluorescence units. Data was fitted with a 4-parameter logistic model.

FIG. 3 shows the blockade of PD-1/PD-L1 interaction by chimeric anti-PD-1 antibodies MAB-19-0202, MAB-19-0208, MAB-19-0217, MAB-19-0223, and MAB-19-0233, which was assessed using a PD-1/PD-L1 blockade bioassay. Chimeric anti-PD-1 antibodies were tested in serial dilution ranging from 9 ng/mL to 6.67 μg/mL. As reference antibodies, anti-hPD-1-Ni-hIgG4 (features the variable region of Nivolumab) and anti-hPD1-Pem-hIgG4 (features the variable region of Pembrolizumab) were used. RLU is Relative light units. Data was fitted with a 4-parameter logistic model.

FIG. 4 shows the release of the PD-1/PD-L1-mediated T-cell inhibition measured by an antigen-specific T cell assay with active PD-1/PD-L1 axis. CFSE-labelled T cells electroporated with a claudin-6-specific TCR- and PD-1-in vitro translated (IVT)-RNA were incubated with autologous claudin-6-IVT-RNA-electroporated immature dendritic cells in the presence of a serial dilution ranging from 0.6 ng/mL to 0.6 μg/mL of chimeric anti-PD1 antibodies MAB-19-0202, MAB-19-0208, MAB-19-0217, MAB-19-0223, and MAB-19-0233 for five days. CD8⁺ T-cell proliferation was measured by flow cytometry. Data shown are the expansion indices as calculated using FlowJo software. Error bars (SD) indicate variation within the experiment (two replicates, using cells from one donor). As reference antibody Pembrolizumab (MSD, PZN 10749897) was used. Data was fitted with a 4-parameter logistic model.

FIG. 5 shows the binding of humanized anti-PD-1 antibodies MAB-19-0603, MAB-19-0608, MAB-19-0613, and MAB-19-0618 and the parental chimeric anti-PD-1 MAB-19-0202 to recombinant human-PD-1 extracellular domain, which was determined by ELISA. Chimeric anti-PD-1 antibodies were tested in serial dilution ranging from 0.15 ng/mL to 2.5 μg/mL. As reference antibodies, anti-hPD-1-Ni-hIgG4 (features the variable region of Nivolumab) and anti-hPD1-Pem-hIgG4 (features the variable region of Pembrolizumab) were used. Data was fitted with a 4-parameter logistic model.

FIG. 6 shows the binding of humanized anti-PD-1 antibodies MAB-19-0583, MAB-19-0594, and MAB-19-0598 and the parental chimeric anti-PD-1 MAB-19-0233 to recombinant human-PD-1 extracellular domain, which was determined by ELISA. Chimeric anti-PD-1 antibodies were tested in serial dilution ranging from 0.15 ng/mL to 2.5 μg/mL. As reference antibodies, anti-hPD-1-Ni-hIgG4 (features the variable region of Nivolumab) and anti-hPD1-Pem-hIgG4 (features the variable region of Pembrolizumab) were used. Data was fitted with a 4-parameter logistic model.

FIG. 7 shows the binding of humanized anti-PD-1 antibodies MAB-19-0603, MAB-19-0608, MAB-19-0613, and MAB-19-0618 and the parental chimeric anti-PD-1 MAB-19-0202 to HEK-293-hPD-1. The binding was assessed using a CellInsight CX5 high content imager device. Chimeric anti-PD-1 antibodies were tested in serial dilution ranging from 0.1 ng/mL to 1 μg/mL. As reference antibodies, anti-hPD-1-Ni-hIgG4 (features the variable region of Nivolumab) and anti-hPD1-Pem-hIgG4 (features the variable region of Pembrolizumab) were used. RFU is Relative fluorescence units. Data was fitted with a 4-parameter logistic model.

FIG. 8 shows the binding of humanized anti-PD-1 antibodies MAB-19-0583, MAB-19-0594, and MAB-19-0598 and the parental chimeric anti-PD-1 MAB-19-0233 to HEK-293-hPD-1, which was assessed using a CellInsight CX5 high content imager device. Chimeric anti-PD-1 antibodies were tested in serial dilution ranging from 0.1 ng/mL to 1 μg/mL. As reference antibodies, anti-hPD-1-Ni-hIgG4 (features the variable region of Nivolumab) and anti-hPD1-Pem-hIgG4 (features the variable region of Pembrolizumab) were used. RFU is Relative fluorescence units. Data was fitted with a 4-parameter logistic model.

FIG. 9 shows the blockade of PD-1/PD-L1 interaction by the humanized anti-PD-1 antibodies MAB-19-0603, MAB-19-0608, MAB-19-0613, and MAB-19-0618 and the parental chimeric anti-PD-1 MAB-19-0202, which was assessed using a PD-1/PD-L1 blockade bioassay. Chimeric anti-PD-1 antibodies were tested in serial dilution ranging from 9 ng/mL to 6.67 μg/mL. As reference antibodies, anti-hPD-1-Ni-hIgG4 (features the variable region of Nivolumab) and anti-hPD1-Pem-hIgG4 (features the variable region of Pembrolizumab) were used. RLU is Relative light units. Data was fitted with a 4-parameter logistic model.

FIG. 10 shows the blockade of PD-1/PD-L1 interaction by the humanized anti-PD-1 antibodies MAB-19-0583, MAB-19-0594, and MAB-19-0598 and the parental chimeric anti-PD-1 MAB-19-0233, which was assessed using a PD-1/PD-L1 blockade bioassay. Chimeric anti-PD-1 antibodies were tested in serial dilution ranging from 9 ng/mL to 6.67 μg/mL. As reference antibodies, anti-hPD-1-Ni-hIgG4 (features the variable region of Nivolumab) and anti-hPD1-Pem-hIgG4 (features the variable region of Pembrolizumab) were used. RLU is Relative light units. Data was fitted with a 4-parameter logistic model.

FIG. 11 shows the release of the PD-1/PD-L1-mediated T-cell inhibition measured by an antigen-specific T cell assay with active PD-1/PD-L1 axis. CFSE-labelled T cells electroporated with a claudin-6-specific TCR- and PD-1-in vitro translated (IVT)-RNA were incubated with autologous claudin-6-IVT-RNA-electroporated immature dendritic cells in the presence of a serial dilution ranging from 0.6 ng/mL to 0.6 μg/mL of humanized anti-PD-1 antibodies MAB-19-0603, MAB-19-0608, MAB-19-0613, and MAB-19-0618 and the parental chimeric anti-PD-1 MAB-19-0202 for five days. CD8⁺ T-cell proliferation was measured by flow cytometry. Data shown are the expansion indices as calculated using FlowJo software. Error bars (SD) indicate variation within the experiment (two replicates, using cells from one donor). As reference antibody Pembrolizumab (MSD, PZN 10749897) was used. Data was fitted with a 4-parameter logistic model.

FIG. 12 shows binding of in vitro expressed anti-PD-1 RiboMab-19-0202 and RiboMab-19-0233 to full-length human PD-1 transfected into K562 cells. Adherent HEK293T/17 cells were lipofected with 3 μg RiboMab-encoding mRNA (2:1 ratio of heavy chain to light chain, 400 ng mRNA complexed per μL Lipofectamine MessengerMAX) and after 20 h of incubation supernatants were collected. K562 cells were electroporated with 1 μg mRNA encoding full-length human PD-1 and treated 20 h after electroporation with serial dilutions of the RiboMab-containing supernatants ranging from 0.006% to 100%. Binding of RiboMabs was detected by flow cytometry using an AlexaFluor488-conjugated goat anti-human IgG Fc-specific (Fab′)₂ fragment. Data are presented as geometric mean fluorescence intensity (gMFI) Alexa Fluor 488 f standard deviation (SD) of n=3 technical replicates.

### DETAILED DESCRIPTION OF THE INVENTION

Although the present invention is described in detail below, it is to be understood that this invention is not limited to the particular methodologies, protocols and reagents described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art.

In the following, the elements of the present invention will be described. These elements are listed with specific embodiments, however, it should be understood that they may be combined in any manner and in any number to create additional embodiments. The variously described examples and preferred embodiments should not be construed to limit the present invention to only the explicitly described embodiments. This description should be understood to support and encompass embodiments which combine the explicitly described embodiments with any number of the disclosed and/or preferred elements. Furthermore, any permutations and combinations of all described elements in this application should be considered disclosed by the description of the present application unless the context indicates otherwise.

Preferably, the terms used herein are defined as described in “A multilingual glossary of biotechnological terms: (IUPAC Recommendations)”, H. G. W. Leuenberger. B. Nagel. and H. Kolbl. Eds., (1995) Helvetica Chimica Acta. CH-4010 Basel. Switzerland.

The practice of the present invention will employ, unless otherwise indicated, conventional methods of biochemistry, cell biology, immunology, and recombinant DNA techniques which are explained in the literature in the field (cf., e.g., Molecular Cloning: A Laboratory Manual, 2^{nd} Edition, J. Sambrook et al. eds., Cold Spring Harbor Laboratory Press, Cold Spring Harbor 1989).

Throughout this specification and the claims which follow, unless the context requires otherwise, the word “comprise”, and variations such as “comprises” and “comprising”, will be understood to imply the inclusion of a stated member, integer or step or group of members, integers or steps but not the exclusion of any other member, integer or step or group of members, integers or steps although in some embodiments such other member, integer or step or group of members, integers or steps may be excluded, i.e., the subject-matter consists in the inclusion of a stated member, integer or step or group of members, integers or steps. The terms “a” and “an” and “the” and similar reference used in the context of describing the invention (especially in the context of the claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein.

All methods described herein can be performed in any suitable order unless otherwise indicated herein or otherwise clearly contradicted by context. The use of any and all examples, or exemplary language (e.g., “such as”), provided herein is intended merely to better illustrate the invention and does not pose a limitation on the scope of the invention otherwise claimed. No language in the specification should be construed as indicating any non-claimed element essential to the practice of the invention.

Several documents are cited throughout the text of this specification. Each of the documents cited herein (including all patents, patent applications, scientific publications, manufacturer's specifications, instructions, etc.), whether supra or infra, are hereby incorporated by reference in their entirety. Nothing herein is to be construed as an admission that the invention is not entitled to antedate such disclosure by virtue of prior invention.

Terms such as “reducing” or “inhibiting” relate to the ability to cause an overall decrease, preferably of 5% or greater, 10% or greater, 20% or greater, more preferably of 50% or greater, and most preferably of 75% or greater, in the level. The term “inhibit” or similar phrases includes a complete or essentially complete inhibition, i.e. a reduction to zero or essentially to zero.

Terms such as “increasing”, “enhancing”, “promoting” or “prolonging” preferably relate to an increase, enhancement, promotion or prolongation by about at least 10%, preferably at least 20%, preferably at least 30%, preferably at least 40%, preferably at least 50%, preferably at least 80%, preferably at least 100%, preferably at least 200% and in particular at least 300%. These terms may also relate to an increase, enhancement, promotion or prolongation from zero or a non-measurable or non-detectable level to a level of more than zero or a level which is measurable or detectable.

The term “PD-1” relates to programmed cell death-1 and includes any variants, conformations, isoforms and species homologs of PD-1 which are naturally expressed by cells or are expressed by cells transfected with the PD-1 gene. Preferably, “PD-1” relates to human PD-1, in particular to a protein having the amino acid sequence (NCBI Reference Sequence: NP_005009.2) as set forth in SEQ ID NO: 71 of the sequence listing, or a protein being preferably encoded by a nucleic acid sequence (NCBI Reference Sequence: NM_005018.2) as set forth in SEQ ID NO: 73 of the sequence listing.

The term “PD-1” includes posttranslationally modified variants, isoforms and species homologs of human PD-1 which are naturally expressed by cells or are expressed in/on cells transfected with the PD-1 gene.

The term “PD-1 variant” shall encompass (i) PD-1 splice variants, (ii) PD-1-posttranslationally modified variants, particularly including variants with different N-glycosylation status, (iii) PD-1 conformation variants. Such variants may include soluble forms of PD-1.

PD-1 is a type I membrane protein that belongs to the immunoglobulin superfamily (The EMBO Journal (1992), vol. 11, issue 11, p. 3887-3895). The human PD-1 protein comprises an extracellular domain composed of the amino acids at positions 24 to 170 of the sequence as set forth in SEQ ID NO: 71 of the sequence listing, a transmembrane domain (amino acids at positions 171 to 191 of the sequence as set forth in SEQ ID NO: 71) and a cytoplasmatic domain (amino acids at positions 192 to 288 of the sequence as set forth in SEQ ID NO: 71). The term “PD-1 fragment” as used herein shall encompass any fragment of a PD-1 protein, preferably an immunogenic fragment. The term also encompasses, for example, the above mentioned domains of the full length protein or any fragment of these domains, in particular immunogenic fragments. The amino acid sequence of a preferred extracellular domain of the human PD-1 protein is set forth in SEQ ID NO: 72 of the sequence listing.

The term “extracellular portion” or “extracellular domain” in the context of the present invention preferably refers to a part of a molecule such as a protein that is facing the extracellular space of a cell and preferably is accessible from the outside of said cell, e.g., by binding molecules such as antibodies located outside the cell. Preferably, the term refers to one or more extracellular loops or domains or a fragment thereof.

The term “antibody” refers to a glycoprotein comprising at least two heavy (H) chains and two light (L) chains inter-connected by disulfide bonds, or an antigen-binding portion thereof. The term “antibody” also includes all recombinant forms of antibodies, in particular of the antibodies described herein, e.g., antibodies expressed in prokaryotes or eukaryotic cells, unglycosylated antibodies, and any antigen-binding antibody fragments and derivatives as described below. Each heavy chain is comprised of a heavy chain variable region (abbreviated herein as VH) and a heavy chain constant region. Each light chain is comprised of a light chain variable region (abbreviated herein as VL) and a light chain constant region. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system.

The term “humanized antibody” refers to a molecule having an antigen-binding site that is substantially derived from an immunoglobulin from a non-human species, wherein the remaining immunoglobulin structure of the molecule is based upon the structure and/or sequence of a human immunoglobulin. The antigen-binding site may either comprise complete variable domains fused onto constant domains or only the complementarity determining regions (CDR) grafted onto appropriate framework regions in the variable domains. Antigen binding sites may be wild-type or modified by one or more amino acid substitutions, e.g., modified to resemble human immunoglobulins more closely. Some forms of humanized antibodies preserve all CDR sequences (for example a humanized mouse antibody which contains all six CDRs from the mouse antibody). Other forms have one or more CDRs which are altered with respect to the original antibody.

The term “chimeric antibody” refers to those antibodies wherein one portion of each of the amino acid sequences of heavy and light chains is homologous to corresponding sequences in antibodies derived from a particular species or belonging to a particular class, while the remaining segment of the chain is homologous to corresponding sequences in another. Typically the variable region of both light and heavy chains mimics the variable regions of antibodies derived from one species of mammals, while the constant portions are homologous to sequences of antibodies derived from another. One clear advantage to such chimeric forms is that the variable region can conveniently be derived from presently known sources using readily available B-cells or hybridomas from non-human host organisms in combination with constant regions derived from, for example, human cell preparations. While the variable region has the advantage of ease of preparation and the specificity is not affected by the source, the constant region being human, is less likely to elicit an immune response from a human subject when the antibodies are injected than would the constant region from a non human source. However, the definition is not limited to this particular example.

The term “antigen-binding portion” of an antibody (or simply “binding portion”), as used herein, refers to one or more fragments of an antibody that retain the ability to specifically bind to an antigen. It has been shown that the antigen-binding function of an antibody can be performed by fragments of a full-length antibody. Examples of binding fragments encompassed within the term “antigen-binding portion” of an antibody include (i) Fab fragments, monovalent fragments consisting of the VL, VH, CL and CH domains; (ii) F(ab′)₂ fragments, bivalent fragments comprising two Fab fragments linked by a disulfide bridge at the hinge region; (iii) Fd fragments consisting of the VH and CH domains; (iv) Fv fragments consisting of the VL and VH domains of a single arm of an antibody, (v) dAb fragments (Ward et al., (1989) Nature 341: 544-546), which consist of a VH domain; (vi) isolated complementarity determining regions (CDR), and (vii) combinations of two or more isolated CDRs which may optionally be joined by a synthetic linker. Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242: 423-426; and Huston et al. (1988) Proc. Natl. Acad. Sci. USA 85: 5879-5883). Such single chain antibodies are also intended to be encompassed within the term “antigen-binding portion” of an antibody. A further example is binding-domain immunoglobulin fusion proteins comprising (i) a binding domain polypeptide that is fused to an immunoglobulin hinge region polypeptide, (ii) an immunoglobulin heavy chain CH2 constant region fused to the hinge region, and (iii) an immunoglobulin heavy chain CH3 constant region fused to the CH2 constant region. The binding domain polypeptide can be a heavy chain variable region or a light chain variable region. The binding-domain immunoglobulin fusion proteins are further disclosed in US 2003/0118592 and US 2003/0133939. These antibody fragments are obtained using conventional techniques known to those with skill in the art, and the fragments are screened for utility in the same manner as are intact antibodies.

The term “epitope” means a protein determinant capable of binding to an antibody, wherein the term “binding” herein preferably relates to a specific binding. Epitopes usually consist of chemically active surface groupings of molecules such as amino acids or sugar side chains and usually have specific three dimensional structural characteristics, as well as specific charge characteristics. Conformational and non-conformational epitopes are distinguished in that the binding to the former but not the latter is lost in the presence of denaturing solvents. The term “epitope” preferably refers to an antigenic determinant in a molecule, i.e., to a part or fragment of a molecule such as an antigen that is recognized by the immune system. For example, the epitope may be recognized by T cells, B cells or antibodies. An epitope of an antigen may include a continuous or discontinuous portion of the antigen and may be between about 5 and about 100, such as between about 5 and about 50, more preferably between about 8 and about 30, most preferably between about 10 and about 25 amino acids in length, for example, the epitope may be preferably 9, 10, 11, 12, 13, 14, 15, 16, 17, 18, 19, 20, 21, 22, 23, 24, or 25 amino acids in length. In one embodiment, an epitope is between about 10 and about 25 amino acids in length. The term “epitope” includes B cell epitopes and T cell epitopes.

The term “T cell epitope” refers to a part or fragment of a protein that is recognized by a T cell when presented in the context of MHC molecules. The term “major histocompatibility complex” and the abbreviation “MHC” includes MHC class I and MHC class II molecules and relates to a complex of genes which is present in all vertebrates. MHC proteins or molecules are important for signaling between lymphocytes and antigen presenting cells or diseased cells in immune reactions, wherein the MHC proteins or molecules bind peptide epitopes and present them for recognition by T cell receptors on T cells. The proteins encoded by the MHC are expressed on the surface of cells, and display both self-antigens (peptide fragments from the cell itself) and non-self-antigens (e.g., fragments of invading microorganisms) to a T cell. In the case of class I MHC/peptide complexes, the binding peptides are typically about 8 to about 10 amino acids long although longer or shorter peptides may be effective. In the case of class II MHC/peptide complexes, the binding peptides are typically about 10 to about 25 amino acids long and are in particular about 13 to about 18 amino acids long, whereas longer and shorter peptides may be effective.

The term “bispecific molecule” is intended to include any agent, e.g., a protein, peptide, or protein or peptide complex, which has two different binding specificities. For example, the molecule may bind to, or interact with (a) a cell surface antigen, and (b) an Fc receptor on the surface of an effector cell. The term “multispecific molecule” or “heterospecific molecule” is intended to include any agent, e.g., a protein, peptide, or protein or peptide complex, which has more than two different binding specificities. For example, the molecule may bind to, or interact with (a) a cell surface antigen, (b) an Fc receptor on the surface of an effector cell, and (c) at least one other component. Accordingly, the invention includes, but is not limited to, bispecific, trispecific, tetraspecific, and other multispecific molecules which are directed to PD-1, and to other targets, such as Fc receptors on effector cells. The term “bispecific antibodies” also includes diabodies. Diabodies are bivalent, bispecific antibodies in which the VH and VL domains are expressed on a single polypeptide chain, but using a linker that is too short to allow for pairing between the two domains on the same chain, thereby forcing the domains to pair with complementary domains of another chain and creating two antigen binding sites (see e.g., Holliger, P., et al. (1993) Proc. Natl. Acad. Sci. USA 90: 6444-6448; Poljak, R. J., et al. (1994) Structure 2: 1121-1123).

The invention also includes derivatives of the antibodies described herein. The term “antibody derivatives” refers to any modified form of an antibody, e.g., a conjugate of the antibody and another agent or antibody. As used herein, an antibody is “derived from” a particular germline sequence if the antibody is obtained from a system by immunizing an animal or by screening an immunoglobulin gene library, and wherein the selected antibody is at least 90%, more preferably at least 95%, even more preferably at least 96%, 97%, 98%, or 99% identical in amino acid sequence to the amino acid sequence encoded by the germline immunoglobulin gene. Typically, an antibody derived from a particular germline sequence will display no more than 10 amino acid differences, more preferably, no more than 5, or even more preferably, no more than 4, 3, 2, or 1 amino acid difference from the amino acid sequence encoded by the germline immunoglobulin gene.

As used herein, the term “heteroantibodies” refers to two or more antibodies, derivatives thereof, or antigen-binding regions linked together, at least two of which have different specificities. These different specificities include a binding specificity for an Fc receptor on an effector cell, and a binding specificity for an antigen or epitope on a target cell, e.g., a tumor cell.

The antibodies described herein may be human antibodies. The term “human antibody”, as used herein, is intended to include antibodies having variable and constant regions derived from human germline immunoglobulin sequences. The human antibodies of the invention may include amino acid residues not encoded by human germline immunoglobulin sequences (e.g., mutations introduced by random or site-specific mutagenesis in vitro or by somatic mutation in vivo).

The term “monoclonal antibody” as used herein refers to a preparation of antibody molecules of single molecular composition. A monoclonal antibody displays a single binding specificity and affinity for a particular epitope. In one embodiment, the monoclonal antibodies are produced by a hybridoma which includes a B cell obtained from a non-human animal, e.g., mouse, fused to an immortalized cell.

The term “recombinant antibody”, as used herein, includes all antibodies that are prepared, expressed, created or isolated by recombinant means, such as (a) antibodies isolated from an animal (e.g., a mouse) that is transgenic or transchromosomal with respect to the immunoglobulin genes or a hybridoma prepared therefrom, (b) antibodies isolated from a host cell transformed to express the antibody, e.g., from a transfectoma, (c) antibodies isolated from a recombinant, combinatorial antibody library, and (d) antibodies prepared, expressed, created or isolated by any other means that involve splicing of immunoglobulin gene sequences to other DNA sequences.

The term “transfectoma”, as used herein, includes recombinant eukaryotic host cells expressing an antibody, such as CHO cells, NS/0 cells, HEK293 cells, HEK293T cells, HEK293T/17 plant cells, or fungi, including yeast cells.

As used herein, a “heterologous antibody” is defined in relation to a transgenic organism producing such an antibody. Ibis term refers to an antibody having an amino acid sequence or an encoding nucleic acid sequence corresponding to that found in an organism not consisting of the transgenic organism, and being generally derived from a species other than the transgenic organism.

As used herein, a “heterohybrid antibody” refers to an antibody having light and heavy chains of different organismal origins. For example, an antibody having a human heavy chain associated with a murine light chain is a heterohybrid antibody.

The antibodies described herein are preferably isolated. An “isolated antibody” as used herein, is intended to refer to an antibody which is substantially free of other antibodies having different antigenic specificities (e.g., an isolated antibody that specifically binds to PD-1 is substantially free of antibodies that specifically bind antigens other than PD-1). An isolated antibody that specifically binds to an epitope, isoform or variant of human PD-1 may, however, have cross-reactivity to other related antigens, e.g., from other species (e.g., PD-1 species homologs). Moreover, an isolated antibody may be substantially free of other cellular material and/or chemicals. In one embodiment of the invention, a combination of “isolated” monoclonal antibodies relates to antibodies having different specificities and being combined in a well defined composition.

As used herein, “isotype” refers to the antibody class (e.g., IgM or IgG1) that is encoded by heavy chain constant region genes.

As used herein, “isotype switching” refers to the phenomenon by which the class, or isotype, of an antibody changes from one Ig class to one of the other Ig classes.

According to the invention, the term “binding” preferably relates to “specific binding”. As used herein, “specific binding” refers to antibody binding to a predetermined antigen. Typically, the antibody binds with an affinity corresponding to a KD of about 1×10⁻⁷ M or less, and binds to the predetermined antigen with an affinity corresponding to a KD that is at least two, preferably at least three, more preferably at least four, orders of magnitude lower than its affinity for binding to a non-specific antigen (e.g., BSA, casein) other than the predetermined antigen or a closely-related antigen. The term “KD” (M), as used herein, is intended to refer to the dissociation equilibrium constant of a particular antibody-antigen interaction.

As used herein the term “naturally occurring” as applied to an object refers to the fact that an object can be found in nature. For example, a polypeptide or polynucleotide sequence that is present in an organism (including viruses) that can be isolated from a source in nature and which has not been intentionally modified by man in the laboratory is naturally occurring.

The term “rearranged” as used herein refers to a configuration of a heavy chain or light chain immunoglobulin locus wherein a V segment is positioned immediately adjacent to a D-J or J segment in a conformation encoding essentially a complete VH or VL domain, respectively. A rearranged immunoglobulin (antibody) gene locus can be identified by comparison to germline DNA; a rearranged locus will have at least one recombined heptamer/nonamer homology element.

The term “unrearranged” or “germline configuration” as used herein in reference to a V segment refers to the configuration wherein the V segment is not recombined so as to be immediately adjacent to a D or J segment.

I. Mechanisms of Antibody Action

Although the following provides considerations regarding the mechanism underlying the therapeutic efficacy of antibodies of the invention it is not to be considered as limiting to the invention in any way.

The antibodies described herein preferably interact with the immune checkpoint PD-1. By binding to PD-1, the interaction of PD-1 with its ligands (PD-L1 and PD-L2) is inhibited. PD-L1 is expressed for example on tumor cells and antigen-presenting cells of the tumor microenvironment. The interaction of PD-1 and PD-L1 would result in abrogation of an immune response, preferably a T-cell mediated immune response, so that by blocking PD-1 with an antibody as described herein such an abrogation of the immune response is prevented or at least reduced, or said in other words an immune response is induced.

Even though PD-1 and its ligands interact with each other in preventing or reducing an immune response, for achieving this effect a PD-1 blockade might be advantageous over a ligand blockade. This is because a blockade of e.g., PD-L1 might still result in a reduced immune response, since an inhibitory signaling between diseased cells expressing PD-L2 and lymphocytes expressing PD-1 could help in inhibiting the immune response by the immune system.

The immune system has the ability to recognize and destroy diseased cells via two separate modalities: innate and adaptive immunity. The innate component consists of macrophages, natural killer (NK) cells, monocytes, and granulocytes. These cells identify molecular patterns involved in cellular transformation and release various cytokines and inflammatory mediators. The innate response lacks the memory capability for foreign antigens, a feature present in adaptive immune response. This latter component of immune system also features specificity for foreign antigens, imparted by presence of receptors on lymphocytes. Antigen presenting cells (APCs) also play a role in the adaptive response—they engulf foreign antigens and present them to the lymphocytes in the context of major histocompatibility complex. CD⁴⁺ T cells bear receptors that recognize antigens in the context of MHC class II molecules, which then enables them to release cytokines and further activate CD8⁺ lymphocytes (CTLs) or B cells. CTLs are part of cell-mediated immunity and are capable of eliminating cells after recognition of antigens presented in the context of MHC class I molecules, via apoptosis or perforin-mediated cell lysis. It is widely accepted that T-cell mediated immunity plays a vital role in the anti-tumor response. B cells are involved in release of immunoglobulins and as such are part of the humoral immune system.

The term “immune response” refers to an integrated bodily response to a target such as an antigen or a cell expressing an antigen and preferably refers to a cellular immune response or a cellular as well as a humoral immune response. The immune response may be protective/preventive/prophylactic and/or therapeutic.

“Inducing an immune response” may mean that there was no immune response before induction, but it may also mean that there was a certain level of immune response before induction and after induction said immune response is enhanced. Thus, “inducing an immune response” also includes “enhancing an immune response”. Preferably, after inducing an immune response in a subject, said subject is protected from developing a disease such as a cancer disease or the disease condition is ameliorated by inducing an immune response. Inducing an immune response in this case may mean that the disease condition of the subject is ameliorated, that the subject does not develop metastases, or that the subject being at risk of developing a cancer disease does not develop a cancer disease.

The terms “cellular immune response” and “cellular response” or similar terms refer to an immune response directed to cells. The innate cellular immune response is driven by macrophages, natural killer (NK) cells, monocytes, and granulocytes. The adaptive cellular immune response is characterized by presentation of an antigen in the context of MHC class I or class II involving T cells or T-lymphocytes which act as either “helpers” or “killers”. The helper T cells (also termed CD4+ T cells) play a central role by regulating the immune response and the killer cells (also termed cytotoxic T cells, cytolytic T cells, CD8⁺ T cells or CTLs) kill diseased cells such as cancer cells, preventing the production of more diseased cells. In preferred embodiments, the present invention involves the stimulation of an anti-tumor CTL response against tumor cells expressing one or more tumor antigens and preferably presenting such tumor antigens on MHC class I.

A “tumor antigen” according to the invention covers any substance, preferably a peptide or protein, that is a target of and/or induces an immune response such as a specific reaction with antibodies or T-lymphocytes (T cells). Preferably, an antigen comprises at least one epitope such as a T cell epitope. The tumor antigen or a T cell epitope thereof is preferably presented by a cell, preferably by an antigen presenting cell which includes a diseased cell, in particular a cancer cell, in the context of MHC molecules, which results in an immune response against the antigen (including cells expressing the antigen).

The antibodies of the present invention are characterized by their binding properties to PD-1 and preferably their ability to inhibit the immunosuppressive signal of PD-1. As detailed in the summary of the invention and the claims as attached, the antibodies of the present invention are characterized by comprising a heavy chain variable region (VH) comprising a complementarity-determining region 3 (HCDR3) having or comprising a sequence as set forth herein, and/or by comprising a light chain variable region (VL) comprising a complementarity-determining region 3 (LCDR3) having or comprising a sequence as set forth herein. In preferred embodiments the complementarity-determining region 1 and 2 of each of VH and VL is further specified.

The terms “a heavy chain variable region” (also referred to as “VH”) and “a light chain variable region” (also referred to as “VL”) are used here in their most general meaning and comprise any sequences that are able to comprise complementarity determining regions (CDR), interspersed with other regions, which also termed framework regions (FR). The framework regions inter alia space the CDRs so that they are able to form the antigen-binding site, in particular after folding and pairing of VH and VL. Preferably each VH and VL is composed of three CDRs and four FRs, arranged from amino-terminus to carboxy-terminus in the following order: FR1, CDR1, FR2, CDR2, FR3, CDR3, FR4. That is, the terms “a heavy chain variable region” and “a light chain variable region” are not to be construed to be limited to such sequences as they can be found in a native antibody or in the VH and VL sequences as exemplified herein (SEQ ID NOs: 52 to 70 of the sequence listing). These terms include any sequences capable of comprising and adequately positioning CDRs, for example such sequences as derived from VL and VH regions of native antibodies or as derived from the sequences as set forth in SEQ ID NOs: 52 to 70 of the sequence listing. It will be appreciated by those skilled in the art that in particular the sequences of the framework regions can be modified (including both variants with regard to amino acid substitutions and variants with regard to the sequence length, i.e., insertion or deletion variants) without losing the charactistics of the VH and VL, respectively. In a preferred embodiment any modification is limited to the framework regions. But, a person skilled in the art is also well aware of the fact that also CDR, hypervariable and variable regions can be modified without losing the ability to bind PD-1. For example, CDR regions will be either identical or highly homologous to the regions specified herein. By “highly homologous” it is contemplated that from 1 to 5, preferably from 1 to 4, such as 1 to 3 or 1 or 2 substitutions may be made in the CDRs. In addition, the hypervariable and variable regions may be modified so that they show substantial homology with the regions specifically disclosed herein.

The CDRs as specified herein have been identified by using two different CDR identification methods. The first numbering scheme used herein is according to Kabat (Wu and Kabat, 1970; Kabat et al., 1991), the second scheme is the IMGT numbering (Lefranc, 1997; Lefranc et al., 2005). In a third approach, the intersection of both identification schemes has been used.

With reference to the specific examples of monoclonal chimeric antibodies (MAB-19-0202, MAB-19-0208, MAB-19-0217, MAB-19-0223 and MAB-19-0233) and monoclonal humanized antibodies of the invention, the respective sequences are shown in Tables 1, 2, 4 and 5 of the Examples. The exemplary humanized antibodies MAB-19-0603, MAB-19-0608, MAB-19-0613 and MAB-19-0618 of the invention are humanized variants of MAB-19-0202, while the exemplary humanized antibodies MAB-19-0583, MAB-19-0594 and MAB-19-0598 of the invention are humanized variants of MAB-19-0233.

The antibodies of the invention can in principle be antibodies of any isotype. The choice of isotype typically will be guided by the desired Fc-mediated effector functions, such as ADCC or CDC induction, or the requirement for an antibody devoid of Fc-mediated effector function (“inert” antibody). Exemplary isotypes are IgG1, IgG2, IgG3, and IgG4. Either of the human light chain constant regions, kappa or lambda, may be used. The effector function of the antibodies of the present invention may be changed by isotype switching to, e.g., an IgG1, IgG2, IgG3, IgG4, IgD, IgA, IgE, or IgM antibody for various therapeutic uses. In one embodiment, the anti-PD-1 antibodies have reduced or depleted effector functions. In one embodiment, the anti-PD-1 antibodies do not mediate ADCC or CDC or both. In one embodiment, the anti-PD-1 antibodies have a constant region of IgG1 isotype, which has reduced or depleted effector function. A reduced or depleted effector function can help to avoid potential toxicity to, e.g., T cells which normally express PD-1.

Antibodies according to the present invention may comprise modifications in the Fc region. When an antibody comprises such modifications, it may become an inert, or non-activating, antibody. The term “inertness”, “inert” or “non-activating” as used herein, refers to an Fc region which is at least not able to bind any Fc-gamma receptors, induce Fc-mediated cross-linking of FcRs, or induce FcR-mediated cross-linking of target antigens via two Fc regions of individual antibodies, or is not able to bind C1q.

Several variants can be constructed to make the Fc region of an antibody inactive for interactions with Fc-gamma receptors and C1q for therapeutic antibody development. Examples of amino acid positions that may be modified, e.g., in an IgG1 isotype antibody, include positions L234, L235 and P331. Combinations thereof, such as L234F/L235E/P331S, can cause a profound decrease in binding to human CD64, CD32, CD16 and C1q (Xu et al., 2000, Cell Immunol. 200(1):16-26; Oganesyan et al., 2008, Acta Cryst. (D64):700-4). Also, L234F and L235E amino acid substitutions can result in Fc regions with abrogated interactions with Fc-gamma receptors and C1q (Canfield et al., 1991, J. Exp. Med. (173):1483-91; Duncan et al., 1988, Nature (332):738-40). A D265A amino acid substitution can decrease binding to all Fcy receptors and prevent ADCC (Shields et al., 2001, J. Biol. Chem. (276):6591-604). Binding to C1q can be abrogated by mutating positions D270, K322, P329, and P331. Mutating these positions to either D270A or K322A or P329A or P331A can make the antibody deficient in CDC activity (Idusogie E E, et al., 2000, J Immunol. 164: 4178-84). Alternatively, human IgG2 and IgG4 subclasses are considered naturally compromised in their interactions with C1q and Fc gamma Receptors although interactions with Fc-gamma receptors were reported (Parren et al., 1992, J. Clin Invest. 90:1537-1546; Bruhns et al., 2009, Blood 113: 3716-3725). Mutations abrogating these residual interactions can be made in both isotypes, resulting in reduction of unwanted side-effects associated with FcR binding. For IgG2, these include L234A and G237A, and for IgG4, L235E. Another suitable inertness mutation is P329G. In one embodiment, a combination of L234, L235 and P329 inertness mutations may be used, for example a combination of L234A, L235A and P329G.

The antibodies of the present invention can be used synergistically with traditional chemotherapeutic agents or other immune therapies attacking tumors, for example by employing other antibodies targeting tumor antigens thereby inducing an immune response against these tumors cells or by employing other checkpoint inhibitors or activators or angiogenesis inhibitors.

Antibody-dependent cell-mediated cytotoxicity is also referred to as “ADCC” herein. ADCC describes the cell-killing ability of effector cells as described herein, in particular lymphocytes, which preferably requires the target cell being marked by an antibody. ADCC preferably occurs when antibodies bind to antigens on tumor cells and the antibody Fc domains engage Fc receptors (FcR) on the surface of immune effector cells. Several families of Fc receptors have been identified, and specific cell populations characteristically express defined Fc receptors. ADCC can be viewed as a mechanism to directly induce a variable degree of immediate tumor destruction that leads to antigen presentation and the induction of tumor-directed T-cell responses. Preferably, in vivo induction of ADCC will lead to tumor-directed T-cell responses and host-derived antibody responses.

Complement-dependent cytotoxicity is also referred to as “CDC” herein. CDC is another cell-killing method that can be directed by antibodies. IgM is the most effective isotype for complement activation. IgG1 and IgG3 are also both very effective at directing CDC via the classical complement-activation pathway. Preferably, in this cascade, the formation of antigen-antibody complexes results in the uncloaking of multiple C1q binding sites in close proximity on the CH2 domains of participating antibody molecules such as IgG molecules (C1q is one of three subcomponents of complement C1). Preferably these uncloaked C1q binding sites convert the previously low-affinity C1q-IgG interaction to one of high avidity, which triggers a cascade of events involving a series of other complement proteins and leads to the proteolytic release of the effector-cell chemotactic/activating agents C3a and C5a.

Preferably, the complement cascade ends in the formation of a membrane attack complex, which creates pores in the cell membrane that facilitate free passage of water and solutes into and out of the cell.

II. Production of Antibodies

Antibodies of the invention can be produced by a variety of techniques, including conventional monoclonal antibody methodology, e.g., the standard somatic cell hybridization technique of Kohler and Milstein, Nature 256: 495 (1975). Although somatic cell hybridization procedures are preferred, in principle, other techniques for producing monoclonal antibodies can be employed, e.g., viral or oncogenic transformation of B-lymphocytes or phage display techniques using libraries of antibody genes.

The preferred animal system for preparing hybridomas that secrete monoclonal antibodies is the murine system. Hybridoma production in the mouse is a very well established procedure. Immunization protocols and techniques for isolation of immunized splenocytes for fusion are known in the art. Fusion partners (e.g., murine myeloma cells) and fusion procedures are also known.

Other preferred animal systems for preparing hybridomas that secrete monoclonal antibodies are the rat and the rabbit system (e.g., described in Spieker-Polet et al., Proc. Natl. Acad. Sci. U.S.A. 92:9348 (1995), see also Rossi et al., Am. J. Clin. Pathol. 124: 295 (2005)).

In yet another preferred embodiment, human monoclonal antibodies directed against PD-1 can be generated using transgenic or transchromosomal mice carrying parts of the human immune system rather than the mouse system. These transgenic and transchromosomic mice include mice known as HuMAb mice and KM mice, respectively, and are collectively referred to herein as “transgenic mice”. The production of human antibodies in such transgenic mice can be performed as described in detail for CD20 in WO 2004/035607.

Yet another strategy for generating monoclonal antibodies is to directly isolate genes encoding antibodies from lymphocytes producing antibodies of defined strategy e.g. see Babcock et al., 1996; A novel strategy for generating monoclonal antibodies from single, isolated lymphocytes producing antibodies of defined strategy. For details of recombinant antibody engineering see also Welschof and Kraus, Recombinant antibodies for cancer therapy ISBN-0-89603-918-8 and Benny K. C. Lo Antibody Engineering ISBN 1-58829-092-1.

Immunizations

To generate antibodies to PD-1, animals, for example rabbits or mice, can be immunized with carrier-conjugated peptides derived from the PD-1 sequence, an enriched preparation of recombinantly expressed PD-1 antigen or fragments thereof and/or cells expressing PD-1, as described. Alternatively, rabbits or mice can be immunized with DNA encoding full length human PD-1 or fragments thereof. In the event that immunizations using a purified or enriched preparation of the PD-1 antigen do not result in antibodies, rabbits or mice can also be immunized with cells expressing PD-1, e.g., a cell line, to promote immune responses.

The immune response can be monitored over the course of the immunization protocol with plasma and serum samples being obtained by tail vein or retroorbital bleeds. Rabbits or mice with sufficient titers of anti-PD-1 immunoglobulin can be used for fusions. Rabbits or mice can be boosted intraperitonealy or intravenously with PD-1 expressing cells 3 days before sacrifice and removal of the spleen to increase the rate of specific antibody secreting hybridomas.

Generation of Hybridomas Producing Monoclonal Antibodies

To generate hybridomas producing monoclonal antibodies to PD-1, splenocytes and lymph node cells from immunized animals, e.g., rabbits or mice, can be isolated and fused to an appropriate immortalized cell line, such as a mouse or rabbit myeloma cell line. The resulting hybridomas can then be screened for the production of antigen-specific antibodies. Individual wells can then be screened by ELISA for antibody secreting hybridomas. By immunofluorescence and FACS analysis using PD-1 expressing cells, antibodies with specificity for PD-1 can be identified. The antibody secreting hybridomas can be replated, screened again, and if still positive for anti-PD-1 monoclonal antibodies can be subcloned by limiting dilution. The stable subclones can then be cultured in vitro to generate antibody in tissue culture medium for characterization.

Generation of Transfectomas Producing Monoclonal Antibodies

Antibodies of the invention also can be produced in a host cell transfectoma using, for example, a combination of recombinant DNA techniques and gene transfection methods as are well known in the art (Morrison, S. (1985) Science 229: 1202).

For example, in one embodiment, the gene(s) of interest, e.g., antibody genes, can be ligated into an expression vector such as a eukaryotic expression plasmid such as used by the GS gene expression system disclosed in WO 87/04462, WO 89/01036 and EP 338 841 or other expression systems well known in the art. The purified plasmid with the cloned antibody genes can be introduced in eukaryotic host cells such as CHO cells, NS/0 cells, Sp2/0 cells, COS cells, Vero cells, HeLa cells, HEK293T cells, HEK293T/17 or HEK293 cells or alternatively other eukaryotic cells like plant derived cells, fungal or yeast cells. The method used to introduce these genes can be methods described in the art such as electroporation, lipofectine, lipofectamine or others. After introduction of these antibody genes in the host cells, cells expressing the antibody can be identified and selected. These cells represent the transfectomas which can then be amplified for their expression level and upscaled to produce antibodies. Recombinant antibodies can be isolated and purified from these culture supernatants and/or cells.

Alternatively, the cloned antibody genes can be expressed in other expression systems, including prokaryotic cells, such as microorganisms, e.g., E. coli. Furthermore, the antibodies can be produced in transgenic non-human animals, such as in milk from sheep and rabbits or in eggs from hens, or in transgenic plants; see e.g. Verma, R., et al. (1998) J. Immunol. Meth. 216: 165-181; Pollock, et al. (1999) J. Immunol. Meth. 231: 147-157; and Fischer, R., et al. (1999) Biol. Chem. 380: 825-839.

Antibodies of the invention also can be produced in genetically modified viruses, such as RNA viruses, using recombinant DNA techniques well known to persons skilled in the art.

Recombinant viral genomes, which can be used to rescue virus particles expressing an antibody or a fragment thereof, can for example be obtained by a method called ‘reverse genetics’.

Use of Partial Antibody Sequences to Express Intact Antibodies (i.e., Humanization and Chimerisation).

a) Chimerization

Murine or rabbit monoclonal antibodies can be used as therapeutic antibodies in humans, but as these antibodies can be highly immunogenic in man when repetitively applied, this may lead to a reduction of the therapeutic effect. The main immunogenicity is mediated by the heavy chain constant regions. The immunogenicity of murine or rabbit antibodies in man can be reduced or completely avoided if respective antibodies are chimerized or humanized. Chimeric antibodies are antibodies, the different portions of which are derived from different animal species, such as those having a variable region derived from a murine or rabbit antibody and a human immunoglobulin constant region. Chimerisation of antibodies is achieved by joining of the variable regions of the murine or rabbit antibody heavy and light chain with the constant region of human heavy and light chain (e.g., as described by Kraus et al., in Methods in Molecular Biology series, Recombinant antibodies for cancer therapy, ISBN-0-89603-918-8). In a preferred embodiment, chimeric antibodies are generated by joining human kappa-light chain constant region to murine or rabbit light chain variable region. In an also preferred embodiment chimeric antibodies can be generated by joining human lambda-light chain constant region to murine or rabbit light chain variable region. The preferred heavy chain constant regions for generation of chimeric antibodies are IgG1, IgG3 and IgG4. Other preferred heavy chain constant regions for generation of chimeric antibodies are IgG2, IgA, IgD and IgM.

b) Humanization

Antibodies interact with target antigens predominantly through amino acid residues that are located in the six heavy and light chain complementarity determining regions (CDRs). For this reason, the amino acid sequences within CDRs are more diverse between individual antibodies than sequences outside of CDRs. Because CDR sequences are responsible for most antibody-antigen interactions, it is possible to express recombinant antibodies that mimic the properties of specific naturally occurring antibodies by constructing expression vectors that include CDR sequences from the specific naturally occurring antibody grafted onto framework sequences from a different antibody with different properties (see, e.g., Riechmann, L. et al. (1998) Nature 332: 323-327; Jones, P. et al. (1986) Nature 321: 522-525; and Queen, C. et al. (1989) Proc. Natl. Acad. Sci. U.S.A 86: 10029-10033). Such framework sequences can be obtained from public DNA databases that include germline antibody gene sequences. These germline sequences will differ from mature antibody gene sequences because they will not include completely assembled variable genes, which are formed by V (D) J joining during B cell maturation. Germline gene sequences will also differ from the sequences of a high affinity secondary repertoire antibody at individual evenly across the variable region. For example, somatic mutations are relatively infrequent in the amino terminal portion of framework region 1 and in the carboxy-terminal portion of framework region 4. Furthermore, many somatic mutations do not significantly alter the binding properties of the antibody. For this reason, it is not necessary to obtain the entire DNA sequence of a particular antibody in order to recreate an intact recombinant antibody having binding properties similar to those of the original antibody (see WO 99/45962). Partial heavy and light chain sequences spanning the CDR regions are typically sufficient for this purpose. The partial sequence is used to determine which germline variable and joining gene segments contributed to the recombined antibody variable genes. The germline sequence is then used to fill in missing portions of the variable regions. Heavy and light chain leader sequences are cleaved during protein maturation and do not contribute to the properties of the final antibody. To add missing sequences, cloned cDNA sequences can be combined with synthetic oligonucleotides by ligation or PCR amplification. Alternatively, the entire variable region can be synthesized as a set of short, overlapping, oligonucleotides and combined by PCR amplification to create an entirely synthetic variable region clone. This process has certain advantages such as elimination or inclusion or particular restriction sites, or optimization of particular codons.

The nucleotide sequences of heavy and light chain transcripts from hybridomas are used to design an overlapping set of synthetic oligonucleotides to create synthetic V sequences with identical amino acid coding capacities as the natural sequences. The synthetic heavy and kappa chain sequences can differ from the natural sequences in three ways: strings of repeated nucleotide bases are interrupted to facilitate oligonucleotide synthesis and PCR amplification; optimal translation initiation sites are incorporated according to Kozak's rules (Kozak, 1991, J. Biol. Chem. 266: 19867-19870); and HindIII sites are engineered upstream of the translation initiation sites.

For both the heavy and light chain variable regions, the optimized coding and corresponding non-coding, strand sequences are broken down into 30-50 nucleotides approximately at the midpoint of the corresponding non-coding oligonucleotide. Thus, for each chain, the oligonucleotides can be assembled into overlapping double stranded sets that span segments of 150-400 nucleotides. The pools are then used as templates to produce PCR amplification products of 150-400 nucleotides. Typically, a single variable region oligonucleotide set will be broken down into two pools which are separately amplified to generate two overlapping PCR products. These overlapping products are then combined by PCR amplification to form the complete variable region. It may also be desirable to include an overlapping fragment of the heavy or light chain constant region in the PCR amplification to generate fragments that can easily be cloned into the expression vector constructs.

The reconstructed chimerized or humanized heavy and light chain variable regions are then combined with cloned promoter, leader, translation initiation, constant region, 3′ untranslated, polyadenylation, and transcription termination sequences to form expression vector constructs. The heavy and light chain expression constructs can be combined into a single vector, co-transfected, serially transfected, or separately transfected into host cells which are then fused to form a host cell expressing both chains. Plasmids for use in construction of expression vectors for human IgGκ are available for the skilled person. The plasmids can be constructed so that PCR amplified V heavy and V kappa light chain cDNA sequences could be used to reconstruct complete heavy and light chain minigenes. These plasmids can be used to express completely human, or chimeric IgG1, Kappa or IgG4, Kappa antibodies. Similar plasmids can be constructed for expression of other heavy chain isotypes, or for expression of antibodies comprising lambda light chains.

Thus, according to the present invention, the structural features of the anti-PD-1 antibodies of the invention, can be used to create structurally related humanized anti-PD-1 antibodies that retain at least one functional property of the antibodies of the invention, such as binding to PD-1. More specifically, one or more CDR regions as disclosed herein can be combined recombinantly with known human framework regions and CDRs to create additional, recombinantly engineered, humanized anti-PD-1 antibodies of the invention.

III. Characterization of Antibodies

Binding to Antigen Expressing Cells

The ability of the antibodies to bind PD-1 and/or to block the PD-1/ligand interaction can be determined using standard binding assays, reporter gene blockade assays, T cell proliferation assays, etc., such as those set forth in the examples.

Characterization of Binding of Antibodies

To purify anti-PD-1 antibodies, selected hybridomas can be grown in two-liter spinner-flasks for monoclonal antibody purification. Alternatively, anti-PD-1 antibodies can be produced in dialysis based bioreactors. Supernatants can be filtered and, if necessary, concentrated before affinity chromatography with protein G-sepharose or protein A-sepharose. Eluted IgG can be checked by gel electrophoresis and high performance liquid chromatography to ensure purity. The buffer solution can be exchanged into PBS, and the concentration can be determined by OD280 using 1.43 extinction coefficient. The monoclonal antibodies can be aliquoted and stored at −80° C. To determine if the selected anti-PD-1 monoclonal antibodies bind to unique epitopes, site-directed or multi-site directed mutagenesis can be used.

Determining the PD-1 Binding Specificity

The binding potency of anti-PD-1 antibodies to PD-1 can be determined by ELISA techniques. For example, PD-1/Fc chimera can be coated on microtiter plates. After blocking, the anti-PD-1-antibodies to be tested can be added and incubated. Then, after performing a washing procedure, anti-human-IgG coupled to e.g., horseradish peroxidase can be added for detection.

The binding ability of anti-PD-1 antibodies to cell surface expressed PD-1 can be analyzed using HEK-293 cells ectopically expressing PD-1. Anti-PD-1 antibodies can be added to these cells at various concentrations and incubated. Anti-Ig antibodies conjugated with a fluorescence tag can be added then and cell-associated immunofluorescent signals can be recorded.

Determining the Blocking Ability

The potency of anti-PD-1 antibodies to block the PD-1/PD-L1 interaction can be analyzed using a PD-1/PD-L1 blockade bioassay. PD-L1 expressing cells can be incubated with the antibodies to be tested at various concentrations. After adding PD-1 expressing effector cells and incubating the thus obtained mixture, for example, a luciferase assay reagent can be added and the luminescene can be determined. A PD-1/PD-L1 blockade bioassay (Promega, Cat No. J12150), or comparable kits, may be used as described by the manufacturer.

For characterizing the ability of the anti-PD1 antibodies to induce T-cell proliferation in an antigen-specific assay with active PD-1/PD-L1 axis, dendritic cells (DCs), expressing a tumor antigen, can be performed. Such an assay is detailed, in a non-limiting manner, in Example 5 below.

Flow Cytometric Analysis and Immunofluorescence Microscopy

In order to demonstrate presence of anti-PD-1 antibodies in sera of immunized animals or binding of monoclonal antibodies to living cells expressing PD-1, flow cytometry or immunofluorescence microscopy analysis can be used in a manner well known to the skilled person.

Epitope Mapping Mapping of epitopes recognized by antibodies of invention can be performed as described in detail in “Epitope Mapping Protocols”, Methods in Molecular Biology by Glenn E. Morris ISBN-089603-375-9 and in “Epitope Mapping: A Practical Approach”, Practical Approach Series, 248 by Olwyn M. R. Westwood, Frank C. Hay.

IV. Bispecific/Multispecific Antibodies which Bind to PD-1

In yet another embodiment of the invention, antibodies to PD-1 can be derivatized or linked to another functional molecule, e.g., another peptide or protein (e.g., a Fab′ fragment) to generate a bispecific or multispecific molecule which binds to multiple binding sites or target epitopes. For example, an antibody of the invention can be functionally linked (e.g., by chemical coupling, genetic fusion, noncovalent association or otherwise) to one or more other binding molecules, such as another antibody, peptide or binding mimetic.

Accordingly, the present invention includes bispecific and multispecific molecules comprising at least one first binding specificity for PD-1 and a second binding specificity (or further binding specifities) for a second target epitope (or for further target epitopes).

The second binding specifity can be directed to another immune checkpoint, thereby either inhibiting or activating/stimulating the respective checkpoint. Other checkpoint inhibitors which may be targeted include, but are not limited to CTLA4, PD-L1, TIM-3, KIR or LAG-3. Checkpoint activators, which may be targeted by the second binding specifity include, but are not limited to CD27, CD28, CD40, CD122, CD137, OX40, GITR, or ICOS. Therefore, the invention includes bispecific and multispecific molecules capable of binding both to at least one other checkpoint and to inhibit PD-1 by a respective binding. The second binding specifity may be antagonistic, such as anti-CTLA4, anti-PD-L1, anti-TIM-3, anti-KIR or anti-LAG-3, or may be agonistic, such as anti-CD27, anti-CD28, anti-CD40, anti-CD122, anti-CD137, anti-OX40, anti-GITR, or anti-ICOS. Also encompassed by the present invention are multispecific molecules capable of binding to PD-1 and in addition to at least one other immune checkpoint. Preferred combinations of binding specifities include anti-PD1 and anti-PD-L 1 or anti-PD-1 and anti-CTLA4.

For example, CD28 provides a stimulative inducement that could be necessary for the activation of T cells. The same applies e.g., for CD137. CD137 (4-1BB, TNFRSF9) is a member of the tumor necrosis factor (TNF) receptor (TNFR) superfamily. CD137 is a costimulatory molecule on CD8⁺ and CD4⁺ T cells, regulatory T cells (Tregs), natural killer (NK) and NKT cells, B cells and neutrophils. On T cells, CD137 is not constitutively expressed, but induced upon T-cell receptor (TCR) activation. Stimulation via its natural ligand 4-1BBL or agonist antibodies leads to signaling using TNFR-associated factor (TRAF)-2 and TRAF-1 as adaptors. Early signaling by CD137 involves K-63 poly-ubiquitination reactions that ultimately result in activation of the nuclear factor (NF)-κB and mitogen-activated protein (MAP)-kinase pathways. Signaling leads to increased T cell co-stimulation, proliferation, cytokine production, maturation and prolonged CD8⁺ T-cell survival. Agonistic antibodies against CD137 have been shown to promote anti-tumor control by T cells in various pre-clinical models (Murillo et al. 2008 Clin. Cancer Res. 14(21): 6895-6906). Antibodies stimulating CD137 can induce survival and proliferation of T cells, thereby enhancing the anti-tumor immune response. Antibodies stimulating CD137 have been disclosed in the prior art, and include urelumab, a human IgG4 antibody (WO 2005/035584) and utomilumab, a human IgG2 antibody (Fisher et al. 2012 Cancer Immunol. Immunother. 61: 1721-1733).

Alternatively, the second binding specifity can provide an antiangiogenesis activity. Thus, the second binding specifity can be capable of targeting vascular endothelial growth factor (VEGF) or its receptor VEGFR (for example VEGFR1, 2, 3). Alternatively or in addition, the second binding specifity may be capable of targeting PDGFR, c-Kit, Raf and/or RET.

It is also encompassed by the present invention that the second or the further binding specifities of the bispecific or multispecific molecules of the present invention can be directed to and are capable of binding to a tumor antigen. The tumor antigen can be a surface antigen or an antigen presented in the context of MHC. The binding specificity could for example be based on a B-cell receptor (antibody) or a T cell receptor.

The term “tumor antigen” as used herein refers to a constituent of cancer cells which may be derived from the cytoplasm, the cell surface and the cell nucleus. In particular, it refers to those antigens which are produced intracellularly or as surface antigens on tumor cells. A tumor antigen is typically expressed preferentially by cancer cells (e.g., it is expressed at higher levels in cancer cells than in non-cancer cells) and in some instances it is expressed solely by cancer cells. Examples of tumor antigens include, without limitation, p53, ART-4, BAGE, beta-catenin/m, Bcr-abL CAMEL, CAP-1, CASP-8, CDC27/m, CDK4/m, CEA, the cell surface proteins of the claudin family, such as CLAUDIN-6, CLAUDIN-18.2 and CLAUDIN-12, c-MYC, CT, Cyp-B, DAM, ELF2M, ETV6-AML1, G250, GAGE, GnT-V, Gap 100, HAGE, HER-2/neu, HPV-E7, HPV-E6, HAST-2, hTERT (or hTRT), LAGE, LDLR/FUT, MAGE-A, preferably MAGE-A1, MAGE-A2, MAGE-A3, MAGE-A4, MAGE-A5, MAGE-A6, MAGE-A7, MAGE-A8, MAGE-A9, MAGE-A10, MAGE-A11, or MAGE-A12, MAGE-B, MAGE-C, MART-1/Melan-A, MC1R, Myosin/m, MUC1, MUM-1, MUM-2, MUM-3, NA88-A, NF1, NY-ESO-1, NY-BR-1, p190 minor BCR-abL, Pml/RARa, PRAME, proteinase 3, PSA, PSM, RAGE, RU1 or RU2, SAGE, SART-1 or SART-3, SCGB3A2, SCP1, SCP2, SCP3, SSX, SURVIVIN, TEL/AML1, TPI/m, TRP-1, TRP-2, TRP-2/INT2, TPTE, WT, and WT-1.

In one embodiment, the second antigen to be targeted is selected from the group consisting of NY-ESO-1 (UniProt P78358), Tyrosinase (UniProt P14679), MAGE-A3 (UniProt P43357), TPTE (UniProt P56180), KLK2 (UniProt P20151), PSA(KLK3) (UniProt P07288), PAP(ACPP, UniProt P15309), HOXB13 (UniProt Q92826), NKX3-1 (UniProt Q99801), HPV16 E6/E7 (UniProt P03126/P03129); HPV18 E6/E7 (UniProt P06463/P06788); HPV31 E6/E7 (UniProt P17386/P17387); HPV33 E6/E7 (UniProt P06427/P06429); HPV45 E6/E7 (UniProt P21735/P21736); HPV58 E6/E7 (UniProt P26555/P26557), PRAME (UniProt P78395), ACTL8 (UniProt Q9H568), CXorf61 (KKLC1, UniProt Q5H943), MAGE-A9B (UniProt P43362), CLDN6 (UniProt P56747), PLAC1 (UniProt Q9HBJ0), and p53 (UniProt P04637).

Methods of treatment involving these antigens may aim at the treatment of cancer, wherein the cancer cells are characterized by expression of the respective antigen. It is also possible to use antigens described herein, in particular NY-ESO-1, Tyrosinase, MAGE-A3, TPTE, KLK2, PSA(KLK3), PAP(ACPP), HOXB13, NKX3-1, HPV16 E6/E7; HPV18 E6/E7; HPV31 E6/E7; HPV33 E6/E7; HPV45 E6/E7; HPV58 E6/E7, PRAME, ACTL8, CXorf61 (KKLC1), MAGE-A9B, CLDN6, PLAC1, and p53, in combination. Methods of treatment involving such combination of antigens may aim at the treatment of cancer, wherein the cancer cells are characterized by expression of two or more antigens of the respective combination of antigens or wherein the cancer cells of a large fraction (e.g., at least 80%, at least 90% or even more) of patients having a certain cancer to be treated express one or more of the respective antigens of a combination. Such combination may comprise a combination of at least 2, at least 3, at least 4, at least 5, or at least 6 antigens. Thus, the combination may comprise 3, 4, 5, 6, 7, or 8 antigens.

For the treatment of cutaneous melanoma the further binding specitity/specifities may at least target one of the following antigens: NY-ESO-1, Tyrosinase, MAGE-A3, and/or TPTE.

For the treatment of prostate cancer the further binding specitity/specifities may at least target one of the following antigens: KLK2, PSA(KLK3), PAP(ACPP), HOXB13, and/or NKX3-1.

For the treatment of breast cancer the further binding specitity/specifities may at least target one of the following antigens: PRAME, ACTL8, CXorf61 (KKLC1), MAGEA3, MAGE-A9B, CLDN6, NY-ESO-1, and/or PLAC1.

For the treatment of ovarian cancer the further binding specitity/specifities may at least target one of the following antigens: CLDN6, p53, and/or PRAME.

Bispecific and multispecific molecules of the invention can further include a third binding specificity, in addition to a tumor antigen specificity and an anti-PD-1 binding specificity. In one embodiment, the third binding specificity is directed to an Fc receptor, e.g., human Fc-gammaRI (CD64) or a human Fc-alpha receptor (CD89). Therefore, the invention includes multispecific molecules capable of binding to PD-1, to Fc-gammaR, Fc-alphaR or Fc-epsilonR expressing effector cells (e.g., monocytes, macrophagesor polymorphonuclear cells (PMNs)), and to target cancer cells expressing a tumor antigen. These multispecific molecules may may trigger Fc receptor-mediated effector cell activities, such as phagocytosis of tumor antigen expressing cells, antibody dependent cellular cytotoxicity (ADCC), cytokine release, or generation of superoxide anion.

In another embodiment, the third binding specificity is an anti-enhancement factor (EF) portion, e.g., a molecule which binds to a surface protein involved in cytotoxic activity and thereby increases the immune response against the target cell. The “anti-enhancement factor portion” can be an antibody, functional antibody fragment or a ligand that binds to a given molecule, e.g., an antigen or a receptor, and thereby results in an enhancement of the effect of the binding determinants for the Fc receptor or target cell antigen. The “anti-enhancement factor portion” can bind an Fc receptor or a target cell antigen. Alternatively, the anti-enhancement factor portion can bind to an entity that is different from the entity to which the first and second binding specificities bind. For example, the anti-enhancement factor portion can bind a cytotoxic T cell (e.g., via CD2, CD3, CD8, CD28, CD4, CD40, ICAM-1 or other immune cell that results in an increased immune response against the target cell).

In one embodiment, the bispecific and multispecific molecules of the invention comprise as a binding specificity at least one antibody, including, e.g., a Fab, Fab′, F(ab′)₂, Fv, or a single chain Fv. The antibody may also be a light chain or heavy chain dimer, or any minimal fragment thereof such as a Fv or a single chain construct as described in Ladner et al., U.S. Pat. No. 4,946,778. The antibody may also be a binding-domain immunoglobulin fusion protein as disclosed in US 2003/0118592 and US 2003/0133939.

As used herein, the term “effector cell” refers to an immune cell which is involved in the effector phase of an immune response, as opposed to the cognitive and activation phases of an immune response. Exemplary immune cells include cells of myeloid or lymphoid origin, e.g, lymphocytes (e.g., B cells and T cells including cytolytic T cells (CTLs), killer cells, natural killer cells, macrophages, monocytes, eosinophils, neutrophils, polymorphonuclear cells, granulocytes, mast cells, and basophils. Some effector cells express specific Fc receptors and carry out specific immune functions. In preferred embodiments, an effector cell is capable of inducing antibody-dependent cellular cytotoxicity (ADCC), e.g., a neutrophil capable of inducing ADCC. For example, natural killer cells, monocytes, macrophages, which express FcR are involved in specific killing of target cells and presenting antigens to other components of the immune system, or binding to cells that present antigens. In other embodiments, an effector cell can phagocytose a target antigen, target cell, or microorganism. The expression of a particular FcR on an effector cell can be regulated by humoral factors such as cytokines. For example, expression of Fc-gammaRI has been found to be up-regulated by interferon gamma (IFN-γ). This enhanced expression increases the cytotoxic activity of Fc-gammaRI-bearing cells against targets. An effector cell can phagocytose or lyse a target antigen or a target cell.

“Target cell” shall mean any undesirable cell in a subject (e.g., a human or animal) that can be targeted by an antibody of the invention. In preferred embodiments, the target cell is a tumor cell.

Bispecific and multispecific molecules of the present invention can be made using chemical techniques (see e.g., D. M. Kranz et al. (1981) Proc. Natl. Acad. Sci. USA 78:5807), “polydoma” techniques (see U.S. Pat. No. 4,474,893, to Reading), or recombinant DNA techniques.

In particular, bispecific and multispecific molecules of the present invention can be prepared by conjugating the constituent binding specificities, e.g., the anti-CTLA4 and anti-PD-1 binding specificities, using methods known in the art. For example, each binding specificity of the bispecific and multispecific molecule can be generated separately and then conjugated to one another. When the binding specificities are proteins or peptides, a variety of coupling or cross-linking agents can be used for covalent conjugation. Examples of cross-linking agents include protein A, carbodiimide, N-succinimidyl-S-acetyl-thioacetate (SATA), 5,5′-dithiobis(2-nitrobenzoic acid) (DTNB), o-phenylenedimaleimide (oPDM), N-succinimidyl-3-(2-pyridyldithio)propionate (SPDP), and sulfosuccinimidyl-4-(N-maleimidomethyl)cyclo-hexane-1-carboxylate (sulfo-SMCC) (see e.g., Karpovsky et al. (1984) J. Exp. Med. 160: 1686; Liu, M A et al. (1985) Proc. Natl. Acad. Sci. USA 82: 8648). Other methods include those described by Paulus (Behring Ins. Mitt. (1985) No. 78,118-132); Brennan et al. (Science (1985) 229: 81-83), and Glennie et al. (J. Immunol. (1987) 139: 2367-2375). Preferred conjugating agents are SATA and sulfo-SMCC, both available from Pierce Chemical Co. (Rockford, IL).

When the binding specificities are antibodies, they can be conjugated via sulfhydryl bonding of the C-terminus hinge regions of the two heavy chains. In a particularly preferred embodiment, the hinge region is modified to contain an odd number of sulfhydryl residues, preferably one, prior to conjugation.

Alternatively, both binding specificities can be encoded in the same vector and expressed and assembled in the same host cell. This method is particularly useful where the bispecific and multispecific molecule is a mAb x mAb, mAb x Fab, Fab x F(ab′)₂ or ligand x Fab fusion protein. A bispecific and multispecific molecule of the invention, e.g., a bispecific molecule, can be a single chain molecule, such as a single chain bispecific antibody, a single chain bispecific molecule comprising one single chain antibody and a binding determinant, or a single chain bispecific molecule comprising two binding determinants. Bispecific and multispecific molecules can also be single chain molecules or may comprise at least two single chain molecules. Methods for preparing bi- and multispecific molecules are described for example in U.S. Pat. Nos. 5,260,203; 5,455,030; 4,881,175; 5,132,405; 5,091,513; 5,476,786; 5,013,653; 5,258,498; and 5,482,858. Accordingly, the present invention encompasses all these antibody formats.

Binding of the bispecific and multispecific molecules to their specific targets can be confirmed by enzyme-linked immunosorbent assay (ELISA), a radioimmunoassay (RIA), FACS analysis, a bioassay (e.g., growth inhibition), or a Western Blot Assay. Each of these assays generally detects the presence of protein-antibody complexes of particular interest by employing a labeled reagent (e.g., an antibody) specific for the complex of interest. For example, the FcR-antibody complexes can be detected using e.g., an enzyme-linked antibody or antibody fragment which recognizes and specifically binds to the antibody-FcR complexes. Alternatively, the complexes can be detected using any of a variety of other immunoassays. For example, the antibody can be radioactively labeled and used in a radioimmunoassay (RIA) (see, for example, Weintraub, B., Principles of Radioimmunoassays, Seventh Training Course on Radioligand Assay Techniques, The Endocrine Society, March, 1986). The radioactive isotope can be detected by such means as the use of a γ-counter or a scintillation counter or by autoradiography.

V. Immunoconjugates

In another aspect, the present invention features an anti-PD-1 antibody conjugated to a moiety or agent. Such conjugates are referred to herein also as “immunoconjugates”.

The moiety or agent can be an enzyme bound to the antibody. Such antibodies can be used for enzyme immunoassays, such as enzyme-linked immunosorbent assays (ELISA) or enzyme multiplied immunoassay technique (EMIT), or Westernblots for example.

Alternatively or in addition, a radionuclide (radioisotope) can be bound to the antibody as a moiety or agent. Such conjugates may be used in therapy but also for diagnostic purposes (radioimmunoassays, positron emission tomography (“immuno-PET”)). The radionuclides may be conjugated to the antibodies via complexing agents. Antibodies of the present invention also can be conjugated to a radioisotope, e.g., iodine-131, yttrium-90 or indium-111, to generate cytotoxic radiopharmaceuticals for treating a disorder, such as a cancer. The antibodies according to the invention may be attached to a linker-chelator, e.g., tiuxetan, which allows for the antibody to be conjugated to a radioisotope.

Alternatively or in addition, the moiety or agent may be a tag, for example a fluorescent tag, also known as fluorescent label or fluorescent probe. Ethidium bromide, fluorescein and green fluorescent protein are common tags.

Also encompassed by the present invention are conjugates comprising a therapeutic moiety or a therapeutic agent. The therapeutic moiety or a therapeutic agent may be a cytokine or CD80, which binds to CD28 resulting in a costimulatory signal in the T cell response. The therapeutic moiety or a therapeutic agent may also be a cytotoxin or a drug (e.g., an immunosuppressant). Immunoconjugates which include one or more cytotoxins are referred to as “immunotoxins”. A cytotoxin or cytotoxic agent includes any agent that is detrimental to and, in particular, kills cells. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof.

Suitable therapeutic agents for forming immunoconjugates of the invention include, but are not limited to, antimetabolites (e.g., methotrexate, 6-mercaptopurine, 6-thioguanine, cytarabine, fludarabin, 5-fluorouracil decarbazine), alkylating agents (e.g., mechlorethamine, thioepa chlorambucil, melphalan, carmustine (BSNU) and lomustine (CCNU), cyclophosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC), and anti-mitotic agents (e.g., vincristine and vinblastine). In a preferred embodiment, the therapeutic agent is a cytotoxic agent or a radiotoxic agent. In another embodiment, the therapeutic agent is an immunosuppressant. In yet another embodiment, the therapeutic agent is GM-CSF. In a preferred embodiment, the therapeutic agent is doxorubicin, cisplatin, bleomycin, sulfate, carmustine, chlorambucil, cyclophosphamide or ricin A.

The antibody conjugates of the invention can be used to modify a given biological response, and the drug moiety is not to be construed as limited to classical chemical therapeutic agents. For example, the drug moiety may be a protein or polypeptide possessing a desired biological activity. Such proteins may include, for example, an enzymatically active toxin, or active fragment thereof, such as abrin, ricin A, pseudomonas exotoxin, or diphtheria toxin; a protein such as tumor necrosis factor or interferon-y; or, biological response modifiers such as, for example, lymphokines, interleukin-1 (“IL-1”), interleukin-2 (“IL-2”), interleukin-6 (“IL-6”), granulocyte macrophage colony stimulating factor (“GM-CSF”), granulocyte colony stimulating factor (“G-CSF”), or other growth factors.

Techniques for conjugating such therapeutic moiety to antibodies are well known, see, e.g., Amon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results. And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., “The Preparation And Cytotoxic Properties Of Antibody-Toxin Conjugates”, Immunol. Rev., 62: 119-58 (1982). The moiety, e.g., the therapeutic moiety, or the agent of the conjugate may be conjugated to the antibody by a linker sequence. Suitable linker sequences are known to the skilled person.

VI. Nucleic Acids Encoding an Antibody

In a further aspect the present invention also relates to nucleic acids or nucleic acid molecules comprising genes or nucleic acid sequences encoding antibodies or parts thereof, e.g., an antibody chain, as described herein.

The term “nucleic acid molecule” or “nucleic acid”, as used herein, is intended to include deoxyribonucleic acid (DNA) or ribonucleic acid (RNA) molecules. Nucleic acids include according to the invention genomic DNA, cDNA, mRNA, recombinantly produced and chemically synthesized molecules. According to the invention, a nucleic acid may be present as a single-stranded or double-stranded and linear or covalently circularly closed molecule. For example, the nucleic acid is double-stranded DNA.

The nucleic acids described according to the invention have preferably been isolated. The term “isolated nucleic acid” means according to the invention that the nucleic acid was (i) amplified in vitro, for example by polymerase chain reaction (PCR), (ii) recombinantly produced by cloning, (iii) purified, for example by cleavage and gel-electrophoretic fractionation, or (iv) synthesized, for example by chemical synthesis. An isolated nucleic acid is a nucleic acid which is available for manipulation by recombinant DNA techniques.

Nucleic acids may, according to the invention, be present alone or in combination with other nucleic acids, which may be homologous or heterologous. In preferred embodiments, a nucleic acid is functionally linked to expression control sequences which may be homologous or heterologous with respect to said nucleic acid. The term “homologous” means that a nucleic acid is also functionally linked to the expression control sequence naturally and the term “heterologous” means that a nucleic acid is not functionally linked to the expression control sequence naturally.

A nucleic acid, such as a nucleic acid expressing RNA and/or protein or peptide, and an expression control sequence are “functionally” linked to one another, if they are covalently linked to one another in such a way that expression or transcription of said nucleic acid is under the control or under the influence of said expression control sequence. If the nucleic acid is to be translated into a functional protein, then, with an expression control sequence functionally linked to a coding sequence, induction of said expression control sequence results in transcription of said nucleic acid, without causing a frame shift in the coding sequence or said coding sequence not being capable of being translated into the desired protein or peptide.

The term “expression control sequence” comprises according to the invention promoters, ribosome binding sites, enhancers and other control elements which regulate transcription of a gene or translation of a mRNA. In particular embodiments of the invention, the expression control sequences can be regulated. The exact structure of expression control sequences may vary as a function of the species or cell type, but generally comprises 5′-untranscribed and 5′- and 3′-untranslated sequences (5′-UTR; 3′-UTR) which are involved in initiation of transcription and translation, respectively, such as TATA box, capping sequence, CAAT sequence, and the like. More specifically, 5′-untranscribed expression control sequences comprise a promoter region which includes a promoter sequence for transcriptional control of the functionally linked nucleic acid. Expression control sequences may also comprise enhancer sequences or upstream activator sequences.

According to the invention the term “promoter” or “promoter region” relates to a nucleic acid sequence which is located upstream (5′) to the nucleic acid sequence being expressed and controls expression of the sequence by providing a recognition and binding site for RNA-polymerase. The “promoter region” may include further recognition and binding sites for further factors which are involved in the regulation of transcription of a gene. A promoter may control the transcription of a prokaryotic or eukaryotic gene. Furthermore, a promoter may be “inducible” and may initiate transcription in response to an inducing agent or may be “constitutive” if transcription is not controlled by an inducing agent. A gene which is under the control of an inducible promoter is not expressed or only expressed to a small extent if an inducing agent is absent. In the presence of the inducing agent the gene is switched on or the level of transcription is increased. This is mediated, in general, by binding of a specific transcription factor.

Promoters which are preferred according to the invention include promoters for SP6, T3 and T7 polymerase, human U6 RNA promoter, CMV promoter, and artificial hybrid promoters thereof (e.g., CMV) where a part or parts are fused to a part or parts of promoters of genes of other cellular proteins such as e.g., human GAPDH (glyceraldehyde-3-phosphate dehydrogenase), and including or not including (an) additional intron(s).

According to the invention, the term “expression” is used in its most general meaning and comprises the production of RNA or of RNA and protein/peptide. It also comprises partial expression of nucleic acids. Furthermore, expression may be carried out transiently or stably.

In a preferred embodiment, a nucleic acid molecule is according to the invention present in a vector, where appropriate with a promoter, which controls expression of the nucleic acid. The term “vector” is used here in its most general meaning and comprises any intermediary vehicle for a nucleic acid which enables said nucleic acid, for example, to be introduced into prokaryotic and/or eukaryotic cells and, where appropriate, to be integrated into a genome. Vectors of this kind are preferably replicated and/or expressed in the cells. Vectors comprise plasmids, phagemids, bacteriophages or viral genomes, but also liposomes. The term “plasmid” as used herein generally relates to a construct of extrachromosomal genetic material, usually a circular DNA duplex, which can replicate independently of chromosomal DNA.

Vectors for cloning or for expression, using recombinant techniques, are known in the art, and comprise, e.g., plasmid-based expression vectors, adenovirus vectors, retroviral vectors or baculovirus vectors. Examples of vectors comprise pGEX, pET, pLexA, pBI, pVITRO, pVIVO, and pST, such as pST4.

The vector may be an IVT vector. IVT vectors may be used in a standardized manner as template for in vitro transcription. Such IVT vectors may have the following structure: a 5′ RNA polymerase promoter enabling RNA transcription, followed by a gene of interest which is flanked by either 3′ and/or 5′ untranslated regions (UTR), and a 3′ polyadenyl cassette containing A nucleotides. Optionally, such vectors may, in addition, comprise a nucleic acid sequence encoding for a signal peptide for secretion of the encoded protein. Prior to in vitro transcription, the circular plasmid can be linearized downstream of the polyadenyl cassette by type II restriction enzymes (recognition sequence corresponds to cleavage site). The polyadenyl cassette thus corresponds to the later poly(A) sequence in the transcript. In one embodiment, the vector is an IVT vector based on pST4, preferably comprising a 5′-UTR, 3′-UTR and a 3′ polyadenyl cassette. Optionally, the IVT vector may further comprise a cassette encoding for a signal peptide.

As the 5′-UTR sequence, the 5′-UTR sequence of a human alpha-globin mRNA, optionally with a ‘Kozak sequence’ or an optimized ‘Kozak sequence’ to increase translational efficiency may be used. The 5′-UTR sequence can be the sequence of Homo sapiens hemoglobin subunit alpha 1. Suitable sequences of a 5′-UTR sequence are exemplified in SEQ ID NOs: 94 and 95 (‘Kozak sequence’) of the sequence listing. Alternatively, the 5′-UTR may be a variant of the sequences as depicted in SEQ ID NOs: 94 and 95 of the sequence listing.

As the 3′-UTR sequence, two re-iterated 3′-UTRs of the human beta-globin mRNA may be used and optionally placed between the coding sequence and the poly(A)-tail to assure higher maximum protein levels and prolonged persistence of the mRNA. Alternatively, the 3′-UTR may be a combination of two sequence elements (FI element) derived from the “amino terminal enhancer of split” (AES) mRNA (called F) and the mitochondrial encoded 12S ribosomal RNA (called I). These were identified by an ex vivo selection process for sequences that confer RNA stability and augment total protein expression (see, WO 2017/060314, herein incorporated by reference). Suitable sequences of a 3′-UTR sequence are exemplified in SEQ ID NOs: 101 and 102 of the sequence listing, which may be used to from a ‘FI’-element. Alternatively, the 3′-UTR may be a variant of the sequences as depicted in SEQ ID NOs: 101 and 102 of the sequence listing.

In one embodiment, the IVT nucleic acid vector may further encode/comprise a poly(A)-tail, preferably a poly(A)-tail as is further specified herein. For example, a poly(A)-tail measuring 110 nucleotides in length may be used, consisting of a stretch of 30 adenosine residues, followed by a 10 nucleotide linker sequence (of random nucleotides) and another 70 adenosine residues. This poly(A)-tail sequence was designed to enhance RNA stability and translational efficiency in dendritic cells (see, WO 2016/005324 A1, herein incorporated by reference).

In one embodiment, the vector may comprise a nucleic acid sequence encoding for a signal peptide for secretion of the protein. The secretory signal peptide may be a Homo sapiens MHC class I complex secretory signal peptide, e.g., husec-HLAI-Cw (opt) (GenBank: BAF96505.1).

The aforementioned elements may be positioned in the vector in the following sequences:
(i) 5′-UTR—‘Kozac sequence’—nucleic acid sequence encoding an antibody or an antibody chain or fragment thereof—3′-UTR-poly(A)-tail; or
(ii) 5′-UTR—‘Kozac sequence’—secretory signal peptide—nucleic acid sequence encoding an antibody or an antibody chain or fragment thereof—3′-UTR—poly(A)-tail.

The type of vector for expression of an antibody either can be a vector type in which the antibody heavy chain and light chain are present in different vectors or a vector type in which the heavy chain and light chain are present in the same vector.

In one embodiment, the antibody encoded by the nucleic acid may be an antibody selected from the group consisting of an IgG1, an IgG2, preferably IgG2a and IgG2b, an IgG3, an IgG4, an IgM, an IgA1, an IgA2, a secretory IgA, an IgD, and an IgE antibody. In one embodiment, the antibody is a Fab fragment, F(ab′)₂ fragment, Fv fragment, or a single chain (scFv) antibody. For example, the nucleic acid sequence encoding an antibody or an antibody chain may comprise a nucleic acid sequence encoding an antibody as described herein, e.g., MAB-19-0202, MAB-19-0208, MAB-19-0217, MAB-19-0223, MAB-19-0233, MAB-19-0603, MAB-19-0608, MAB-19-0613, MAB-19-0618, MAB-19-0583, MAB-19-0594, MAB-19-0598), or a heavy chain or a light chain, of one of these antibodies. In one embodiment, the nucleic acid comprises a nucleic acid sequence encoding an antibody chain as described herein.

The antibody chain can be a heavy chain (H chain) or a light chain (L chain), each preferably as described herein. In one embodiment, the H chain comprises a heavy chain variable region (VH) and a heavy chain constant region, wherein the heavy chain constant region can comprise a heavy chain CH₁ constant region or a combination of a heavy chain CH₁ constant region, a heavy chain CH₂ constant region and a heavy chain CH₃ constant region. In one embodiment, the CH₁ constant domain and the CH₂ constant domain can be connected by a hinge region positioned between the CH₁ constant domain and the CH₂ constant domain.

In one embodiment, the L chain comprises a light chain variable region (VL) and a light chain constant region, wherein the light chain constant region can be a CL kappa constant domain or a CL lambda constant domain.

In one embodiment, the nucleic acid encoding an antibody or an antibody chain comprises a nucleic acid sequence encoding a heavy chain variable region (VH) comprising at least one of a HCDR1, HCDR2, and HCDR3 sequence as exemplied herein (SEQ ID NOs: 1-32 of the sequence listing, SYN, RYY). That is the nucleic acid can comprise a nucleic acid sequence encoding HCDR1, HCDR2 or HCDR3 sequence as exemplied herein or the nucleic aid can comprise a nucleic acid sequence encoding for a heavy chain variable region (VH) comprising any of the combination of the HCDR1, HCDR2 and HCDR3 sequence as defined herein. Preferred combinations of the individual HCDR1 to HCDR3 sequences are as specified above with regard to the respective amino acid sequences. This teaching applies accordingly to the nucleic acid sequences.

In one embodiment, the nucleic acid comprises a nucleic acid sequence encoding a light chain variable region (VL) comprising at least one of a LCDR1, LCDR2, and LCDR3 sequence as exemplied herein (SEQ ID NOs: 33-51 of the sequence listing, QAS, DAS). That is the nucleic acid can comprise a nucleic acid sequence encoding LCDR1, LCDR2 or LCDR3 sequence as exemplied herein or the nucleic aid can comprise a nucleic acid sequence encoding for a light chain variable region (VL) comprising any of the combination of the LCDR1, LCDR2 and LCDR3 sequence as defined herein. Preferred combinations of the individual LCDR1 to LCDR3 sequences are as specified above with regard to the respective amino acid sequences. This teaching applies accordingly to the nucleic acid sequences.

In one embodiment, the nucleic acid comprises a nucleic acid sequence encoding VH and VL sequences as exemplified herein (SEQ ID NOs: 52-70 of the sequence listing).

In one embodiment, the nucleic acid comprises a nucleic acid sequence as depicted in SEQ ID NOs: 74-92 of the sequence listing.

In one embodiment, there is provided a nucleic acid or a vector comprising a nucleic acid, such as RNA or an RNA-based vector, or a vector suitable for in vitro transcription, comprising a nucleic acid sequence encoding a heavy chain variable region (VH) and/or a light chain variable region (VL) of an antibody that binds to PD-1, wherein the nucleic acid has at least 70% identity to one of the nucleic acid sequences as depicted in SEQ ID NOs: 74-92 of the sequence listing and encodes for the respective HCDR1, HCDR2 and HCDR3 amino acid sequences and/or LCDR1, LCDR2 and LCDR3 amino acid sequences as depicted in SEQ ID NOs: 1-32 and SEQ ID NOs: 33-51 of the sequence listing.

In one embodiment, the variant nucleic acid sequence has at least 75%, at least 80%, at least 85%, at least 90%, at least 95%, at least 97%, or at least 99% identity to a nucleic acid sequence as set forth in SEQ ID NO: 74 to SEQ ID NO: 92. In one embodiment, nucleotides and nucleotide analogs are considered as identical for determining the degree of identity. For example, uridine (U) and a pseudouridine, e.g., m1ψ, are considered to be identical for determining the degree of identity.

In one embodiment, the variant nucleic acid sequence comprises/encodes for one or more of the respective CDR1, CDR2 and CDR3 amino acid sequences as specified herein. That is, the variant nucleic acid sequence encoding a heavy chain variable region (VH) may comprise/encodes for one or more of a HCDR1, HCDR2 and HCDR3 amino acid sequence as specified herein, wherein for the specific combinations of the CDR sequences reference is made to the respective disclosure herein. For example, the variant nucleic acid sequence can comprise/encode for a HCDR1, HCDR2, and HCDR3 amino acid sequence as specified herein.

The variant nucleic acid sequence encoding a light chain variable region (VL) may comprise/encodes for one or more of a LCDR1, LCDR2 and LCDR3 amino acid sequence as specified herein, wherein for the specific combinations of the CDR sequences reference is made to the respective disclosure herein. For example, the variant nucleic acid sequence can comprise/encode for a LCDR1, LCDR2, and LCDR3 amino acid sequence as specified herein.

The variant nucleic acid sequence may encode for a heavy chain variable region (VH) or a light chain variable region (VL) capable of providing the same binding specificity and/or functionality provided by the heavy chain variable region (VH) or the light chain variable region (VL) of the parent sequence, respectively.

In one embodiment, there is provided a nucleic acid encoding a heavy chain variable region (VH) of an antibody that binds to PD-1, which heavy chain variable region (VH) has the amino acid sequence comprising the amino acid sequences of HCDR1, HCDR2 and HCDR3, wherein the nucleic acid sequence encoding the VH has at least 70% identity to SEQ ID NO: 74 and wherein the encoded HCDR1, HCDR2 and HCDR3 amino acid sequences are selected from:
(i) SYN, SEQ ID NO: 11 and SEQ ID NO: 1, respectively;
(ii) SEQ ID NO: 23, SEQ ID NO: 16 and SEQ ID NO: 1, respectively; or
(iii) SEQ ID NO: 28, SEQ ID NO: 11 and SEQ ID NO: 6, respectively.

In one embodiment, there is provided a nucleic acid encoding a light chain variable region (VL) of an antibody that binds to PD-1, which light chain variable region (VL) has the amino acid sequence comprising the amino acid sequences of LCDR1, LCDR2 and LCDR3, wherein the nucleic acid sequence encoding the VL has at least 70% identity to SEQ ID NO: 79 and wherein the encoded LCDR1, LCDR2 and LCDR3 amino acids sequences are selected from:
(i) SEQ ID NO: 42, QAS, and SEQ ID NO: 33, respectively; or
(ii) SEQ ID NO: 47, SEQ ID NO: 38, and SEQ ID NO: 33, respectively.

In one embodiment, the above VH variant has at least 70%, at least 75%, at least 80%, at least 85%, at least 90%, at least 95%, at least 97%, or at least 99% identity to a nucleic acid sequence as set forth in SEQ ID NO: 74. In one embodiment, the above VL variant has at least 70%, at least 75%, at least 80%, at least 85%, at least 90%, at least 95%, at least 97%, or at least 99% identity to a nucleic acid sequence as set forth in SEQ ID NO: 79.

In one embodiment, there is provided a nucleic acid encoding a heavy chain variable region (VH) of an antibody that binds to PD-1, which heavy chain variable region (VH) has the amino acid sequence comprising the amino acid sequences of HCDR1, HCDR2 and HCDR3, wherein the nucleic acid sequence encoding the VH has at least 70% identity to SEQ ID NO: 75 and wherein the encoded HCDR1, HCDR2 and HCDR3 amino acid sequences are selected from:
(i) RYY, SEQ ID NO: 12 and SEQ ID NO: 2, respectively;
(ii) SEQ ID NO: 24, SEQ ID NO: 17 and SEQ ID NO: 2, respectively; or
(iii) SEQ ID NO: 29, SEQ ID NO: 12 and SEQ ID NO: 7, respectively.

In one embodiment, there is provided a nucleic acid encoding a light chain variable region (VL) of an antibody that binds to PD-1, which light chain variable region (VL) has the amino acid sequence comprising the amino acid sequences of LCDR1, LCDR2 and LCDR3, wherein the nucleic acid sequence encoding the VL has at least 70% identity to SEQ ID NO: 80 and wherein the encoded LCDR1, LCDR2 and LCDR3 amino acid sequences are selected from:
(i) SEQ ID NO: 43, DAS, and SEQ ID NO: 34, respectively; or
(ii) SEQ ID NO: 48, SEQ ID NO: 39, and SEQ ID NO: 34, respectively.

In one embodiment, the above VH variant has at least 70%, at least 75%, at least 80%, at least 85%, at least 90%, at least 95%, at least 97%, or at least 99% identity to a nucleic acid sequence as set forth in SEQ ID NO: 75. In one embodiment, the above VL variant has at least 70%, at least 75%, at least 80%, at least 85%, at least 90%, at least 95%, at least 97%, or at least 99% identity to a nucleic acid sequence as set forth in SEQ ID NO: 80.

In one embodiment, there is provided a nucleic acid encoding a heavy chain variable region (VH) of an antibody that binds to PD-1, which heavy chain variable region (VH) has the amino acid sequence comprising the amino acid sequences of HCDR1, HCDR2 and HCDR3, wherein the nucleic acid sequence encoding the VH has at least 70% identity to SEQ ID NO: 76 and wherein the encoded HCDR1, HCDR2 and HCDR3 amino acid sequences are selected from:
(i) RYY, SEQ ID NO: 13 and SEQ ID NO: 3, respectively;
(ii) SEQ ID NO: 25, SEQ ID NO: 18 and SEQ ID NO: 3, respectively; or
(iii) SEQ ID NO: 30, SEQ ID NO: 13 and SEQ ID NO: 8, respectively.

In one embodiment, there is provided a nucleic acid encoding a light chain variable region (VL) of an antibody that binds to PD-1, which light chain variable region (VL) has the amino acid sequence comprising the amino acid sequences of LCDR1, LCDR2 and LCDR3, wherein the nucleic acid sequence encoding the VL has at least 70% identity to SEQ ID NO: 81 and wherein the encoded LCDR1, LCDR2 and LCDR3 amino acid sequences are selected from:
(i) SEQ ID NO: 44, DAS, and SEQ ID NO: 35, respectively; or
(ii) SEQ ID NO: 49, SEQ ID NO: 39, and SEQ ID NO: 35, respectively.

In one embodiment, the above VH variant has at least 70%, at least 75%, at least 80%, at least 85%, at least 90%, at least 95%, at least 97%, or at least 99% identity to a nucleic acid sequence as set forth in SEQ ID NO: 76. In one embodiment, the above VL variant has at least 70%, at least 75%, at least 80%, at least 85%, at least 90%, at least 95%, at least 97%, or at least 99% identity to a nucleic acid sequence as set forth in SEQ ID NO: 81.

In one embodiment, there is provided a nucleic acid encoding a heavy chain variable region (VH) of an antibody that binds to PD-1, which heavy chain variable region (VH) has the amino acid sequence comprising the amino acid sequences of HCDR1, HCDR2 and HCDR3, wherein the nucleic acid sequence encoding the VH has at least 70% identity to SEQ ID NO: 77 and wherein the encoded HCDR1, HCDR2 and HCDR3 amino acid sequences are selected from:
(i) SEQ ID NO: 21, SEQ ID NO: 14 and SEQ ID NO: 4, respectively;
(ii) SEQ ID NO: 26, SEQ ID NO: 19 and SEQ ID NO: 4, respectively; or
(iii) SEQ ID NO: 31, SEQ ID NO: 14 and SEQ ID NO: 9, respectively.

In one embodiment, there is provided a nucleic acid encoding a light chain variable region (VL) of an antibody that binds to PD-1, which light chain variable region (VL) has the amino acid sequence comprising the amino acid sequences of LCDR1, LCDR2 and LCDR3, wherein the nucleic acid sequence encoding the VL has at least 70% identity to SEQ ID NO: 82 and wherein the encoded LCDR1, LCDR2 and LCDR3 amino acid sequences are selected from:
(i) SEQ ID NO: 45, DAS, and SEQ ID NO: 36, respectively; or
(ii) SEQ ID NO: 50, SEQ ID NO: 40, and SEQ ID NO: 36, respectively.

In one embodiment, the above VH variant has at least 70%, at least 75%, at least 80%, at least 85%, at least 90%, at least 95%, at least 97%, or at least 99% identity to a nucleic acid sequence as set forth in SEQ ID NO: 77. In one embodiment, the above VL variant has at least 70%, at least 75%, at least 80%, at least 85%, at least 90%, at least 95%, at least 97%, or at least 99% identity to a nucleic acid sequence as set forth in SEQ ID NO: 82.

In one embodiment, there is provided a nucleic acid encoding a heavy chain variable region (VH) of an antibody that binds to PD-1, which heavy chain variable region (VH) has the amino acid sequence comprising the amino acid sequences of HCDR1, HCDR2 and HCDR3, wherein the nucleic acid sequence encoding the VH has at least 70% identity to SEQ ID NO: 78 and wherein the encoded HCDR1, HCDR2 and HCDR3 amino acid sequences are selected from:
(i) SEQ ID NO: 22, SEQ ID NO: 15 and SEQ ID NO: 5, respectively;
(ii) SEQ ID NO: 27, SEQ ID NO: 20 and SEQ ID NO: 5, respectively; or
(iii) SEQ ID NO: 32, SEQ ID NO: 15 and SEQ ID NO: 10, respectively.

In one embodiment, there is provided a nucleic acid encoding a light chain variable region (VL) of an antibody that binds to PD-1, which light chain variable region (VL) has the amino acid sequence comprising the amino acid sequences of LCDR1, LCDR2 and LCDR3, wherein the nucleic acid sequence encoding the VL has at least 70% identity to SEQ ID NO: 83 and wherein the encoded LCDR1, LCDR2 and LCDR3 amino acid sequences are selected from:
(i) SEQ ID NO: 46, DAS, and SEQ ID NO: 37, respectively; or
(ii) SEQ ID NO: 51, SEQ ID NO: 41, and SEQ ID NO: 37, respectively.

In one embodiment, the above VH variant has at least 70%, at least 75%, at least 80%, at least 85%, at least 90%, at least 95%, at least 97%, or at least 99% identity to a nucleic acid sequence as set forth in SEQ ID NO: 78. In one embodiment, the above VL variant has at least 70%, at least 75%, at least 80%, at least 85%, at least 90%, at least 95%, at least 97%, or at least 99% identity to a nucleic acid sequence as set forth in SEQ ID NO: 83.

In one embodiment, the nucleic acid comprises a nucleic acid sequence encoding a heavy chain variable region (VH) as shown below and as depicted in SEQ ID NO: 74 of the sequence listing or is a fragment thereof. In one embodiment, the heavy chain variable region (VH) is a variant of the sequence depicted in SEQ ID NO: 74.

```
cag agc gtg gaa gaa tct ggc ggc aga ctg gtc aca cct ggc aca cct ctg aca ctg acc
  Q S V E E S G G R L V T P G T P L T L T
                                                                                    CDR1
                                                                        ~~~~~~~~~~~~~~~~~~~
tgt acc gtg tcc ggc ttc agc ctg tac agc tac aac atg ggc tgg gtc cga cag gcc cct
  C T V S G F S L Y S Y N M G W V R Q A P
                                                                                                            CDR2
                                                                ~~~~~~~~~~~~~~~~~~~~~~~~~~~~~~~~~~~~~~~~~~~~~~~
gga aag gga ctc gag tac atc ggc atc atc agc ggc ggc aca atc ggc cac tat gcc tct
  G K G L E Y I G I I S G G T I G H Y A S
~~~~~~~~~~~~~~~
tgg gcc aag ggc aga ttc acc atc agc aag acc agc agc acc acc gtg gac ctg aag atg
  W A K G I S R F T K T S S T T V D L K M
                                                                                                                                    CDR3
                                                                                                        ~~~~~~~~~~~~~~~~~~~~~~~~~~~
acc agc ctg acc acc gag gac acc gcc acc tac ttt tgc gcc aga gcc ttc tac gac gac
  T S L T T E D T A T Y F C A R A F Y D D
~~~~~~~~~~~~~~~~~~~
tac gac tac aac gtg tgg ggc cca ggc aca ctc gtg aca gtc tcc tct
  Y D Y N V W G P G T L V T V S S
```

In the above nucleic acid sequence and the corresponding amino acid sequence, the complementarity determining regions (CDRs) according to Kabat numbering are indicated by a serpentine line, the underlined nucleotides or amino acids indicate the CDRs according to the IMGT numbering. The bold letters indicate the intersection of Kabat and IMGT numbering.

In one embodiment, the nucleic acid comprises a nucleic acid sequence encoding a heavy chain variable region (VH) as shown below and as depicted in SEQ ID NO: 75 of the sequence listing or is a fragment thereof. In one embodiment, the heavy chain variable region (VH) is a variant of the sequence depicted in SEQ ID NO: 75.

```
cag agc gtg gaa gaa tct ggc ggc aga ctg gtc aca cct ggc aca cct ctg aca ctg acc
  Q S V E E S G G R L V T P G T P L T L T
                                                                                    CDR1
                                                                        ~~~~~~~~~~~~~~~~~~~
tgt acc gtg tcc ggc ttc agc ctg agc cgg tac tac atc agc tgg gtc cga cag gcc cct
  C T V S G F S L S R Y Y I S W V R Q A P
                                                                                                            CDR2
                                                                ~~~~~~~~~~~~~~~~~~~~~~~~~~~~~~~~~~~~~~~~~~~~~~~
ggc aaa gga ctg gaa tgg atc ggc agc ttc tac gcc gat agc ggc aca act tgg tac gcc
  G K G L E W I G S F Y A D S G T T W Y A
~~~~~~~~~~~~~~~
acc tgg gtc aag ggc aga ttc acc ttt agc acc gcc agc agc acc acc gtg gac ctg aag
  T V W K G R F T F S T A S S T T V D L K
                                                                                                                                                    CDR3
                                                                                                                                ~~~~~~~~~~~~~~~
atg aca agc ccc acc acc gag gac acc gcc acc tac ttt tgc gcc aga aac agc ggc gac
  M T S P T T E D T A T Y F C A R N S G D
~~~~~~~~~~~~~~~~~~~
gcc cag ttc aat atc tgg ggc cct gga aca ctg gtc acc gtg tca tct
  A Q F N I W G P G T L V T V S S
```

In the above nucleic acid sequence and the corresponding amino acid sequence, the complementarity determining regions (CDRs) according to Kabat numbering are indicated by a serpentine line, the underlined nucleotides or amino acids indicate the CDRs according to the IMGT numbering. The bold letters indicate the intersection of Kabat and IMGT numbering.

In one embodiment, the nucleic acid comprises a nucleic acid sequence encoding a heavy chain variable region (VH) as shown below and as depicted in SEQ ID NO: 76 of the sequence listing or is a fragment thereof. In one embodiment, the heavy chain variable region (VH) is a variant of the sequence depicted in SEQ ID NO: 76.

```
cag agc gtg gaa gaa tct ggc ggc aga ctg gtc aca cct ggc aca cct ctg aca ctg acc
  Q S V E EVS G G R L V T P G T P L T L T
                                                                                CDR1
                                                                        ~~~~~~~~~~~~~~~~~~~
tgt acc gtg tcc ggc ttc agc ctg agc cgg tac tac atg acc tgg gtc cga cag gcc cct
  C T V S G F S L S R Y Y M T W V R Q A P
                                                                                                            CDR2
                                                                ~~~~~~~~~~~~~~~~~~~~~~~~~~~~~~~~~~~~~~~~~~~~~~~
ggc aaa gga ctg gaa tgg atc ggc atc atc tac ccc gac acc ggc aca act tgg tac gcc
  G K G L E W I G I I Y P D T G T T W Y A
~~~~~~~~~~~~~~~~~~~
tct tgg gtc aag ggc aga ttc acc ttc agc aag acc agc agc acc acc gtg gac ctg aag
  S W V K G R F T F S K T S S T T I D L K
                                                                                                                                            CDR3
                                                                                                                                ~~~~~~~~~~~~~~~
atg aca agc ccc acc acc gag gac acc gcc acc tac ttt tgt gcc aga agc acc aca gac
  M T S P T T E D T A T Y F C A R S T T D
~~~~~~~~~~~~~~~~~~~
gcc cag ttc aac atc tgg ggc cct gga aca ctg gtc acc gtg tca tct
  A Q F N I W G P G T L V T V S S
```

In the above nucleic acid sequence and the corresponding amino acid sequence, the complementarity determining regions (CDRs) according to Kabat numbering are indicated by a serpentine line, the underlined nucleotides or amino acids indicate the CDRs according to the IMGT numbering. The bold letters indicate the intersection of Kabat and IMGT numbering.

In one embodiment, the nucleic acid comprises a nucleic acid sequence encoding a heavy chain variable region (VH) as shown below and as depicted in SEQ ID NO: 77 of the sequence listing or is a fragment thereof. In one embodiment, the heavy chain variable region (VH) is a variant of the sequence depicted in SEQ ID NO: 77.

```
caa gag cac ctg gtg gaa tct ggc gga gga ctg gtt cag cct gag ggc tct ctg acc ctg
  Q E H L V E S G G G L V Q P E G S L T L
                                                                                                CDR1
                                                                                ~~~~~~~~~~~~~~~~~~~~~~~
acc tgt aaa gcc agc ggc atc gac ttc agc gac acc tac tgg atc tgc tgg gtc cga cag
  T C K A S G I D F S D T Y W I C W V R Q
                                                                                                                                  CDR2
                                                                                ~~~~~~~~~~~~~~~~~~~~~~~~~~~~~~~~~~~~~~~
cct cct ggc aaa ggc ctg gaa tgg atc ggc tgt atc ggc atc ggc ggc agc ggc agc aca
  P P G K G L E W I G C I G I G G S G S T
~~~~~~~~~~~~~~~~~~~~~~~~~~~~~~~
tat tat gcc gga tgg gcc aag ggc aga ttc acc atc agc aag acc agc agc acc acc gtg
  Y Y A G W A K G R F T I S K T S S T T V
                                                                                                                                                        ~~~
aca ctg cag atg acc aca ctg acc gac gcc gac acc gcc acc tat ttc tgt gcc acc gag
  T L Q M T T L T D A D T A T Y F C A T E
          CDR3
~~~~~~~~~~~~~~~~~~~~~~~
att ccc tac ttc aac gtg tgg ggc cct ggc aca ctg gtc aca gtc tct tct
  I P Y F N V W G P G T L V T V S S
```

In the above nucleic acid sequence and the corresponding amino acid sequence, the complementarity determining regions (CDRs) according to Kabat numbering are indicated by a serpentine line, the underlined nucleotides or amino acids indicate the CDRs according to the IMGT numbering. The bold letters indicate the intersection of Kabat and IMGT numbering.

In one embodiment, the nucleic acid comprises a nucleic acid sequence encoding a heavy chain variable region (VH) as shown below and as depicted in SEQ ID NO: 78 of the sequence listing or is a fragment thereof. In one embodiment, the heavy chain variable region (VH) is a variant of the sequence depicted in SEQ ID NO: 78.

```
cag agc ctg gaa gaa tct ggc ggc gat ctt gtg aaa cct ggc gcc tct ctg acc ctg aca
  Q S L E E S G G D L V K P G A S L T L T
                                                                                        CDR1
                                                                        ~~~~~~~~~~~~~~~~~~~~~~~
tgt aaa gcc agc ggc atc gac ttc agc agc gtg tac tac atg tgt tgg gtc cga cag gcc
  C K A S G I D F S S V Y Y M C W V R Q A
                                                                                                                            CDR2
                                                                        ~~~~~~~~~~~~~~~~~~~~~~~~~~~~~~~~~~~~~~~~~~~
cct ggc aaa ggc ctg gaa tgg atc gcc tgt atc tac gtg ggc agc agc ggc gtg tcc tac
  P G K G L E W I A C I Y V G S S G V S Y
~~~~~~~~~~~~~~~~~~~~~~~~~~~
tat gcc aca tgg gcc aag ggc aga ttc acc atc agc aag acc agc agc acc acc gtg aca
  Y A T W A K G R F T I S K T S S T T V T
                                                                                                                                                ~~~~~~~
ctg cag atg aca tct ctg aca gcc gcc gac acc gcc acc tac ttt tgt gcc aga gcc gga
  L Q M T S L T A A D T A T Y F C A R A G
                                  CDR3
~~~~~~~~~~~~~~~~~~~~~~~~~~~~~~~~~~~~~~~~~~~~~~~~~~~~~~~
tat gtg ggc gcc gtg tat gtg aca ctg acc aga ctg gat ctg tgg ggc cag ggc aca ctg
  Y V G A V Y V T L T R L D L W G Q G T L
gtc aca gtc tcc tct
  V T V S S
```

In the above nucleic acid sequence and the corresponding amino acid sequence, the complementarity determining regions (CDRs) according to Kabat numbering are indicated by a serpentine line, the underlined nucleotides or amino acids indicate the CDRs according to the IMGT numbering. The bold letters indicate the intersection of Kabat and IMGT numbering.

In one embodiment, the nucleic acid comprises a nucleic acid sequence encoding a heavy chain variable region (VH) as shown below and as depicted in SEQ ID NO: 84 of the sequence listing or is a fragment thereof. In one embodiment, the heavy chain variable region (VH) is a variant of the sequence depicted in SEQ ID NO: 84.

```
cag gtg cag ctg gtt gaa tct ggc gga gga ctg gtg cag cct ggc aca tct ctg aga ctg
  Q V Q L V E S G G G L V Q P G T S L R L
                                                                                            CDR1
                                                                                ~~~~~~~~~~~~~~~~~~~
agc tgt agc gtg tcc ggc ttc agc ctg tac agc tac aac atg ggc tgg gtc cga cag gcc
  S C S V S G F S L Y S Y N M G W V R Q A
                                                                                                                                    CDR2
                                                                        ~~~~~~~~~~~~~~~~~~~~~~~~~~~~~~~~~~~~~~~~~~~
cct gga aag gga ctc gag tac atc ggc atc atc agc ggc ggc aca atc ggc cac tat gcc
  P G K G L E Y I G I I S G G T I G H Y A
~~~~~~~~~~~~~~~~~~~
tct tgg gcc aag ggc aga ttc acc atc agc cgg gac acc agc aag acc aca ctg tac ctg
  S W A K G R F T I S R D T S K T T L Y L
                                                                                                                                        ~~~~~~~~~~~
cag atg aac agc ctg acc acc gag gac acc gcc acc tac ttt tgc gcc aga gcc ttc tac
  Q M N S L T T E D T A T Y F C A R A F Y
      CDR3
~~~~~~~~~~~~~~~~~~~~~~~~~~~
gac gac tac gac tac aac gtg tgg ggc cct ggc aca ctg gtc aca gtc tct tct
  D D Y D Y N V W G P G T L V T V S S
```

In the above nucleic acid sequence and the corresponding amino acid sequence, the complementarity determining regions (CDRs) according to Kabat numbering are indicated by a serpentine line, the underlined nucleotides or amino acids indicate the CDRs according to the IMGT numbering. The bold letters indicate the intersection of Kabat and IMGT numbering.

In one embodiment, the nucleic acid comprises a nucleic acid sequence encoding a heavy chain variable region (VH) as shown below and as depicted in SEQ ID NO: 85 of the sequence listing or is a fragment thereof. In one embodiment, the heavy chain variable region (VH) is a variant of the sequence depicted in SEQ ID NO: 85.

```
cag gtg cag ctg gtt gag tct ggc gga gat gtg gtc aag cct ggc aga agc ctg aga ctg
  Q V Q L V E S G G D V V K P G R S L R L
                                                                                                CDR1
                                                                                ~~~~~~~~~~~~~~~~~~~~~~~
agc tgt aaa gcc agc ggc atc gac ttc agc agc gtg tac tac atg tgc tgg gtc cga cag
  S C K A S G I D F S S V Y Y M C W V R Q
                                                                                                                                    CDR2
                                                                                ~~~~~~~~~~~~~~~~~~~~~~~~~~~~~~~~~~~~~~~
gcc cct ggc aaa gga ctg gaa tgg atc gcc tgt atc tac gtg ggc agc agc ggc gtg tcc
  A P G K G L E W I A C I Y V G S S G V S
~~~~~~~~~~~~~~~~~~~~~~~~~~~~~~~
tac tat gcc aca tgg gcc aag ggc aga ttc acc atc agc cgg gac acc tct acc agc aca
  Y Y A T W A K G R F T I S R D T S T S T
ctg ttt ctg cag atg aac agc ctg aga gcc ggc gac aca gcc acc tac tat tgt gcc aga
  L F L Q M N S L R A G D T A T Y Y C A R
                                              CDR3
~~~~~~~~~~~~~~~~~~~~~~~~~~~~~~~~~~~~~~~~~~~~~~~~~~~~~~~~~~~~~~~
gcc ggc tat gtg ggc gcc gtg tat gtg acc ctg acc aga ctg gat ctg tgg ggc cag gga
  A G Y V G A V Y V T L T R L D L W G Q G
aca ctg gtc aca gtg tca tct
  T L V T V S S
```

In the above nucleic acid sequence and the corresponding amino acid sequence, the complementarity determining regions (CDRs) according to Kabat numbering are indicated by a serpentine line, the underlined nucleotides or amino acids indicate the CDRs according to the IMGT numbering. The bold letters indicate the intersection of Kabat and IMGT numbering.

In one embodiment, the nucleic acid comprises a nucleic acid sequence encoding a heavy chain variable region (VH) as shown below and as depicted in SEQ ID NO: 86 of the sequence listing or is a fragment thereof. In one embodiment, the heavy chain variable region (VH) is a variant of the sequence depicted in SEQ ID NO: 86.

```
gag gtg cag ctg gaa gaa tct ggc ggc gga ctt gtg aag cct ggc gga tct ctg aga ctg
  E V Q L E E S G G G L V K P G G S L R L
                                                                                                CDR1
                                                                                ~~~~~~~~~~~~~~~~~~~~~~~
agc tgt gcc gcc tct ggc atc gat ttc agc agc gtg tac tac atg tgc tgg gtc cga cag
  S C A A S G I D F S S V Y Y M C W V R Q
                                                                                                                                        CDR2
                                                                                ~~~~~~~~~~~~~~~~~~~~~~~~~~~~~~~~~~~~~~~
gcc cct ggc aaa gga ctt gaa tgg gtg tcc tgc atc tac gtg ggc agc agc ggc gtg tcc
  A P G K G L E W V S C I Y V G S S G V S
~~~~~~~~~~~~~~~~~~~~~~~~~~~~~~~
tac tat gcc aca tgg gcc aag ggc aga ttc acc atc agc cgg gac aac agc aag aac acc
  Y Y A T W A K G R F T I S R D N S K N T
ctg tac ctg cag atg aac agc ctg aga gcc gag gac acc gcc gtg tac tat tgt gcc aga
  L Y L Q M N S L R A E D T A V Y Y C A R
                                              CDR3
~~~~~~~~~~~~~~~~~~~~~~~~~~~~~~~~~~~~~~~~~~~~~~~~~~~~~~~~~~~~~~~
gcc gga tat gtg ggc gcc gtg tat gtg acc ctg acc aga ctg gat ctg tgg ggc aga ggc
  A G Y V G A V Y V T L T R L D L W G R G
aca ctg gtc aca gtg tca tct
  T L V T V S S
```

In the above nucleic acid sequence and the corresponding amino acid sequence, the complementarity determining regions (CDRs) according to Kabat numbering are indicated by a serpentine line, the underlined nucleotides or amino acids indicate the CDRs according to the IMGT numbering. The bold letters indicate the intersection of Kabat and IMGT numbering.

In one embodiment, the nucleic acid comprises a nucleic acid sequence encoding a light chain variable region (VL) as shown below and as depicted in SEQ ID NO: 79 of the sequence listing or is a fragment thereof. In one embodiment, the light chain variable region (VH) is a variant of the sequence depicted in SEQ ID NO: 79.

```
gct gct gtg ctg acc cag aca cct tct cca gtg tct gcc gcc gtt ggc ggc aca gtg aca
  A A V L T Q T P S P V S A A V G G T V T
                                                                  CDR1
                        ~~~~~~~~~~~~~~~~~~~~~~~~~~~~~~~~~~~~~~~~~~~~~~~~~~
atc agc tgt cag agc agc cag agc gtg tac ggc aac aac cag ctg tcc tgg tat cag cag
  I S C Q S S Q S V Y G N N Q L S W Y Q Q
                                                                                                          CDR2
                                                                                        ~~~~~~~~~~~~~~~~~~~~~~~~~~~
aag ccc ggc cag cct cct aag ctg ctg atc tac cag gcc agc aag ctg gaa aca ggc gtg
  K P G Q P P K L L I Y Q A S K L E T G V
ccc agc aga ttc aaa ggc agc ggc tct ggc acc cag ttc acc ctg aca atc tcc gac ctg
  P S R F K G S G S G T Q F T L T I S D L
                                                                                                                                      CDR3
                                                                                ~~~~~~~~~~~~~~~~~~~~~~~~~~~~~~~~~~~~~~~
gaa agc gac gat gcc gcc acc tac tat tgt gcc ggc gga tac agc agc agc tcc gac aca
  E S D D A A T Y Y C A G G Y S S S S D T
~~~
aca ttt ggc ggc gga aca gag gtg gtg gtc aag
  T F G G G T E V V V K
```

In the above nucleic acid sequence and the corresponding amino acid sequence, the complementarity determining regions (CDRs) according to Kabat numbering are indicated by a serpentine line, the underlined nucleotides or amino acids indicate the CDRs according to the IMGT numbering. The bold letters indicate the intersection of Kabat and IMGT numbering.

In one embodiment, the nucleic acid comprises a nucleic acid sequence encoding a light chain variable region (VL) as shown below and as depicted in SEQ ID NO: 80 of the sequence listing or is a fragment thereof. In one embodiment, the light chain variable region (VH) is a variant of the sequence depicted in SEQ ID NO: 80.

```
gct gct gtg ctg acc cag aca cct tct cca gtg tct gcc gct gtt ggc ggc aca gtg tct
  A A V L T Q T P S P V S A A V G G T V S
                                                                CDR1
                        ~~~~~~~~~~~~~~~~~~~~~~~~~~~~~~~~~~~~~~~~~~~~~~~~~~~
atc agc tgt cag agc agc gag agc gtg tac aac aag aac cag ctg tgc tgg tat cag cag
  I S C Q S S E S V Y N K N Q L C W Y Q Q
                                                                                                                CDR2
                                                                                        ~~~~~~~~~~~~~~~~~~~~~~~~~~~
aag ccc ggc cag agg cct aag ctg ctg atc tac gat gcc agc aca ctg gcc agc gga gtg
  K P G Q R P K L L I Y D A S T L A S G V
cct agc aga ttt tct ggc agc ggc tct ggc acc cag ttc acc ctg aca atc tcc gac gtg
  P S R F S G S G S G T Q F T L T I S D V
                                                                                                                                CDR3
                                                                                ~~~~~~~~~~~~~~~~~~~~~~~~~~~~~~~~~~~~~~~
cag tct gat gcc gcc gct acc tac tat tgt gcc ggc gga tac agc gtg acc agc gac aca
  Q S D A A A T Y Y C A G G Y S V T S D T
~~~
aca ttt ggc ggc gga aca gag gtg gtc gtc aga
  T F G G G T E V V V R
```

In the above nucleic acid sequence and the corresponding amino acid sequence, the complementarity determining regions (CDRs) according to Kabat numbering are indicated by a serpentine line, the underlined nucleotides or amino acids indicate the CDRs according to the IMGT numbering. The bold letters indicate the intersection of Kabat and IMGT numbering.

In one embodiment, the nucleic acid comprises a nucleic acid sequence encoding a light chain variable region (VL) as shown below and as depicted in SEQ ID NO: 81 of the sequence listing or is a fragment thereof. In one embodiment, the light chain variable region (VH) is a variant of the sequence depicted in SEQ ID NO: 81.

```
gct gct gtg ctg acc cag aca cct tct cca gtg tct gcc gct gtt ggc ggc aca gtg tct
  A A V L T Q T P S P V S A A V G G T V S
                                                                CDR1
                        ~~~~~~~~~~~~~~~~~~~~~~~~~~~~~~~~~~~~~~~~~~~~~~~~~~~
atc agc tgt cag agc agc gag aac gtg tac acc gac aac cag ctg tgc tgg tat cag cag
  I S C Q S S E N V Y T D N Q L C W Y Q Q
                                                                                                                CDR2
                                                                                        ~~~~~~~~~~~~~~~~~~~~~~~~~~~
aag cct ggc cag agg cct aag ctg ctg atc tac gat gcc agc aca ctg gcc agc gga gtg
  K P G Q R P K L L I Y D A S T L A S G V
cct agc aga ttt tct ggc agc ggc tct ggc acc cag ttc acc ctg aca att agc ggc gtg
  P S R F S G S G S G T Q F T L T I S G V
                                                                                                                                CDR3
                                                                                ~~~~~~~~~~~~~~~~~~~~~~~~~~~~~~~~~~~~~~~
cag tcc gat gat gcc gcc acc tat tat tgc gct ggc ggc tac agc acc acc agc gat aca
  Q S D D A A T Y Y C A G G Y S T T S D T
~~~
aca ttt ggc ggc gga acc gag gtg gtg gtc aaa
  T F G G G T E V V V K
```

In the above nucleic acid sequence and the corresponding amino acid sequence, the complementarity determining regions (CDRs) according to Kabat numbering are indicated by a serpentine line, the underlined nucleotides or amino acids indicate the CDRs according to the IMGT numbering. The bold letters indicate the intersection of Kabat and IMGT numbering.

In one embodiment, the nucleic acid comprises a nucleic acid sequence encoding a light chain variable region (VL) as shown below and as depicted in SEQ ID NO: 82 of the sequence listing or is a fragment thereof. In one embodiment, the light chain variable region (VH) is a variant of the sequence depicted in SEQ ID NO: 82.

```
gct cag gtg ctg aca cag aca cct agc tct gtg tct gcc gcc gtt ggc ggc acc gtg acc
  A Q V L T Q T P S S V S A A V G G T V T
                                                                  CDR1
                        ~~~~~~~~~~~~~~~~~~~~~~~~~~~~~~~~~~~~~~~~~~~~~~~~~~~
atc aat tgt cag agc agc cag agc gtg tac aac aag aac tgg ctg gcc tgg tat cag cag
  I N C Q S S Q S V Y N K N W L A W Y Q Q
                                                                                                                CDR2
                                                                                        ~~~~~~~~~~~~~~~~~~~~~~~~~~~
aag cct ggc cag cct cct aag ctg ctg atc tac gat gcc agc aag ctg acc agc ggc gtg
  K P G Q P P K L L I Y D A S K L T S G V
ccc tct aga ttc aaa ggc tct ggc agc ggc acc cag ttc acc ctg aca att tct ggc gtg
  P S R F K G S G S G T Q F T L T I S G V
                                                                                                                                      CDR3
                                                                                ~~~~~~~~~~~~~~~~~~~~~~~~~~~~~~~~~~~~~~~
cag agc gac gac gcc gcc acc tat tat tgc caa ggc acc tac gac gtg aac ggc tgg ctg
  Q S D D A A T Y Y C Q G T Y D V N G W L
~~~~~~~
gtt gct ttt gga ggc gga gcc gaa gtg gtg gtc aaa
  V A F G G G A E V V V K
```

In the above nucleic acid sequence and the corresponding amino acid sequence, the complementarity determining regions (CDRs) according to Kabat numbering are indicated by a serpentine line, the underlined nucleotides or amino acids indicate the CDRs according to the IMGT numbering. The bold letters indicate the intersection of Kabat and IMGT numbering.

In one embodiment, the nucleic acid comprises a nucleic acid sequence encoding a light chain variable region (VL) as shown below and as depicted in SEQ ID NO: 83 of the sequence listing or is a fragment thereof. In one embodiment, the light chain variable region (VH) is a variant of the sequence depicted in SEQ ID NO: 83.

```
gct gct gtg ctg acc cag aca cct tct cca gtg tct gcc gcc gtt ggc ggc aca gtg aca
  A A V L T Q T P S P V S A A V G G T V T
                                                                CDR1
                        ~~~~~~~~~~~~~~~~~~~~~~~~~~~~~~~~~~~~~~~~~~~~~~~~~~~
atc agc tgt cag agc agc cag agc atc tac acc aac aac gac ctg gcc tgg tat cag cag
  I S C Q S S Q S I Y T N N D L A W Y Q Q
                                                                                                                CDR2
                                                                                        ~~~~~~~~~~~~~~~~~~~~~~~~~~~
aag cct ggc cag cct cct aag ctg ctg atc tac gat gcc agc aag ctg gcc tct ggc gtg
  K P G Q P P K L L I Y D A S K L A S G V
cca agc aga ttt tct ggc agc ggc tct ggc acc cag ttc acc ctg aca att agc ggc gtg
  P S R F S G S G S G T Q F T L T I S G V
                                                                                                                                      CDR3
                                                                                ~~~~~~~~~~~~~~~~~~~~~~~~~~~~~~~~~~~~~~~
cag tcc gat gat gcc gcc acc tat tat tgc ctc ggc ggc tac gat gac gac gcc gac aat
  Q S D D A A T Y Y C L G G Y D D D A D N
~~~
gct ttt ggc ggc gga aca gag gtg gtg gtc aaa
  A F G G G T E V V V K
```

In the above nucleic acid sequence and the corresponding amino acid sequence, the complementarity determining regions (CDRs) according to Kabat numbering are indicated by a serpentine line, the underlined nucleotides or amino acids indicate the CDRs according to the IMGT numbering. The bold letters indicate the intersection of Kabat and IMGT numbering.

In one embodiment, the nucleic acid comprises a nucleic acid sequence encoding a light chain variable region (VL) as shown below and as depicted in SEQ ID NO: 87 of the sequence listing or is a fragment thereof. In one embodiment, the light chain variable region (VH) is a variant of the sequence depicted in SEQ ID NO: 87.

```
gac atc gtg atg aca cag agc cct agc agc ctg tct gcc agc gtg gga gac aga gtg acc
  D I V M T Q S P S S L S A S V G D R V T
                                                              CDR1
                        ~~~~~~~~~~~~~~~~~~~~~~~~~~~~~~~~~~~~~~~~~~~~~~~~~~~
atc acc tgt cag agc agc cag agc gtg tac ggc aac aac cag ctg tcc tgg tat cag cag
  I T C Q S S Q S V Y G N N Q L S W Y Q Q
                                                                                                              CDR2
                                                                                        ~~~~~~~~~~~~~~~~~~~~~~~~~~~
aag ccc ggc aag gcc cct aag ctg ctg atc tac cag gcc agc aag ctg gaa aca ggc gtg
  K P G K A P K L L I Y Q A S K L E T G V
ccc agc aga ttt tct ggc agc ggc tct ggc acc gac ttc acc ctg acc ata tct agc ctg
  P S R F S G S G S G T D F T L T I S S L
                                                                                                                                CDR3
                                                                                ~~~~~~~~~~~~~~~~~~~~~~~~~~~~~~~~~~~~~~~
cag cct gag gac ttc gcc acc tac tat tgt gcc ggc gga tac agc agc agc tcc gac aca
  Q P E D F A Ţ Y Y C A G G Y S S S S D T
~~~
aca ttt ggc gga ggc acc aag gtg gtc atc aag
  T F G G G T K V V I K
```

In the above nucleic acid sequence and the corresponding amino acid sequence, the complementarity determining regions (CDRs) according to Kabat numbering are indicated by a serpentine line, the underlined nucleotides or amino acids indicate the CDRs according to the IMGT numbering. The bold letters indicate the intersection of Kabat and IMGT numbering.

In one embodiment, the nucleic acid comprises a nucleic acid sequence encoding a light chain variable region (VL) as shown below and as depicted in SEQ ID NO: 88 of the sequence listing or is a fragment thereof. In one embodiment, the light chain variable region (VH) is a variant of the sequence depicted in SEQ ID NO: 88.

```
gac atc cag atg aca cag agc ccc agc aca ctg tct gcc agc gtg gga gac aga gtg acc
  D I V M T Q S P S S L S A S V G D R V T
                                                                CDR1
                        ~~~~~~~~~~~~~~~~~~~~~~~~~~~~~~~~~~~~~~~~~~~~~~~~~~~
atc acc tgt cag agc agc cag agc gtg tac ggc aac aac cag ctg tcc tgg tat cag cag
  I T C Q S S Q S V Y G N N Q L S W Y Q Q
                                                                                                                CDR2
                                                                                        ~~~~~~~~~~~~~~~~~~~~~~~~~~~
aag ccc ggc aag gcc cct aag ctg ctg atc tac cag gcc agc aag ctg gaa aca ggc gtg
  K P G K A P K L L I Y Q A S K L E T G V
ccc agc aga ttt tct ggc agc ggc tct ggc acc gac ttc acc ctg acc ata tct agc ctg
  P S R F S G S G S G T D F T L T I S S L
                                                                                                                              CDR3
                                                                                ~~~~~~~~~~~~~~~~~~~~~~~~~~~~~~~~~~~~~~~
cag cct gac gac ttc gcc agc tac tat tgt gcc ggc gga tac agc agc agc tcc gat acc
  Q P D D F A S Y Y C A G G Y S S S S D T
~~~
aca ttt ggc cag ggc acc aag gtg gaa atc aag
  T F G Q G T K V E I K
```

In the above nucleic acid sequence and the corresponding amino acid sequence, the complementarity determining regions (CDRs) according to Kabat numbering are indicated by a serpentine line, the underlined nucleotides or amino acids indicate the CDRs according to the IMGT numbering. The bold letters indicate the intersection of Kabat and IMGT numbering.

In one embodiment, the nucleic acid comprises a nucleic acid sequence encoding a light chain variable region (VL) as shown below and as depicted in SEQ ID NO: 89 of the sequence listing or is a fragment thereof. In one embodiment, the light chain variable region (VH) is a variant of the sequence depicted in SEQ ID NO: 89.

```
gac atc cag atg aca cag agc cct agc agc ctg tct gcc agc gtg gga gac aga gtg acc
  D I Q M T Q S P S S L S A S V G D R V T
                                                                  CDR1
                        ~~~~~~~~~~~~~~~~~~~~~~~~~~~~~~~~~~~~~~~~~~~~~~~~~~~
atc acc tgt cag agc agc cag agc gtg tac ggc aac aac cag ctg tcc tgg tat cag aag
  I T C Q S S Q S V Y G N N Q L S W Y Q K
                                                                                                                CDR2
                                                                                        ~~~~~~~~~~~~~~~~~~~~~~~~~~~
aag ccc gga cag gcc cct aag ctg ctg atc tac cag gcc agc aag ctg gaa aca ggc gtg
  K P G Q A P K L L I Y Q A S K L E T G V
ccc agc aga ttt tct ggc agc ggc tct ggc acc gac ttc acc ctg acc ata tct agc ctg
  P S R F S G S G S G T D E T L T I S S L
                                                                                                                                CDR3
                                                                                ~~~~~~~~~~~~~~~~~~~~~~~~~~~~~~~~~~~~~~~
cag cct gag gac ttc gcc acc tac tat tgt gcc ggc gga tac agc agc agc tcc gac aca
  Q P E D F A T Y Y C A G G Y S S S S D T
~~~
aca ttt ggc cct ggc acc aag gtg gac atc aag
  T F G P G T K V D I K
```

In the above nucleic acid sequence and the corresponding amino acid sequence, the complementarity determining regions (CDRs) according to Kabat numbering are indicated by a serpentine line, the underlined nucleotides or amino acids indicate the CDRs according to the IMGT numbering. The bold letters indicate the intersection of Kabat and IMGT numbering.

In one embodiment, the nucleic acid comprises a nucleic acid sequence encoding a light chain variable region (VL) as shown below and as depicted in SEQ ID NO: 90 of the sequence listing or is a fragment thereof. In one embodiment, the light chain variable region (VH) is a variant of the sequence depicted in SEQ ID NO: 90.

```
gcc att cag ctg aca cag agc cct tet agc ctg agc gcc tct gtt ggc ggc acc gtg aca
  A I Q L T Q S P S S L S A S V G G T V T
                                                                  CDR1
                      ~~~~~~~~~~~~~~~~~~~~~~~~~~~~~~~~~~~~~~~~~~~~~~~~~~~~
atc acc tgt cag agc agc cag agc gtg tac ggc aac aac cag ctg tcc tgg tat cag cag
  I T C Q S S Q S V Y G N N Q L S W Y Q Q
                                                                                                              CDR2
                                                                                        ~~~~~~~~~~~~~~~~~~~~~~~~~~~
aag ccc ggc cag cct cct aag ctg ctg atc tac cag gcc agc aag ctg gaa aca ggc gtg
  K P G Q P P K L L I Y Q A S K L E T G V
ccc tct aga ttc aga ggc agc ggc tct ggc acc cag ttc aca ctg aca atc agc agc ctg
  S P R F R G S G S G T Q F T L T I S S L
                                                                                                                                        CDR3
                                                                                ~~~~~~~~~~~~~~~~~~~~~~~~~~~~~~~~~~~~~~~
cag agc gag gac ttc gcc acc tac tat tgt gcc ggc gga tac agc agc agc toc gac aca
  Q S E D F A T Y Y C A G G Y S S S S D T
~~~
aca ttt ggc ggc gga aca gag gtg gtg gtc aag
  T F G G G T E V V V K
```

In the above nucleic acid sequence and the corresponding amino acid sequence, the complementarity determining regions (CDRs) according to Kabat numbering are indicated by a serpentine line, the underlined nucleotides or amino acids indicate the CDRs according to the IMGT numbering. The bold letters indicate the intersection of Kabat and IMGT numbering.

In one embodiment, the nucleic acid comprises a nucleic acid sequence encoding a light chain variable region (VL) as shown below and as depicted in SEQ ID NO: 91 of the sequence listing or is a fragment thereof. In one embodiment, the light chain variable region (VH) is a variant of the sequence depicted in SEQ ID NO: 91.

```
gac gtg gtc atg aca cag agc cct agc aca gtg tct gcc agc gtg ggc gat aga gtg acc
  D V V M T Q S P S T V S A S V G D R V T
                                                                    CDR1
                        ~~~~~~~~~~~~~~~~~~~~~~~~~~~~~~~~~~~~~~~~~~~~~~~~~~~
ctg acc tgt cag agc agc cag agc atc tac acc aac aac gac ctg gcc tgg tat cag cag
  L T C Q S S Q S I Y T N N D L A W Y Q Q
                                                                                                                CDR2
                                                                                        ~~~~~~~~~~~~~~~~~~~~~~~~~~~
aag cct ggc cag cct cct aag ctg ctg atc tac gat gcc agc aag ctg gcc tct ggc gtg
  K P G Q P P K L L I Y D A S K L A S G V
ccc gat aga ttt tct ggc agc ggc tct ggc acc gac ttc acc ctg aca att agc tcc ctg
  P D R F S G S G S G T D F T L T I S S L
                                                                                                                                          CDR3
                                                                                ~~~~~~~~~~~~~~~~~~~~~~~~~~~~~~~~~~~~~~
cag gcc gac gac ttc gcc acc tat tat tgt ctc ggc ggc tac gac gac gac gcc gat aat
  Q A D D F A T Y Y C L G G Y D D D A D N
~~~
gct ttt ggc cag ggc acc aag gtg gaa atc aag
  A F G Q G T K V E I K
```

In the above nucleic acid sequence and the corresponding amino acid sequence, the complementarity determining regions (CDRs) according to Kabat numbering are indicated by a serpentine line, the underlined nucleotides or amino acids indicate the CDRs according to the IMGT numbering. The bold letters indicate the intersection of Kabat and IMGT numbering.

In one embodiment, the nucleic acid comprises a nucleic acid sequence encoding a light chain variable region (VL) as shown below and as depicted in SEQ ID NO: 92 of the sequence listing or is a fragment thereof. In one embodiment, the light chain variable region (VH) is a variant of the sequence depicted in SEQ ID NO: 92.

```
gac atc cag atg aca cag agc cct agc agc ctg tct gcc tct gtt ggc ggc acc gtg aca
  D I Q M T Q S P S S L S A S V G G T V T
                                                                  CDR1
                        ~~~~~~~~~~~~~~~~~~~~~~~~~~~~~~~~~~~~~~~~~~~~~~~~~~~
atc acc tgt cag agc agc cag agc atc tac acc aac aac gac ctg gcc tgg tat cag cag
  I T C Q S S Q S I Y T N N D L A W Y Q Q
                                                                                                              CDR2
                                                                                          ~~~~~~~~~~~~~~~~~~~~~~~~~~
aag cct ggc cag cct cct aag ctg ctg atc tac gat gcc agc aag ctg gcc tct ggc gtg
  K P G Q P P K L L I Y D A S K L A S G V
cca agc aga ttt tct ggc agc ggc tct ggc acc cag ttc acc ctg aca atc tct agc ctg
  P S R F S G S G S G T Q F T L T I S S L
                                                                                                                                        CDR3
                                                                                ~~~~~~~~~~~~~~~~~~~~~~~~~~~~~~~~~~~~~~~
cag agc gag gat gcc gcc acc tac tat tgt ctc ggc ggc tac gac gac gac gcc gac aat
  Q S E D A A T Y Y C L G G Y D D D A D N
~~~
gct ttt ggc ggc gga aca gag gtg gtg gtc aaa
  A F G G G T E V V V K
```

In the above nucleic acid sequence and the corresponding amino acid sequence, the complementarity determining regions (CDRs) according to Kabat numbering are indicated by a serpentine line, the underlined nucleotides or amino acids indicate the CDRs according to the IMGT numbering. The bold letters indicate the intersection of Kabat and IMGT numbering.

The teaching given herein with respect to specific nucleic acid and amino acid sequences, e.g., those shown in the sequence listing, is to be construed so as to also relate to modifications of said specific sequences resulting in sequences which are functionally equivalent to said specific sequences, e.g., amino acid sequences exhibiting properties identical or similar to those of the specific amino acid sequences and nucleic acid sequences encoding amino acid sequences exhibiting properties identical or similar to those of the amino acid sequences encoded by the specific nucleic acid sequences. One important property is to retain binding of an antibody to its target or to sustain the desired effector functions of an antibody. Preferably, a sequence modified with respect to a specific sequence, when it replaces the specific sequence in an antibody retains binding of said antibody to PD-1 and preferably functions of said antibody as described herein, e.g., inhibiting the immunosuppressive of PD-1 on cells expressing PD-1, CDC mediated lysis or ADCC mediated lysis.

For example, variants of nucleic acid and amino acid sequences, as described herein, encode or provide antibody or antigen-binding fragments, which provide at least one of the following properties:
(i) being capable of binding, preferably specifically binding to PD-1, e.g., human PD-1;
(ii) being capable of blocking binding of PD-1 to its ligand;
(iii) being capable of binding to the same antigen, to which the parent antibody binds, preferably with an affinity that is sufficient to provide for diagnostic and/or therapeutic use; and/or
(iv) being capable of providing reduced or depleted effector functions.

It will be appreciated by those skilled in the art that in particular the sequences of the CDR, hypervariable and variable regions can be modified without losing the ability to bind PD-1. For example, CDR regions will be either identical or highly homologous to the regions specified herein. By “highly homologous” it is contemplated that from 1 to 5, preferably from 1 to 4, such as 1 to 3 or 1 or 2 substitutions may be made in the CDRs. In addition, the hypervariable and variable regions may be modified so that they show substantial homology with the regions specifically disclosed herein.

It is to be understood that the nucleic acids described herein also include nucleic acids modified for the sake of optimizing the codon usage in a particular host cell or organism. Differences in codon usage among organisms can lead to a variety of problems concerning heterologous gene expression. Codon optimization by changing one or more nucleotides of the original sequence can result in an optimization of the expression of a nucleic acid, in particular in optimization of translation efficacy, in a homologous or heterologous host in which said nucleic acid is to be expressed. For example, if nucleic acids derived from human and encoding constant regions and/or framework regions of antibodies are to be used according to the present invention, e.g., for preparing chimeric or humanised antibodies, it may be preferred to modify said nucleic acids for the sake of optimization of codon usage, in particular if said nucleic acids, optionally fused to heterologous nucleic acids such as nucleic acids derived from other organisms as described herein, are to be expressed in cells from an organism different from human such as mouse or hamster. For example, the nucleic acid sequences encoding human light and heavy chain constant regions, can be modified to include one or more, preferably, at least 1, 2, 3, 4, 5, 10, 15, 20 and preferably up to 10, 15, 20, 25, 30, 50, 70 or 100 or more nucleotide replacements resulting in an optimized codon usage but not resulting in a change of the amino acid sequence.

A “nucleic acid” according to the invention can be RNA, more preferably in vitro transcribed RNA (IVT RNA) or synthetic RNA. A nucleic can be employed for introduction into, i.e., transfection of, cells, in particular, in the form of RNA which can be prepared by in vitro transcription from a DNA template. The RNA can moreover be modified before application by stabilizing sequences, capping, and polyadenylation.

The term “genetic material” includes isolated nucleic acid, either DNA or RNA, a section of a double helix, a section of a chromosome, or an organism's or cell's entire genome, in particular its exome or transcriptome.

The term “mutation” refers to a change of or difference in the nucleic acid sequence (nucleotide substitution, addition or deletion) compared to a reference. A “somatic mutation” can occur in any of the cells of the body except the germ cells (sperm and egg) and therefore are not passed on to children. These alterations can (but do not always) cause cancer or other diseases. Preferably a mutation is a non-synonymous mutation. The term “non-synonymous mutation” refers to a mutation, preferably a nucleotide substitution, which does result in an amino acid change such as an amino acid substitution in the translation product.

According to the invention, the term “mutation” includes point mutations, Indels, fusions, chromothripsis and RNA edits.

According to the invention, the term “Indel” describes a special mutation class, defined as a mutation resulting in a colocalized insertion and deletion and a net gain or loss in nucleotides. In coding regions of the genome, unless the length of an indel is a multiple of 3, they produce a frameshift mutation. Indels can be contrasted with a point mutation; where an Indel inserts and deletes nucleotides from a sequence, a point mutation is a form of substitution that replaces one of the nucleotides.

According to the invention, the term “chromothripsis” refers to a genetic phenomenon by which specific regions of the genome are shattered and then stitched together via a single devastating event.

According to the invention, the term “RNA edit” or “RNA editing” refers to molecular processes in which the information content in an RNA molecule is altered through a chemical change in the base makeup. RNA editing includes nucleoside modifications such as cytidine (C) to uridine (U) and adenosine (A) to inosine (I) deaminations, as well as non-templated nucleotide additions and insertions. RNA editing in mRNAs effectively alters the amino acid sequence of the encoded protein so that it differs from that predicted by the genomic DNA sequence.

According to the invention, a “reference” may be used to correlate and compare the results obtained from a tumor specimen. Typically the “reference” may be obtained on the basis of one or more normal specimens, in particular specimens which are not affected by a cancer disease, either obtained from a patient or one or more different individuals, preferably healthy individuals, in particular individuals of the same species. A “reference” can be determined empirically by testing a sufficiently large number of normal specimens.

In the context of the present invention, the term “RNA” relates to a molecule which comprises ribonucleotide residues and preferably being entirely or substantially composed of ribonucleotide residues. “Ribonucleotide” relates to a nucleotide with a hydroxyl group at the 2′-position of a P-D-ribofuranosyl group. The term “RNA” comprises double-stranded RNA, single-stranded RNA, isolated RNA such as partially or completely purified RNA, essentially pure RNA, synthetic RNA, and recombinantly generated RNA such as modified RNA which differs from naturally occurring RNA by addition, deletion, substitution and/or alteration of one or more nucleotides. Such alterations can include addition of non-nucleotide material, such as to the end(s) of a RNA or internally, for example at one or more nucleotides of the RNA. Nucleotides in RNA molecules can also comprise non-standard nucleotides, such as non-naturally occurring nucleotides or chemically synthesized nucleotides or deoxynucleotides. These altered RNAs can be referred to as analogs or analogs of naturally-occurring RNA.

According to the present invention, the term “RNA” includes and preferably relates to “mRNA”. The term “mRNA” means “messenger-RNA” and relates to a “transcript” which is generated by using a DNA template and encodes a peptide or polypeptide. The promoter for controlling transcription can be any promoter for any RNA polymerase. A DNA template for in vitro transcription may be obtained by cloning of a nucleic acid, in particular cDNA, and introducing it into an appropriate vector for in vitro transcription. The cDNA may be obtained by reverse transcription of RNA. Typically, an mRNA comprises a 5′-UTR, a protein coding region, and a 3′-UTR. mRNA only possesses limited half-life in cells and in vitro. In the context of the present invention, mRNA may be generated by in vitro transcription from a DNA template. The in vitro transcription methodology is known to the skilled person. For example, there is a variety of in vitro transcription kits commercially available.

According to the invention, the stability and translation efficiency of RNA may be modified as required. RNA molecules with increased stability and improved translation efficiency may for example be advantageous for the RNA encoded antibodies of the present invention. For example, RNA may be stabilized and its translation increased by one or more modifications having stabilizing effects and/or increasing translation efficiency of RNA. Such modifications are described, for example, in PCT/EP2006/009448 incorporated herein by reference. In order to increase expression of the RNA used according to the present invention, it may be modified within the coding region, i.e., the sequence encoding the expressed peptide or protein, preferably without altering the sequence of the expressed peptide or protein, so as to increase the GC-content to increase mRNA stability and to perform a codon optimization and, thus, enhance translation in cells.

The term “modification” in the context of the RNA used in the present invention includes any modification of an RNA which is not naturally present in said RNA.

In one embodiment of the invention, the RNA used according to the invention does not have uncapped 5′-triphosphates. Removal of such uncapped 5′-triphosphates can be achieved by treating RNA with a phosphatase.

The RNA according to the invention may have modified ribonucleotides in order to increase its stability and/or decrease cytotoxicity. For example, in one embodiment, in the RNA used according to the invention 5-methylcytidine is substituted partially or completely, preferably completely, for cytidine. Alternatively or additionally, in one embodiment, in the RNA used according to the invention pseudouridine (ψ), N1-methyl-pseudouridine (m1ψ), or 5-methyl-uridine (m5U) is substituted partially or completely, preferably completely, for uridine.

The term “uridine,” as used herein, describes one of the nucleosides that can occur in RNA. The structure of uridine is:

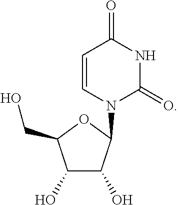 [SMILES: O=c1ccn([C@@H]2O[C@H](CO)[C@@H](O)[C@H]2O)c(=O)[nH]1]

UTP (uridine 5′-triphosphate) has the following structure:

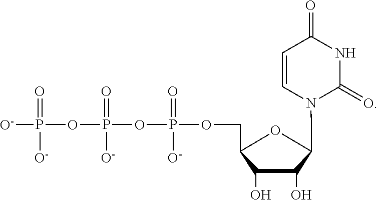

Pseudo-UTP (pseudouridine w-triphosphate) has the following structure:

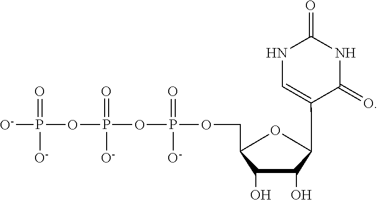

“Pseudouridine” is one example of a modified nucleoside that is an isomer of uridine, where the uracil is attached to the pentose ring via a carbon-carbon bond instead of a nitrogen-carbon glycosidic bond.

Another exemplary modified nucleoside is Ni-methyl-pseudouridine (m1Ψ), which has the structure:

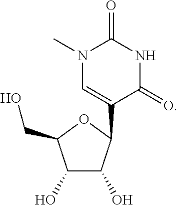 [SMILES: Cn1cc([C@@H]2O[C@H](CO)[C@@H](O)[C@H]2O)c(=O)[nH]c1=O]

N1-methyl-pseudo-UTP has the following structure:

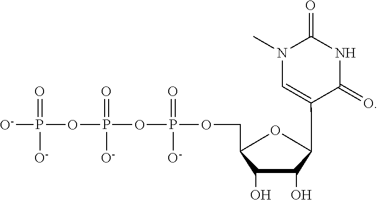

Another exemplary modified nucleoside is 5-methyl-uridine (m5U), which has the structure:

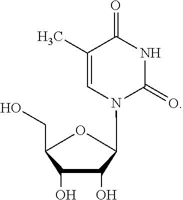

In some embodiments, one or more uridine in the RNA described herein is replaced by a modified nucleoside. In some embodiments, the modified nucleoside is a modified uridine.

In some embodiments, the modified uridine replacing uridine is pseudouridine (ψ), N1-methyl-pseudouridine (m1ψ), or 5-methyl-uridine (m5U).

In some embodiments, the modified nucleoside replacing one or more uridine in the RNA may be any one or more of 3-methyl-uridine (m³U), 5-methoxy-uridine (mo⁵U), 5-aza-uridine, 6-aza-uridine, 2-thio-5-aza-uridine, 2-thio-uridine (s2U), 4-thio-uridine (s⁴U), 4-thio-pseudouridine, 2-thio-pseudouridine, 5-hydroxy-uridine (ho5U), 5-aminoallyl-uridine, 5-halo-uridine (e.g., 5-iodo-uridineor 5-bromo-uridine), uridine 5-oxyacetic acid (cmo⁵U), uridine 5-oxyacetic acid methyl ester (mcmo⁵U), 5-carboxymethyl-uridine (cm⁵U), 1-carboxymethyl-pseudouridine, 5-carboxyhydroxymethyl-uridine (chm⁵U), 5-carboxyhydroxymethyl-uridine methyl ester (mchm⁵U), 5-methoxycarbonylmethyl-uridine (mcm⁵U), 5-methoxycarbonylmethyl-2-thio-uridine (mcm⁵s²U), 5-aminomethyl-2-thio-uridine (nm⁵s2U), 5-methylaminomethyl-uridine (mnm⁵U), 1-ethyl-pseudouridine, 5-methylaminomethyl-2-thio-uridine (mnm⁵s2U), 5-methylaminomethyl-2-seleno-uridine (mnm⁵se²U), 5-carbamoylmethyl-uridine (ncm⁵U), 5-carboxymethylaminomethyl-uridine (cmnm⁵U), 5-carboxymethylaminomethyl-2-thio-uridine (cmnm⁵s2U), 5-propynyl-uridine, 1-propynyl-pseudouridine, 5-taurinomethyl-uridine (τm⁵U), 1-taurinomethyl-pseudouridine, 5-taurinomethyl-2-thio-uridine(τm⁵s²U), 1-taurinomethyl-4-thio-pseudouridine), 5-methyl-2-thio-uridine (m⁵s²U), 1-methyl-4-thio-pseudouridine (m¹s⁴ψ), 4-thio-1-methyl-pseudouridine, 3-methyl-pseudouridine (m³ψ), 2-thio-1-methyl-pseudouridine, 1-methyl-1-deaza-pseudouridine, 2-thio-1-methyl-1-deaza-pseudouridine, dihydrouridine (D), dihydropseudouridine, 5,6-dihydrouridine, 5-methyl-dihydrouridine (m5D), 2-thio-dihydrouridine, 2-thio-dihydropseudouridine, 2-methoxy-uridine, 2-methoxy-4-thio-uridine, 4-methoxy-pseudouridine, 4-methoxy-2-thio-pseudouridine, N1-methyl-pseudouridine, 3-(3-amino-3-carboxypropyl)uridine (acp³ψ), 1-methyl-3-(3-amino-3-carboxypropyl)pseudouridine (acp³ψ), 5-(isopentenylaminomethyl)uridine (inm⁵U), 5-(isopentenylaminomethyl)-2-thio-uridine (inm⁵s²U), α-thio-uridine, 2′-O-methyl-uridine (Um), 5,2′-O-dimethyl-uridine (m⁵Um), 2′-O-methyl-pseudouridine (ψM), 2-thio-2′-O-methyl-uridine (s²Um), 5-methoxycarbonylmethyl-2′-O-methyl-uridine (mcm⁵Um), 5-carbamoylmethyl-2′-O-methyl-uridine (ncm⁵Um), 5-carboxymethylaminomethyl-2′-O-methyl-uridine (cmnm⁵Um), 3,2′-O-dimethyl-uridine (m³Um), 5-(isopentenylaminomethyl)-2′-O-methyl-uridine (inm⁵Um), 1-thio-uridine, deoxythymidine, 2′-F-ara-uridine, 2′-F-uridine, 2′-OH-ara-uridine, 5-(2-carbomethoxyvinyl) uridine, 5-[3-(1-E-propenylamino)uridine, or any other modified uridine known in the art.

In some embodiments, at least one RNA comprises a modified nucleoside in place of at least one uridine. In some embodiments, at least one RNA comprises a modified nucleoside in place of each uridine. In some embodiments, each RNA comprises a modified nucleoside in place of at least one uridine. In some embodiments, each RNA comprises a modified nucleoside in place of each uridine.

In some embodiments, the modified nucleoside is independently selected from pseudouridine (ψ), N1-methyl-pseudouridine (m¹ψ), and 5-methyl-uridine (msU). In some embodiments, the modified nucleoside comprises pseudouridine (ψ). In some embodiments, the modified nucleoside comprises N1-methyl-pseudouridine (m¹ψ). In some embodiments, the modified nucleoside comprises 5-methyl-uridine (m⁵U). In some embodiments, at least one RNA may comprise more than one type of modified nucleoside, and the modified nucleosides are independently selected from pseudouridine (ψ), N1-methyl-pseudouridine (m¹ψ), and 5-methyl-uridine (m⁵U). In some embodiments, the modified nucleosides comprise pseudouridine (ψ) and N1-methyl-pseudouridine (m¹ψ). In some embodiments, the modified nucleosides comprise pseudouridine (ψ) and 5-methyl-uridine (m⁵U). In some embodiments, the modified nucleosides comprise N1-methyl-pseudouridine (m¹ψ) and 5-methyl-uridine (m5U). In some embodiments, the modified nucleosides comprise pseudouridine (ψ), N1-methyl-pseudouridine (m¹ψ), and 5-methyl-uridine (m⁵U).

In one embodiment, the RNA comprises other modified nucleosides or comprises further modified nucleosides, e.g., modified cytidine. For example, in one embodiment, in the RNA 5-methylcytidine is substituted partially or completely, preferably completely, for cytidine. In one embodiment, the RNA comprises 5-methylcytidine and one or more selected from pseudouridine (ψ), N1-methyl-pseudouridine (m¹ψ), and 5-methyl-uridine (m⁵U). In one embodiment, the RNA comprises 5-methylcytidine and N1-methyl-pseudouridine (m¹ψ). In some embodiments, the RNA comprises 5-methylcytidine in place of each cytidine and N1-methyl-pseudouridine (m¹ψ) in place of each uridine.

In one embodiment, the term “modification” relates to providing an RNA with a 5′-cap or 5′-cap analog. The term “5′-cap” refers to a cap structure found on the 5′-end of an mRNA molecule and generally consists of a guanosine nucleotide connected to the mRNA via an unusual 5′ to 5′ triphosphate linkage. In one embodiment, this guanosine is methylated at the 7-position. The term “conventional 5′-cap” refers to a naturally occurring RNA 5′-cap, preferably to the 7-methylguanosine cap (m⁷G). In the context of the present invention, the term “5′-cap” includes a 5′-cap analog that resembles the RNA cap structure and is modified to possess the ability to stabilize RNA and/or enhance translation of RNA if attached thereto, preferably in vivo and/or in a cell.

Providing an RNA with a 5′-cap or 5′-cap analog may be achieved by in vitro transcription of a DNA template in presence of said 5′-cap or 5′-cap analog, wherein said 5′-cap is co-transcriptionally incorporated into the generated RNA strand, or the RNA may be generated, for example, by in vitro transcription, and the 5′-cap may be attached to the RNA post-transcriptionally using capping enzymes, for example, capping enzymes of vaccinia virus.

In some embodiments, the building block cap for RNA is m₂^{7,3′-O}Gppp(m₁^{2′-O})ApG (also sometimes referred to as m₂^{7,3′O}G(5′)ppp(5′)m^{2′-O}ApG), which has the following structure:

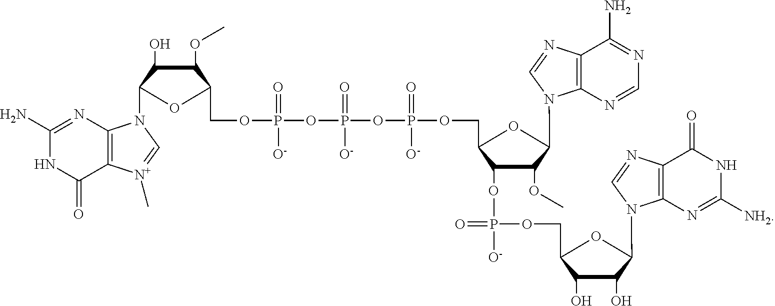

Below is an exemplary Cap1 RNA, which comprises RNA and m₂^{7,3′O}G(5′)ppp(5′)m^{2′-O}ApG:

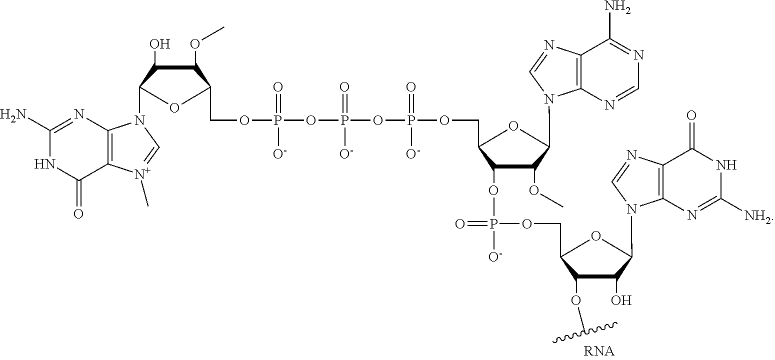

Below is another exemplary Cap1 RNA (no cap analog):

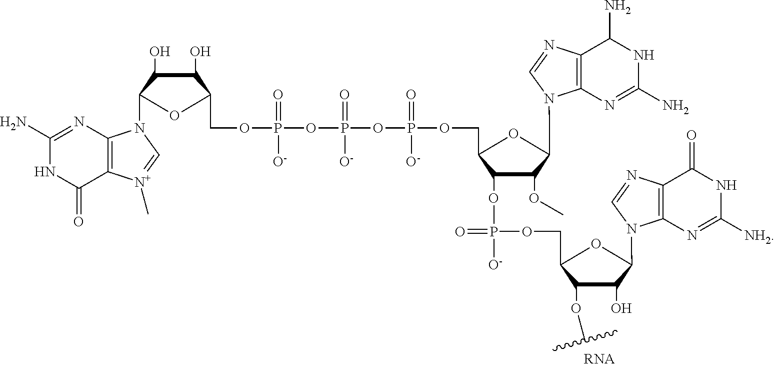

In some embodiments, the RNA is modified with “Cap0” structures using, in one embodiment, the cap analog anti-reverse cap (ARCA Cap (m₂^{7,3′O}G(5′)ppp(5′)G)) with the structure:

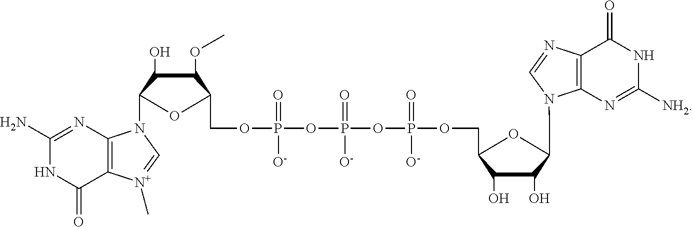

Below is an exemplary Cap0 RNA comprising RNA and m₂^{7,3′O}G(5′)ppp(5′)G:

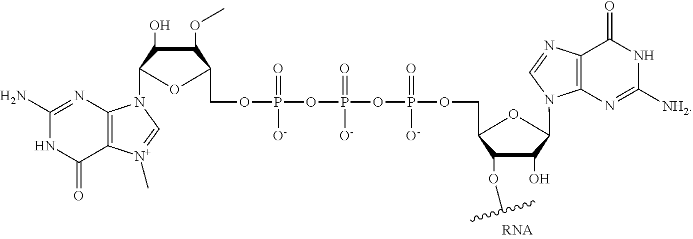

In some embodiments, the “Cap0” structures are generated using the cap analog Beta-S-ARCA (m₂^{7,2′O}G(5′)ppSp(5′)G) with the structure:

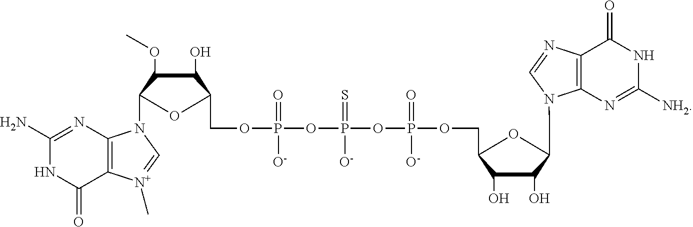

Below is an exemplary Cap0 RNA comprising Beta-S-ARCA (m₂^{7,2′O}G(5′)ppSp(5′)G) and RNA:

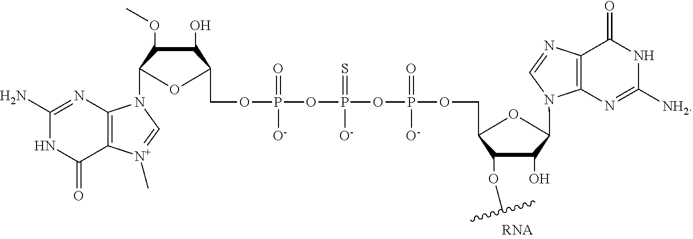

A particularly preferred Cap comprises the 5′-cap m₂^{7,2′O}G(5′)ppSp(5′)G. In some embodiments, at least one RNA described herein comprises the 5′-cap m₂^{7,2′O}G(5′)ppSp(5′)G.

In some embodiments, each RNA described herein comprises the 5′-cap m₂^{7,2′O}G(5′)ppSp(5′)G.

In some embodiments, RNA according to the present disclosure comprises a 5′-UTR and/or a 3′-UTR.

The RNA may comprise further modifications. For example, a further modification of the RNA used in the present invention may be an extension or truncation of the naturally occurring poly(A) tail or an alteration of the 5′- or 3′-untranslated regions (UTR) such as introduction of a UTR which is not related to the coding region of said RNA, for example, the exchange of the existing 3′-UTR with or the insertion of one or more, preferably two copies of a 3′-UTR derived from a globin gene, such as alpha2-globin, alpha1-globin, beta-globin, preferably beta-globin, more preferably human beta-globin.

The term “untranslated region” or “UTR” relates to a region in a DNA molecule which is transcribed but is not translated into an amino acid sequence, or to the corresponding region in an RNA molecule, such as an mRNA molecule. An untranslated region (UTR) can be present 5′ (upstream) of an open reading frame (5′-UTR) and/or 3′ (downstream) of an open reading frame (3′-UTR). A 5′-UTR, if present, is located at the 5′-end, upstream of the start codon of a protein-encoding region. A 5′-UTR is downstream of the 5′-cap (if present), e.g., directly adjacent to the 5′-cap. A 3′-UTR, if present, is located at the 3′-end, downstream of the termination codon of a protein-encoding region, but the term “3′-UTR” does preferably not include the poly-A sequence. Thus, the 3′-UTR is upstream of the poly-A sequence (if present), e.g., directly adjacent to the poly-A sequence. Examples of preferred 5′-UTR and 3′-UTR sequence elements are described herein in detail, are exemplified by SEQ ID NOs: 94, 95, 101 and 102 of the sequence listing, and are referred to in this disclosure.

RNA having an unmasked poly-A sequence is translated more efficiently than RNA having a masked poly-A sequence. The term “poly(A) tail” or “poly-A sequence” relates to an uninterrupted or interrupted sequence of adenyl (A) residues which typically is located on the 3′-end of a RNA molecule and “unmasked poly-A sequence” means that the poly-A sequence at the 3′-end of an RNA molecule ends with an A of the poly-A sequence and is not followed by nucleotides other than A located at the 3′-end, i.e., downstream, of the poly-A sequence. An uninterrupted poly-A tail is characterized by consecutive adenylate residues. In nature, an uninterrupted poly-A tail is typical. RNAs disclosed herein can have a poly-A tail attached to the free 3′-end of the RNA by a template-independent RNA polymerase after transcription or a poly-A tail encoded by DNA and transcribed by a template-dependent RNA polymerase. Furthermore, a long poly-A sequence of about 120 base pairs results in an optimal transcript stability and translation efficiency of RNA.

Therefore, in order to increase stability and/or expression of the RNA used according to the present invention, it may be modified so as to be present in conjunction with a poly-A sequence, preferably having a length of 10 to 500, more preferably 30 to 300, even more preferably 65 to 200 and especially 100 to 150 adenosine residues. In an especially preferred embodiment the poly-A sequence has a length of approximately 120 adenosine residues. To further increase stability and/or expression of the RNA used according to the invention, the poly-A sequence can be unmasked.

In some embodiments, a poly-A tail is attached during RNA transcription, e.g., during preparation of in vitro transcribed RNA, based on a DNA template comprising repeated dT nucleotides (deoxythymidylate) in the strand complementary to the coding strand. The DNA sequence encoding a poly-A tail (coding strand) is referred to as poly(A) cassette.

In some embodiments, the poly(A) cassette present in the coding strand of DNA essentially consists of dA nucleotides, but is interrupted by a random sequence of the four nucleotides (dA, dC, dG, and dT). Such random sequence may be 5 to 50, 10 to 30, or 10 to 20 nucleotides in length. Such a cassette is disclosed in WO 2016/005324 A1, hereby incorporated by reference. Any poly(A) cassette disclosed in WO 2016/005324 A1 may be used in the present invention. A poly(A) cassette that essentially consists of dA nucleotides, but is interrupted by a random sequence having an equal distribution of the four nucleotides (dA, dC, dG, dT) and having a length of e.g., 5 to 50 nucleotides shows, on DNA level, constant propagation of plasmid DNA in E. coli and is still associated, on RNA level, with the beneficial properties with respect to supporting RNA stability and translational efficiency is encompassed. Consequently, in some embodiments, the poly-A tail contained in an RNA molecule described herein essentially consists of A nucleotides, but is interrupted by a random sequence of the four nucleotides (A, C, G, U). Such random sequence may be 5 to 50, to 30, or 10 to 20 nucleotides in length. In one embodiment, the poly(A) cassette comprises or consists of 30 adenine nucleotides, a linker (L) and further 70 adenine nucleotides, also referred to herein as a “A30LA70” poly(A) tail (as exemplified in SEQ ID NO. 103 of the sequence listing).

An RNA for generating a heavy chain of an anti-PD-1 antibody may have the following structure:
(i) 5′-cap—5′-UTR—‘Kozac sequence’—nucleic acid sequence encoding a heavy chain variable region—nucleic sequence encoding a heavy chain constant region—3′-UTR-poly(A)-tail; or
(ii) 5′-cap—5′-UTR—‘Kozac sequence’—secretory signal peptide—nucleic acid sequence encoding a heavy chain variable region—nucleic sequence encoding a heavy chain constant region—3′-UTR—poly(A)-tail.

An RNA for generating a light chain of an anti-PD-1 antibody may have the following structure:
(i) 5′-cap—5′-UTR—‘Kozac sequence’—nucleic acid sequence encoding a light chain variable region—nucleic sequence encoding a light chain constant region—3′-UTR—poly(A)-tail; or
(ii) 5′-cap—5′-UTR—‘Kozac sequence’—secretory signal peptide—nucleic acid sequence encoding a light chain variable region—nucleic sequence encoding a light chain constant region—3′-UTR—poly(A)-tail.

Preferred embodiments of the individual elements are as described hereinabove. For example, the 3′-UTR can be an FI-element and the poly(A) tail can be a A30LA70 element.

In this context, “essentially consists of” means that most nucleotides in the poly-A tail, typically at least 75%, at least 80%, at least 85%, at least 90%, at least 95%, at least 96%, at least 97%, at least 98%, or at least 99% by number of nucleotides in the poly-A tail are A nucleotides, but permits that remaining nucleotides are nucleotides other than A nucleotides, such as U nucleotides (uridylate), G nucleotides (guanylate), or C nucleotides (cytidylate). In this context, “consists of” means that all nucleotides in the poly-A tail, i.e., 100% by number of nucleotides in the poly-A tail, are A nucleotides. The term “A nucleotide” or “A” refers to adenylate.

In some embodiments, no nucleotides other than A nucleotides flank a poly-A tail at its 3′-end, i.e., the poly-A tail is not masked or followed at its 3′-end by a nucleotide other than A.

In some embodiments, at least one RNA comprises a poly-A tail. In some embodiments, each RNA comprises a poly-A tail. In some embodiments, the poly-A tail may comprise at least 20, at least 30, at least 40, at least 80, or at least 100 and up to 500, up to 400, up to 300, up to 200, or up to 150 nucleotides. In some embodiments, the poly-A tail may essentially consist of at least 20, at least 30, at least 40, at least 80, or at least 100 and up to 500, up to 400, up to 300, up to 200, or up to 150 nucleotides. In some embodiments, the poly-A tail may consist of at least 20, at least 30, at least 40, at least 80, or at least 100 and up to 500, up to 400, up to 300, up to 200, or up to 150 nucleotides. In some embodiments, the poly-A tail comprises at least 100 nucleotides. In some embodiments, the poly-A tail comprises about 150 nucleotides. In some embodiments, the poly-A tail comprises about 120 nucleotides.

In addition, incorporation of a 3′-non translated region (UTR) into the 3′-non translated region of an RNA molecule can result in an enhancement in translation efficiency. A synergistic effect may be achieved by incorporating two or more of such 3′-non translated regions. The 3′-non translated regions may be autologous or heterologous to the RNA into which they are introduced. In one particular embodiment the 3′-non translated region is derived from the human β-globin gene.

A combination of the above described modifications, i.e., incorporation of a poly-A sequence, unmasking of a poly-A sequence and incorporation of one or more 3′-non translated regions, has a synergistic influence on the stability of RNA and increase in translation efficiency.

The term “stability” of RNA relates to the “half-life” of RNA. “Half-life” relates to the period of time which is needed to eliminate half of the activity, amount, or number of molecules. In the context of the present invention, the half-life of an RNA is indicative for the stability of said RNA. The half-life of RNA may influence the “duration of expression” of the RNA. It can be expected that RNA having a long half-life will be expressed for an extended time period.

Of course, if according to the present invention it is desired to decrease stability and/or translation efficiency of RNA, it is possible to modify RNA so as to interfere with the function of elements as described above increasing the stability and/or translation efficiency of RNA.

The term “expression” is used according to the invention in its most general meaning and comprises the production of RNA and/or peptides, polypeptides or proteins, e.g., by transcription and/or translation. With respect to RNA, the term “expression” or “translation” relates in particular to the production of peptides, polypeptides or proteins. It also comprises partial expression of nucleic acids. Moreover, expression can be transient or stable. According to the invention, an antibody is expressed in a cell if the antibody can be detected in the cell or a lysate thereof by conventional techniques for protein detection such as techniques using antibodies specifically binding to the PD-1 antibody.

In the context of the present invention, the term “transcription” relates to a process, wherein the genetic code in a DNA sequence is transcribed into RNA. Subsequently, the RNA may be translated into protein. According to the present invention, the term “transcription” comprises “in vitro transcription”, wherein the term “in vitro transcription” relates to a process wherein RNA, in particular mRNA, is in vitro synthesized in a cell-free system, preferably using appropriate cell extracts. Preferably, cloning vectors are applied for the generation of transcripts. These cloning vectors are generally designated as transcription vectors and are according to the present invention encompassed by the term “vector”. According to the present invention, the RNA used in the present invention preferably is in vitro transcribed RNA (IVT-RNA) and may be obtained by in vitro transcription of an appropriate DNA template. The promoter for controlling transcription can be any promoter for any RNA polymerase. Particular examples of RNA polymerases are the T7, T3, and SP6 RNA polymerases. Preferably, the in vitro transcription according to the invention is controlled by a T7 or SP6 promoter. A DNA template for in vitro transcription may be obtained by cloning of a nucleic acid, in particular cDNA, and introducing it into an appropriate vector for in vitro transcription. The cDNA may be obtained by reverse transcription of RNA.

The term “translation” according to the invention relates to the process in the ribosomes of a cell by which a strand of messenger RNA directs the assembly of a sequence of amino acids to make a peptide, polypeptide or protein.

Expression control sequences or regulatory sequences, which according to the invention may be linked functionally with a nucleic acid, can be homologous or heterologous with respect to the nucleic acid. A coding sequence and a regulatory sequence are linked together “functionally” if they are bound together covalently, so that the transcription or translation of the coding sequence is under the control or under the influence of the regulatory sequence. If the coding sequence is to be translated into a functional protein, with functional linkage of a regulatory sequence with the coding sequence, induction of the regulatory sequence leads to a transcription of the coding sequence, without causing a reading frame shift in the coding sequence or inability of the coding sequence to be translated into the desired protein or peptide.

The term “expression control sequence” or “regulatory sequence” comprises, according to the invention, promoters, ribosome-binding sequences and other control elements, which control the transcription of a nucleic acid or the translation of the derived RNA. In certain embodiments of the invention, the regulatory sequences can be controlled. The precise structure of regulatory sequences can vary depending on the species or depending on the cell type, but generally comprises 5′-untranscribed and 5′- and 3′-untranslated sequences, which are involved in the initiation of transcription or translation, such as TATA-box, capping-sequence, CAAT-sequence and the like. In particular, 5′-untranscribed regulatory sequences comprise a promoter region that includes a promoter sequence for transcriptional control of the functionally bound gene. Regulatory sequences can also comprise enhancer sequences or upstream activator sequences.

Preferably, according to the invention, RNA to be expressed in a cell is introduced into said cell. In one embodiment of the methods according to the invention, the RNA that is to be introduced into a cell is obtained by in vitro transcription of an appropriate DNA template.

According to the invention, terms such as “RNA capable of expressing” and “RNA encoding” are used interchangeably herein and with respect to a particular peptide or polypeptide mean that the RNA, if present in the appropriate environment, preferably within a cell, can be expressed to produce said peptide or polypeptide. Preferably, RNA according to the invention is able to interact with the cellular translation machinery to provide the peptide or polypeptide it is capable of expressing.

Terms such as “transferring”, “introducing” or “transfecting” are used interchangeably herein and relate to the introduction of nucleic acids, in particular exogenous or heterologous nucleic acids, in particular RNA into a cell. According to the present invention, the cell can form part of an organ, a tissue and/or an organism. According to the present invention, the administration of a nucleic acid is either achieved as naked nucleic acid or in combination with an administration reagent. Preferably, administration of nucleic acids is in the form of naked nucleic acids. Preferably, the RNA is administered in combination with stabilizing substances such as RNase inhibitors. The present invention also envisions the repeated introduction of nucleic acids into cells to allow sustained expression for extended time periods.

Cells can be transfected with any carriers with which the nucleic acid, for example the RNA can be associated, e.g., by forming complexes with the RNA or forming vesicles in which the RNA is enclosed or encapsulated, resulting in increased stability of the RNA compared to naked RNA. Carriers useful according to the invention include, for example, lipid-containing carriers such as cationic lipids, liposomes, in particular cationic liposomes, and micelles, and nanoparticles, such as lipoplex particles. Cationic lipids may form complexes with negatively charged nucleic acids. Any cationic lipid may be used according to the invention.

Cells which can be transfected also comprise host cells, which will become recombinant. The term “recombinant host cell”, as used herein, is intended to refer to a cell into which a recombinant expression vector has been introduced. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “recombinant host cell” as used herein. Host cells and recombinant host cells include, for example, transfectomas, such as CHO cells, NS/0 cells, Sp2/0 cells, COS cells, Vero cells, HeLa cells, HEK293 cells, HEK293T cells, HEK293T/17 cells, and lymphocytic cells.

The host cells used to produce the antibodies as defined herein may be cultured in a variety of media, which are commerialy available and well known to the skilled person. Any of these media may be supplemented as necessary with hormones and/or other growth factors.

In certain embodiments of the present disclosure, the RNA described herein may be present in RNA lipoplex particles. The RNA lipoplex particles and compositions comprising RNA lipoplex particles described herein are useful for delivery of RNA to a target tissue after parenteral administration, in particular after intravenous administration. The RNA lipoplex particles may be prepared using liposomes that may be obtained by injecting a solution of the lipids in ethanol into water or a suitable aqueous phase. In one embodiment, the aqueous phase has an acidic pH. In one embodiment, the aqueous phase comprises acetic acid, e.g., in an amount of about 5 mM. In one embodiment, the liposomes and RNA lipoplex particles comprise at least one cationic lipid and at least one additional lipid. In one embodiment, the at least one cationic lipid comprises 1,2-di-O-octadecenyl-3-trimethylammonium propane (DOTMA) and/or 1,2-dioleoyl-3-trimethylammonium-propane (DOTAP). In one embodiment, the at least one additional lipid comprises 1,2-di-(9Z-octadecenoyl)-sn-glycero-3-phosphoethanolamine (DOPE), cholesterol (Chol) and/or 1,2-dioleoyl-sn-glycero-3-phosphocholine (DOPC). In one embodiment, the at least one cationic lipid comprises 1,2-di-0-octadecenyl-3-trimethylammonium propane (DOTMA) and the at least one additional lipid comprises 1,2-di-(9Z-octadecenoyl)-sn-glycero-3-phosphoethanolamine (DOPE). In one embodiment, the liposomes and RNA lipoplex particles comprise 1,2-di-O-octadecenyl-3-trimethylammonium propane (DOTMA) and 1,2-di-(9Z-octadecenoyl)-sn-glycero-3-phosphoethanolamine (DOPE). Liposomes may be used for preparing RNA lipoplex particles by mixing the liposomes with RNA.

RNA lipoplex particles may have an average diameter that in one embodiment ranges from about 200 nm to about 1000 nm, from about 200 nm to about 800 nm, from about 250 to about 700 nm, from about 400 to about 600 nm, from about 300 nm to about 500 nm, or from about 350 nm to about 400 nm. In an embodiment, the RNA lipoplex particles have an average diameter that ranges from about 250 nm to about 700 nm. In another embodiment, the RNA lipoplex particles have an average diameter that ranges from about 300 nm to about 500 nm. In an exemplary embodiment, the RNA lipoplex particles have an average diameter of about 400 nm.

The RNA lipoplex particles can exhibit a polydispersity index less than about 0.5, less than about 0.4, or less than about 0.3. By way of example, the RNA lipoplex particles can exhibit a polydispersity index in a range of about 0.1 to about 0.3.

The lipid solutions, liposomes and RNA lipoplex particles can include a cationic lipid. As used herein, a “cationic lipid” refers to a lipid having a net positive charge. Cationic lipids bind negatively charged RNA by electrostatic interaction to the lipid matrix. Generally, cationic lipids possess a lipophilic moiety, such as a sterol, an acyl or diacyl chain, and the head group of the lipid typically carries the positive charge. Examples of cationic lipids include, but are not limited to 1,2-di-O-octadecenyl-3-trimethylammonium propane (DOTMA), dimethyldioctadecylammonium (DDAB); 1,2-dioleoyl-3-trimethylammonium propane (DOTAP); 1,2-dioleoyl-3-dimethylammonium-propane (DODAP); 1,2-diacyloxy-3-dimethylammonium propanes; 1,2-dialkyloxy-3-dimethylammonium propanes; dioctadecyldimethyl ammonium chloride (DODAC), 2,3-di(tetradecoxy)propyl-(2-hydroxyethyl)-dimethylazanium (DMRIE), 1,2-dimyristoyl-sn-glycero-3-ethylphosphocholine (DMEPC), 1,2-dimyristoyl-3-trimethylammonium propane (DMTAP), 1,2-dioleyloxypropyl-3-dimethyl-hydroxyethyl ammonium bromide (DORIE), and 2,3-dioleoyloxy-N-[2(spermine carboxamide)ethyl]-N,N-dimethyl-1-propanamium trifluoroacetate (DOSPA). Preferred are DOTMA, DOTAP, DODAC, and DOSPA. In specific embodiments, the cationic lipid is DOTMA and/or DOTAP.

An additional lipid may be incorporated to adjust the overall positive to negative charge ratio and physical stability of the RNA lipoplex particles. In certain embodiments, the additional lipid is a neutral lipid. As used herein, a “neutral lipid” refers to a lipid having a net charge of zero. Examples of neutral lipids include, but are not limited to, 1,2-di-(9Z-octadecenoyl)-sn-glycero-3-phosphoethanolamine (DOPE), 1,2-dioleoyl-sn-glycero-3-phosphocholine (DOPC), diacylphosphatidyl choline, diacylphosphatidyl ethanol amine, ceramide, sphingoemyelin, cephalin, cholesterol, and cerebroside. In specific embodiments, the additional lipid is DOPE, cholesterol and/or DOPC.

In certain embodiments, the RNA lipoplex particles include both a cationic lipid and an additional lipid. In an exemplary embodiment, the cationic lipid is DOTMA and the additional lipid is DOPE. Without wishing to be bound by theory, the amount of the at least one cationic lipid compared to the amount of the at least one additional lipid may affect important RNA lipoplex particle characteristics, such as charge, particle size, stability, tissue selectivity, and bioactivity of the RNA. Accordingly, in some embodiments, the molar ratio of the at least one cationic lipid to the at least one additional lipid is from about 10:0 to about 1:9, about 4:1 to about 1:2, or about 3:1 to about 1:1. In specific embodiments, the molar ratio may be about 3:1, about 2.75:1, about 2.5:1, about 2.25:1, about 2:1, about 1.75:1, about 1.5:1, about 1.25:1, or about 1:1. In an exemplary embodiment, the molar ratio of the at least one cationic lipid to the at least one additional lipid is about 2:1.

The electric charge of the RNA lipoplex particles is the sum of the electric charges present in the at least one cationic lipid and the electric charges present in the RNA. The charge ratio is the ratio of the positive charges present in the at least one cationic lipid to the negative charges present in the RNA. The charge ratio of the positive charges present in the at least one cationic lipid to the negative charges present in the RNA is calculated by the following equation: charge ratio=[(cationic lipid concentration (mol))*(the total number of positive charges in the cationic lipid)]/[(RNA concentration (mol))*(the total number of negative charges in RNA)]. The concentration of RNA and the at least one cationic lipid amount can be determined using routine methods by one skilled in the art. In one embodiment, at physiological pH the charge ratio of positive charges to negative charges in the RNA lipoplex particles is from about 1.6:2 to about 1:2, or about 1.6:2 to about 1.1:2. In specific embodiments, the charge ratio of positive charges to negative charges in the RNA lipoplex particles at physiological pH is about 1.6:2.0, about 1.5:2.0, about 1.4:2.0, about 1.3:2.0, about 1.2:2.0, about 1.1:2.0, or about 1:2.0.

RNA lipoplex particles can, for example, be obtained by mixing the RNA with liposomes or with at least one cationic lipid for example by using an ethanol injection technique. The obtained compositions may according to the present invention comprise salts such as sodium chloride. Without wishing to be bound by theory, sodium chloride functions as an ionic osmolality agent for preconditioning RNA prior to mixing with the at least one cationic lipid. Certain embodiments contemplate alternative organic or inorganic salts to sodium chloride in the present disclosure. Alternative salts include, without limitation, potassium chloride, dipotassium phosphate, monopotassium phosphate, potassium acetate, potassium bicarbonate, potassium sulfate, potassium acetate, disodium phosphate, monosodium phosphate, sodium acetate, sodium bicarbonate, sodium sulfate, sodium acetate, lithium chloride, magnesium chloride, magnesium phosphate, calcium chloride, and sodium salts of ethylenediaminetetraacetic acid (EDTA).

Generally, compositions comprising RNA lipoplex particles may comprise sodium chloride at a concentration that preferably ranges from 0 mM to about 500 mM, from about 5 mM to about 400 mM, or from about 10 mM to about 300 mM. In one embodiment, compositions comprising RNA lipoplex particles comprise an ionic strength corresponding to such sodium chloride concentrations.

The term “ionic strength” refers to the mathematical relationship between the number of different kinds of ionic species in a particular solution and their respective charges. Thus, ionic strength I is represented mathematically by the formula

$$l = \frac{1}{2} \cdot {\sum }_{i} {z}_{i}^{2} \cdot {c}_{i}$$

in which c is the molar concentration of a particular ionic species and z the absolute value of its charge. The sum Σ is taken over all the different kinds of ions (i) in solution.

According to the disclosure, the term “ionic strength” in one embodiment relates to the presence of monovalent ions. Regarding the presence of divalent ions, in particular divalent cations, their concentration or effective concentration (presence of free ions) due to the presence of chelating agents is in one embodiment sufficiently low so as to prevent degradation of the RNA. In one embodiment, the concentration or effective concentration of divalent ions is below the catalytic level for hydrolysis of the phosphodiester bonds between RNA nucleotides. In one embodiment, the concentration of free divalent ions is 20 μM or less. In one embodiment, there are no or essentially no free divalent ions.

These compositions may alternatively or in addition comprise a stabilizer to avoid substantial loss of the product quality and, in particular, substantial loss of RNA activity during freezing, lyophilization, spray-drying or storage such as storage of the frozen, lyophilized or spray-dried composition. Lyophilized or spray-dried compositions can be reconstituted before use. In an embodiment the stabilizer is a carbohydrate. The term “carbohydrate”, as used herein refers to and encompasses monosaccharides, disaccharides, trisaccharides, oligosaccharides, and polysaccharides. In embodiments of the disclosure, the stabilizer is mannose, glucose, sucrose or trehalose. According to the present invention, the RNA lipoplex particle compositions may have a stabilizer concentration suitable for the stability of the composition, in particular for the stability of the RNA lipoplex particles and for the stability of the RNA.

The term “freezing” relates to the solidification of a liquid, usually with the removal of heat.

The term “lyophilizing” or “lyophilization” refers to the freeze-drying of a substance by freezing it and then reducing the surrounding pressure to allow the frozen medium in the substance to sublimate directly from the solid phase to the gas phase.

The term “spray-drying” refers to spray-drying a substance by mixing (heated) gas with a fluid that is atomized (sprayed) within a vessel (spray dryer), where the solvent from the formed droplets evaporates, leading to a dry powder.

The term “reconstitute” relates to adding a solvent such as water to a dried product to return it to a liquid state such as its original liquid state.

According to the present invention, the RNA lipoplex particle compositions may have a pH suitable for the stability of the RNA lipoplex particles and, in particular, for the stability of the RNA. In one embodiment, the RNA lipoplex particle compositions described herein have a pH from about 5.5 to about 7.5.

According to the present invention, the compositions may include at least one buffer. Without wishing to be bound by theory, the use of buffer maintains the pH of the composition during manufacturing, storage and use of the composition. In certain embodiments of the present invention, the buffer may be sodium bicarbonate, monosodium phosphate, disodium phosphate, monopotassium phosphate, dipotassium phosphate, [tris(hydroxymethyl)methyl-amino]propanesulfonic acid (TAPS), 2-(Bis(2-hydroxyethyl)amino)acetic acid (Bicine), 2-Amino-2-(hydroxymethyl)propane-1,3-diol (Tris), N-(2-Hydroxy-1,1-bis(hydroxy-methyl)ethyl)glycine (Tricine), 3-[[1,3-dihydroxy-2-(hydroxymethyl)propan-2-yl]amino]-2-hydroxypropane-1-sulfonic acid (TAPSO), 2-[4-(2-hydroxyethyl)piperazin-1-yl]ethanesulfonic acid (HEPES), 2-[[1,3-dihydroxy-2-(hydroxymethyl)propan-2-yl]amino]ethanesulfonic acid (TES), 1,4-piperazinediethanesulfonic acid (PIPES), dimethylarsinic acid, 2-morpholin-4-ylethanesulfonic acid (MES), 3-morpholino-2-hydroxypropanesulfonic acid (MOPSO), or phosphate buffered saline (PBS). Other suitable buffers may be acetic acid in a salt, citric acid in a salt, boric acid in a salt and phosphoric acid in a salt. In one embodiment, the buffer is HEPES. In one embodiment, the buffer has a concentration from about 2.5 mM to about 15 mM.

Certain embodiments of the present invention contemplate the use of a chelating agent. Chelating agents refer to chemical compounds that are capable of forming at least two coordinate covalent bonds with a metal ion, thereby generating a stable, water-soluble complex. Without wishing to be bound by theory, chelating agents reduce the concentration of free divalent ions, which may otherwise induce accelerated RNA degradation. Examples of suitable chelating agents include, without limitation, ethylenediaminetetraacetic acid (EDTA), a salt of EDTA, desferrioxamine B, deferoxamine, dithiocarb sodium, penicillamine, pentetate calcium, a sodium salt of pentetic acid, succimer, trientine, nitrilotriacetic acid, trans-diaminocyclohexanetetraacetic acid (DCTA), diethylenetriaminepentaacetic acid (DTPA), bis(aminoethyl)glycolether-N,N,N′,N′-tetraacetic acid, iminodiacetic acid, citric acid, tartaric acid, fumaric acid, or a salt thereof. In certain embodiments, the chelating agent is EDTA or a salt of EDTA. In an exemplary embodiment, the chelating agent is EDTA disodium dihydrate. In some embodiments, the EDTA is at a concentration from about 0.05 mM to about 5 mM.

The composition comprising the RNA lipoplex particles can be in a liquid or a solid. Non-limiting examples of a solid include a frozen form or a lyophilized form. In a preferred embodiment, the composition is a liquid.

If provided as lipoplex particles, the RNA encoding an antibody is co-formulated as particles such as lipoplex particles with the RNA encoding an amino acid sequence which breaks immunological tolerance at a ratio of about 4:1 to about 16:1, about 6:1 to about 14:1, about 8:1 to about 12:1, or about 10:1.

In the context of the present disclosure, the term “particle” relates to a structured entity formed by molecules or molecule complexes. In one embodiment, the term “particle” relates to a micro- or nano-sized structure, such as a micro- or nano-sized compact structure.

In the context of the present disclosure, the term “RNA lipoplex particle” relates to a particle that contains lipid, in particular cationic lipid, and RNA. Electrostatic interactions between positively charged liposomes and negatively charged RNA results in complexation and spontaneous formation of RNA lipoplex particles. Positively charged liposomes may be generally synthesized using a cationic lipid, such as DOTMA, and additional lipids, such as DOPE. In one embodiment, a RNA lipoplex particle is a nanoparticle.

As used in the present disclosure, “nanoparticle” refers to a particle comprising RNA and at least one cationic lipid and having an average diameter suitable for intravenous administration.

The term “average diameter” refers to the mean hydrodynamic diameter of particles as measured by dynamic light scattering (DLS) with data analysis using the so-called cumulant algorithm, which provides as results the so-called Zaverage with the dimension of a length, and the polydispersity index (PI), which is dimensionless (Koppel, D., J. Chem. Phys. 57, 1972, pp 4814-4820, ISO 13321). Here “average diameter”, “diameter” or “size” for particles is used synonymously with this value of the Zaverage.

The term “polydispersity index” is used herein as a measure of the size distribution of an ensemble of particles, e.g., nanoparticles. The polydispersity index is calculated based on dynamic light scattering measurements by the so-called cumulant analysis.

The term “ethanol injection technique” refers to a process, in which an ethanol solution comprising lipids is rapidly injected into an aqueous solution through a needle. This action disperses the lipids throughout the solution and promotes lipid structure formation, for example lipid vesicle formation such as liposome formation. Generally, the RNA lipoplex particles described herein are obtainable by adding RNA to a colloidal liposome dispersion. Using the ethanol injection technique, such colloidal liposome dispersion is, in one embodiment, formed as follows: an ethanol solution comprising lipids, such as cationic lipids like DOTMA and additional lipids, is injected into an aqueous solution under stirring. In one embodiment, the RNA lipoplex particles described herein are obtainable without a step of extrusion.

The term “extruding” or “extrusion” refers to the creation of particles having a fixed, cross-sectional profile. In particular, it refers to the downsizing of a particle, whereby the particle is forced through filters with defined pores.

Instead of providing/administering the nucleic acids by using carriers as include, for example, lipid-containing carriers such as cationic lipids, liposomes, in particular cationic liposomes, and micelles, and nanoparticles, such as lipoplex particles, according to the present invention the nucleic acids of interest can be provided/administered also by using recombinant host cells, preferably those as specified above, or recombinant viruses encoding the antibody or an antibody fragment derived from the antibody.

These viruses may be DNA or RNA viruses. Several viral vectors have shown promising results with regard to their potential to enhance immunotherapy of malignant diseases. Replication competent and replication incompetent viruses can be used, with the latter group being preferred. Herpes virus, adenovirus, vaccinia, reovirus, and New Castle Disease viruses are examples of preferred viruses useful according to the present invention. In one embodiment the virus or viral vector is selected from the group consisting of adenoviruses, adeno-associated viruses, pox viruses, including vaccinia virus and attenuated pox viruses, Semliki Forest virus, reoviruses, retroviruses, New Castle Disease viruses, Sindbis virus and Ty virus-like particles. Particular preference is given to adenoviruses and retroviruses. The retroviruses are typically replication-deficient (i.e., they are incapable of generating infectious particles).

Methods of introducing nucleic acids into cells in vitro or in vivo comprise transfection of nucleic acid calcium phosphate precipitates, transfection of nucleic acids associated with DEAE, transfection or infection with the above viruses carrying the nucleic acids of interest, liposome-mediated transfection, and the like. In particular embodiments, preference is given to directing the nucleic acid to particular cells. In such embodiments, a carrier used for administering a nucleic acid to a cell (e.g., a retrovirus or a liposome) may have a bound target control molecule. For example, a molecule such as an antibody specific for a surface membrane protein on the target cell or a ligand for a receptor on the target cell may be incorporated into or attached to the nucleic acid carrier. Preferred antibodies comprise antibodies which bind selectively a tumor antigen. If administration of a nucleic acid via liposomes is desired, proteins binding to a surface membrane protein associated with endocytosis may be incorporated into the liposome formulation in order to make target control and/or uptake possible. Such proteins comprise capsid proteins or fragments thereof which are specific for a particular cell type, antibodies to proteins which are internalized, proteins addressing an intracellular site, and the like.

Preferably, the introduction of RNA which encodes a peptide or polypeptide into a cell, in particular into a cell present in vivo, results in expression of said peptide or polypeptide in the cell. In particular embodiments, the targeting of the nucleic acids to particular cells is preferred. In such embodiments, a carrier which is applied for the administration of the nucleic acid to a cell (for example, a retrovirus or a liposome), exhibits a targeting molecule. For example, a molecule such as an antibody which is specific for a surface membrane protein on the target cell or a ligand for a receptor on the target cell may be incorporated into the nucleic acid carrier or may be bound thereto. In case the nucleic acid is administered by liposomes, proteins which bind to a surface membrane protein which is associated with endocytosis may be incorporated into the liposome formulation in order to enable targeting and/or uptake. Such proteins encompass capsid proteins of fragments thereof which are specific for a particular cell type, antibodies against proteins which are internalized, proteins which target an intracellular location etc.

It is to be understood that, unless indicated otherwise herein or clearly contradicted by context, that the teaching provided with regard to nucleic acids encoding an antibody under point VI herein is applicable accordingly to nucleic acids/polynucleotides encoding a peptide or protein comprising an epitope of an antigen. Spleen targeting RNA lipoplex particles, which may be beneficially used for expressing RNA in antigen presenting cells, are described in WO 2013/143683, herein incorporated by reference.

The nucleic acids or vectors (such as RNA or RNA-based vectors), as provided herein, for generating anti-PD-1 antibody may be produced by an in vitro transcription method.

Such a method comprises a step of inserting a DNA sequence of a heavy chain variable region (VH) or a light chain variable region (VL), as defined hereinabove, e.g., SEQ ID NOs: 74 to 92 of the sequence listing), optionally N-terminally of the immunoglobulin constant part(s) into the IVT-vector (e.g., a pST4 vector) using standard cloning techniques. The vector may comprise a 5′-UTR as defined herein, a 3′-UTR as defined herein, e.g., a FI-element, a poly(A) tail as defined hereinabove, e.g., a poly (A) tail comprising of 30 adenine nucleotides, a linker (L) and further (A30LA70). In addition, the IVT vector may optionally comprise a nucleic acid sequence encoding for a secretory signal peptide, e.g., a secretory signal peptide as defined herein.

To generate templates for in vitro transcription, the plasmid DNAs can be linearized downstream of the poly(A) tail-encoding region using, e.g., a restriction endonuclease, thereby generating a template to transcribe mRNA, e.g., by using a T7 RNA polymerase.

During in vitro transcription, the RNA may be modified to minimize immunogenicity, and the RNA may be capped at its 5′-end.

The thus obtained, optionally capped, RNA is used to transfect host cells, e.g., NS0 cells, Sp2/0 cells, HEK293 cells or derivates thereof, such as HEK293T, HEK293T/17 and/or HEK293F, COS cells, Vero cells and/or HeLa cells. In one embodiment, the mammalian host cell is selected from HEK293, HEK293T and/or HEK293T/17 cells. For transfection, liposomes, e.g., as described hereinabove, may be used.

The transfected cells are used to express the antibodies or antibody chains or fragments thereof. In order to express both the H chain and the L chain of the anti-PD-1 antibody, the host cells are preferably transfected with both types of RNA, i.e., individual RNAs, each encoding the H chain and the L chain of the anti-PD-1 antibody.

The anti-PD-1 antibody can be produced intracellularly, in the periplasmic space, or can be directly secreted into the medium. If the antibody is produced intracellularly, the cells may be lysed afterwards and the cell debris is to be removed, e.g., by centrifugation or ultrafiltration. The skilled person is familiar with suitable methods for isolating intracellularly produced antibodies. The same applies for methods for isolating antibodies which are secreted to the periplasmic space. Where the antibody is secreted into the medium, e.g., by using a secretoty signal peptide, supernatants from such expression systems may be first concentrated, e.g., by using a commercially available protein concentration filter. A protease inhibitor, e.g, PMSF may be included in any of the foregoing steps to inhibit proteolysis and antibiotics may be included to prevent the growth of contaminants. The anti-PD-1 antibodies prepared from the transfected host cells can be purified, e.g., by using chromatography, such as affinity chromatography, gel electrophoresis, flow cytometry and/or dialysis.

VII. Pharmaceutical Compositions

In another aspect, the present invention provides a composition, e.g., a pharmaceutical composition, comprising one or a combination of antibodies, including the conjugates and/or multimers, of the present invention and/or comprising one or a combination of nucleic acids comprising a nucleic acid sequence encoding an antibody, including host cells or vectors comprising the said nucleic acid, of the present invention. The pharmaceutical compositions may be formulated with pharmaceutically acceptable carriers or diluents as well as any other known adjuvants and excipients in accordance with conventional techniques such as those disclosed in Remington: The Science and Practice of Pharmacy, 19th Edition, Gennaro, Ed., Mack Publishing Co., Easton, P A, 1995. In one embodiment, the compositions include a combination of multiple (e.g., two or more) isolated antibodies. In another embodiment, the compositions include a combination of multiple (e.g., two or more) nucleic acids, vectors or host cells.

As used herein, “pharmaceutically acceptable carrier” includes any and all solvents, dispersion media, coatings, antibacterial and antifungal agents, isotonic and absorption delaying agents, and the like that are physiologically compatible. Preferably, the carrier is suitable for cardiovascular (e.g., intravenous or intraarterial), intramuscular, subcutaneous, parenteral, spinal or epidermal administration (e.g., by injection or infusion). Depending on the route of administration, the active compound, i.e., antibody, bispecific and multispecific molecule, nucleic acids, vectors, may be coated in a material to protect the compound from the action of acids and other natural conditions that may inactivate the compound.

A “pharmaceutically acceptable substance” refers to a substance that retains the desired biological activity of the parent compound and does not impart any undesired toxicological effects (see e.g., Berge, S. M., et al. (1977) J. Pharm. Sci. 66: 1-19).

The carrier can be a solvent or dispersion medium comprising, for example, water, ethanol, polyol (for example, glycerol, propylene glycol, and liquid polyethylene glycol, and the like), saline and aqueous buffer solutions, vegetable oils, such as olive oil, and injectable organic esters, such as ethyl oleate and suitable mixtures thereof. The proper fluidity can be maintained, for example, by the use of a coating such as lecithin, by the maintenance of the required particle size in the case of dispersion and by the use of surfactants.

The carrier or the composition of the present invention can also comprise pharmaceutically acceptable salts. Examples of pharmaceutically acceptable salts that may be comprised include acid addition salts and base addition salts. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like.

The composition of the present invention may also comprise antioxidants. Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

The compositions of the present invention may also contain adjuvants such as preservatives, wetting agents, emulsifying agents and dispersing agents. Prevention of the presence of microorganisms may be ensured both by sterilization procedures, and by the inclusion of various antibacterial and antifungal agents, for example, paraben, chlorobutanol, phenol sorbic acid, and the like. It may also be desirable to include isotonic agents, for example, sugars, polyalcohols such as mannitol, sorbitol, or sodium chloride in the composition. In addition, prolonged absorption of the injectable pharmaceutical form may be brought about by the inclusion of agents which delay absorption, for example, monostearate salts and gelatin.

Pharmaceutically acceptable carriers include sterile aqueous solutions or dispersions and sterile powders for the extemporaneous preparation of sterile injectable solutions or dispersions. The use of such media and agents for pharmaceutically active substances is known in the art. Except insofar as any conventional media or agent is incompatible with the active compound, use thereof in the pharmaceutical compositions of the invention is contemplated. Supplementary active compounds can also be incorporated into the compositions.

Pharmaceutically compositions typically must be sterile and stable under the conditions of manufacture and storage. The composition can be formulated as a solution, microemulsion, liposome, or other ordered structure suitable to high drug concentration.

A composition of the present invention can be administered by a variety of methods known in the art. As will be appreciated by the skilled artisan, the route and/or mode of administration will vary depending upon the desired results. The active compounds can be prepared with carriers that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Methods for the preparation of such formulations are generally known to those skilled in the art. See, e.g., Sustained and Controlled Release Drug Delivery Systems, J. R. Robinson, ed., Marcel Dekker, Inc., New York, 1978.

To administer a compound (e.g., an antibody or a nucleic acid or a vector or a combination of nucleic acids or vectors) of the invention by certain routes of administration, it may be necessary to coat the compound with, or co-administer the compound with, a material to prevent its inactivation. For example, the compound may be administered to a subject in an appropriate carrier, for example, liposomes, or a diluent. Pharmaceutically acceptable diluents include saline and aqueous buffer solutions. Liposomes include water-in-oil-in-water CGF emulsions as well as conventional liposomes (Strejan et al. (1984) J. Neuroimmunol. 7: 27).

Sterile injectable solutions can be prepared by incorporating the active compound in the required amount in an appropriate solvent with one or a combination of ingredients enumerated above, as required, followed by sterilization microfiltration.

Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying (lyophilization) that yield a powder of the active ingredient plus any additional desired ingredient from a previously sterile-filtered solution thereof.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms of the invention are dictated by and directly dependent on (a) the unique characteristics of the active compound and the particular therapeutic effect to be achieved, and (b) the limitations inherent in the art of compounding such an active compound for the treatment of sensitivity in individuals.

Pharmaceutical formulations of the present invention include those suitable for oral, nasal, topical (including buccal and sublingual), rectal, vaginal and/or parenteral administration. The formulations may conveniently be presented in unit dosage form and may be prepared by any methods known in the art of pharmacy. The amount of active ingredient to produce a single dosage form will vary depending upon the subject being treated, and the particular mode of administration. The amount of active ingredient to produce a single dosage form will generally be that amount of the composition which produces a therapeutic effect.

Dosage forms for the topical or transdermal administration of compositions of this invention include powders, sprays, ointments, pastes, creams, lotions, gels, solutions, patches and inhalants. The active compound may be mixed under sterile conditions with a pharmaceutically acceptable carrier, and with any preservatives or other adjuvants or excipients which may be required.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

In one embodiment, the anti-PD-1 antibody is to be administered as protein, wherein the antibody can have been obtained from hybridomas, transfectomas or by in vitro transcription, as described herein. In one embodiment, the anti-PD-1 antibody is to be administered as one or more nucleic acids or as one or more vectors as defined herein, e.g., as RNA or liposomes comprising the RNA or one or more RNAs which encode for the antibody or a chain of the antibody or a fragment of such antibody or chain.

In one embodiment the antibodies of the invention are administered in crystalline form by subcutaneous injection, see, Yang et al. (2003) PNAS, 100 (12): 6934-6939.

When the compounds of the present invention are administered as pharmaceuticals, to humans and animals, they can be given alone or as a pharmaceutical composition comprising, for example, from about 0.01 percent to about ninety-nine percent of active ingredient, preferably from about 0.1 percent to about 90 percent, most preferably from about 1 percent to about 50 percent, in combination with a pharmaceutically acceptable carrier, preferably a pharmaceutically acceptable carrier as specified above. In addition, adjuvants and/or excipients, such as antioxidants or preservatives, may be comprised in addition.

Regardless of the route of administration selected, the compounds of the present invention, which may be used in a suitable hydrated form, and/or the pharmaceutical compositions of the present invention, are formulated into pharmaceutically acceptable dosage forms by conventional methods known to those of skill in the art.

Pharmaceutical compositions can be administered with medical devices known in the art. For example, in a preferred embodiment, a pharmaceutical composition of the invention can be administered with a needleless hypodermic injection device, such as the devices disclosed in U.S. Pat. Nos. 5,399,163; 5,383,851; 5,312,335; 5,064,413; 4,941,880; 4,790,824; or U.S. Pat. No. 4,596,556. Examples of well-known implants and modules useful in the present invention include those described in: U.S. Pat. No. 4,487,603, which discloses an implantable micro-infusion pump for dispensing medication at a controlled rate; U.S. Pat. No. 4,486,194, which discloses a therapeutic device for administering medicants through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system.

Many other such implants, delivery systems, and modules are known to those skilled in the art. In certain embodiments, the antibodies of the invention can be formulated to ensure proper distribution in vivo. For example, the blood-brain barrier (BBB) excludes many highly hydrophilic compounds. To ensure that the therapeutic compounds of the invention cross the BBB (if desired), they can be formulated, for example, in liposomes. For methods of manufacturing liposomes, see, e.g., U.S. Pat. Nos. 4,522,811; 5,374,548; and 5,399,331. The liposomes may comprise one or more moieties which are selectively transported into specific cells or organs, and thus enhance targeted drug delivery (see, e.g., V. V. Ranade (1989) J. Clin. Pharmacol. 29: 685). Exemplary targeting moieties include folate or biotin (see, e.g., U.S. Pat. No. 5,416,016 to Low et al.); mannosides (Umezawa et al., (1988) Biochem. Biophys. Res. Commun. 153: 1038); antibodies (P. G. Bloeman et al. (1995) FEBS Lett. 357: 140; M. Owais et al. (1995) Antimicrob. Agents Chemother. 39: 180); and surfactant protein A receptor (Briscoe et al. (1995) Am. J. Physiol. 1233: 134).

In one embodiment of the invention, the therapeutic compounds of the invention are formulated in liposomes. In a more preferred embodiment, the liposomes include a targeting moiety. In a most preferred embodiment, the therapeutic compounds in the liposomes are delivered by bolus injection to a site proximal to the desired area, e.g., the site of a tumor. The composition must be fluid to the extent that easy syringability exists. It must be stable under the conditions of manufacture and storage and must be preserved against the contaminating action of microorganisms such as bacteria and fungi.

In a further embodiment, antibodies of the invention can be formulated to prevent or reduce their transport across the placenta. This can be done by methods known in the art, e.g., by PEGylation of the antibodies or by use of F(ab)2′ fragments. Further references can be made to Cunningham-Rundles C, Zhuo Z, Griffith B, Keenan J. (1992), “Biological activities of polyethylene-glycol immunoglobulin conjugates. Resistance to enzymatic degradation.” J. Immunol. Methods, 152: 177-190; and to Landor M. (1995), “Maternal-fetal transfer of immunoglobulins”, Ann. Allergy Asthma Immunol. 74: 279-283.

The composition must be sterile and fluid to the extent that the composition is deliverable by syringe. In addition to water, the carrier can be an isotonic buffered saline solution, ethanol, polyol (for example, glycerol, propylene glycol, and liquid polyetheylene glycol, and the like), and suitable mixtures thereof. Proper fluidity can be maintained, for example, by use of coating such as lecithin, by maintenance of required particle size in the case of dispersion and by use of surfactants. In many cases, it is preferable to include isotonic agents, for example, sugars, polyalcohols such as mannitol or sorbitol, and sodium chloride in the composition. Long-term absorption of the injectable compositions can be brought about by including in the composition an agent which delays absorption, for example, aluminum monostearate or gelatin.

When the active compound is suitably protected, as described above, the compound may be orally administered, for example, with an inert diluent or an assimilable edible carrier.

VIII. Uses and Methods of the Invention

The antibodies, conjugates, multimers, nucleic acids, vectors, host cells and viruses of the present invention have numerous therapeutic utilities involving the treatment of diseases involving cells expressing PD-1 or its ligands (PD-L 1 and/or PD-L2).

Therefore, in a further aspect the present invention is concerned with the medical use of the antibodies, conjugates, multimers, nucleic acids, vectors, host cells, viruses or compositions of the present invention. In this regard the invention provides antibodies, conjugates, multimers, nucleic acids, vectors, host cells, viruses or compositions, preferably pharmaceutical compositions, for use in the treatment of a disease, e.g., for use in tumor/cancer treatment. The expression “for use in the treatment of a disease, e.g., for use in tumor/cancer treatment” is used herein also replaceable with “for use as a medicament, especially in a method of treatment of cancer”; or the use of said products in the preparation of a pharmaceutical formulation for use in said method of treatment in humans (or more generically a subject in need thereof).

In following, when describing preferred uses and methods of the present invention, reference is made to the antibodies of the present invention. But, it is to be understood that, unless indicated otherwise herein or clearly contradicted by context, that this teaching is also applicable to the other active agents comprising the antibodies or encoding the same, i.e., the conjugates, multimers, nucleic acids, vectors, host cells, viruses or compositions of the present invention.

For example, the antibodies or nucleic acids can be administered to cells in culture, e.g., in vitro or ex vivo, or to subjects, preferably human subjects, e.g., in vivo, to treat or prevent a variety of diseases such as those described herein.

As used herein, the term “subject” is intended to include human and non-human animals which respond to the antibodies against PD-1. The term “non-human animal” includes all vertebrates, e.g., mammals and non-mammals, such as non-human primates, sheep, dog, cow, chickens, amphibians, reptiles, etc. Preferred subjects include human patients having disorders that can be corrected or ameliorated by killing diseased cells.

According to the invention, the term “disease” refers to any pathological state, including cancer or tumor, in particular those forms of tumors or cancer described herein, or autoimmune diseases.

By “tumor” or “cancer” is meant an abnormal group of cells or tissue that grows by a rapid, uncontrolled cellular proliferation and continues to grow after the stimuli that initiated the new growth cease. Tumors show partial or complete lack of structural organization and functional coordination with the normal tissue, and usually form a distinct mass of tissue, which may be either benign or malignant. These terms according to the disclosure also comprise metastases. For purposes of the present invention, the terms “cancer” and “cancer disease” are used interchangeably with the terms “tumor” and “tumor disease”.

By “metastasis” is meant the spread of cancer cells from its original site to another part of the body. The formation of metastasis is a very complex process and depends on detachment of malignant cells from the primary tumor, invasion of the extracellular matrix, penetration of the endothelial basement membranes to enter the body cavity and vessels, and then, after being transported by the blood, infiltration of target organs. Finally, the growth of a new tumor at the target site depends on angiogenesis. Tumor metastasis often occurs even after the removal of the primary tumor because tumor cells or components may remain and develop metastatic potential. In one embodiment, the term “metastasis” according to the invention relates to “distant metastasis” which relates to a metastasis which is remote from the primary tumor and the regional lymph node system.

The term “treatment of a disease” includes curing, shortening the duration, ameliorating, preventing, slowing down or inhibiting progression or worsening, or preventing or delaying the onset of a disease or the symptoms thereof.

According to the invention, a sample may be any sample useful according to the present invention, in particular a biological sample such a tissue sample, including bodily fluids, and/or a cellular sample and may be obtained in the conventional manner such as by tissue biopsy, including punch biopsy, and by taking blood, bronchial aspirate, sputum, urine, feces or other body fluids. According to the invention, the term “biological sample” also includes fractions of biological samples.

A therapeutic effect in the treatments and uses discussed herein is preferably achieved through the functional properties of the antibodies of the invention to mediate killing of cells e.g. by inhibiting the immunosuppressive signal of PD-1 on cells expressing PD-1, preferably by forming a complex of the antibody and PD-1 and/or by inducing an immune response, more preferably a T cell mediated immune response.

In one embodiment, the anti-PD-1 antibody is administered as protein, wherein the antibody can have been obtained from hybridomas, transfectomas or by in vitro transcription, as described herein. In one embodiment, the anti-PD-1 antibody is administered as one or more nucleic acids or as one or more vectors as defined herein, e.g., as RNA or liposomes comprising the RNA or one or more RNAs which encode for the antibody or a chain of the antibody or a fragment of such antibody or chain.

Antibodies of the invention can be initially tested for their binding activity associated with therapeutic or diagnostic uses in vitro. For example, the antibodies can be tested using bindings assays, reporter gene blockade assays, and/or T cell proliferation assays as described herein.

The antibodies of the invention can be used to elicit in vivo or in vitro one or more of the following biological activities: to bind to, preferably specifically bind to PD-1; to have binding properties to PD-1 on either cancer cells or normal cells; to have binding properties to PD-1 epitopes; to have binding properties to a non-human PD-1 variant, particularly PD-1 variants from mice, rats, rabbits and primates; to prevent or reduce the induction of inhibitory signals by PD-1; to inhibit the interaction/binding of ligands of PD-1 with PD-1, preferably of the ligand PD-L1, for example, inhibiting the binding of human PD-L1 to human PD-1; to inhibit the immunosuppressive signal of PD-L1 or PD-L2; to enhancing or initiating the immune function (through this mechanism), preferably by enhancing or initiating a T-cell mediated immune response; to inhibit cancer proliferation; and/or to deplete tumor cells and/or suppress cancer metastasis.

The antibodies may also mediate phagocytosis or ADCC, mediate CDC in the presence of complement and/or mediate apoptosis of diseased cells.

In one embodiment, antibodies of the present invention can be used to treat a subject with a tumor disease. These tumors include solid tumors and/or hematological malignancies. Examples of tumor diseases which can be treated and/or prevented encompass all cancers and tumor entities which include, but are not limited to, carcinoma, lymphoma, blastoma, sarcoma, and leukemia. More particularly, examples of such cancers include bone cancer, blood cancer, lung cancer, liver cancer, pancreatic cancer, skin cancer, cancer of the head or neck, cutaneous or intraocular melanoma, uterine cancer, ovarian cancer, rectal cancer, cancer of the anal region, stomach cancer, colon cancer, breast cancer, prostate cancer, uterine cancer, carcinoma of the sexual and reproductive organs, Hodgkin's Disease (Hodgkin's lymphoma), cancer of the esophagus, cancer of the small intestine, cancer of the endocrine system, cancer of the thyroid gland, cancer of the parathyroid gland, cancer of the adrenal gland, sarcoma of soft tissue, cancer of the bladder, cancer of the kidney, renal cell carcinoma, carcinoma of the renal pelvis, neoplasms of the central nervous system (CNS), neuroectodermal cancer, spinal axis tumors, glioma, meningioma, and pituitary adenoma. These cancers may be in early, intermediate or advanced stages, e.g. metastasis. In one embodiment, the cancer to be treated is in an advanced stage.

Examples of cancers which are particularly susceptible for a PD-1 pathway blockade therapy include, but are not limited to, melanoma, including metastatic melanomas, lymphomas, including Hodgkin's lymphomas, lung cancer, including non-small cell lung cancer (NSCLC), for example advanced NSCLC, and small cell lung cancer, renal cell carcinoma, bladder cancer, breast cancer, including advanced triple negative breast cancer, gastric and gastroesophageal junction cancers, pancreatic adenocarcinoma, and ovarian cancer.

Suitable routes of administering the compositions of the invention in vivo and in vitro are well known in the art and can be selected by those of ordinary skill. The compositions of the invention can be administered systemically or locally. For example, they may be adminstered orally or parenterally. In this regard, reference to the respective disclosure above is made also. Combination strategies in cancer treatment may be desirable due to a resulting synergistic effect, which may be considerably stronger than the impact of a monotherapeutic approach. Therefore, it is also encompassed by the present invention that the antibodies or pharmaceutical compositions of the invention also can be administered in combination therapy, i.e., combined with other agents.

The anti-PD-1 antibodies of the invention can be co-administered with one or other more therapeutic agents, e.g., a cytotoxic agent, a radiotoxic agent, antiangiogenic agent or and immunosuppressive agent to reduce the induction of immune responses against the antibodies of invention. The antibody can be linked to the agent (as an immunocomplex) or can be administered separate from the agent. In the latter case (separate administration), the antibody can be administered before, after or concurrently with the agent or can be co-administered with other known therapies, e.g., an anti-cancer therapy, e.g., radiation. Such therapeutic agents include, among others, anti-neoplastic agents such as listed above. Co-administration of the anti-PD-1 antibodies of the present invention with chemotherapeutic agents provides two anti-cancer agents which operate via different mechanisms yielding a cytotoxic effect to tumor cells. Such co-administration can solve problems due to development of resistance to drugs or a change in the antigenicity of the tumor cells which would render them unreactive with the antibody.

The antibodies or compositions of the present invention can be used in conjunction with chemotherapy. Therapeutic agents for chemotherapy include, but are not limited to one or more chemotherapeutics, such as Taxol derivatives, taxotere, gemcitabin, antimetabolites (e.g., methotrexate, 6-mercaptopurine, 6-thioguanine, cytarabine, fludarabin, 5-fluorouracil decarbazine), alkylating agents (e.g., mechlorethamine, thioepa chlorambucil, melphalan, carmustine (BSNU) and lomustine (CCNU), cyclophosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin (Adriamycin)), antibiotics (e.g., dactinomycin (formerly actinomycin), bleomycin, mithramycin, and anthramycin (AMC), and anti-mitotic agents (e.g., vincristine and vinblastine). In a preferred embodiment, the therapeutic agent is a cytotoxic agent or a radiotoxic agent. In another embodiment, the therapeutic agent is an immunosuppressant. In yet another embodiment, the therapeutic agent is GM-CSF. In a preferred embodiment, the therapeutic agent is doxorubicin, cisplatin (Platinol), bleomycin, sulfate, carmustine, chlorambucil, cyclophosphamide (Cytoxan, Procytox, Neosar) or ricin A.

In another embodiment, antibodies of the present invention may be administered in combination with chemotherapeutic agents, which preferably show therapeutic efficacy in patients suffering from cancers which are particulary susceptible for a PD-1 pathway blockade, such as melanoma, including metastatic melanomas, Hodgkin's lymphomas, lung cancer, including non-small cell lung cancer (NSCLC), for example advanced NSCLC, and small cell lung cancer, renal cell carcinoma, bladder cancer, advanced triple negative breast cancer, including advanced triple negative breast cancer, gastric and gastroesophageal junction cancers, pancreatic adenocarcinoma, or ovarian cancer.

In one embodiment, the antibodies or the pharmaceutical composition of the present invention is administered with an immunotherapeutic agent. As used herein “immunotherapeutic agent” relates to any agent that may be involved in activating a specific immune response and/or immune effector function(s). The present disclosure contemplates the use of an antibody as an immunotherapeutic agent. Without wishing to be bound by theory, antibodies are capable of achieving a therapeutic effect against cancer cells through various mechanisms, including inducing apoptosis, block components of signal transduction pathways or inhibiting proliferation of tumor cells. In certain embodiments, the antibody is a monoclonal antibody. A monoclonal antibody may induce cell death via antibody-dependent cell mediated cytotoxicity (ADCC), or bind complement proteins, leading to direct cell toxicity, known as complement dependent cytotoxicity (CDC). Non-limiting examples of anti-cancer antibodies and potential antibody targets (in brackets) which may be used in combination with the present disclosure include: Abagovomab (CA-125), Abciximab (CD41), Adecatumumab (EpCAM), Afutuzumab (CD20), Alacizumab pegol (VEGFR2), Altumomab pentetate (CEA), Amatuximab (MORAb-009), Anatumomab mafenatox (TAG-72), Apolizumab (HLA-DR), Arcitumomab (CEA), Atezolizumab (PD-L1), Bavituximab (phosphatidylserine), Bectumomab (CD22), Belimumab (BAFF), Bevacizumab (VEGF-A), Bivatuzumab mertansine (CD44 v6), Blinatumomab (CD 19), Brentuximab vedotin (CD30 TNFRSF8), Cantuzumab mertansin (mucin CanAg), Cantuzumab ravtansine (MUC1), Capromab pendetide (prostatic carcinoma cells), Carlumab (CNT0888), Catumaxomab (EpCAM, CD3), Cetuximab (EGFR), Citatuzumab bogatox (EpCAM), Cixutumumab (IGF-1 receptor), Claudiximab (Claudin), Clivatuzumab tetraxetan (MUC1), Conatumumab (TRAIL-R2), Dacetuzumab (CD40), Dalotuzumab (insulin-like growth factor I receptor), Denosumab (RANKL), Detumomab (B-lymphoma cell), Drozitumab (DR5), Ecromeximab (GD3 ganglioside), Edrecolomab (EpCAM), Elotuzumab (SLAMF7), Enavatuzumab (PDL192), Ensituximab (NPC-1C), Epratuzumab (CD22), Ertumaxomab (HER2/neu, CD3), Etaracizumab (integrin αvβ3), Farletuzumab (folate receptor 1), FBTA05 (CD20), Ficlatuzumab (SCH 900105), Figitumumab (IGF-1 receptor), Flanvotumab (glycoprotein 75), Fresolimumab (TGF-0), Galiximab (CD80), Ganitumab (IGF-I), Gemtuzumab ozogamicin (CD33), Gevokizumab (ILIP), Girentuximab (carbonic anhydrase 9 (CA-IX)), Glembatumumab vedotin (GPNMB), Ibritumomab tiuxetan (CD20), Icrucumab (VEGFR-1), Igovoma (CA-125), Indatuximab ravtansine (SDC1), Intetumumab (CD51), Inotuzumab ozogamicin (CD22), Ipilimumab (CD 152), Iratumumab (CD30), Labetuzumab (CEA), Lexatumumab (TRAIL-R2), Libivirumab (hepatitis B surface antigen), Lintuzumab (CD33), Lorvotuzumab mertansine (CD56), Lucatumumab (CD40), Lumiliximab (CD23), Mapatumumab (TRAIL-R1), Matuzumab (EGFR), Mepolizumab (IL5), Milatuzumab (CD74), Mitumomab (GD3 ganglioside), Mogamulizumab (CCR4), Moxetumomab pasudotox (CD22), Nacolomab tafenatox (C242 antigen), Naptumomab estafenatox (5T4), Namatumab (RON), Necitumumab (EGFR), Nimotuzumab (EGFR), Nivolumab (IgG4), Ofatumumab (CD20), Olaratumab (PDGF-R a), Onartuzumab (human scatter factor receptor kinase), Oportuzumab monatox (EpCAM), Oregovomab (CA-125), Oxelumab (OX-40), Panitumumab (EGFR), Patritumab (HER3), Pemtumoma (MUC1), Pertuzuma (HER2/neu), Pintumomab (adenocarcinoma antigen), Pritumumab (vimentin), Racotumomab (N-glycolylneuraminic acid), Radretumab (fibronectin extra domain-B), Rafivirumab (rabies virus glycoprotein), Ramucirumab (VEGFR2), Rilotumumab (HGF), Rituximab (CD20), Robatumumab (IGF-1 receptor), Samalizumab (CD200), Sibrotuzumab (FAP), Siltuximab (IL6), Tabalumab (BAFF), Tacatuzumab tetraxetan (alpha-fetoprotein), Taplitumomab paptox (CD 19), Tenatumomab (tenascin C), Teprotumumab (CD221), Ticilimumab (CTLA4), Tigatuzumab (TRAIL-R2), TNX-650 (IL13), Tositumomab (CD20), Trastuzumab (HER2/neu), TRBS07 (GD2), Tremelimumab (CTLA4), Tucotuzumab celmoleukin (EpCAM), Ublituximab (MS4A1), Urelumab (4-1 BB), Volociximab (integrin α5β1), Votumumab (tumor antigen CTAA 16.88), Zalutumumab (EGFR), and Zanolimumab (CD4).

For example, according to the present invention, the subject being administered the antibodies of the present invention is additionally treated with one or more antibodies targeting another immune checkpoint. Immune checkpoint inhibitors activating the tumor defense by interrupting inhibitory interactions between antigen-presenting cells and T lymphocytes include, but are not limited to anti-PD-L1, anti-CTLA4, anti-TIM-3, anti-KIR and/or anti-LAG-3. Also encompassed are immunotherapeutic agents which stimulate activating checkpoints, such as CD27, CD28, CD40, CD122, CD137, OX40, GITR, or ICOS, i.e., for example anti-CD27, anti-CD28, anti-CD40, anti-CD122, anti-CD137, anti-OX40, anti-GITR, and/or anti-ICOS. Particularly preferred combinations therapies include, but are not limited to the combination of anti-PD1 and anti-PD-L1, thereby increasing the efficiency and the blockade of the PD1 pathway by targeting both components, or the combination of anti-PD-1 and anti-CTLA4 in order to prevent the blockade of both the PD1 patway and the CTLA4 pathway.

In another particular embodiment of the invention, the subject being administered the antibody is additionally treated with an antiangiogenesis agent, including antibodies targeting vascular endothelial growth factor (VEGF) or its receptor VEGFR, and one or more chemical compounds inhibiting angiogenesis. Pretreatment with or parallel application of these drugs may improve the penetration of antibodies in bulk tumors.

For example, the antiangiogenesis agents may target VEGF. A suitable VEGF inhibitor is Bevacizumab. Other examples include, but are not limited to, multikinase inhibitors that inhibits VEGFR1, 2, 3, PDGFR, c-Kit, Raf and/or RET (e.g., Sunitinib, Sorafenib, Pazopanib).

In another particular embodiment of the invention, the subject being administered the antibody is additionally treated with a compound inhibiting growth factor receptor signaling including monoclonal antibodies binding to the EGFR receptor as well as chemical compounds inhibiting signaling initiated by the EGFR receptor.

In another embodiment, such therapeutic agents include agents leading to the depletion or functional inactivation of regulatory T cells like low dose cyclophosphamid, and/or anti-IL2 or anti-IL2-receptor antibodies.

In still another embodiment, the antibodies of the invention may be administered in combination with one or more antibodies selected from anti-CD25 antibodies, anti-EPCAM antibodies, and anti-CD40 antibodies.

In yet a further embodiment, the antibodies of the invention may be administered in combination with an anti-C3b(i) antibody in order to enhance complement activation.

In another embodiment, the antibodies of the invention may be administered in combination with a vaccination therapy, i.e., in combination with at least one peptide or protein comprising an epitope for inducing an immune response against an antigen in the subject, or at least one polynucleotide/nucleic acid encoding the peptide or protein.

The term “antigen” relates to an agent comprising an epitope against which an immune response or an immune effector molecule such as antibody is directed and/or is to be directed. The term “antigen” includes, in particular, proteins and peptides. In one embodiment, an antigen is a disease-associated antigen, such as a tumor antigen.

The term “disease-associated antigen” is used in its broadest sense to refer to any antigen associated with a disease which preferably contains an epitope that will stimulate a host's immune system to make a cellular antigen-specific immune response and/or a humoral antibody response against the disease. The disease-associated antigen, an epitope thereof, or an agent, such as peptide or protein inducing an immune response, targeting the disease-associated antigen or epitope may therefore be used for therapeutic purposes, in particular for vaccination. Disease-associated antigens may be associated with infection by microbes, typically microbial antigens, or associated with cancer, typically tumors.

In one embodiment, the antigen against which an immune response is to be directed (i.e., disease associated antigen) is a tumor antigen, preferably as specified herein. More preferably, the at least one tumor antigen is selected from the group consisting of NY-ESO-1 (UniProt P78358), Tyrosinase (UniProt P14679), MAGE-A3 (UniProt P43357), TPTE (UniProt P56180), KLK2 (UniProt P20151), PSA(KLK3) (UniProt P07288), PAP(ACPP, UniProt P15309), HOXB13 (UniProt Q92826), NKX3-1 (UniProt Q99801), HPV16 E6/E7 (UniProt P03126/P03129); HPV18 E6/E7 (UniProt P06463/P06788); HPV31 E6/E7 (UniProt P17386/P17387); HPV33 E6/E7 (UniProt P06427/P06429); HPV45 E6/E7 (UniProt P21735/P21736); HPV58 E6/E7 (UniProt P26555/P26557), PRAME (UniProt P78395), ACTL8 (UniProt Q9H568), CXorf61 (KKLC1, UniProt Q5H943), MAGE-A9B (UniProt P43362), CLDN6 (UniProt P56747), PLAC1 (UniProt Q9HBJ0), and p53 (UniProt P04637).

The peptide or protein that is used for vaccination (i.e., vaccine antigen) may comprise said antigen or an epitope thereof. The vaccine antigen in one embodiment is administered in the form of RNA encoding the vaccine antigen. Methods of treatment involving these antigens may aim at the treatment of cancer, wherein the cancer cells are characterized by expression of the respective antigen. It is also possible to use antigens described herein, in particular NY-ESO-1, Tyrosinase, MAGE-A3, TPTE, KLK2, PSA(KLK3), PAP(ACPP), HOXB13, NKX3-1, HPV16 E6/E7; HPV18 E6/E7; HPV31 E6/E7; HPV33 E6/E7; HPV45 E6/E7; HPV58 E6/E7, PRAME, ACTL8, CXorf61 (KKLC1), MAGE-A9B, CLDN6, PLAC1, and p53, in combination. Methods of treatment involving such combination of antigens may aim at the treatment of cancer, wherein the cancer cells are characterized by expression of two or more antigens of the respective combination of antigens or wherein the cancer cells of a large fraction (e.g., at least 80%, at least 90% or even more) of patients having a certain cancer to be treated express one or more of the respective antigens of a combination. Such combination may comprise a combination of at least 2, at least 3, at least 4, at least 5, or at least 6 antigens. Thus, the combination may comprise 3, 4, 5, 6, 7, or 8 antigens. In this case, each antigen of the combination may be addressed by administering peptide or protein (i.e., vaccine antigen) comprising said antigen or an epitope thereof, or RNA encoding the peptide or protein. In one particularly preferred embodiment, each antigen of the combination is addressed by administering RNA encoding a peptide or protein comprising the antigen. Thus, vaccination may encompass the administration of different RNA molecules, wherein each of said different RNA molecules encodes a peptide or protein comprising an antigen of a combination of antigens. The different vaccine antigens or RNAs encoding different vaccine antigens of a combination may be administered in a mixture, sequentially, or a combination thereof.

In one embodiment, the antigen combination comprises, preferably consists of NY-ESO-1, Tyrosinase, MAGE-A3, and TPTE. This combination may be used for the treatment of cutaneous melanoma.

In one embodiment, the antigen combination comprises, preferably consists of KLK2, PSA(KLK3), PAP(ACPP), HOXB13, and NKX3-1. This combination may be used for the treatment of prostate cancer.

In one embodiment, the antigen combination comprises, preferably consists of PRAME, ACTL8, CXorf61 (KKLC1), MAGEA3, MAGE-A9B, CLDN6, NY-ESO-1, and PLAC1.

This combination may be used for the treatment of breast cancer such as triple negative breast cancer, in particular estrogen receptor negative & progesteron receptor negative & HER2 negative breast cancer.

In one embodiment, the antigen combination comprises, preferably consists of CLDN6, p53, and PRAME. This combination may be used for the treatment of ovarian cancer, such as epithelial ovarian cancer.

The vaccine described herein may consist of one or more RNAs targeting one or more antigens expressed in a disease such as cancer. The active principle may be single-stranded mRNA that is translated into the respective protein upon entering antigen-presenting cells (APCs). In addition to wildtype or codon-optimized sequences encoding the antigen sequence, the RNA may contain one or more structural elements optimized for maximal efficacy of the RNA with respect to stability and translational efficiency (5′-cap, 5′-UTR, 3′-UTR, poly(A)-tail). In one embodiment, the RNA contains all of these elements. In one embodiment, beta-S-ARCA(D1) may be utilized as specific capping structure at the 5′-end of the RNA drug substances. As 5′-UTR sequence, the 5′-UTR sequence of the human alpha-globin mRNA, optionally with an optimized ‘Kozak sequence’ to increase translational efficiency may be used. As 3′-UTR sequence, two re-iterated 3′-UTRs of the human beta-globin mRNA placed between the coding sequence and the poly(A)-tail to assure higher maximum protein levels and prolonged persistence of the mRNA may be used. Alternatively, the 3′-UTR may be a combination of two sequence elements (FI element) derived from the “amino terminal enhancer of split” (AES) mRNA (called F) and the mitochondrial encoded 12S ribosomal RNA (called I). These were identified by an ex vivo selection process for sequences that confer RNA stability and augment total protein expression (see, WO 2017/060314, herein incorporated by reference). Furthermore, a poly(A)-tail measuring 110 nucleotides in length, consisting of a stretch of 30 adenosine residues, followed by a 10 nucleotide linker sequence (of random nucleotides) and another 70 adenosine residues may be used. This poly(A)-tail sequence was designed to enhance RNA stability and translational efficiency in dendritic cells.

Furthermore, sec (secretory signal peptide) and/or MITD (MHC class I trafficking domain) may be fused to the antigen-encoding regions in a way that the respective elements are translated as N- or C-terminal tag, respectively. Fusion-protein tags derived from the sequence encoding the human MHC class I complex (HLA-B51, haplotype A2, B27/B51, Cw2/Cw3), have been shown to improve antigen processing and presentation. Sec may correspond to the 78 bp fragment coding for the secretory signal peptide, which guides translocation of the nascent polypeptide chain into the endoplasmatic reticulum. MITD may correspond to the transmembrane and cytoplasmic domain of the MHC class I molecule, also called MHC class I trafficking domain. Antigens such as CLDN6 having their own secretory signal peptide and transmembrane domain may not require addition of fusion tags. Sequences coding for short linker peptides predominantly consisting of the amino acids glycine (G) and serine (S), as commonly used for fusion proteins may be used as GS/Linkers.

The antigen may be administered in combination with helper epitopes to break immunological tolerance. The helper epitopes may be tetanus toxoid-derived, e.g., P2P16 amino acid sequences derived from the tetanus toxoid (T) of Clostridium tetani. These sequences may support to overcome self-tolerance mechanisms for efficient induction of immune responses to self-antigens by providing tumor-unspecific T-cell help during priming. The tetanus toxoid heavy chain includes epitopes that can bind promiscuously to MHC class II alleles and induce CD4⁺ memory T cells in almost all tetanus vaccinated individuals. In addition, the combination of TT helper epitopes with tumor-associated antigens is known to improve the immune stimulation compared to the application of tumor-associated antigen alone by providing CD4⁺ mediated T-cell help during priming. To reduce the risk of stimulating CD8⁺ T cells, two peptide sequences known to contain promiscuously binding helper epitopes may be used to ensure binding to as many MHC class II alleles as possible, e.g., P2 and P16.

In one embodiment, a vaccine antigen comprises an amino acid sequence which breaks immunological tolerance. In one embodiment, the amino acid sequence which breaks immunological tolerance comprises helper epitopes, preferably tetanus toxoid-derived helper epitopes. The amino acid sequence which breaks immunological tolerance may be fused to the C-terminus of the vaccine sequence, e.g., antigen sequence, either directly or separated by a linker. Optionally, the amino acid sequence which breaks immunological tolerance may link the vaccine sequence and the MITD. In case the vaccine antigen is administered in the form of RNA encoding the vaccine antigen, the amino acid sequence which breaks immunological tolerance may be RNA encoded. In one embodiment, the antigen-targeting RNAs are applied together with RNA coding for a helper-epitope to boost the resulting immune response. This RNA coding for a helper-epitope may contain structural elements optimized for maximal efficacy of the RNA with respect to stability and translational efficiency (5′-cap, 5′-UTR, 3′-UTR, poly(A)-tail) described above for the antigen-encoding RNA. Furthermore, sec (secretory signal peptide) and/or MITD (MHC class I trafficking domain) may be fused to the helper-epitope-encoding regions in a way that the respective elements are translated as N- or C-terminal tag, respectively, as described above for the antigen-encoding RNA. In one embodiment, RNAs are co-administered with an additional RNA coding for the tetanus toxoid (TT) derived helper epitopes P2 and P16 (P2P16) in order to boost the resulting immune response.

The vaccine RNA may be complexed with liposomes to generate serum-stable RNA-lipoplexes (RNA_{(LIP)}) for intravenous (i.v.) administration. If a combination of different RNAs is used, the RNAs may be separately complexed with liposomes to generate serum-stable RNA-lipoplexes (RNA_{(LIP)}) for intravenous (i.v.) administration. RNA_{(LIP)} targets antigen-presenting cells (APCs) in lymphoid organs which results in an efficient stimulation of the immune system.

In one embodiment, vaccine RNA is co-formulated as lipoplex particles with an RNA encoding an amino acid sequence which breaks immunological tolerance.

As used herein, “tumor antigen” or “cancer antigen” includes (i) tumor-specific antigens, (ii) tumor-associated antigens, (iii) embryonic antigens on tumors, (iv) tumor-specific membrane antigens, (v) tumor-associated membrane antigens, (vi) growth factor receptors, and (xi) any other type of antigen or material that is associated with a cancer.

Any tumor antigen (preferably expressed by a tumor cell) can be targeted by the vaccination disclosed herein. In one embodiment, the tumor antigen is presented by a tumor cell and thus can be targeted by T cells. Vaccination as disclosed herein preferably activates T cells specific for MHC presented tumor antigens. The tumor antigen may be a tumor-specific antigen (TSA) or a tumor-associated antigen (TAA). A TSA is unique to tumor cells and does not occur on other cells in the body. A TAA is not unique to a tumor cell and instead is also expressed on a normal cell under conditions that fail to induce a state of immunologic tolerance to the antigen. The expression of the antigen on the tumor may occur under conditions that enable the immune system to respond to the antigen. TAAs may be antigens that are expressed on normal cells during fetal development when the immune system is immature and unable to respond or they may be antigens that are normally present at extremely low levels on normal cells but which are expressed at much higher levels on tumor cells.

The peptide and protein antigen can be 2-100 amino acids, including for example, at least 5 amino acids, at least 10 amino acids, at least 15 amino acids, at least 20 amino acids, at least amino acids, at least 30 amino acids, at least 35 amino acids, at least 40 amino acids, at least 45 amino acids, or at least 50 amino acids in length. In some embodiments, a peptide can be greater than 50 amino acids. In some embodiments, the peptide can be greater than 100 amino acids.

The peptide or protein antigen can be any peptide or protein that can induce or increase the ability of the immune system to develop antibodies and T cell responses to a target antigen, e.g., disease-associated antigen.

In yet another embodiment, the antibodies of the invention may be administered in conjunction with radiotherapy and/or autologous peripheral stem cell or bone marrow transplantation.

Also encompassed by the present invention is a combination therapy including a composition of the present invention with at least one anti-inflammatory agent or at least one immunosuppressive agent. In one embodiment such therapeutic agents include one or more anti-inflammatory agents, such as a steroidal drug or a NSAID (nonsteroidal anti-inflammatory drug). Preferred agents include, for example, aspirin and other salicylates, Cox-2 inhibitors, such as rofecoxib (Vioxx) and celecoxib (Celebrex), NSAIDs such as ibuprofen (Motrin, Advil), fenoprofen (Nalfon), naproxen (Naprosyn), sulindac (Clinoril), diclofenac (Voltaren), piroxicam (Feldene), ketoprofen (Orudis), diflunisal (Dolobid), nabumetone (Relafen), etodolac (Lodine), oxaprozin (Daypro), and indomethacin (Indocin). A combination therapy according to the present invention may also comprise a combination of (i) the antibodies of the present invention with (ii) a vaccination treatment/therapy as specified above, and (iii) at least one anti-inflammatory agent or at least one immunosuppressive agent.

Bispecific and multispecific molecules of the invention can be used to interact with another immune checkpoint. Thereby either inhibiting or activating/stimulating the respective other checkpoint. Other checkpoint inhibitors which may be targeted include, but are not limited to CTLA4, PD-L1, TIM-3, KIR or LAG-3, checkpoint activators which may be targeted by the second binding specificity include, but are not limited to CD27, CD28, CD40, CD122, CD137, OX40, GITR, or ICOS. Preferred combinations of binding specificities include anti-PD1 and anti-PD-L1 or anti-PD-1 and anti-CTLA4.

Alternatively or in addition, bispecific or multispecific molecules of the invention can be used to provide an antiangiogenesis activity by targeting for example the vascular endothelial growth factor (VEGF) or its receptor VEGFR (for example VEGFR1, 2, 3). The second binding specifity may also be capable of targeting PDGFR, c-Kit, Raf and/or RET.

Alternatively or in addition, bispecific or multispecific molecules of the invention can be used to target a tumor antigen, preferably a tumor antigen as specified supra, which enables a specificity of the antibody of the present invention for cancer cells.

Preferably in addition to a tumor antigen specificity and an anti-PD-1 binding specificity, a multispecific antibody of the present invention can also be used to modulate Fc-gammaR or Fc-alphaR levels on effector cells, such as by capping and eliminating receptors on the cell surface. Mixtures of anti-Fc receptors can also be used for this purpose.

For the uses and methods of the present invention actual dosage levels of the active ingredients, which may be comprised in a pharmaceutical composition, preferably a pharmaceutical composition as described above, may be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the activity of the particular compositions of the present invention employed, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved. In general, a suitable daily dose of a composition of the invention will be that amount of the compound which is the lowest dose effective to produce a therapeutic effect. Such an effective dose will generally depend upon the factors described above. It is preferred that administration be intravenous, intramuscular, intraperitoneal, or subcutaneous, preferably administered proximal to the site of the target. If desired, the effective daily dose of a therapeutic composition may be administered as two, three, four, five, six or more sub-doses administered separately at appropriate intervals throughout the day, optionally, in unit dosage forms. While it is possible for a compound of the present invention to be administered alone, it is preferable to administer the compound as a pharmaceutical composition (formulation).

In one embodiment, the antibodies of the invention may be administered by infusion, preferably slow continuous infusion over a long period, such as more than 24 hours, in order to reduce toxic side effects. The administration may also be performed by continuous infusion over a period of from 2 to 24 hours, such as of from 2 to 12 hours. Such regimen may be repeated one or more times as necessary, for example, after 6 months or 12 months. The dosage can be determined or adjusted by measuring the amount of circulating anti-PD-1 antibodies upon administration in a biological sample by using anti-idiotypic antibodies which target the anti-PD-1 antibodies.

In yet another embodiment, the antibodies are administered by maintenance therapy, such as, e.g., once a week for a period of 6 months or more.

A “therapeutically effective dosage” for tumor therapy can be measured by objective tumor responses which can either be complete or partial. A complete response (CR) is defined as no clinical, radiological or other evidence of disease. A partial response (PR) results from a reduction in aggregate tumor size of greater than 50%. Median time to progression is a measure that characterizes the durability of the objective tumor response.

A “therapeutically effective dosage” for tumor therapy can also be measured by its ability to stabilize the progression of disease. The ability of a compound to inhibit cancer can be evaluated in an animal model system predictive of efficacy in human tumors. Alternatively, this property of a composition can be evaluated by examining the ability of the compound to inhibit cell growth or apoptosis by in vitro assays known to the skilled practitioner. A therapeutically effective amount of a therapeutic compound can decrease tumor size, or otherwise ameliorate symptoms in a subject. One of ordinary skill in the art would be able to determine such amounts based on such factors as the subject's size, the severity of the subject's symptoms, and the particular composition or route of administration selected.

Therefore, in a further aspect the present invention is concerned with the medical use of the antibodies, conjugates, multimers, nucleic acids, vectors, host cells, viruses or compositions of the present invention. In this regard the invention provides antibodies, conjugates, multimers, nucleic acids, vectors, host cells, viruses or compositions, preferably pharmaceutical compositions, for use in the treatment of a disease, e.g., for use in tumor/cancer treatment. The expression “for use in the treatment of a disease, e.g., for use in tumor/cancer treatment” is used herein also replaceable with “for use as a medicament, especially in a method of treatment of cancer”; or the use of said products in the preparation of a pharmaceutical formulation for use in said method of treatment in humans (or more generically a subject in need thereof).

Alternative to the use of the antibodies, conjugates, multimers, nucleic acids, vectors, host cells, viruses or compositions of the present invention in tumor/cancer treatment, the antibodies, conjugates, multimers, nucleic acids, vectors, host cells, viruses or compositions of the present invention can be used in the treatment of other diseases for which treatment an induction of an immune response is required. Accordingly, the antibodies, conjugates, multimers, nucleic acids, vectors, host cells, viruses or compositions of the present invention may be effective on infection treatment. Infection treatment may include, for example, infections with human hepatitis virus (hepatitis B, Hepatitis C, hepatitis A, or hepatitis E), human retrovirus, human immunodeficiency virus (HIV1, HIV2), human T leukemia virus (HTLV1, HTLV2), or human lymphocytic cell type virus, simple herpes virus type 1 or 2, epstein-barr virus, cytomegalovirus, varicella-zoster virus, human herpesvirus including human herpesvirus 6, poliovirus, measles virus, rubella virus, Japanese encephalitis virus, mumps virus, influenza virus, adenovirus, enterovirus, rhinovirus, virus developing severely acute respiratory syndrome (SARS), ebola virus, west nile virus, or of these virus modified artificially.

Still further alternative to the use of the antibodies, conjugates, multimers, nucleic acids, vectors, host cells, viruses or compositions of the present invention in tumor/cancer treatment, the antibodies, conjugates, multimers, nucleic acids, vectors, host cells, viruses or compositions of the present invention can be used in the treatment of other diseases for which treatment a depletion of activated immune cells is required. Accordingly, the antibodies, conjugates, multimers, nucleic acids, vectors, host cells, viruses or compositions of the present invention may be effective for the treatment of an autoimmune disease. Autoimmune diseases may include, for example, coeliac disease, inflammatory bowel disease, multiple sclerosis, rheumatoid arthritis and systemic lupus erythematosus.

Unless the context indicates otherwise, the disclosure with regard to preferred embodiments of the uses and methods of the invention disclosed above relative to the treatment of cancer, applies also for the treatment of infection diseases or autoimmune diseases.

Also within the scope of the present invention are kits comprising the antibodies, conjugates or multimers of the invention and instructions for use. The kit can further contain one or more additional reagents, such as antibodies targeting the anti-PD-1 antibody of the present invention, enzyme substrates or other substrates, enzymes for obtaining a color development, etc. A kit of the present invention may be used for qualitative or quantitative detection of PD-1 in a sample.

In a particular embodiment, the invention provides methods for detecting the presence of PD-1 antigen in a sample, or measuring the amount of PD-1 antigen, comprising contacting the sample, and a control sample, with an antibody which specifically binds to PD-1, the antibody being preferably an antibody as disclosed herein, under conditions that allow for formation of a complex between the antibody or portion thereof and PD-1. The formation of a complex is then detected, wherein a difference complex formation between the sample compared to the control sample is indicative for the presence of PD-1 antigen in the sample.

In still another embodiment, the invention provides a method for detecting the presence or quantifying the amount of PD-1-expressing cells in vivo or in vitro. The method comprises (i) administering to a subject an antibody of the invention conjugated to a detectable marker; (ii) exposing the subject to a means for detecting said detectable marker to identify areas containing PD-1-expressing cells.

Methods as described above are useful, in particular, for diagnosing PD-1-related diseases and/or the localization of PD-1-related diseases. Preferably an amount of PD-1 in a sample which is higher than the amount of PD-1 in a control sample is indicative for the presence of a PD-1-related disease in a subject, in particular a human, from which the sample is derived.

In yet another embodiment conjugates of the invention can be used to target compounds (e.g., therapeutic agents, labels, etc.) to cells which have PD-1 expressed on their surface by linking such compounds to the antibody. Thus, the invention also provides methods for localizing ex vivo or in vitro cells expressing PD-1.

In describing a protein or peptide, structure and function herein, reference is made to amino acids. In the present specification, amino acid residues are expressed by using the following abbreviations. Also, unless explicitly otherwise indicated, the amino acid sequences of peptides and proteins are identified from N-terminal to C-terminal (left terminal to right terminal), the N-terminal being identified as a first residue. Amino acids are designated by their 3-letter abbreviation, 1-letter abbreviation, or full name, as follows. Ala: A: alanine; Asp: D: aspartic acid; Glu: E: glutamic acid; Phe: F: phenylalanine; Gly: G: glycine; His: H: histidine; Ile: I: isoleucine; Lys: K: lysine; Leu: L: leucine; Met: M: methionine; Asn: N: asparagine; Pro: P: proline; Gln: Q: glutamine; Arg: R: arginine; Ser: S: serine; Thr: T: threonine; Val: V: valine; Trp: W: tryptophan; Tyr: Y: tyrosine; Cys: C cysteine.

The teaching given herein with respect to specific amino acid sequences, e.g. those shown in the sequence listing, is to be construed so as to also relate to variants of said specific sequences resulting in sequences which are functionally equivalent to said specific sequences, e.g. amino acid sequences exhibiting properties identical or similar to those of the specific amino acid sequences.

The term “variant” according to the invention refers, in particular, to mutants, splice variants, conformations, isoforms, allelic variants, species variants and species homologs, in particular those which are naturally present. An allelic variant relates to an alteration in the normal sequence of a gene, the significance of which is often unclear. Complete gene sequencing often identifies numerous allelic variants for a given gene. A species homolog is a nucleic acid or amino acid sequence with a different species of origin from that of a given nucleic acid or amino acid sequence. The term “variant” shall encompass any posttranslationally modified variants and conformation variants.

For the purposes of the present invention, “variants” of an amino acid sequence comprise amino acid insertion variants, amino acid addition variants, amino acid deletion variants and/or amino acid substitution variants.

Preferably the degree of similarity, preferably identity between a given amino acid sequence and an amino acid sequence which is a variant of said given amino acid sequence will be at least about 60%, 65%, 70%, 80%, 81%, 82%, 83%, 84%, 85%, 86%, 87%, 88%, 89%, 90%, 91%, 92%, 93%, 94%, 95%, 96%, 97%, 98%, or 99%. The degree of similarity or identity is given preferably for an amino acid region which is at least about 10%, at least about 20%, at least about 30%, at least about 40%, at least about 50%, at least about 60%, at least about 70%, at least about 80%, at least about 90% or about 100% of the entire length of the reference amino acid sequence. For example, if the reference amino acid sequence consists of 200 amino acids, the degree of similarity or identity is given preferably for at least about 20, at least about 40, at least about 60, at least about 80, at least about 100, at least about 120, at least about 140, at least about 160, at least about 180, or about 200 amino acids, preferably continuous amino acids. In preferred embodiments, the degree of similarity or identity is given for the entire length of the reference amino acid sequence. The alignment for determining sequence similarity, preferably sequence identity can be done with art known tools, preferably using the best sequence alignment, for example, using Align, using standard settings, preferably EMBOSS::needle, Matrix: Blosum62, Gap Open 10.0, Gap Extend 0.5.

“Sequence similarity” indicates the percentage of amino acids that either are identical or that represent conservative amino acid substitutions. “Sequence identity” between two amino acid sequences indicates the percentage of amino acids that are identical between the sequences.

The term “percentage identity” is intended to denote a percentage of amino acid residues which are identical between the two sequences to be compared, obtained after the best alignment, this percentage being purely statistical and the differences between the two sequences being distributed randomly and over their entire length. Sequence comparisons between two amino acid sequences are conventionally carried out by comparing these sequences after having aligned them optimally, said comparison being carried out by segment or by “window of comparison” in order to identify and compare local regions of sequence similarity. The optimal alignment of the sequences for comparison may be produced, besides manually, by means of the local homology algorithm of Smith and Waterman, 1981, Ads App. Math. 2, 482, by means of the local homology algorithm of Needleman and Wunsch, 1970, J. Mol. Biol. 48, 443, by means of the similarity search method of Pearson and Lipman, 1988, Proc. Natl Acad. Sci. USA 85, 2444, or by means of computer programs which use these algorithms (GAP, BESTFIT, FASTA, BLAST P, BLAST N and TFASTA in Wisconsin Genetics Software Package, Genetics Computer Group, 575 Science Drive, Madison, Wis.).

The percentage identity is calculated by determining the number of identical positions between the two sequences being compared, dividing this number by the number of positions compared and multiplying the result obtained by 100 so as to obtain the percentage identity between these two sequences.

With respect to nucleic acid molecules, the term “variant” includes degenerate nucleic acid sequences, wherein a degenerate nucleic acid according to the invention is a nucleic acid that differs from a reference nucleic acid in codon sequence due to the degeneracy of the genetic code.

Furthermore, a “variant” of a given nucleic acid sequence according to the invention includes nucleic acid sequences comprising single or multiple such as at least 2, at least 4, or at least 6 and preferably up to 3, up to 4, up to 5, up to 6, up to 10, up to 15, or up to 20 nucleotide substitutions, deletions and/or additions.

Preferably the degree of identity between a given nucleic acid sequence and a nucleic acid sequence which is a variant of said given nucleic acid sequence will be at least 70%, preferably at least 75%, preferably at least 80%, more preferably at least 85%, even more preferably at least 90% or most preferably at least 95%, 96%, 97%, 98% or 99%. The degree of identity is preferably given for a region of at least about 30, at least about 50, at least about 70, at least about 90, at least about 100, at least about 150, at least about 200, at least about 250, at least about 300, or at least about 400 nucleotides. In preferred embodiments, the degree of identity is given for the entire length of the reference nucleic acid sequence.

“Sequence identity” between two nucleic acid sequences indicates the percentage of nucleotides that are identical between the sequences.

The term “percentage identity” is intended to denote a percentage of nucleotides which are identical between the two sequences to be compared, obtained after the best alignment, this percentage being purely statistical and the differences between the two sequences being distributed randomly and over their entire length. Sequence comparisons between two nucleotide sequences are conventionally carried out by comparing these sequences after having aligned them optimally, said comparison being carried out by segment or by “window of comparison” in order to identify and compare local regions of sequence similarity. The optimal alignment of the sequences for comparison may be produced, besides manually, by means of the local homology algorithm of Smith and Waterman, 1981, Ads App. Math. 2, 482, by means of the local homology algorithm of Needleman and Wunsch, 1970, J. Mol. Biol. 48, 443, by means of the similarity search method of Pearson and Lipman, 1988, Proc. Natl Acad. Sci. USA 85, 2444, or by means of computer programs which use these algorithms (GAP, BESTFIT, FASTA, BLAST P, BLAST N and TFASTA in Wisconsin Genetics Software Package, Genetics Computer Group, 575 Science Drive, Madison, Wis.).

The percentage identity is calculated by determining the number of identical positions between the two sequences being compared, dividing this number by the number of positions compared and multiplying the result obtained by 100 so as to obtain the percentage identity between these two sequences.

The terms “part”, “fragment” and “portion” are used interchangeably herein and refer to a continuous or discontinuous fraction of a structure. With respect to a particular structure such as an amino acid sequence or protein or a nucleic acid sequence the terms “part”, “fragment” and “portion” thereof may designate a continuous or a discontinuous fraction of said structure. Preferably, a “part”, “fragment” and “portion” of a structure such as an amino acid sequence or a nucleic acid sequence preferably comprises, preferably consists of at least 10%, at least 20%, at least 30%, at least 40%, at least 50%, at least 60%, at least 70%, at least 80%, at least 85%, at least 90%, at least 92%, at least 94%, at least 96%, at least 98%, at least 99% of the entire structure or amino acid sequence or nucleic acid sequence. A portion, a part or a fragment of a structure preferably comprises one or more functional properties of said structure. For example, a portion, a part or a fragment of an epitope, peptide or protein is preferably immunologically equivalent to the epitope, peptide or protein it is derived from. If the portion, part or fragment is a discontinuous fraction said discontinuous fraction is preferably composed of 2, 3, 4, 5, 6, 7, 8, or more parts of a structure, each part being a continuous element of the structure. For example, a discontinuous fraction of an amino acid sequence may be composed of 2, 3, 4, 5, 6, 7, 8, or more, preferably not more than 4 parts of said amino acid sequence, wherein each part preferably comprises at least 5 continuous amino acids, at least 10 continuous amino acids, preferably at least 20 continuous amino acids, preferably at least 30 continuous amino acids of the amino acid sequence.

The present invention is described in detail by the figures and examples below, which are used only for illustration purposes and which are not be construed as limiting the scope of the invention. Owing to the description and the examples, further embodiments which are likewise included in the invention are accessible to the skilled worker.

### SEQUENCES

Within this disclosure reference to the following sequences and SEQ ID NOs is made:

| | | |
|---|---|---|
| SEQ ID NO: 1 | HCDR3 (MAB-19-0202) | intersection of Kabat and IMGT (=Kabat) |
| SEQ ID NO: 2 | HCDR3 (MAB-19-0208) | intersection of Kabat and IMGT (=Kabat) |
| SEQ ID NO: 3 | HCDR3 (MAB-19-0217) | intersection of Kabat and IMGT (=Kabat) |
| SEQ ID NO: 4 | HCDR3 (MAB-19-0223) | intersection of Kabat and IMGT (=Kabat) |
| SEQ ID NO: 5 | HCDR3 (MAB-19-0233) | intersection of Kabat and IMGT (=Kabat) |
| SEQ ID NO: 6 | HCDR3 (MAB-19-0202) | IMGT |
| SEQ ID NO: 7 | HCDR3 (MAB-19-0208) | IMGT |
| SEQ ID NO: 8 | HCDR3 (MAB-19-0217) | IMGT |
| SEQ ID NO: 9 | HCDR3 (MAB-19-0223) | IMGT |
| SEQ ID NO: 10 | HCDR3 (MAB-19-0233) | IMGT |
| SEQ ID NO: 11 | HCDR2 (MAB-19-0202) | intersection of Kabat and IMGT (=Kabat) |
| SEQ ID NO: 12 | HCDR2 (MAB-19-0208) | intersection of Kabat and IMGT (=Kabat) |
| SEQ ID NO: 13 | HCDR2 (MAB-19-0217) | intersection of Kabat and IMGT (=Kabat) |
| SEQ ID NO: 14 | HCDR2 (MAB-19-0223) | intersection of Kabat and IMGT (=Kabat) |
| SEQ ID NO: 15 | HCDR2 (MAB-19-0233) | intersection of Kabat and IMGT (=Kabat) |
| SEQ ID NO: 16 | HCDR2 (MAB-19-0202) | Kabat |
| SEQ ID NO: 17 | HCDR2 (MAB-19-0208) | Kabat |
| SEQ ID NO: 18 | HCDR2 (MAB-19-0217) | Kabat |
| SEQ ID NO: 19 | HCDR2 (MAB-19-0223) | Kabat |
| SEQ ID NO: 20 | HCDR2 (MAB-19-0233) | Kabat |
| SYN | HCDR1 (MAB-19-0202) | intersection of Kabat and IMGT |
| RYY | HCDR1 (MAB-19-0208) | intersection of Kabat and IMGT |
| RYY | HCDR1 (MAB-19-0217) | intersection of Kabat and IMGT |
| SEQ ID NO: 21 | HCDR1 (MAB-19-0223) | intersection of Kabat and IMGT |
| SEQ ID NO: 22 | HCDR1 (MAB-19-0233) | intersection of Kabat and IMGT |
| SEQ ID NO: 23 | HCDR1 (MAB-19-0202) | Kabat |
| SEQ ID NO: 24 | HCDR1 (MAB-19-0208) | Kabat |
| SEQ ID NO: 25 | HCDR1 (MAB-19-0217) | Kabat |
| SEQ ID NO: 26 | HCDR1 (MAB-19-0223) | Kabat |
| SEQ ID NO: 27 | HCDR1 (MAB-19-0233) | Kabat |
| SEQ ID NO: 28 | HCDR1 (MAB-19-0202) | IMGT |
| SEQ ID NO: 29 | HCDR1 (MAB-19-0208) | IMGT |
| SEQ ID NO: 30 | HCDR1 (MAB-19-0217) | IMGT |
| SEQ ID NO: 31 | HCDR1 (MAB-19-0223) | IMGT |
| SEQ ID NO: 32 | HCDR1 (MAB-19-0233) | IMGT |
| SEQ ID NO: 33 | LCDR3 (MAB-19-0202) | intersection = Kabat = IMGT |
| SEQ ID NO: 34 | LCDR3 (MAB-19-0208) | intersection = Kabat = IMGT |
| SEQ ID NO: 35 | LCDR3 (MAB-19-0217) | intersection = Kabat = IMGT |
| SEQ ID NO: 36 | LCDR3 (MAB-19-0223) | intersection = Kabat = IMGT |
| SEQ ID NO: 37 | LCDR3 (MAB-19-0233) | intersection = Kabat = IMGT |
| QAS | LCDR2 (MAB-19-0202) | intersection of Kabat and IMGT (=IMGT) |
| DAS | LCDR2 (MAB-19-0208) | intersection of Kabat and IMGT (=IMGT) |
| DAS | LCDR2 (MAB-19-0217) | intersection of Kabat and IMGT (=IMGT) |
| DAS | LCDR2 (MAB-19-0223) | intersection of Kabat and IMGT (=IMGT) |
| DAS | LCDR2 (MAB-19-0233) | intersection of Kabat and IMGT (=IMGT) |
| SEQ ID NO: 38 | LCDR2 (MAB-19-0202) | Kabat |
| SEQ ID NO: 39 | LCDR2 (MAB-19-0208) | Kabat |
| SEQ ID NO: 39 | LCDR2 (MAB-19-0217) | Kabat |
| SEQ ID NO: 40 | LCDR2 (MAB-19-0223) | Kabat |
| SEQ ID NO: 41 | LCDR2 (MAB-19-0233) | Kabat |
| SEQ ID NO: 42 | LCDRI (MAB-19-0202) | intersection of Kabat and IMGT (=IMGT) |
| SEQ ID NO: 43 | LCDRI (MAB-19-0208) | intersection of Kabat and IMGT (=IMGT) |
| SEQ ID NO: 44 | LCDR1 (MAB-19-0217) | intersection of Kabat and IMGT (=IMGT) |
| SEQ ID NO: 45 | LCDR1 (MAB-19-0223) | intersection of Kabat and IMGT (=IMGT) |
| SEQ ID NO: 46 | LCDR1 (MAB-19-0233) | intersection of Kabat and IMGT (=IMGT) |
| SEQ ID NO: 47 | LCDR1 (MAB-19-0202) | Kabat |
| SEQ ID NO: 48 | LCDR1 (MAB-19-0208) | Kabat |
| SEQ ID NO: 49 | LCDRI (MAB-19-0217) | Kabat |
| SEQ ID NO: 50 | LCDR1 (MAB-19-0223) | Kabat |
| SEQ ID NO: 51 | LCDR1 (MAB-19-0233) | Kabat |

| | |
|---|---|
| SEQ ID NO: 52 | VH (MAB-19-0202) |
| SEQ ID NO: 53 | VH (MAB-19-0208) |
| SEQ ID NO: 54 | VH (MAB-19-0217) |
| SEQ ID NO: 55 | VH (MAB-19-0223) |
| SEQ ID NO: 56 | VH (MAB-19-0233) |
| SEQ ID NO: 57 | VL (MAB-19-0202) |
| SEQ ID NO: 58 | VL (MAB-19-0208) |
| SEQ ID NO: 59 | VL (MAB-19-0217) |
| SEQ ID NO: 60 | VL (MAB-19-0223) |
| SEQ ID NO: 61 | VL (MAB-19-0233) |
| SEQ ID NO: 62 | H5 (derived from MAB-19-0202) |
| SEQ ID NO: 63 | H5 (derived from MAB-19-0233) |
| SEQ ID NO: 64 | H1 (derived from MAB-19-0233) |
| SEQ ID NO: 65 | L1 (derived from MAB-19-0202) |
| SEQ ID NO: 66 | L2 (derived from MAB-19-0202) |
| SEQ ID NO: 67 | L3 (derived from MAB-19-0202) |
| SEQ ID NO: 68 | L4 (derived from MAB-19-0202) |
| SEQ ID NO: 69 | L1 (derived from MAB-19-0233) |
| SEQ ID NO: 70 | L4 (derived from MAB-19-0233) |
| SEQ ID NO: 71 | human PD-1 complete |
| SEQ ID NO: 72 | human PD-1 extracellular domain |
| SEQ ID NO: 73 | nucleic acid human PD-1 |
| SEQ ID NO: 74 | nucleic acid VH (MAB-19-0202) |
| SEQ ID NO: 75 | nucleic acid VH (MAB-19-0208) |
| SEQ ID NO: 76 | nucleic acid VH (MAB-19-0217) |
| SEQ ID NO: 77 | nucleic acid VH (MAB-19-0223) |
| SEQ ID NO: 78 | nucleic acid VH (MAB-19-0233) |
| SEQ ID NO: 79 | nucleic acid VL (MAB-19-0202) |
| SEQ ID NO: 80 | nucleic acid VL (MAB-19-0208) |
| SEQ ID NO: 81 | nucleic acid VL (MAB-19-0217) |
| SEQ ID NO: 82 | nucleic acid VL (MAB-19-0223) |
| SEQ ID NO: 83 | nucleic acid VL (MAB-19-0233) |
| SEQ ID NO: 84 | nucleic acid H5 (derived from MAB-19-0202) |
| SEQ ID NO: 85 | nucleic acid H5 (derived from MAB-19-0233) |
| SEQ ID NO: 86 | nucleic acid H1 (derived from MAB-19-0233) |
| SEQ ID NO: 87 | nucleic acid LI (derived from MAB-19-0202) |
| SEQ ID NO: 88 | nucleic acid L2 (derived from MAB-19-0202) |
| SEQ ID NO: 89 | nucleic acid L3 (derived from MAB-19-0202) |
| SEQ ID NO: 90 | nucleic acid L4 (derived from MAB-19-0202) |
| SEQ ID NO: 91 | nucleic acid Ll (derived from MAB-19-0233) |
| SEQ ID NO: 92 | nucleic acid L4 (derived from MAB-19-0233) |
| SEQ ID NO: 93 | pST4-hAg-husec(opt)-anti-PD1-0202-HC-hIgG1- |
| | LALA-PG-FI-A30LA70 (HC; RiboMab-19-0202) |
| SEQ ID NO: 94 | 5′-UTR |
| SEQ ID NO: 95 | Kozac sequence |
| SEQ ID NO: 96 | signal peptide sequence |
| SEQ ID NO: 97 | constant domain CH1 |
| SEQ ID NO: 98 | hinge region |
| SEQ ID NO: 99 | constant domain CH2 |
| SEQ ID NO: 100 | constant domain CH3 |
| SEQ ID NO: 101 | F-element |
| SEQ ID NO: 102 | I-element |
| SEQ ID NO: 103 | poly(A) tail (A30LA70) |
| SEQ ID NO: 104 | pST4-hAg-husec(opt)-anti-PD1-0202-LC-hIgG1- |
| | FI-A30LA70 (LC; RiboMab-19-0202) |
| SEQ ID NO: 105 | constant domain CL kappa |
| SEQ ID NO: 106 | pST4-hAg-husec(opt)-anti-PD1-0233-HC-hIgG1- |
| | LALA-PG-FI-A30LA70 (HC; RiboMab-19-0233) |
| SEQ ID NO: 107 | pST4-hAg-husec(opt)-anti-PD1-0233-LC-hIgG1- |
| | FI-A30LA70 (LC; RiboMab-19-0233) |

### EXAMPLES

The techniques and methods used herein are described herein or carried out in a manner known per se and as described, for example, in Sambrook et al., Molecular Cloning: A Laboratory Manual, 2^{nd} Edition (1989) Cold Spring Harbor Laboratory Press, Cold Spring Harbor, N.Y. All methods including the use of kits and reagents are carried out according to the manufacturers' information unless specifically indicated.

### Example 1: Generation of Anti-Human PD-1 Antibodies

Three New Zealand White rabbits were immunized with recombinant human His-tagged PD-1 protein (R&D Systems, cat. no. 8986-PD). Single B cells from blood were sorted and supernatants screened for production of PD-1 specific antibodies by human PD-1 enzyme-linked immunosorbent assay (ELISA), cellular human PD-1 binding assay and by human PD-1/PD-L1 blockade bioassay as described in Examples 2-4. From screening-positive B cells, RNA was extracted and sequencing was performed. The variable regions of heavy and light chain were gene synthesized and cloned N-terminal of human immunoglobulin constant parts (IgG1/h) containing mutations L234A and L235A (LALA) to minimize interactions with Fcg receptors in a pCEP4 expression vector (Thermo Fisher, cat. no. V04450). The variable region sequences of the chimeric PD-1 antibodies are shown in the following tables. Table 1 shows the variable regions of the heavy chain, while table 2 shows the variable regions of the light chain. In both cases the framing regions (FRs) as well as the complementarity determining regions (CDRs) according to Kabat numbering are defined. The underlined amino acids indicate the CDRs according to the IMGT numbering. The bold letters indicate the intersection of Kabat and IMGT numbering.

TABLE 1
HEAVY CHAIN

| | | | SEQ | | | SEQ | | | SEQ | |
|---|---|---|---|---|---|---|---|---|---|---|
| Sequence ID | FR1 | CDR1 | ID# | FR2 | CDR2 | ID# | FR3 | CDR3 | ID# | FR4 |
| MAB-19-0202- | QSVEE | SYN | 23 | WV | IIS | 16 | RFTIS | AFY | 1 | WG |
| HC | SGGRL | MG | | RQ | GGT | | KTSST | DDY | | PG |
| SEQ ID NO: 52 | VTPGT | | | AP | IGH | | TVDLK | DYN | | TL |
| | PLTLT | | | GK | YAS | | MTSLT | V | | VT |
| | CTVSG | | | GL | WAK | | TEDTA | | | VS |
| | FSLY | | | EY | G | | TYFCA | | | S |
| | | | | IG | | | R | | | |
| MAB-19-0208- | QSVEE | RYY | 24 | WV | SFY | 17 | RFTFS | NSG | 2 | WG |
| HC | SGGRL | IS | | RQ | ADS | | TASST | DAQ | | PG |
| SEQ ID NO: 53 | VTPGT | | | AP | GTT | | TVDLK | FNI | | TL |
| | PLTLT | | | GK | WYA | | MTSPT | | | VT |
| | CTVSG | | | GL | TWV | | TEDTA | | | VS |
| | FSLS | | | EW | KG | | TYFCA | | | S |
| | | | | IG | | | R | | | |
| MAB-19-0217- | QSVEE | RYY | 25 | WV | IIY | 18 | RFTFS | STT | 3 | WG |
| HC | SGGRL | MT | | RQ | PDT | | KTSST | DAQ | | PG |
| SEQ ID NO: 54 | VTPGT | | | AP | GTT | | TVDLK | FNI | | TL |
| | PLTLT | | | GK | WYA | | MTSPT | | | VT |
| | CTVSG | | | GL | SWV | | TEDTA | | | VS |
| | FSLS | | | EW | KG | | TYFCA | | | S |
| | | | | IG | | | R | | | |
| MAB-19-0223- | QEHLV | DTY | 26 | WV | CIG | 19 | RFTIS | EIP | 4 | WG |
| HC | ESGGG | WIC | | RQ | IGG | | KTSST | YFN | | PG |
| SEQ ID NO: 55 | LVQPE | | | PP | SGS | | TVTLQ | V | | TL |
| | GSLTL | | | GK | TYY | | MTTLT | | | VT |
| | TCKAS | | | GL | AGW | | DADTA | | | VS |
| | GIDFS | | | EW | AKG | | TYFCA | | | S |
| | | | | IG | | | T | | | |
| MAB-19-0233- | QSLEE | SVY | 27 | WV | CIY | 20 | RFTIS | AGY | 5 | WG |
| HC | SGGDL | YMC | | RQ | VGS | | KTSST | VGA | | QG |
| SEQ ID NO: 56 | VKPGA | | | AP | SGV | | TVTLQ | VYV | | TL |
| | SLTLT | | | GK | SYY | | MTSLT | TLT | | VT |
| | CKASG | | | GL | ATW | | AADTA | RLD | | VS |
| | IDFS | | | EW | AKG | | TYFCA | L | | S |
| | | | | IA | | | R | | | |

TABLE 2
LIGHT CHAIN

| Se- | | | SEQ | | | SEQ | | | SEQ | |
|---|---|---|---|---|---|---|---|---|---|---|
| quence | | | ID | | | ID | | | ID | |
| ID | FR1 | CDR1 | # | FR2 | CDR2 | # | FR3 | CDR3 | # | FR4 |
| MAB-19- | AAVLT | QSS | 47 | WY | QAS | 38 | GVPSR | AGG | 33 | FG |
| 0202-LC | QTPSP | QSV | | QQ | KLE | | FKGSG | YSS | | GG |
| SEQ ID | VSAAV | YGN | | KP | T | | SGTQF | SSD | | TE |
| NO: 57 | GGTVT | NQL | | GQ | | | TLTIS | TT | | VV |
| | ISC | S | | PP | | | DLESD | | | VK |
| | | | | KL | | | DAATY | | | |
| | | | | LI | | | YC | | | |
| | | | | Y | | | | | | |
| MAB-19- | AAVLT | QSS | 48 | WY | DAS | 39 | GVPSR | AGG | 34 | FG |
| 0208-LC | QTPSP | ESV | | QQ | TLA | | FSGSG | YSV | | GG |
| SEQ ID | VSAAV | YNK | | KP | S | | SGTQF | TSD | | TE |
| NO: 58 | GGTVS | NQL | | GQ | | | TLTIS | TT | | VV |
| | ISC | C | | RP | | | DVQSD | | | VR |
| | | | | KL | | | AAATY | | | |
| | | | | LI | | | YC | | | |
| | | | | Y | | | | | | |
| MAB-19- | AAVLT | QSS | 49 | WY | DAS | 39 | GVPSR | AGG | 35 | FG |
| 0217-LC | QTPSP | ENV | | QQ | TLA | | FSGSG | YST | | GG |
| SEQ ID | VSAAV | YTD | | KP | S | | SGTQF | TSD | | TE |
| NO: 59 | GGTVS | NQL | | GQ | | | TLTIS | TT | | VV |
| | ISC | C | | RP | | | GVQSD | | | VK |
| | | | | KL | | | DAATY | | | |
| | | | | LI | | | YC | | | |
| | | | | Y | | | | | | |
| MAB-19- | AQVLT | QSS | 50 | WY | DAS | 40 | GVPSR | QGT | 36 | FG |
| 0223-LC | QTPSS | QSV | | QQ | KLT | | FKGSG | YDV | | GG |
| SEQ ID | VSAAV | YNK | | KP | S | | SGTQF | NGW | | AE |
| NO: 60 | GGTVT | NWL | | GQ | | | TLTIS | LVA | | VV |
| | INC | A | | PP | | | GVQSD | | | VK |
| | | | | KL | | | DAATY | | | |
| | | | | LI | | | YC | | | |
| | | | | Y | | | | | | |
| MAB-19- | AAVLT | QSS | 51 | WY | DAS | 41 | GVPSR | LGG | 37 | FG |
| 0233-LC | QTPSP | QSI | | QQ | KLA | | FSGSG | YDD | | GG |
| SEQ ID | VSAAV | YTN | | KP | S | | SGTQF | DAD | | TE |
| NO: 61 | GGTVT | NDL | | GQ | | | TLTIS | NA | | VV |
| | ISC | A | | PP | | | GVQSD | | | VK |
| | | | | KL | | | DAATY | | | |
| | | | | LI | | | YC | | | |
| | | | | Y | | | | | | |

HEK293-FreeStyle cell transient transfections using 293-free transfection reagent (Novagen/Merck) were executed by Tecan Freedom Evo device. Chimeric antibodies were purified from cell supernatant using affinity chromatography on a Dionex Ultimate 3000 HPLC with plate autosampler. Produced chimeric antibodies were purified from cell culture supernatants using protein-A affinity chromatography. Antibodies were eluted with 100 mM glycin pH 2.5 and neutralized with 1M Tris pH 9 to achieve a final pH between 6 and 7. Purified antibodies were used for further analysis in particular retesting by human PD-1 ELISA, cellular human PD-1 binding assay, human PD-1/PD-L1 blockade bioassay, and the T-cell proliferation assay as described in Examples 2-5. The two chimeric rabbit antibodies MAB-19-0202 and MAB-19-0233 were identified as best performing clones and subsequently humanized. Humanized antibody sequences were generated at Fusion Antibodies (Belfast, Ireland). The allocation of the humanized light and heavy chains to antibody ID of the recombinant humanized sequences are listed in Table 3. The variable region sequences of the humanized light and heavy chains are shown in Table 4 and 5. Table 4 shows the variable regions of the heavy chain, while table 5 shows the variable regions of the light chain. In both cases the framing regions (FRs) as well as the complementarity determining regions (CDRs) according to Kabat numbering are defined. The underlined amino acids indicate the CDRs according to the IMGT numbering.

TABLE 3

| | light chain | | heavy chain | |
|---|---|---|---|---|
| | humanized | Light | humanized | Heavy |
| | variant | chain | variant | chain |
| | derived from | SEQ ID | derived from | SEQ ID |
| antibody ID | MAB-19-0233 | NO: | MAB-19-0233 | NO: |
| MAB-19- | MAB-19-0233- | 69 | MAB-19-0233- | 63 |
| 0583 | L1 | | H5 | |
| MAB-19- | MAB-19-0233- | 70 | MAB-19-0233- | 64 |
| 0594 | L4 | | H1 | |
| MAB-19- | MAB-19-0233- | 70 | MAB-19-0233- | 63 |
| 0598 | L4 | | H5 | |
| | humanized | | humanized | |
| | variant | | variant | |
| | derived from | | derived from | |
| | MAB-19-0202 | | MAB-19-0202 | |
| MAB-19- | MAB-19-0202- | 65 | MAB-19-0202- | 62 |
| 0603 | L1 | | H5 | |
| MAB-19- | MAB-19-0202- | 66 | MAB-19-0202- | 62 |
| 0608 | L2 | | H5 | |
| MAB-19- | MAB-19-0202- | 67 | MAB-19-0202- | 62 |
| 0613 | L3 | | H5 | |
| MAB-19- | MAB-19-0202- | 68 | MAB-19-0202- | 62 |
| 0618 | L4 | | H5 | |

TABLE 4
HEAVY CHAIN

| Sequence ID | FR1 | CDR1 | FR2 | CDR2 | FR3 | CDR3 | FR4 |
|---|---|---|---|---|---|---|---|
| MAB-19-0202- | QVQLV | SYN | WV | IIS | RFTIS | AFY | WG |
| H5 | ESGGG | MG | RQ | GGT | RDTSK | DDY | PG |
| SEQ ID NO: 62 | LVQPG | | AP | IGH | TTLYL | DYN | TI |
| | TSLRL | | GK | YAS | QMNSL | V | VT |
| | SCSVS | | GL | WAK | TTEDT | | VS |
| | GFSLY | | EY | G | ATYFC | | S |
| | | | IG | | AR | | |
| MAB-19-0233- | QVQLV | SVY | WV | CIY | RFTIS | AGY | WG |
| H5 | ESGGD | YMC | RQ | VGS | RDTST | VGA | QG |
| SEQ ID NO: 63 | VVKPG | | AP | SGV | STLFL | VYV | TI |
| | RSLRL | | GK | SYY | QMNSL | TLT | VT |
| | SCKAS | | GL | ATW | RAGDT | RLD | VS |
| | GIDFS | | EW | AKG | ATYYC | L | S |
| | | | IA | | AR | | |
| MAB-19-0233- | EVQLE | SVY | WV | CIY | RFTIS | AGY | WG |
| H1 | ESGGG | YMC | RQ | VGS | RDNSK | VGA | RG |
| SEQ ID NO: 64 | LVKPG | | AP | SGV | NTLYL | VYV | TL |
| | GSLRL | | GK | SYY | QMNSL | TLT | VT |
| | SCAAS | | GL | ATW | RAEDT | RLD | VS |
| | GIDFS | | EW | AKG | AVYYC | L | S |
| | | | VS | | AR | | |

TABLE 5
LIGHT CHAIN

| Sequence ID | FR1 | CDR1 | FR2 | CDR2 | FR3 | CDR3 | FR4 |
|---|---|---|---|---|---|---|---|
| MAB-19-0202- | DIVMT | QSS | WY | QAS | GVPSR | AGG | FG |
| L1 | QSPSS | QSV | QQ | KLE | FSGSG | YSS | GG |
| SEQ ID NO: 65 | LSASV | YGN | KP | T | SGTDE | SSD | TK |
| | GDRVT | NQL | GK | | TLTIS | TT | VV |
| | ITC | S | AP | | SLQPE | | IK |
| | | | KI | | DFATY | | |
| | | | LI | | YC | | |
| | | | Y | | | | |
| MAB-19-0202- | DIQMT | QSS | WY | QAS | GVPSR | AGG | FG |
| L2 | QSPST | QSV | QQ | KLE | FSGSG | YSS | QG |
| SEQ ID NO: 66 | LSASV | YGN | KP | T | SGTQF | SSD | TK |
| | GDRVT | NQL | GK | | TLTIS | TT | VE |
| | ITC | S | AP | | SLQPD | | IK |
| | | | KL | | DEASY | | |
| | | | LI | | YC | | |
| | | | Y | | | | |
| MAB-19-0202- | DIQMT | QSS | WY | QAS | GVPSR | AGG | FG |
| L3 | QSPSS | QSV | QK | KLE | FSGSG | YSS | PG |
| SEQ ID NO: 67 | LSASV | YGN | KE | T | SGTDE | SSD | TK |
| | GDRVT | NQL | GQ | | TLTIS | TT | VD |
| | ITC | S | AP | | SLQPE | | IK |
| | | | KL | | DFATY | | |
| | | | LI | | YC | | |
| | | | Y | | | | |
| MAB-19-0202- | AIQLT | QSS | WY | QAS | GVPSR | AGG | FG |
| L4 | QSPSS | QSV | QQ | KLE | FRGSG | YSS | GG |
| SEQ ID NO: 68 | LSASV | YGN | KP | T | SGTQF | SSD | TE |
| | GGTVT | NQL | GQ | | TLTIS | TT | VV |
| | ITC | S | PP | | SLQSE | | VK |
| | | | KL | | DFATY | | |
| | | | LI | | YC | | |
| | | | Y | | | | |
| MAB-19-0233- | DVVMT | QSS | WY | DAS | GVPDR | LGG | FG |
| L1 | QSPST | QSI | QQ | KLA | FSGSG | YDD | QG |
| SEQ ID NO: 69 | VSASV | YTN | KP | S | SGTDF | DAD | TK |
| | GDRVT | NDL | GQ | | TLTIS | NA | VE |
| | LTC | A | PP | | SLQAD | | IK |
| | | | KL | | DFATY | | |
| | | | LI | | YC | | |
| | | | Y | | | | |
| MAB-19-0233- | DIQMT | QSS | WY | DAS | GVPSR | LGG | FG |
| L4 | QSPSS | QSI | QQ | KLA | FSGSG | YDD | GG |
| SEQ ID NO: 70 | LSASV | YTN | KP | S | SGTQF | DAD | TE |
| | GGTVT | NDL | GQ | | TLTIS | NA | VV |
| | ITC | A | PP | | SLQSE | | VK |
| | | | KL | | DAATY | | |
| | | | LI | | YC | | |
| | | | Y | | | | |

Recombinant humanized hIgG1-LALA antibodies were cloned and produced as described above and analyzed as well by human PD-1 ELISA, cellular human PD-1 binding assay, PD-1/PD-L1 blockade bioassay, and the T-cell proliferation assay as described in Examples 2-5.

### Example 2: Human-PD-1 ELISA

The binding potency of chimeric and humanized anti-PD-1 antibodies to recombinant human-PD-1 extracellular domain was determined by ELISA. Recombinant human PD-1 human-FC Chimera (R&D Systems) was coated on 384-well MaxiSorp™ flat bottom plates (Nunc) at a concentration of 0.625 μg/mL in PBS (Vendor) for 60 minutes at room temperature. Coated plates were washed three times with PBS, 0.1% Tween (PBS-T), blocked by incubation with PBS, 2% BSA, 0.05% Tween for 60 minutes at room temperature, and washed for an additional three times with PBS-T. Anti-PD-1-antibodies were added in PBS, 0.5% BSA, 0.05% Tween (ELISA buffer) in concentrations ranging from 1,000 to 0.06 ng/mL or 2,500 to 0.15 ng/mL and the plate was incubated for 60 minutes at room temperature. As reference antibodies, anti-hPD-1-Ni-hIgG4 (InvivoGen; Cat. No. hpd1ni-mab114; features the variable region of Nivolumab) and anti-hPD1-Pem-hIgG4 (InvivoGen; Cat. No. hpd1pe-mab14; features the variable region of Pembrolizumab) were used. After 3 washes with PBS-T, horseradish peroxidase coupled goat-anti-human-IgG (Fab′)₂ fragment (AbD Serotec; Cat. No. STAR126P) was added in ELISA buffer at a dilution of 1:5,000. The plate was incubated for 60 minutes at room temperature, and washed 6 times with PBS-T before TMB solution (Thermo Fisher Scientific) was added. After 10 minutes HCl was added and the absorbance at wavelengths of 450 and 620 nm recorded using a Tecan Infinite M1000 reader. Data was fitted with a 4-parameter logistic model and EC50 values calculated using GraphPad Prism 8.4.3 (GraphPad Software, San Diego, CA, USA).

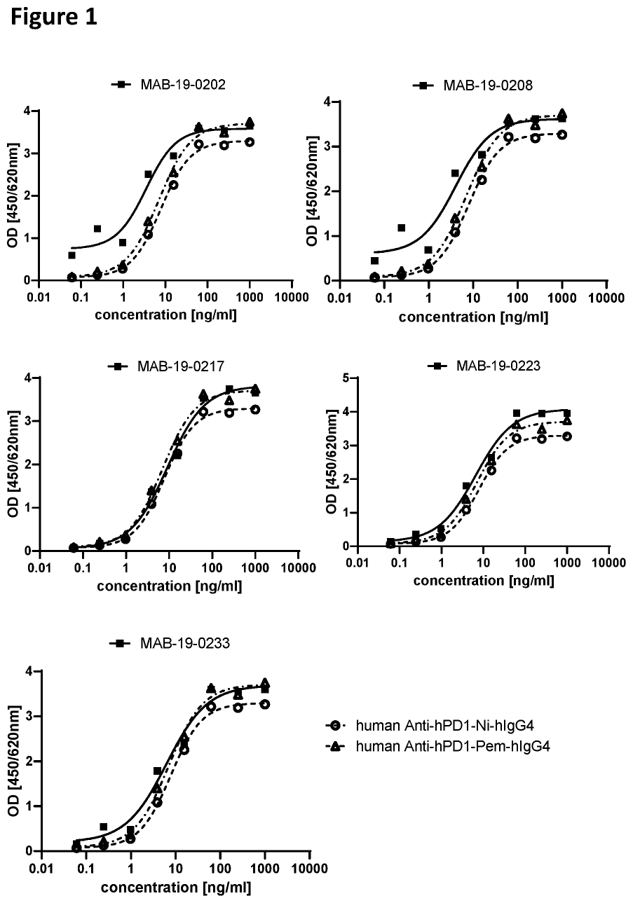

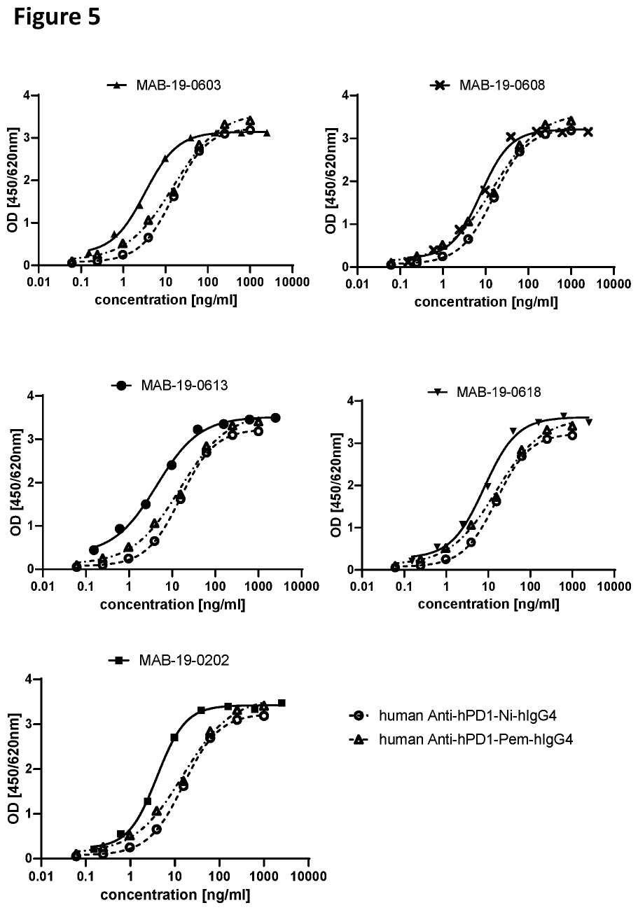

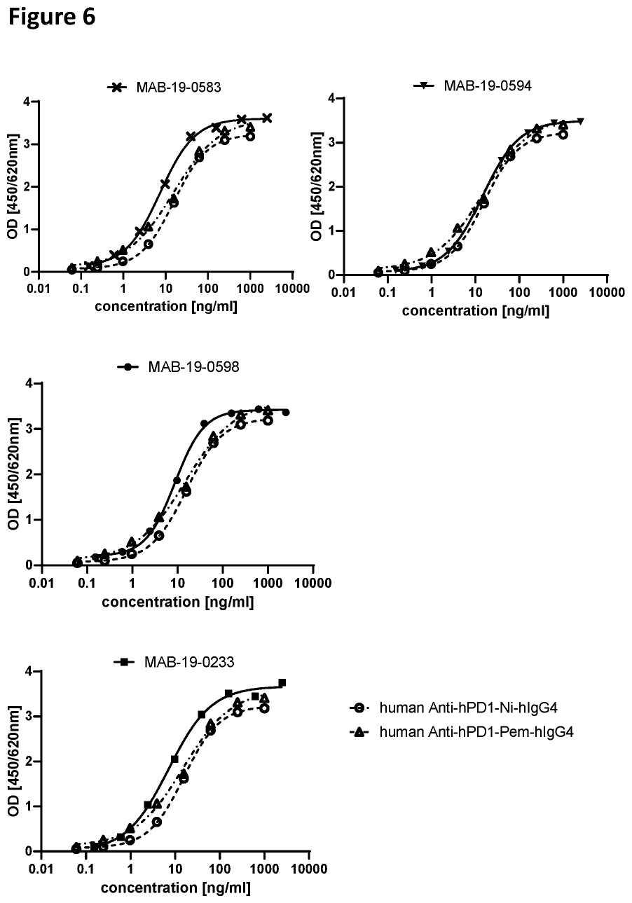

Binding curves for the chimeric anti-PD-1 antibodies MAB-19-0202, MAB-19-0208, MAB-19-0217, MAB-19-0223, and MAB-19-0233 to human-PD-1 were comparable to the reference antibodies anti-hPD-1-Ni-hIgG4 and anti-hPD1-Pem-hIgG4 as shown in FIG. 1. Analysis of the EC50 values revealed lower EC50 values of the antibodies MAB-19-0202, MAB-19-0208, MAB-19-0223, and MAB-19-0233 (Table 6). After humanization of the chimeric antibodies MAB-19-0202 and MAB-19-0233 the assay was repeated with the humanized variants and the parental chimeric antibody (FIGS. 5 and 6). EC50 values of two chimeric and the humanized anti-hPD-1 antibodies were all lower than the EC50 values of the two reference antibodies (Table 7).

### Example 3: HEK-293-hPD-1 Cell Binding

Binding of chimeric and humanized anti-PD-1 antibodies to cell surface expressed hPD-1 was analyzed using HEK-293 cells ectopically expressing full-length human-PD1 (BPS Biosciences; Cat. No. 60680). Cell cultures were grown in MEM containing 10% FCS, ix MEM NEAA, 1 mM Na pyruvate and 100 μg/mL Hygromycin B. Hygromycin B was omitted when cells were plated for testing antibody binding. 1,000 cells in 20 μL medium were seeded per well in black 384-well cell-culture treated plates with clear bottom and were incubated for 2 hours at 37° C. and 5% CO₂. Anti-PD-1 antibodies were added in 5 μL medium to final concentrations ranging from 1,000 to 0.06 ng/mL or 620 to 0.45 ng/mL. As reference antibodies, anti-hPD-1-Ni-hIgG4 and anti-hPD1-Pem-hIgG4 were used. After an 18 hours incubation at 37° C. and 5% CO₂, plates were washed once with 25 μL PBS, 0.05% Tween 20 (cell wash buffer) and Alexa-Fluor-488-conjugated AffiniPure goat-anti-human-IgG F(ab′)₂ fragment (Vendor) was added at a concentration of 0.8 μg/mL in 20 μL medium. Plates were incubated for 4 hours at 37° C. and 5% CO₂ in the dark, washed once with 25 μL cell wash buffer and incubated for 10 minutes with 20 μL medium containing 5 μg/ml Hoechst (Invitrogen). Cell-associated immunofluorescent signals were recorded using a CellInsight CX5 high content imager device (Thermo Fisher). Data was fitted with a 4-parameter logistic model and EC50 values calculated using GraphPad Prism 8.4.3.

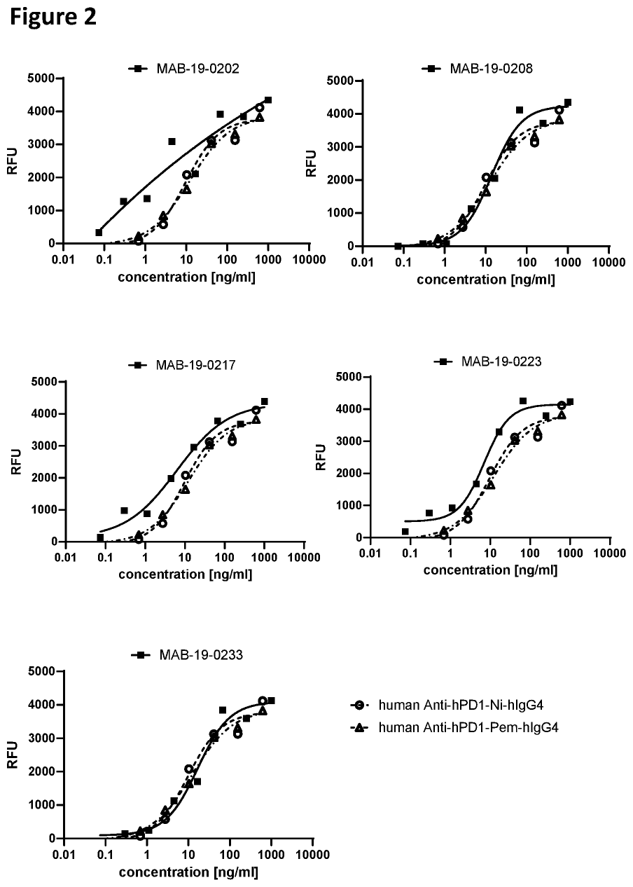

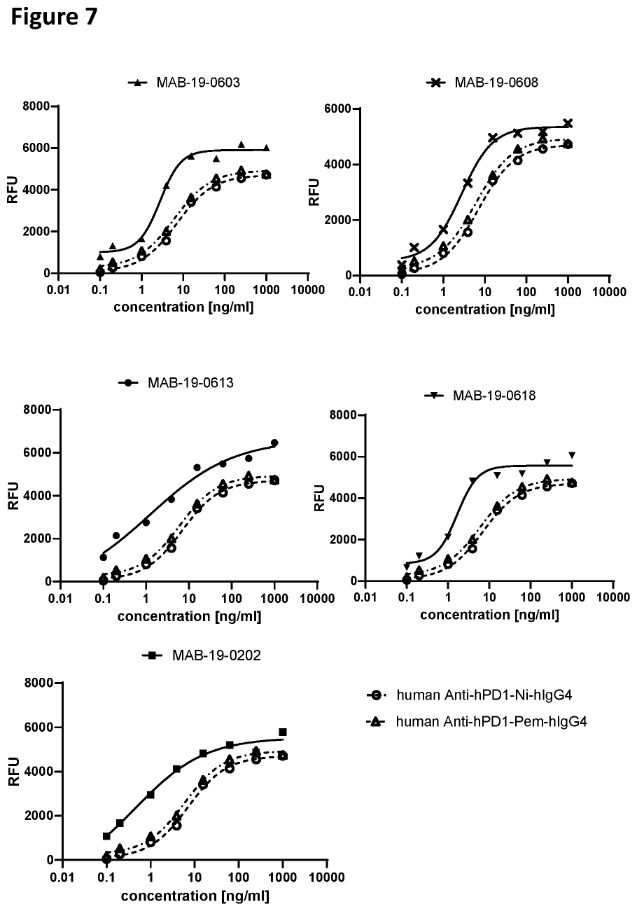

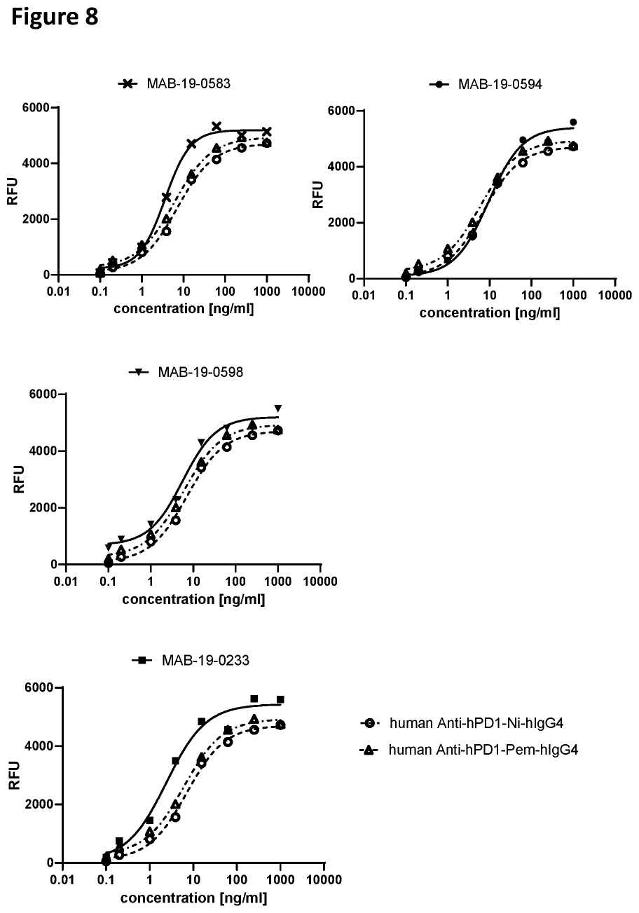

Binding curves for the cellular binding of the chimeric anti-PD-1 antibodies MAB-19-0202, MAB-19-0208, MAB-19-0217, MAB-19-0223, and MAB-19-0233 to human-PD-1 were comparable to the reference antibodies anti-hPD-1-Ni-hIgG4 and anti-hPD1-Pem-hIgG4 as shown in FIG. 2. Analysis of the EC50 values revealed a lower EC50 values of the antibodies MAB-19-0217, and MAB-19-0223 (Table 6). EC50 value for MAB-19-0202 could not be calculated due to an incomplete fit (n.a. not applicable). After humanization of the chimeric antibodies MAB-19-0202 and MAB-19-0233 the assay was repeated with the humanized variants and the parental chimeric antibody (FIGS. 7 and 8). EC50 values of two chimeric and the humanized anti-hPD-1 antibodies (except for MAB-19-0594) were all lower than the two reference antibodies in this experiment (Table 7).

### Example 4: Human PD-1/PD-L1 Blockade Bioassay

The potency of chimeric and humanized anti-PD-1 antibodies to block the PD-1/PD-L1 interaction was analyzed using a PD-1/PD-L1 blockade bioassay (Promega; cat. no. #J1250) according to the manufacturer's instructions. Briefly, 500 μL PD-L1 expressing artificial APC aAPC/CHO-K1 cell suspension was added to 14.5 mL cell recovery medium (90% Ham's F-12 (Promega; cat. no. J123A)+10% Fetal Bovine Serum (Promega; cat. no. J121A)) and μL cell suspension were seeded per well of a flat-bottom 384-well assay plate. After an overnight incubation at 37° C. and 5% CO₂, medium was removed and antibodies were added in 10 μL HAM's F-12, 1% FBS at concentrations ranging from 40,000 to 18 ng/mL or 20,000 to 9 ng/mL. As reference antibodies, anti-hPD-1-Ni-hIgG4 and anti-hPD1-Pem-hIgG4 were used. PD-1 expressing effector cells (Promega; cat. no. J115A) were thawed and resuspended in HAM's F-12, 1% FBS. 10 μL effector cell suspension were added to each well and the plate incubated for 6 hours at 37° C. and 5% CO₂. Plates were equilibrated to room temperature for 10 minutes and 20 μL Bio-Glo Luciferase assay reagent were added to each well. After 15 minutes of incubation at room temperature, the luminescence was measured using a Tecan Infinite M1000 reader. Data was fitted with a 4-parameter logistic model and EC50 values calculated using GraphPad Prism 8.4.3.

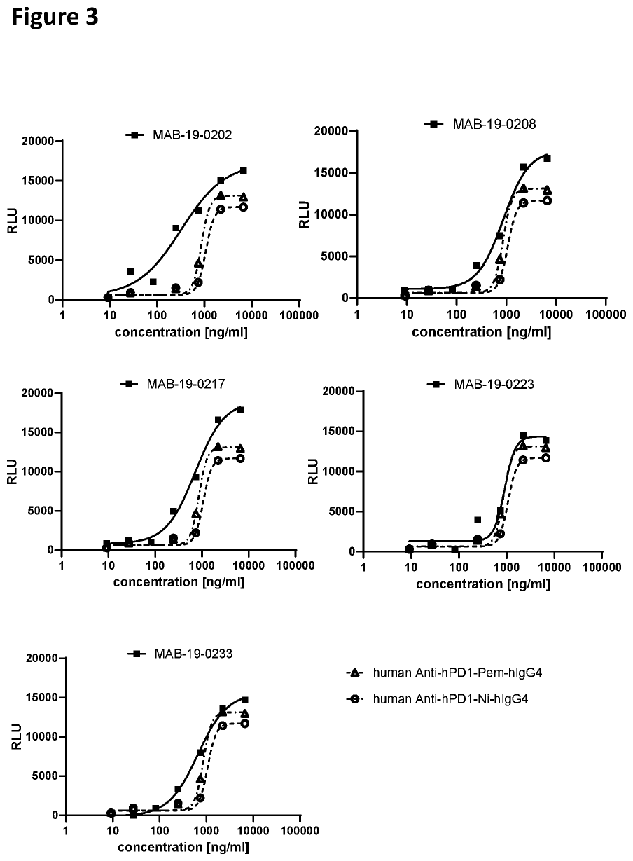

PD-1:PD-L1 blocking activity of the chimeric anti-PD-1 antibodies MAB-19-0202, MAB-19-0208, MAB-19-0217, MAB-19-0223, and MAB-19-0233 was comparable to the reference antibodies anti-hPD-1-Ni-hIgG4 and anti-hPD1-Pem-hIgG4 as shown in FIG. 3. This was also reflected in the IC50 values (Table 6). MAB-19-0202 and MAB-19-0233 performed clearly better than the two reference antibodies resulting in lower IC50 values compared to the two reference antibodies.

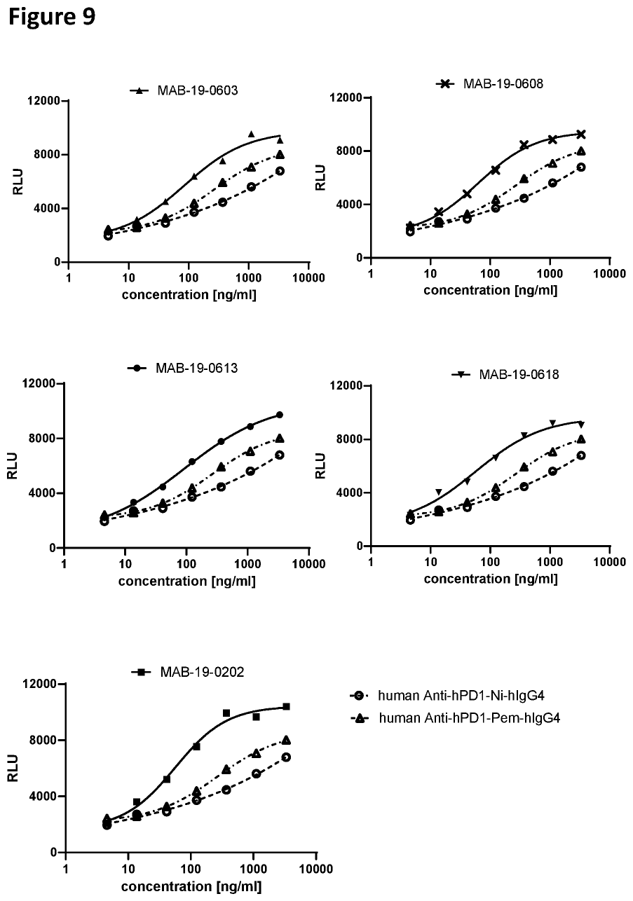

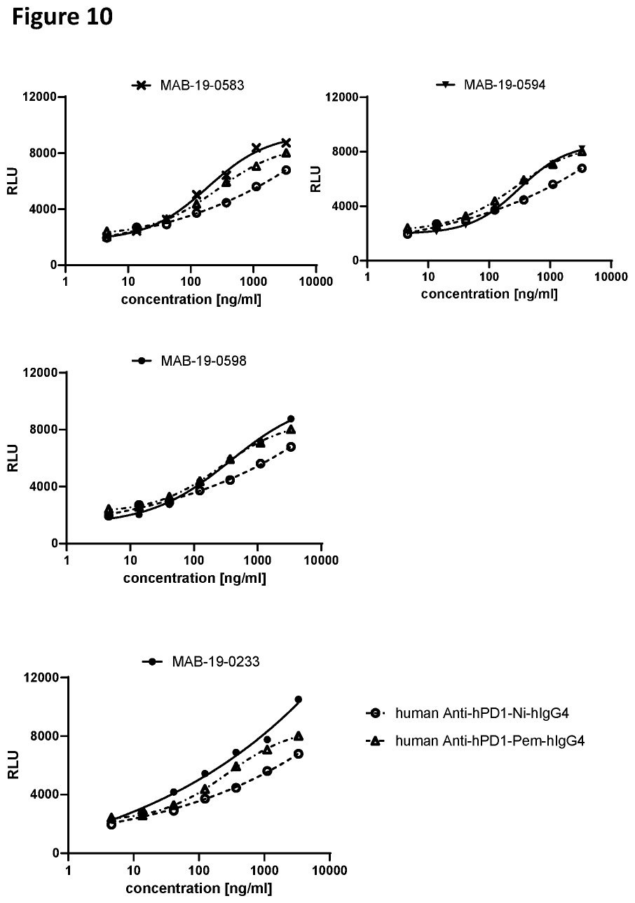

After humanization of the chimeric antibodies MAB-19-0202 and MAB-19-0233 the assay was repeated with the humanized variants and the parental chimeric antibody (FIGS. 9 and 10). Again MAB-19-0202 as well as the derived humanized antibodies outperformed the two reference antibodies (FIG. 9 and Table 2). MAB-19-0233 and the derived humanized antibodies performed comparable to the reference antibodies (FIG. 10 and Table 7).

### Example 5: Antigen-Specific CD8⁺ T Cell Proliferation Assay with Active PD-1/PD-L1 Axis to Measure Functional Activity of the Anti-Human PD-1 Antibodies in a Primary Cell Based

To measure induction of T-cell proliferation by chimeric and humanized anti-PD1 antibodies in an antigen-specific assay with active PD-1/PD-L1 axis, dendritic cells (DCs) were transfected with claudin-6 in vitro-transcribed RNA (IVT-RNA) to express the claudin-6 antigen. T cells were transfected with PD-1 IVT-RNA and with the claudin-6-specific, HLA-A2-restricted T cell receptor (TCR). This TCR can recognize the claudin-6-derived epitope presented in HLA-A2 on the DC. The anti-PD1 antibodies can block the inhibitory PD-1/PD-L1 interaction between PD-L1 endogenously expressed on monocyte-derived DCs and PD-1 on T cells resulting in enhanced T-cell proliferation.

HLA-A²⁺ peripheral blood mononuclear cells (PBMCs) were obtained from healthy donors (Transfusionszentrale, University Hospital, Mainz, Germany). Monocytes were isolated from PBMCs by magnetic-activated cell sorting (MACS) technology using anti-CD14 MicroBeads (Miltenyi; cat. no. 130-050-201), according to the manufacturer's instructions. The peripheral blood lymphocytes (PBLs, CD14-negative fraction) were frozen for future T-cell isolation. For differentiation into immature DCs (iDCs), 1×10⁶ monocytes/ml were cultured for five days in RPMI GlutaMAX (Life technologies GmbH, cat. no. 61870-044) containing 5% human AB serum (Sigma-Aldrich Chemie GmbH, cat. no. H4522-100ML), sodium pyruvate (Life technologies GmbH, cat. no. 11360-039), non-essential amino acids (Life technologies GmbH, cat. no. 11140-035), 100 IU/mL penicillin-streptomycin (Life technologies GmbH, cat. no. 15140-122), 1,000 IU/mL granulocyte-macrophage colony-stimulating factor (GM-CSF; Miltenyi, cat. no. 130-093-868) and 1,000 IU/mL interleukin-4 (IL-4; Miltenyi, cat. no. 130-093-924). Once during these five days, half of the medium was replaced with fresh medium. iDCs were harvested by collecting non-adherent cells and adherent cells were detached by incubation with PBS containing 2 mM EDTA for 10 min at 37°. After washing iDCs were frozen in RPMI GlutaMAX containing 10% v/v DMSO (AppliChem GmbH, cat. no A3672,0050)+50% v/v human AB serum for future antigen-specific T cell assays.

One day prior to the start of an antigen-specific CD8⁺ T-cell proliferation assay, frozen PBLs and iDCs, from the same donor, were thawed. CD8⁺ T cells were isolated from PBLs by MACS technology using anti-CD8 MicroBeads (Miltenyi, cat. no. 130-045-201), according to the manufacturer's instructions. About 10-15×10⁶ CD8⁺ T cells were electroporated with 10 μg of in vitro translated (IVT)-RNA encoding the alpha-chain plus 10 μg of IVT-RNA encoding the beta-chain of a claudin-6-specific murine TCR (HLA-A2-restricted; described in WO 2015/150327 A1) plus 10 μg IVT-RNA encoding PD-1 in 250 μL X-Vivol5 (Biozym Scientific GmbH, cat. no. 881026) in a 4-mm electroporation cuvette (VWR International GmbH, cat. no. 732-0023) using the BTX ECM® 830 Electroporation System device (BTX; 500 V, 1×3 ms pulse). Immediately after electroporation, cells were transferred into fresh IMDM medium (Life Technologies GmbH, cat. no. 12440-061) supplemented with 5% human AB serum and rested at 37° C., 5% CO₂ for at least 1 hour. T cells were labeled using 1.6 μM carboxyfluorescein succinimidyl ester (CFSE; Invitrogen, cat. no. C34564) in PBS according to the manufacturer's instructions, and incubated in IMDM medium supplemented with 5% human AB serum, O/N.

Up to 5×10⁶ thawed iDCs were electroporated with 3 μg IVT-RNA encoding full length claudin-6, in 250 μL X-Vivo15 medium, using the electroporation system as described above (300 V, 1×12 ms pulse) and incubated in IMDM medium supplemented with 5% human AB serum, O/N.

The next day, cells were harvested. Cell surface expression of claudin-6 and PD-L1 on DCs and TCR and PD-1 on T cells was checked by flow cytometry. DCs were stained with an Alexa647-conjugated CLDN6-specific antibody (non-commercially available; in-house production) and with anti-human CD274 antibody (PD-L1, eBioscienes, cat. no. 12-5983) and T cells were stained with an anti-Mouse TCR B Chain antibody (Becton Dickinson GmbH, cat. no. 553174) and with anti-human CD279 antibody (PD-1, eBioscienes, cat. no. 17-2799). 5,000 electroporated DCs were incubated with 50,000 electroporated, CFSE-labeled T cells in the presence of chimeric and humanized anti-hPD-1 antibodies and reference antibody Pembrolizumab (MSD; PZN 10749897 purchased from Phoenix Apotheke Mainz) in IMDM GlutaMAX supplemented with 5% human AB serum in a 96-well round-bottom plate. T-cell proliferation was measured after 5 days by flow cytometry. Data were acquired on a FACSCanto™ or a FACSCelesta™ flow cytometer (BD Biosciences). Data were analyzed using FlowJo™ software V10.3. Proliferation analysis based on CFSE dilution was performed using the proliferation modeling tool from FlowJo, the generation peaks were automatically fitted and expansion index values were calculated. Data was fitted with a 4-parameter logistic model and EC50 values calculated using GraphPad Prism 8.4.3.

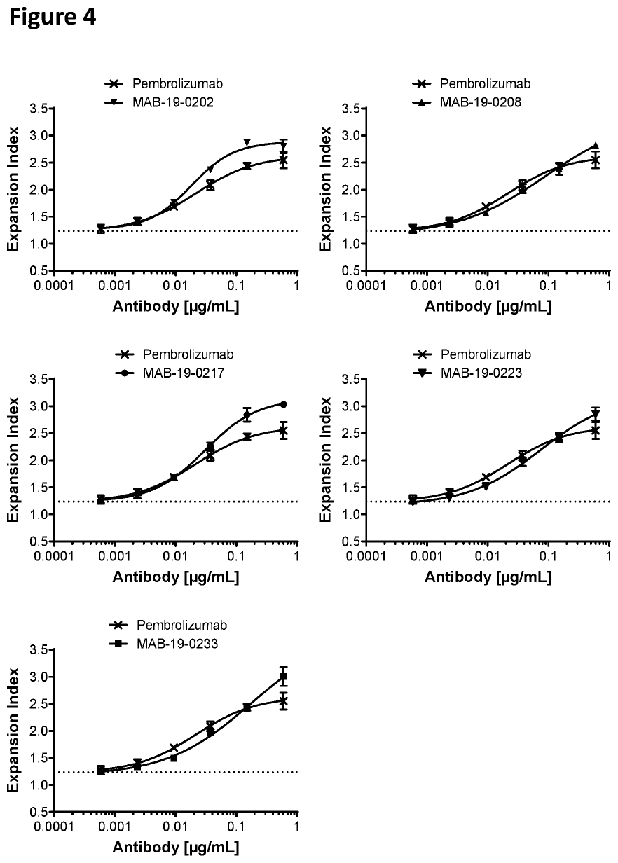

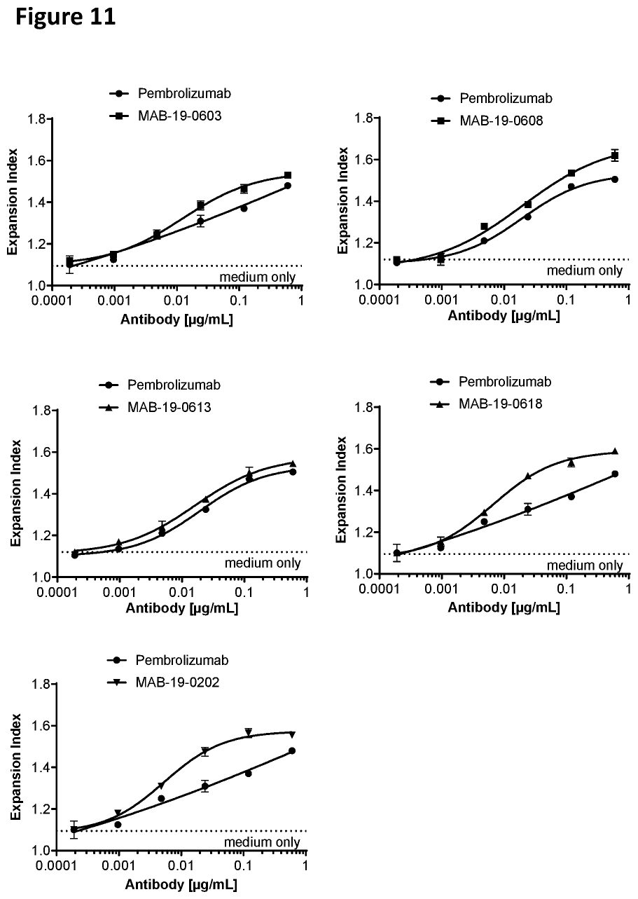

All chimeric antibodies and the humanized anti-hPD-1 antibodies derived from MAB-19-0202 were tested in a concentration ranging from 0.2 ng/mL and 0.6 μg/mL by this T-cell proliferation assay. All of them were able to block the inhibitory PD-1/PD-L1 axis and induced strong proliferation of CD8⁺ T cells. This was reflected by an increase in the expansion index. Fitted dose-response curves revealed a comparable increase of the proliferation induced by all tested chimeric and humanized antibodies to Pembrolizumab as shown in FIG. 4 and FIG. 11.

TABLE 6
EC50 values of the hPD-1 binding as measured by ELISA (FIG.
1) and by the HEK-293-hPD-1 cell binding assay (FIG. 2)
and IC50 values of the PD-1: PDL-1 blockade as measured
by the reporter assay (FIG. 3) for the chimeric antibodies.
N.a. not applicable: EC50 values was not calculable.

| | | HEK-293- | hPD-1 |
|---|---|---|---|
| | hPD-1 | hPD-1 cell | blockade |
| | ELISA | binding | assay |
| | EC50 | EC50 | IC50 |
| | [ng/mL] | [ng/mL] | [ng/mL] |
| MAB-19-0202 | 3.5 | n.a. | 324 |
| MAB-19-0208 | 4.0 | 13.9 | 870 |
| MAB-19-0217 | 9.2 | 6.3 | 720 |
| MAB-19-0223 | 6.6 | 7.0 | 913 |
| MAB-19-0233 | 6.3 | 16.8 | 657 |
| human Anti-hPD1-Ni-hIgG4 | 7.8 | 9.7 | 1069 |
| human Anti-hPD1-Pem-hIgG4 | 7.0 | 12.2 | 862 |

TABLE 7
EC50 values of the hPD-1 binding as measured by ELISA (FIG.
5 and 6) and by the HEK-293-hPD-1 cell binding assay (FIG.
7 and 8) and IC50 values of the PD-1: PDL-1 blockade as
measured by the reporter assay (FIG. 9 and 10) for the
humanized antibodies and the parental chimeric antibody.
N.a. not applicable: EC50 values was not calculable.

| | | HEK-293- | hPD-1 |
|---|---|---|---|
| | hPD-1 | hPD-1 cell | blockade |
| | ELISA | binding | assay |
| | EC50 | EC50 | IC50 |
| | [ng/mL] | [ng/mL] | [ng/mL] |
| MAB-19-0233 | 7.2 | 2.4 | n.a. |
| MAB-19-0583 | 7.5 | 3.6 | 188 |
| MAB-19-0594 | 14.6 | 9.5 | 311 |
| MAB-19-0598 | 8.8 | 5.7 | 368 |
| MAB-19-0202 | 4.0 | 0.5 | 56 |
| MAB-19-0603 | 3.3 | 2.8 | 85 |
| MAB-19-0608 | 7.7 | 2.8 | 63 |
| MAB-19-0613 | 4.4 | 1.2 | 93 |
| MAB-19-0618 | 8.0 | 1.7 | 57 |
| human Anti-hPD1-Ni-hIgG4 | 15.7 | 7.1 | n.a. |
| human Anti-hPD1-Pem- | 15.3 | 6.0 | 266 |
| hIgG4 | | | |

### Example 6: Generation of Anti-Human PD-1 RiboMabs by In Vitro Transcription

For the generation of anti-PD-1 RiboMabs via in vitro transcribed messenger RNA (IVT-mRNA), we inserted the DNA sequences of MAB-19-0202-LC (SEQ ID NO: 79), MAB-19-0233-LC (SEQ ID NO: 83), MAB-19-0202-HC (SEQ ID NO: 74) and MAB-19-0233-HC (SEQ ID NO: 78)N-terminally of human immunoglobulin constant parts (IgG1κ with mutations L234A, L235A and P329G into the IVT-mRNA template vector pST4-hAg-MCS-FI-A30LA70 (BioNTech SE) using standard cloning techniques. This vector contains a human alpha globin (hAg) 5′ untranslated region (UTR) leader sequence as described elsewhere and a 3′ FI element as described in patent application PCT/EP2016/073814. The poly(A) tail consists of 30 adenine nucleotides, a linker (L) and further 70 adenine nucleotides (A30LA70, PCT/EP2015/065357). The following constructs were cloned for the formation of anti-PD-1 RiboMabs:

RiboMab-19-0202:

Heavy chain: pST4-hAg-husec(opt)-anti-PD1-0202-HC-hIgG1-LALA-PG-FI-A30LA70, having a nucleic acid sequence as shown below and as depicted in SEQ ID NO: 93 of the

### SEQUENCE LISTING

(SEQ ID NO: 93)
ATTCTTCTGGTCCCCACAGACTCAGAGAGAACCCGCCACC
ATGAGAGTGATGGCCCCCAGAACCCTGATCCTGCTGCTGT
CTGGCGCCCTGGCCCTGACAGAGACATGGGCCGGAAGCCA
GAGCGTGGAAGAATCTGGCGGCAGACTGGTCACACCTGGC
ACACCTCTGACACTGACCTGTACCGTGTCCGGCTTCAGCC
TGTACAGCTACAACATGGGCTGGGTCCGACAGGCCCCTGG
AAAGGGACTCGAGTACATCGGCATCATCAGCGGCGGCACA
ATCGGCCACTATGCCTCTTGGGCCAAGGGCAGATTCACCA
TCAGCAAGACCAGCAGCACCACCGTGGACCTGAAGATGAC
CAGCCTGACCACCGAGGACACCGCCACCTACTTTTGCGCC
AGAGCCTTCTACGACGACTACGACTACAACGTGTGGGGCC
CAGGCACACTCGTGACAGTCTCCTCTGCCTCTACAAAGGG
CCCTAGCGTGTTCCCTCTGGCTCCTAGCAGCAAGTCTACA
AGCGGAGGAACAGCCGCTCTGGGCTGCCTGGTCAAGGATT
ACTTTCCCGAGCCTGTGACCGTGTCCTGGAATTCTGGCGC
TCTGACAAGCGGCGTGCACACCTTTCCAGCTGTGCTGCAA
AGCAGCGGCCTGTACTCTCTGAGCAGCGTGGTCACAGTGC
CAAGCTCTAGCCTGGGCACCCAGACCTACATCTGCAATGT
GAACCACAAGCCTAGCAACACCAAGGTGGACAAGAAGGTG
GAACCCAAGAGCTGCGACAAGACCCACACCTGTCCTCCAT
GTCCTGCTCCAGAAGCTGCTGGCGGCCCTTCCGTGTTTCT
GTTCCCTCCAAAGCCTAAGGACACCCTGATGATCAGCAGA
ACCCCTGAAGTGACCTGCGTGGTGGTGGATGTGTCCCACG
AGGATCCCGAAGTGAAGTTCAATTGGTACGTGGACGGCGT
GGAAGTGCACAACGCCAAGACCAAGCCTAGAGAGGAACAG
TACAACAGCACCTACAGAGTGGTGTCCGTGCTGACCGTGC
TGCACCAGGATTGGCTGAACGGCAAAGAGTACAAGTGCAA
GGTGTCCAACAAGGCCCTGGGCGCTCCCATCGAGAAAACC
ATCTCTAAGGCCAAGGGACAGCCCCGCGAACCTCAGGTTT
ACACACTGCCTCCAAGCCGGGAAGAAATGACCAAGAACCA
GGTGTCCCTGACCTGCCTCGTGAAGGGCTTCTACCCTTCC
GATATCGCCGTGGAATGGGAGAGCAATGGCCAGCCTGAGA
ACAACTACAAGACAACCCCTCCTGTGCTGGACTCCGATGG
CTCATTCTTCCTGTACAGCAAGCTGACAGTGGACAAGTCC
AGATGGCAGCAGGGCAACGTGTTCAGCTGCAGCGTGATGC
ACGAGGCCCTGCACAACCACTACACCCAGAAGTCCCTGAG
CCTGTCTCCTGGATGATGACTCGAGCTGGTACTGCATGCA
CGCAATGCTAGCTGCCCCTTTCCCGTCCTGGGTACCCCGA
GTCTCCCCCGACCTCGGGTCCCAGGTATGCTCCCACCTCC
ACCTGCCCCACTCACCACCTCTGCTAGTTCCAGACACCTC
CCAAGCACGCAGCAATGCAGCTCAAAACGCTTAGCCTAGC
CACACCCCCACGGGAAACAGCAGTGATTAACCTTTAGCAA
TAAACGAAAGTTTAACTAAGCTATACTAACCCCAGGGTTG
GTCAATTTCGTGCCAGCCACACCGAGACCTGGTCCAGAGT
CGCTAGCCGCGTCGCTAAAAAAAAAAAAAAAAAAAAAAAA
AAAAAAAAAAAAAAAAAAAAAAAAAAAAAAAAAAAAAAAA
AAAAAAAAAAAAAAAAAAAAAAAAAAAAAAAAAAAAAAAA
AAAAAA

Within the sequence as depicted in SEQ ID NO: 93 of the sequence listing, the following elements are comprised:

A 5′-UTR (“hAg”) having a nucleic acid sequence as shown below and as depicted in SEQ ID NO: 94 of the sequence listing.

(SEQ ID NO: 94)
ATTCTTCTGGTCCCCACAGACTCAGAGAGAACCC

A ‘Kozac sequence’ having a nucleic acid sequence as shown below and as depicted in SEQ ID NO: 95 of the sequence listing.
GCCACC (SEQ ID NO: 95)

A secretory signal peptide sequence (“husec(opt)”) having a nucleic acid sequence as shown below and as depicted in SEQ ID NO: 96 of the sequence listing.

(SEQ ID NO: 96)
ATGAGAGTGATGGCCCCCAGAACCCTGATCCTGCTGCTG
TCTGGCGCCCTGGCCCTGACAGAGACATGGGCCGGAAGC

A heavy chain variable domain (“anti-PD1-0202-HC”) having a nucleic acid sequence as depicted in SEQ ID NO: 74 of the sequence listing.

A constant domain CH₁ having a nucleic acid sequence as shown below and as depicted in SEQ ID NO: 97 of the sequence listing.

(SEQ ID NO: 97)
GCCTCTACAAAGGGCCCTAGCGTGTTCCCTCTGGCTC
CTAGCAGCAAGTCTACAAGCGGAGGAACAGCCGCTCT
GGGCTGCCTGGTCAAGGATTACTTTCCCGAGCCTGTG
ACCGTGTCCTGGAATTCTGGCGCTCTGACAAGCGGCG
TGCACACCTTTCCAGCTGTGCTGCAAAGCAGCGGCCT
GTACTCTCTGAGCAGCGTGGTCACAGTGCCAAGCTCT
AGCCTGGGCACCCAGACCTACATCTGCAATGTGAACC
ACAAGCCTAGCAACACCAAGGTGGACAAGAAGGTG

A hinge region having a nucleic acid sequence as shown below and as depicted in SEQ ID NO: 98 of the sequence listing.

(SEQ ID NO: 98)
GAACCCAAGAGCTGCGACAAGACCCACACCTGTCCTCCATGTCCT

A constant domain CH2 having a nucleic acid sequence as shown below and as depicted in SEQ ID NO: 99 of the sequence listing.

(SEQ ID NO: 99)
GCTCCAGAAGCTGCTGGCGGCCCTTCCGTGTTTCTGTTCC
CTCCAAAGCCTAAGGACACCCTGATGATCAGCAGAACCCC
TGAAGTGACCTGCGTGGTGGTGGATGTGTCCCACGAGGAT
CCCGAAGTGAAGTTCAATTGGTACGTGGACGGCGTGGAAG
TGCACAACGCCAAGACCAAGCCTAGAGAGGAACAGTACAA
CAGCACCTACAGAGTGGTGTCCGTGCTGACCGTGCTGCAC
CAGGATTGGCTGAACGGCAAAGAGTACAAGTGCAAGGTGT
CCAACAAGGCCCTGGGCGCTCCCATCGAGAAAACCATCTC
TAAGGCCAAG

A constant domain CH₃ having a nucleic acid sequence as shown below and as depicted in SEQ ID NO: 100 of the sequence listing.

(SEQ ID NO: 100)
GGACAGCCCCGCGAACCTCAGGTTTACACACTGCCTCCAA
GCCGGGAAGAAATGACCAAGAACCAGGTGTCCCTGACCTG
CCTCGTGAAGGGCTTCTACCCTTCCGATATCGCCGTGGAA
TGGGAGAGCAATGGCCAGCCTGAGAACAACTACAAGACAA
CCCCTCCTGTGCTGGACTCCGATGGCTCATTCTTCCTGTA
CAGCAAGCTGACAGTGGACAAGTCCAGATGGCAGCAGGGC
AACGTGTTCAGCTGCAGCGTGATGCACGAGGCCCTGCACA
ACCACTACACCCAGAAGTCCCTGAGCCTGTCTCCTGGA

A ‘F-element’ having a nucleic acid sequence as shown below and as depicted in SEQ ID NO: 101 of the sequence listing.

(SEQ ID NO: 101)
CTGGTACTGCATGCACGCAATGCTAGCTGCCCCTTTCCCG
TCCTGGGTACCCCGAGTCTCCCCCGACCTCGGGTCCCAGG
TATGCTCCCACCTCCACCTGCCCCACTCACCACCTCTGCT
AGTTCCAGACACCTCC

An ‘I-element’ having a nucleic acid sequence as shown below and as depicted in SEQ ID NO: 102 of the sequence listing.

(SEQ ID NO: 102)
CAAGCACGCAGCAATGCAGCTCAAAACGCTTAGCCTAGCC
ACACCCCCACGGGAAACAGCAGTGATTAACCTTTAGCAAT
AAACGAAAGTTTAACTAAGCTATACTAACCCCAGGGTTGG
TCAATTTCGTGCCAGCCACACC

A poly(A) tail (“A30LA70”) having a nucleic acid sequence as shown below and as depicted in SEQ ID NO: 103 of the sequence listing.

(SEQ ID NO: 103)
AAAAAAAAAAAAAAAAAAAAAAAAAAAAAAGCATATG
ACTAAAAAAAAAAAAAAAAAAAAAAAAAAAAAAAAAA
AAAAAAAAAAAAAAAAAAAAAAAAAAAAAAAAAAAA

Light chain: pST4-hAg-husec(opt)-anti-PD1-0202-LC-hIgG1-FI-A30LA70 having a nucleic acid sequence as shown below and as depicted in SEQ ID NO: 104 of the sequence listing:

(SEQ ID NO: 104)
ATTCTTCTGGTCCCCACAGACTCAGAGAGAACCCGCCACC
ATGAGAGTGATGGCCCCCAGAACCCTGATCCTGCTGCTGT
CTGGCGCCCTGGCCCTGACAGAGACATGGGCCGGAAGCGC
TGCTGTGCTGACCCAGACACCTTCTCCAGTGTCTGCCGCC
GTTGGCGGCACAGTGACAATCAGCTGTCAGAGCAGCCAGA
GCGTGTACGGCAACAACCAGCTGTCCTGGTATCAGCAGAA
GCCCGGCCAGCCTCCTAAGCTGCTGATCTACCAGGCCAGC
AAGCTGGAAACAGGCGTGCCCAGCAGATTCAAAGGCAGCG
GCTCTGGCACCCAGTTCACCCTGACAATCTCCGACCTGGA
AAGCGACGATGCCGCCACCTACTATTGTGCCGGCGGATAC
AGCAGCAGCTCCGACACAACATTTGGCGGCGGAACAGAGG
TGGTGGTCAAGCGTACGGTGGCCGCTCCTAGCGTGTTCAT
CTTCCCACCTTCCGACGAGCAGCTGAAGTCTGGCACAGCC
AGCGTTGTGTGCCTGCTGAACAACTTCTACCCCAGAGAAG
CCAAGGTGCAGTGGAAGGTGGACAACGCCCTGCAGAGCGG
CAATAGCCAAGAGAGCGTGACCGAGCAGGACAGCAAGGAC
TCTACCTACAGCCTGAGCAGCACCCTGACACTGAGCAAGG
CCGACTACGAGAAGCACAAAGTGTACGCCTGCGAAGTGAC
CCACCAGGGCCTTTCTAGCCCTGTGACCAAGAGCTTCAAC
CGGGGCGAATGTTGATGACTCGAGCTGGTACTGCATGCAC
GCAATGCTAGCTGCCCCTTTCCCGTCCTGGGTACCCCGAG
TCTCCCCCGACCTCGGGTCCCAGGTATGCTCCCACCTCCA
CCTGCCCCACTCACCACCTCTGCTAGTTCCAGACACCTCC
CAAGCACGCAGCAATGCAGCTCAAAACGCTTAGCCTAGCC
ACACCCCCACGGGAAACAGCAGTGATTAACCTTTAGCAAT
AAACGAAAGTTTAACTAAGCTATACTAACCCCAGGGTTGG
TCAATTTCGTGCCAGCCACACCGAGACCTGGTCCAGAGTC
GCTAGCCGCGTCGCTAAAAAAAAAAAAAAAAAAAAAAAAA
AAAAAGCATATGACTAAAAAAAAAAAAAAAAAAAAAAAAA
AAAAAAAAAAAAAAAAAAAAAAAAAAAAAAAAAAAAAAAA
AAAAA

Within this sequence the elements are as follows: A 5′-UTR, including a ‘Kozac sequence’, as depicted in SEQ ID NOs: 94 and 95 of the sequence listing, a secretory signal peptide sequence (“husec(opt)”) as depicted in SEQ ID NO: 96 of the sequence listing, a light chain variable domain (“anti-PD1-0202-LC”) having a nucleic acid sequence as depicted in SEQ ID NO: 79 of the sequence listing, a constant domain (CL kappa) having a nucleic acid sequence as shown below and as depicted in SEQ ID NO: 105 of the sequence listing, a ‘F-element’ as depicted in SEQ ID NO: 101 of the sequence listing, an ‘I-element’ as depicted in SEQ ID NO: 102 of the sequence listing, and a poly(A) tail (“A30LA70”) as depicted in SEQ ID NO: 103 of the sequence listing.

(SEQ ID NO: 105)
CGTACGGTGGCCGCTCCTAGCGTGTTCATCTTCCCACCTT
CCGACGAGCAGCTGAAGTCTGGCACAGCCAGCGTTGTGTG
CCTGCTGAACAACTTCTACCCCAGAGAAGCCAAGGTGCAG
TGGAAGGTGGACAACGCCCTGCAGAGCGGCAATAGCCAAG
AGAGCGTGACCGAGCAGGACAGCAAGGACTCTACCTACAG
CCTGAGCAGCACCCTGACACTGAGCAAGGCCGACTACGAG
AAGCACAAAGTGTACGCCTGCGAAGTGACCCACCAGGGCC
TTTCTAGCCCTGTGACCAAGAGCTTCAACCGGGGCGAATG
T

RiboMab-19-0233:

Heavy chain: pST4-hAg-husec(opt)-anti-PD1-0233-HC-hIgG1-LALA-PG-FI-A30LA70, having a nucleic acid sequence as shown below and as depicted in SEQ ID NO: 106 of the sequence listing:

(SEQ ID NO: 106)
ATTCTTCTGGTCCCCACAGACTCAGAGAGAACCCGCCACC
ATGAGAGTGATGGCCCCCAGAACCCTGATCCTGCTGCTGT
CTGGCGCCCTGGCCCTGACAGAGACATGGGCCGGAAGCCA
GAGCCTGGAAGAATCTGGCGGCGATCTTGTGAAACCTGGC
GCCTCTCTGACCCTGACATGTAAAGCCAGCGGCATCGACT
TCAGCAGCGTGTACTACATGTGTTGGGTCCGACAGGCCCC
TGGCAAAGGCCTGGAATGGATCGCCTGTATCTACGTGGGC
AGCAGCGGCGTGTCCTACTATGCCACATGGGCCAAGGGCA
GATTCACCATCAGCAAGACCAGCAGCACCACCGTGACACT
GCAGATGACATCTCTGACAGCCGCCGACACCGCCACCTAC
TTTTGTGCCAGAGCCGGATATGTGGGCGCCGTGTATGTGA
CACTGACCAGACTGGATCTGTGGGGCCAGGGCACACTGGT
CACAGTCTCCTCTGCCTCTACAAAGGGCCCTAGCGTGTTC
CCTCTGGCTCCTAGCAGCAAGTCTACAAGCGGAGGAACAG
CCGCTCTGGGCTGCCTGGTCAAGGATTACTTTCCCGAGCC
TGTGACCGTGTCCTGGAATTCTGGCGCTCTGACAAGCGGC
GTGCACACCTTTCCAGCTGTGCTGCAAAGCAGCGGCCTGT
ACTCTCTGAGCAGCGTGGTCACAGTGCCAAGCTCTAGCCT
GGGCACCCAGACCTACATCTGCAATGTGAACCACAAGCCT
AGCAACACCAAGGTGGACAAGAAGGTGGAACCCAAGAGCT
GCGACAAGACCCACACCTGTCCTCCATGTCCTGCTCCAGA
AGCTGCTGGCGGCCCTTCCGTGTTTCTGTTCCCTCCAAAG
CCTAAGGACACCCTGATGATCAGCAGAACCCCTGAAGTGA
CCTGCGTGGTGGTGGATGTGTCCCACGAGGATCCCGAAGT
GAAGTTCAATTGGTACGTGGACGGCGTGGAAGTGCACAAC
GCCAAGACCAAGCCTAGAGAGGAACAGTACAACAGCACCT
ACAGAGTGGTGTCCGTGCTGACCGTGCTGCACCAGGATTG
GCTGAACGGCAAAGAGTACAAGTGCAAGGTGTCCAACAAG
GCCCTGGGCGCTCCCATCGAGAAAACCATCTCTAAGGCCA
AGGGACAGCCCCGCGAACCTCAGGTTTACACACTGCCTCC
AAGCCGGGAAGAAATGACCAAGAACCAGGTGTCCCTGACC
TGCCTCGTGAAGGGCTTCTACCCTTCCGATATCGCCGTGG
AATGGGAGAGCAATGGCCAGCCTGAGAACAACTACAAGAC
AACCCCTCCTGTGCTGGACTCCGATGGCTCATTCTTCCTG
TACAGCAAGCTGACAGTGGACAAGTCCAGATGGCAGCAGG
GCAACGTGTTCAGCTGCAGCGTGATGCACGAGGCCCTGCA
CAACCACTACACCCAGAAGTCCCTGAGCCTGTCTCCTGGA
TGATGACTCGAGCTGGTACTGCATGCACGCAATGCTAGCT
GCCCCTTTCCCGTCCTGGGTACCCCGAGTCTCCCCCGACC
TCGGGTCCCAGGTATGCTCCCACCTCCACCTGCCCCACTC
ACCACCTCTGCTAGTTCCAGACACCTCCCAAGCACGCAGC
AATGCAGCTCAAAACGCTTAGCCTAGCCACACCCCCACGG
GAAACAGCAGTGATTAACCTTTAGCAATAAACGAAAGTTT
AACTAAGCTATACTAACCCCAGGGTTGGTCAATTTCGTGC
CAGCCACACCGAGACCTGGTCCAGAGTCGCTAGCCGCGTC
AAAACGCTTAGCCTAGCCACACCCCCACGGGAAACAGCAG
TGATTAACCTTTAGCAATAAACGAAAGTTTAACTAAGCTA
TACTAACCCCAGGGTTGGTCAATTTCGTGCCAGCCACACC
GAGACCTGGTCCAGAGTCGCTAGCCGCGTCGCTAAAAAAA
AAAAAAAAAAAAAAAAAAAAAAAGCATATGACTAAAAAAA
AAAAAAAAAAAAAAAAAAAAAAAAAAAAAAAAAAAAAAAA
AAAAAAAA

Light chain: pST4-hAg-husec(opt)-anti-PD1-0233-LC-hIgG1-FI-A30LA70, having a nucleic acid sequence as shown below and as depicted in SEQ ID NO: 107 of the sequence listing:

(SEQ ID NO: 107)
ATTCTTCTGGTCCCCACAGACTCAGAGAGAACCCGCCACC
ATGAGAGTGATGGCCCCCAGAACCCTGATCCTGCTGCTGT
CTGGCGCCCTGGCCCTGACAGAGACATGGGCCGGAAGCGC
TGCTGTGCTGACCCAGACACCTTCTCCAGTGTCTGCCGCC
GTTGGCGGCACAGTGACAATCAGCTGTCAGAGCAGCCAGA
GCATCTACACCAACAACGACCTGGCCTGGTATCAGCAGAA
GCCTGGCCAGCCTCCTAAGCTGCTGATCTACGATGCCAGC
AAGCTGGCCTCTGGCGTGCCAAGCAGATTTTCTGGCAGCG
GCTCTGGCACCCAGTTCACCCTGACAATTAGCGGCGTGCA
GTCCGATGATGCCGCCACCTATTATTGCCTCGGCGGCTAC
GATGACGACGCCGACAATGCTTTTGGCGGCGGAACAGAGG
TGGTGGTCAAACGTACGGTGGCCGCTCCTAGCGTGTTCAT
CTTCCCACCTTCCGACGAGCAGCTGAAGTCTGGCACAGCC
AGCGTTGTGTGCCTGCTGAACAACTTCTACCCCAGAGAAG
CCAAGGTGCAGTGGAAGGTGGACAACGCCCTGCAGAGCGG
CAATAGCCAAGAGAGCGTGACCGAGCAGGACAGCAAGGAC
TCTACCTACAGCCTGAGCAGCACCCTGACACTGAGCAAGG
CCGACTACGAGAAGCACAAAGTGTACGCCTGCGAAGTGAC
CCACCAGGGCCTTTCTAGCCCTGTGACCAAGAGCTTCAAC
CGGGGCGAATGTTGATGACTCGAGCTGGTACTGCATGCAC
GCAATGCTAGCTGCCCCTTTCCCGTCCTGGGTACCCCGAG
TCTCCCCCGACCTCGGGTCCCAGGTATGCTCCCACCTCCA
CCTGCCCCACTCACCACCTCTGCTAGTTCCAGACACCTCC
CAAGCACGCAGCAATGCAGCTCAAAACGCTTAGCCTAGCC
ACACCCCCACGGGAAACAGCAGTGATTAACCTTTAGCAAT
AAACGAAAGTTTAACTAAGCTATACTAACCCCAGGGTTGG
TCAATTTCGTGCCAGCCACACCGAGACCTGGTCCAGAGTC
GCTAGCCGCGTCGCTAAAAAAAAAAAAAAAAAAAAAAAAA
AAAAAGCATATGACTAAAAAAAAAAAAAAAAAAAAAAAAA
AAAAAAAAAAAAAAAAAAAAAAAAAAAAAAAAAAAAAAAA
AAAAA

To generate templates for in vitro transcription, plasmid DNAs were linearized downstream of the poly(A) tail-encoding region using a class IIs restriction endonuclease, thereby generating a template to transcribe mRNAs with no additional nucleotides past the poly(A)-tail (Holtkamp et al. (2006) Blood 108 (13), 4009-4017). Linearized template DNAs were purified and subjected to in vitro transcription with T7 RNA polymerase essentially as previously described (Grudzien-Nogalska et al. (2013) Methods Mol Biol. 969:55-72). To minimize immunogenicity, N1-Methylpseudouridine-5′-Triphosphate (TriLink Biotechnologies), m¹ΨTP, was incorporated instead of UTP (Kariko et al. (2008) Mol. Ther. 16 (11), 1833-1840) and double-stranded RNA was removed by cellulose purification (Baiersdorfer et al. (2019) Nucleic acids 15, 26-35). RNA was capped with CleanCap413, a Cap1-structure, followed by purification using magnetic particles. Purified mRNA was eluted in H₂O and stored at −80° C. until further use.

### Example 7: Expression and PD-1 Binding of Anti-Human PD-1 RiboMabs

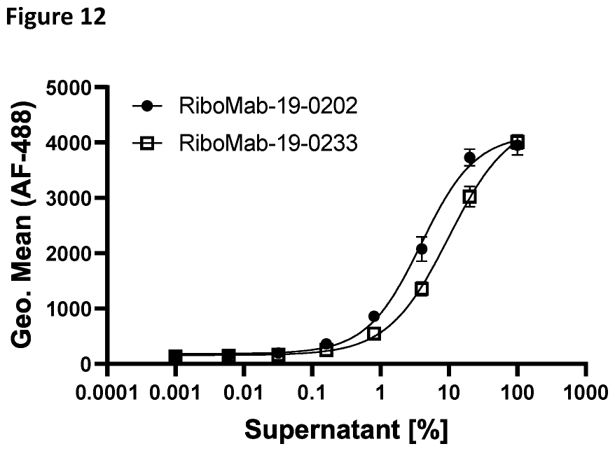

The generated RiboMab-encoding mRNAs were in vitro expressed by lipofection of the mRNA into HEK293T/17 cells and binding of RiboMab-containing supernatants to human PD-1 expressing K562 cells was determined by flow cytometry (FIG. 12).

For the expression of RiboMab-19-0202, the mRNAs encoding for the Mab-19-0202 light chain and the Mab-19-0202 heavy chain (cf., SEQ ID NOs: 93 and 104) were expressed, while for the expression of RiboMab-19-0233, the mRNAs encoding for the Mab-19-0233 light chain and the Mab-19-0233 heavy chain (cf., SEQ ID NOs: 106 and 107) were expressed.

One day prior to lipofection, 1.2×10⁶ HEK293T/17 cells were seeded in 3 mL DMEM (Life Technologies GmbH, cat. no. 31966-021)+10% fetal bovine serum (FBS, Sigma, cat. no. F7524) in 6-well plates. For lipofection, 3 μg mRNA was formulated under sterile and RNase-free conditions at a 2:1 mass ratio of heavy chain and light chain-encoding mRNA using 400 ng mRNA per μL Lipofectamine MessengerMax (Thermo Fisher Scientific, cat. No. LMRNA015) and applied per 10 cm² culture dish to the HEK293T/17 cells at approximately 80% confluence. After 20 h of expression, supernatants were collected under sterile conditions and stored at −20° C. until further use.

20×10⁶ cells K562 cells growing in log-phase were used for electroporation of full-length human PD-1. Cells in 250 μL X-Vivo 15 medium (LONZA Technologies, cat. no. BE02-060F) were combined in 4 mm gap cuvettes with 10 μg human PD-1-encoding IVT-mRNA. Cells were immediately electroporated with a BTX ECM830 (BTX Harvard Apparatus) with the following setting: 200 V, 3 pulses, 8 ms. Electroporated cells were subsequently seeded in RPMI (Life Technologies GmbH, cat. no. 61870-010)+10% FBS at a density of 0.5×10⁶/mL in a T175 flask (Cellstar, Greiner Bio-One, cat. no. 660175) and incubated over night at 37° C., 5% CO₂. The next day, PD-1 expression was verified by flow cytometry using APC-conjugated CD279 (PD-1) monoclonal antibody (eBioJ105, ThermoFisher Scientific, cat. no. 17-2799-42).

Binding of RiboMabs to K562 cells expressing PD-1 was analyzed by flow cytometry. 7.5×10⁴ cells/well were incubated in polystyrene 96-well round-bottom plates (Greiner bio-one, cat. no. 650180) with serial dilutions of RiboMab-containing supernatants (range 0.006 to 100% in 4-fold dilution steps) in 100 μL PBS/0.1% BSA/0.02% azide (FACS buffer) at 4° C. for 1 h. After washing twice in FACS buffer, cells were incubated in 50 μL Alexa Fluor 488 (AF488)-conjugated goat-anti-human IgG F(ab′)₂ (1:500 in FACS buffer; Jackson ImmunoResearch Laboratories, cat. no. 109-546-098) at 4° C. for 30 min. Cells were washed twice with FACS buffer, re-suspended in 60 μL FACS buffer and analyzed on a BD FACSCanto™ II flow cytometer (BD Biosciences). Binding curves were analyzed by non-linear regression (log(agonist) vs. response—variable slope (four parameters)) using GraphPad Prism V9.1.0 software.

FIG. 12 shows dose-dependent binding of RiboMab-19-0202 and RiboMab-19-0233 to K562 cells transfected with full length human PD-1. Binding curves were highly comparable with only an approx. 2.5-fold difference in EC50 values (3.9%-supernatant for RiboMab-19-0202 and 9.9%-supernatant for RiboMab-19-0233), indicating that mRNA encoding RiboMabs is translated into comparable amounts of PD-1 binding antibodies.

1. An antibody having the ability of binding to PD-1, wherein the antibody comprises a heavy chain variable region (VH) comprising a complementarity-determining region 3 (HCDR3) having or comprising a sequence as set forth in any one of SEQ ID NO: 1, SEQ ID NO: 2, SEQ ID NO: 3, SEQ ID NO: 4 or SEQ ID NO: 5.

2. The antibody of claim 1, wherein the HCDR3 has or comprises a sequence as set forth in any one of SEQ ID NO: 6, SEQ ID NO: 7, SEQ ID NO: 8, SEQ ID NO: 9 or SEQ ID NO: 10.

3. The antibody of claim 1 or 2, wherein the heavy chain variable region (VH) comprises a complementarity-determining region 2 (HCDR2) having or comprising a sequence as set forth in any one of SEQ ID NO: 11, SEQ ID NO: 12, SEQ ID NO: 13, SEQ ID NO: 14 or SEQ ID NO: 15.

4. The antibody of claim 3, wherein the HCDR2 has or comprises a sequence as set forth in any one of SEQ ID NO: 16, SEQ ID NO: 17, SEQ ID NO: 18, SEQ ID NO: 19 or SEQ ID NO: 20.

5. The antibody of any one of claims 1 to 4, wherein the heavy chain variable region (VH) comprises a complementarity-determining region 1 (HCDR1) having or comprising a sequence selected from SYN, RYY, as set forth in SEQ ID NO: 21 or SEQ ID NO: 22.

6. The antibody of claim 5, wherein the HCDR1 has or comprises a sequence as set forth in any one of SEQ ID NO: 23, SEQ ID NO: 24, SEQ ID NO: 25, SEQ ID NO: 26 or SEQ ID NO: 27.

7. The antibody of claim 5, wherein the HCDR1 has or comprises a sequence as set forth in any one of SEQ ID NO: 28, SEQ ID NO: 29, SEQ ID NO: 30, SEQ ID NO: 31 or SEQ ID NO: 32.

8. The antibody of any one of claims 1 to 7, wherein the antibody comprises a heavy chain variable region (VH) comprising a HCDR1, HCDR2 and HCDR3 sequence, wherein
(i) the HCDR1 sequence is selected from a sequence having or comprising SYN, SEQ ID NO: 23 or SEQ ID NO: 28, the HCDR2 sequence is selected from a sequence having or comprising SEQ ID NO: 11 or SEQ ID NO: 16, and the HCDR3 sequence is selected from a sequence having or comprising SEQ ID NO: 1 or SEQ ID NO: 6;
(ii) the HCDR1 sequence is selected from a sequence having or comprising RYY, SEQ ID NO: 24 or SEQ ID NO: 29, the HCDR2 sequence is selected from a sequence having or comprising SEQ ID NO: 12 or SEQ ID NO: 17, and the HCDR3 sequence is selected from a sequence having or comprising SEQ ID NO: 2 or SEQ ID NO: 7;
(iii) the HCDR1 sequence is selected from a sequence having or comprising RYY, SEQ ID NO: 25 or SEQ ID NO: 30, the HCDR2 sequence is selected from a sequence having or comprising SEQ ID NO: 13 or SEQ ID NO: 18, and the HCDR3 sequence is selected from a sequence having or comprising SEQ ID NO: 3 or SEQ ID NO: 8;
(iv) the HCDR1 sequence is selected from a sequence having or comprising SEQ ID NO: 21, SEQ ID NO: 26 or SEQ ID NO: 31, the HCDR2 sequence is selected from a sequence having or comprising SEQ ID NO: 14 or SEQ ID NO: 19, and the HCDR3 sequence is selected from a sequence having or comprising SEQ ID NO: 4 or SEQ ID NO: 9;
(v) the HCDR1 sequence is selected from a sequence having or comprising SEQ ID NO: 22, SEQ ID NO: 27 or SEQ ID NO: 32, the HCDR2 sequence is selected from a sequence having or comprising SEQ ID NO: 15 or SEQ ID NO: 20, and the HCDR3 sequence is selected from a sequence having or comprising SEQ ID NO: 5 or SEQ ID NO: 10.

9. The antibody of any one of claims 1 to 8, wherein the antibody comprises a heavy chain variable region (VH) comprising a HCDR1, HCDR2, and HCDR3 sequence, wherein the HCDR1, HCDR2 and HCDR3 sequence is or comprises
(i) SYN, SEQ ID NO: 11 and SEQ ID NO: 1, respectively;
(ii) RYY, SEQ ID NO: 12 and SEQ ID NO: 2, respectively;
(iii) RYY, SEQ ID NO: 13 and SEQ ID NO: 3, respectively;
(iv) SEQ ID NO: 21, SEQ ID NO: 14 and SEQ ID NO: 4, respectively; or
(v) SEQ ID NO: 22, SEQ ID NO: 15 and SEQ ID NO: 5, respectively.

10. The antibody of any one of claims 1 to 8, wherein the antibody comprises a heavy chain variable region (VH) comprising a HCDR1, HCDR2, and HCDR3 sequence, wherein the HCDR1, HCDR2 and HCDR3 sequence is or comprises
(i) SEQ ID NO: 23, SEQ ID NO: 16 and SEQ ID NO: 1, respectively;
(ii) SEQ ID NO: 24, SEQ ID NO: 17 and SEQ ID NO: 2, respectively;
(iii) SEQ ID NO: 25, SEQ ID NO: 18 and SEQ ID NO: 3, respectively;
(iv) SEQ ID NO: 26, SEQ ID NO: 19 and SEQ ID NO: 4, respectively; or
(v) SEQ ID NO: 27, SEQ ID NO: 20 and SEQ ID NO: 5, respectively.

11. The antibody of any one of claims 1 to 8, wherein the antibody comprises a heavy chain variable region (VH) comprising a HCDR1, HCDR2, and HCDR3 sequence, wherein the HCDR1, HCDR2 and HCDR3 sequence is or comprises
(i) SEQ ID NO: 28, SEQ ID NO: 11 and SEQ ID NO: 6, respectively;
(ii) SEQ ID NO: 29, SEQ ID NO: 12 and SEQ ID NO: 7, respectively;
(iii) SEQ ID NO: 30, SEQ ID NO: 13 and SEQ ID NO: 8, respectively;
(iv) SEQ ID NO: 31, SEQ ID NO: 14 and SEQ ID NO: 9, respectively; or
(v) SEQ ID NO: 32, SEQ ID NO: 15 and SEQ ID NO: 10, respectively.

12. An antibody having the ability of binding to PD-1, wherein the antibody comprises a light chain variable region (VL) comprising a complementarity-determining region 3 (LCDR3) having or comprising a sequence as set forth in any one of SEQ ID NO: 33, SEQ ID NO: 34, SEQ ID NO: 35, SEQ ID NO: 36 or SEQ ID NO: 37.

13. The antibody of claim 12, wherein the light chain variable region (VL) comprises a complementarity-determining region 2 (LCDR2) having or comprising a sequence selected from QAS or DAS.

14. The antibody of claim 12, wherein the light chain variable region (VL) comprises a complementarity-determining region 2 (LCDR2) having or comprising a sequence as set forth in any one of SEQ ID NO: 38, SEQ ID NO: 39, SEQ ID NO: 40 or SEQ ID NO: 41.

15. The antibody of any one of claims 12 to 14, wherein the light chain variable region (VL) comprises a complementarity-determining region 1 (LCDR1) having or comprising a sequence as set forth in any one of SEQ ID NO: 42, SEQ ID NO: 43, SEQ ID NO: 44, SEQ ID NO: 45 or SEQ ID NO: 46.

16. The antibody of any one of claims 12 to 14, wherein the light chain variable region (VL) comprises a complementarity-determining region 1 (LCDR1) having or comprising a sequence as set forth in any one of SEQ ID NO: 47, SEQ ID NO: 48, SEQ ID NO: 49, SEQ ID NO: 50 or SEQ ID NO: 51.

17. The antibody of any one of claims 12 to 16, wherein the antibody comprises a light chain variable region (VL) comprising a LCDR1, LCDR2, and LCDR3 sequence, wherein
(i) the LCDR1 sequence is selected from a sequence having or comprising SEQ ID NO: 42 or SEQ ID NO: 47, the LCDR2 sequence is selected from a sequence having or comprising QAS or SEQ ID NO: 38, and the LCDR3 sequence is a sequence having or comprising SEQ ID NO: 33;
(ii) the LCDR1 sequence is selected from a sequence having or comprising SEQ ID NO: 43 or SEQ ID NO: 48, the LCDR2 sequence is selected from a sequence having or comprising DAS or SEQ ID NO: 39, and the LCDR3 sequence is a sequence having or comprising SEQ ID NO: 34;
(iii) the LCDR1 sequence is selected from a sequence having or comprising SEQ ID NO: 44 or SEQ ID NO: 49, the LCDR2 sequence is selected from a sequence having or comprising DAS or SEQ ID NO: 39, and the LCDR3 sequence is a sequence having or comprising SEQ ID NO: 35;
(iv) the LCDR1 sequence is selected from a sequence having or comprising SEQ ID NO: 45 or SEQ ID NO: 50, the LCDR2 sequence is selected from a sequence having or comprising DAS or SEQ ID NO: 40, and the LCDR3 sequence is a sequence having or comprising SEQ ID NO: 36;
(v) the LCDR1 sequence is selected from a sequence having or comprising SEQ ID NO: 46 or SEQ ID NO: 51, the LCDR2 sequence is selected from a sequence having or comprising DAS or SEQ ID NO: 41, and the LCDR3 sequence is a sequence having or comprising SEQ ID NO: 37.

18. The antibody of any one of claims 12 to 17, wherein the antibody comprises a light chain variable region (VL) comprising a LCDR1, LCDR2, and LCDR3 sequence, wherein the LCDR1, LCDR2 and LCDR3 sequence is or comprises:
(i) SEQ ID NO: 42, QAS, and SEQ ID NO: 33, respectively;
(ii) SEQ ID NO: 43, DAS, and SEQ ID NO: 34, respectively;
(iii) SEQ ID NO: 44, DAS, and SEQ ID NO: 35, respectively;
(iv) SEQ ID NO: 45, DAS, and SEQ ID NO: 36, respectively; or
(v) SEQ ID NO: 46, DAS, and SEQ ID NO: 37, respectively.

19. The antibody of any one of claims 12 to 17, wherein the antibody comprises a light chain variable region (VL) comprising a LCDR1, LCDR2, and LCDR3 sequence, wherein the LCDR1, LCDR2 and LCDR3 sequence is or comprises:
(i) SEQ ID NO: 47, SEQ ID NO: 38, and SEQ ID NO: 33, respectively;
(ii) SEQ ID NO: 48, SEQ ID NO: 39, and SEQ ID NO: 34, respectively;
(iii) SEQ ID NO: 49, SEQ ID NO: 39, and SEQ ID NO: 35, respectively;
(iv) SEQ ID NO: 50, SEQ ID NO: 40, and SEQ ID NO: 36, respectively; or
(v) SEQ ID NO: 51, SEQ ID NO: 41, and SEQ ID NO: 37, respectively.

20. An antibody having the ability of binding to PD-1, wherein the antibody comprises a heavy chain variable region (VH) of any one of claims 1 to 11 and/or a light chain variable region (VL) of any one of claims 12 to 19.

21. The antibody of any one of claims 1 to 20, wherein the antibody comprises a heavy chain variable region (VH) comprising a HCDR1, HCDR2, and HCDR3 sequence and a light chain variable region (VL) comprising a LCDR1, LCDR2, and LCDR3 sequence, wherein the HCDR1, HCDR2 and HCDR3 sequence comprises or has the sequence SYN, as set forth in SEQ ID NO: 11 and SEQ ID NO: 1, respectively, and the LCDR1, LCDR2 and LCDR3 sequence comprises or has the sequence as set forth in SEQ ID NO: 42, QAS, and SEQ ID NO: 33, respectively.

22. The antibody of any one of claims 1 to 20, wherein the antibody comprises a heavy chain variable region (VH) comprising a HCDR1, HCDR2, and HCDR3 sequence and a light chain variable region (VL) comprising a LCDR1, LCDR2, and LCDR3 sequence, wherein the HCDR1, HCDR2 and HCDR3 sequence comprises or has the sequence as set forth in SEQ ID NO: 23, SEQ ID NO: 16, and SEQ ID NO: 1, respectively, and the LCDR1, LCDR2 and LCDR3 sequence comprises or has the sequence as set forth in SEQ ID NO: 47, SEQ ID NO: 38, and SEQ ID NO: 33, respectively.

23. The antibody of any one of claims 1 to 20, wherein the antibody comprises a heavy chain variable region (VH) comprising a HCDR1, HCDR2, and HCDR3 sequence and a light chain variable region (VL) comprising a LCDR1, LCDR2, and LCDR3 sequence, wherein the HCDR1, HCDR2 and HCDR3 sequence comprises or has the sequence as set forth in SEQ ID NO: 28, SEQ ID NO: 11, and SEQ ID NO: 6, respectively, and the LCDR1, LCDR2 and LCDR3 sequence comprises or has the sequence as set forth in SEQ ID NO: 42, QAS, and SEQ ID NO: 33, respectively.

24. The antibody of any one of claims 1 to 20, wherein the antibody comprises a heavy chain variable region (VH) comprising a HCDR1, HCDR2, and HCDR3 sequence and a light chain variable region (VL) comprising a LCDR1, LCDR2, and LCDR3 sequence, wherein the HCDR1, HCDR2 and HCDR3 sequence comprises or has the sequence RYY, as set forth in SEQ ID NO: 12 and SEQ ID NO: 2, respectively, and the LCDR1, LCDR2 and LCDR3 sequence comprises or has the sequence as set forth in SEQ ID NO: 43, DAS, and SEQ ID NO: 34, respectively.

25. The antibody of any one of claims 1 to 20, wherein the antibody comprises a heavy chain variable region (VH) comprising a HCDR1, HCDR2, and HCDR3 sequence and a light chain variable region (VL) comprising a LCDR1, LCDR2, and LCDR3 sequence, wherein the HCDR1, HCDR2 and HCDR3 sequence comprises or has the sequence as set forth in SEQ ID NO: 24, SEQ ID NO: 17, and SEQ ID NO: 2, respectively, and the LCDR1, LCDR2 and LCDR3 sequence comprises or has the sequence as set forth in SEQ ID NO: 48, SEQ ID NO: 39, and SEQ ID NO: 34, respectively.

26. The antibody of any one of claims 1 to 20, wherein the antibody comprises a heavy chain variable region (VH) comprising a HCDR1, HCDR2, and HCDR3 sequence and a light chain variable region (VL) comprising a LCDR1, LCDR2, and LCDR3 sequence, wherein the HCDR1, HCDR2 and HCDR3 sequence comprises or has the sequence as set forth in SEQ ID NO: 29, SEQ ID NO: 12, and SEQ ID NO: 7, respectively, and the LCDR1, LCDR2 and LCDR3 sequence comprises or has the sequence as set forth in SEQ ID NO: 43, DAS, and SEQ ID NO: 34, respectively.

27. The antibody of any one of claims 1 to 20, wherein the antibody comprises a heavy chain variable region (VH) comprising a HCDR1, HCDR2, and HCDR3 sequence and a light chain variable region (VL) comprising a LCDR1, LCDR2, and LCDR3 sequence, wherein the HCDR1, HCDR2 and HCDR3 sequence comprises or has the sequence RYY, as set forth in SEQ ID NO: 13 and SEQ ID NO: 3, respectively, and the LCDR1, LCDR2 and LCDR3 sequence comprises or has the sequence as set forth in SEQ ID NO: 44, DAS, and SEQ ID NO: 35, respectively.

28. The antibody of any one of claims 1 to 20, wherein the antibody comprises a heavy chain variable region (VH) comprising a HCDR1, HCDR2, and HCDR3 sequence and a light chain variable region (VL) comprising a LCDR1, LCDR2, and LCDR3 sequence, wherein the HCDR1, HCDR2 and HCDR3 sequence comprises or has the sequence as set forth in SEQ ID NO: 25, SEQ ID NO: 18, and SEQ ID NO: 3, respectively, and the LCDR1, LCDR2 and LCDR3 sequence comprises or has the sequence as set forth in SEQ ID NO: 49, SEQ ID NO: 39, and SEQ ID NO: 35, respectively.

29. The antibody of any one of claims 1 to 20, wherein the antibody comprises a heavy chain variable region (VH) comprising a HCDR1, HCDR2, and HCDR3 sequence and a light chain variable region (VL) comprising a LCDR1, LCDR2, and LCDR3 sequence, wherein the HCDR1, HCDR2 and HCDR3 sequence comprises or has the sequence as set forth in SEQ ID NO: 30, SEQ ID NO: 13, and SEQ ID NO: 8, respectively, and the LCDR1, LCDR2 and LCDR3 sequence comprises or has the sequence as set forth in SEQ ID NO: 44, DAS, and SEQ ID NO: 35, respectively.

30. The antibody of any one of claims 1 to 20, wherein the antibody comprises a heavy chain variable region (VH) comprising a HCDR1, HCDR2, and HCDR3 sequence and a light chain variable region (VL) comprising a LCDR1, LCDR2, and LCDR3 sequence, wherein the HCDR1, HCDR2 and HCDR3 sequence comprises or has the sequence as set forth in SEQ ID NO: 21, SEQ ID NO: 14 and SEQ ID NO: 4, respectively, and the LCDR1, LCDR2 and LCDR3 sequence comprises or has the sequence as set forth in SEQ ID NO: 45, DAS, and SEQ ID NO: 36, respectively.

31. The antibody of any one of claims 1 to 20, wherein the antibody comprises a heavy chain variable region (VH) comprising a HCDR1, HCDR2, and HCDR3 sequence and a light chain variable region (VL) comprising a LCDR1, LCDR2, and LCDR3 sequence, wherein the HCDR1, HCDR2 and HCDR3 sequence comprises or has the sequence as set forth in SEQ ID NO: 26, SEQ ID NO: 19, and SEQ ID NO: 4, respectively, and the LCDR1, LCDR2 and LCDR3 sequence comprises or has the sequence as set forth in SEQ ID NO: 50, SEQ ID NO: 40, and SEQ ID NO: 36, respectively.

32. The antibody of any one of claims 1 to 20, wherein the antibody comprises a heavy chain variable region (VH) comprising a HCDR1, HCDR2, and HCDR3 sequence and a light chain variable region (VL) comprising a LCDR1, LCDR2, and LCDR3 sequence, wherein the HCDR1, HCDR2 and HCDR3 sequence comprises or has the sequence as set forth in SEQ ID NO: 31, SEQ ID NO: 14, and SEQ ID NO: 9, respectively, and the LCDR1, LCDR2 and LCDR3 sequence comprises or has the sequence as set forth in SEQ ID NO: 45, DAS, and SEQ ID NO: 36, respectively.

33. The antibody of any one of claims 1 to 20, wherein the antibody comprises a heavy chain variable region (VH) comprising a HCDR1, HCDR2, and HCDR3 sequence and a light chain variable region (VL) comprising a LCDR1, LCDR2, and LCDR3 sequence, wherein the HCDR1, HCDR2 and HCDR3 sequence comprises or has the sequence as set forth in SEQ ID NO: 22, SEQ ID NO: 15 and SEQ ID NO: 5, respectively, and the LCDR1, LCDR2 and LCDR3 sequence comprises or has the sequence as set forth in SEQ ID NO: 46, DAS, and SEQ ID NO: 37, respectively.

34. The antibody of any one of claims 1 to 20, wherein the antibody comprises a heavy chain variable region (VH) comprising a HCDR1, HCDR2, and HCDR3 sequence and a light chain variable region (VL) comprising a LCDR1, LCDR2, and LCDR3 sequence, wherein the HCDR1, HCDR2 and HCDR3 sequence comprises or has the sequence as set forth in SEQ ID NO: 27, SEQ ID NO: 20, and SEQ ID NO: 5, respectively, and the LCDR1, LCDR2 and LCDR3 sequence comprises or has the sequence as set forth in SEQ ID NO: 51, SEQ ID NO: 41, and SEQ ID NO: 37, respectively.

35. The antibody of any one of claims 1 to 20, wherein the antibody comprises a heavy chain variable region (VH) comprising a HCDR1, HCDR2, and HCDR3 sequence and a light chain variable region (VL) comprising a LCDR1, LCDR2, and LCDR3 sequence, wherein the HCDR1, HCDR2 and HCDR3 sequence comprises or has the sequence as set forth in SEQ ID NO: 32, SEQ ID NO: 15, and SEQ ID NO: 10, respectively, and the LCDR1, LCDR2 and LCDR3 sequence comprises or has the sequence as set forth in SEQ ID NO: 46, DAS, and SEQ ID NO: 37, respectively.

36. The antibody of any one of claims 1 to 35, wherein the antibody comprises a heavy chain variable region (VH) comprising a sequence having at least 70%, at least 75%, at least 80%, at least 85%, at least 90%, at least 95%, at least 97%, at least 99%, or 100% identity to the amino acid sequence of the VH sequence as set forth in any one of SEQ ID NO: 52 to SEQ ID NO: 56.

37. The antibody of any one of claims 1 to 36, wherein the antibody comprises a heavy chain variable region (VH), wherein the VH comprises the sequence as set forth in any one of SEQ ID NO: 52 to SEQ ID NO: 56.

38. The antibody of any one of claims 12 to 37, wherein the antibody comprises a light chain variable region (VL) comprising a sequence having at least 70%, at least 75%, at least 80%, at least 85%, at least 90%, at least 95%, at least 97%, at least 99%, or 100% identity to the amino acid sequence of the VL sequence as set forth in any one of SEQ ID NO: 57 to SEQ ID NO: 61.

39. The antibody of any one of claims 12 to 38, wherein the antibody comprises a light chain variable region (VL), wherein the VL comprises the sequence as set forth in any one of SEQ ID NO: 57 to SEQ ID NO: 61.

40. The antibody of any one of claims 1 to 39, wherein the antibody comprises a heavy chain variable region (VH) and a light chain variable region (VL), wherein the VH comprises or has the sequence as set forth in SEQ ID NO: 52 and the VL comprises or has the sequence as set forth in SEQ ID NO: 57.

41. The antibody of any one of claims 1 to 39, wherein the antibody comprises a heavy chain variable region (VH) and a light chain variable region (VL), wherein the VH comprises or has the sequence as set forth in SEQ ID NO: 53 and the VL comprises or has the sequence as set forth in SEQ ID NO: 58.

42. The antibody of any one of claims 1 to 39, wherein the antibody comprises a heavy chain variable region (VH) and a light chain variable region (VL), wherein the VH comprises or has the sequence as set forth in SEQ ID NO: 54 and the VL comprises or has the sequence as set forth in SEQ ID NO: 59.

43. The antibody of any one of claims 1 to 39, wherein the antibody comprises a heavy chain variable region (VH) and a light chain variable region (VL), wherein the VH comprises or has the sequence as set forth in SEQ ID NO: 55 and the VL comprises or has the sequence as set forth in SEQ ID NO: 60.

44. The antibody of any one of claims 1 to 39, wherein the antibody comprises a heavy chain variable region (VH) and a light chain variable region (VL), wherein the VH comprises or has the sequence as set forth in SEQ ID NO: 56 and the VL comprises or has the sequence as set forth in SEQ ID NO: 61.

45. The antibody of any one of claims 1 to 35, wherein the antibody comprises a heavy chain variable region (VH) comprising a sequence having at least 70%, at least 75%, at least 80%, at least 85%, at least 90%, at least 95%, at least 97%, at least 99%, or 100% identity to the amino acid sequence of the VH sequence as set forth in any one of SEQ ID NO: 62 to SEQ ID NO: 64.

46. The antibody of claim 45, wherein the antibody comprises a heavy chain variable region (VH), wherein the VH comprises the sequence as set forth in any one of SEQ ID NO: 62 to SEQ ID NO: 64.

47. The antibody of any one of claims 12 to 35, wherein the antibody comprises a light chain variable region (VL) comprising a sequence having at least 70%, at least 75%, at least 80%, at least 85%, at least 90%, at least 95%, at least 97%, at least 99%, or 100% identity to the amino acid sequence of the VL sequence as set forth in any one of SEQ ID NO: 65 to SEQ ID NO: 70.

48. The antibody of claim 47, wherein the antibody comprises a light chain variable region (VL), wherein the VL comprises the sequence as set forth in any one of SEQ ID NO: 65 to SEQ ID NO: 70.

49. The antibody of any one of claims 1 to 35 or any one of claims 45 to 48, wherein the antibody comprises a heavy chain variable region (VH) and a light chain variable region (VL), wherein the VH comprises or has the sequence as set forth in SEQ ID NO: 62 and the VL comprises or has the sequence as set forth in SEQ ID NO: 65 or SEQ ID NO: 66 or SEQ ID NO: 67 or SEQ ID NO: 68.

50. The antibody of any one of claims 1 to 35 or any one of claims 45 to 48, wherein the antibody comprises a heavy chain variable region (VH) and a light chain variable region (VL), wherein the VH comprises or has the sequence as set forth in SEQ ID NO: 63 and the VL comprises or has the sequence as set forth in SEQ ID NO: 69 or SEQ ID NO: 70, or wherein the VH comprises or has the sequence as set forth in SEQ ID NO: 64 and the VL comprises or has the sequence as set forth in SEQ ID NO: 70.

51. The antibody of any one of claims 1 to 50, wherein the antibody is selected from the group consisting of an IgG1, an IgG2, preferably IgG2a and IgG2b, an IgG3, an IgG4, an IgM, an IgA1, an IgA2, a secretory IgA, an IgD, and an IgE antibody.

52. The antibody of any one of claims 1 to 51, which is a monoclonal, chimeric or humanized antibody or a fragment of such an antibody.

53. The antibody of any one of claims 1 to 52, wherein the antibody is a Fab fragment, F(ab′)₂ fragment, Fv fragment, or a single chain (scFv) antibody.

54. The antibody of any one of claims 1 to 53, wherein PD-1 is human PD-1.

55. The antibody of claim 54, wherein the PD-1 has or comprises the amino acid sequence as set forth in SEQ ID NO: 71 or SEQ ID NO: 72, or the amino acid sequence of PD-1 has at least 70%, at least 75%, at least 80%, at least 85%, at least 90%, at least 95%, at least 97%, at least 99%, or 100% identity to the amino acid sequence as set forth in SEQ ID NO: 71 or SEQ ID NO: 72, or is an immunogenic fragment thereof.

56. The antibody of any one of claims 1 to 55, which binds to a native epitope of PD-1 present on the surface of living cells.

57. The antibody of any one of claims 1 to 56, wherein the antibody is a multispecific antibody comprising a first antigen-binding region binding to PD-1 and at least one further antigen-binding region binding to another antigen.

58. The antibody of claim 57, wherein the antibody is a bispecific antibody comprising a first antigen-binding region binding to PD-1 and a second antigen-binding region binding to another antigen.

59. The antibody of claim 57 or 58, wherein the first antigen-binding region binding to PD-1 comprises the heavy chain variable region (VH) and/or the light chain variable region (VL) as set forth in any one of claims 1 to 50.

60. The antibody of any one of claims 1 to 59, which is obtainable by a method comprising the step of immunizing an animal with a protein or peptide having an amino acid sequence as set forth in SEQ ID NO: 71 or SEQ ID NO: 72, or an immunogenic fragment thereof, or a nucleic acid or host cell or virus expressing said protein or peptide, or an immunogenic fragment thereof.

61. A hybridoma capable of producing the antibody of any one of claims 1 to 60.

62. A conjugate comprising an antibody of any one of claims 1 to 60 coupled to a moiety or agent.

63. The conjugate of claim 62, wherein the moiety or agent is selected from the group consisting of a radioisotope, an enzyme, a dye, a drug, a toxin and a cytotoxic agent.

64. A multimer, comprising at least two antibodies of any one of claims 1 to 60 or at least two conjugates of claim 62 or 63 or a mixture of one or more antibodies of any one of claims 1 to 60 and one or more conjugates of claim 62 or 63.

65. The multimer of claim 64, comprising 4 to 8 antibodies of any one of claims 1 to 60 or conjugates of claim 62 or 63.

66. A nucleic acid comprising a nucleic acid sequence encoding an antibody of any one of claims 1 to 60 or a fragment thereof.

67. The nucleic acid of claim 66, wherein the nucleic acid is RNA.

68. A vector comprising the nucleic acid of claim 66 or 67.

69. The vector of claim 68, wherein the vector is a multilamellar vesicle, an unilamellar vesicle, or a mixture thereof.

70. The vector of claim 68 or 69, wherein the vector is a liposome.

71. The vector of claim 70, wherein the liposome is a cationic liposome.

72. The vector of claim 70 or 71, wherein the liposome has a particle diameter in the range of from about 50 nm to about 200 nm.

73. A host cell comprising a nucleic acid of claim 66 or 67 or comprising a vector of any one of claims 68 to 72.

74. A virus comprising a nucleic acid of claim 66 or 67 or comprising a vector of any one of claims 68 to 72.

75. A pharmaceutical composition comprising an active agent and a pharmaceutically acceptable carrier, wherein the active agent is at least one selected from:
(i) an antibody of any one of claims 1 to 60;
(ii) a conjugate of claim 62 or 63;
(iii) a multimer of claim 64 or 65;
(iv) a nucleic acid of claim 66 or 67;
(v) a vector of any one of claims 68 to 72;
(vi) a host cell of claim 73; and/or
(vii) a virus of claim 74.

76. The pharmaceutical composition of claim 75, which is formulated for parenteral administration.

77. The pharmaceutical composition of claim 76, which is formulated for cardiovascular, in particular intravenous or intraarterial administration.

78. The pharmaceutical composition of any one of claims 75 to 77 for use in a prophylactic and/or therapeutic treatment of a disease.

79. The pharmaceutical composition of claim 78, wherein the disease is cancer growth and/or cancer metastasis.

80. The pharmaceutical composition of claim 78 or 79, wherein the disease is characterized by comprising diseased cells or cancer cells which are characterized by expressing PD-L 1 and/or being characterized by association of PD-L 1 with their surface.

81. The pharmaceutical composition of any one of claims 75 to 80 for use in a method of preventing or treating cancer.

82. The pharmaceutical composition of any one of claims 79 to 81, wherein the cancer is selected from the group consisting of melanoma, lung cancer, renal cell carcinoma, bladder cancer, breast cancer, gastric and gastroesophageal junction cancers, pancreatic adenocarcinoma, ovarian cancer and lymphomas.

83. The pharmaceutical composition of any one of claims 75 to 82, wherein the pharmaceutical composition is to be specifically delivered to, accumulated in and/or are retained in a target organ or tissue.

84. The pharmaceutical composition of any one of claims 75 to 83, wherein the vector or the virus releases the nucleic acid at the target organ or tissue and/or enters cells at the target organ or tissue.

85. The pharmaceutical composition of any one of claims 75 to 84, wherein the antibody is to be expressed in cells of the target organ or tissue.

86. The pharmaceutical composition of any one of claims 75 to 85, wherein the treatment is a monotherapy or a combination therapy.

87. The pharmaceutical composition of claim 86, wherein the combinatorial treatment is at least one treatment selected from the group consisting chemotherapy, molecular-targeted therapy, radiation therapy, and other forms of immune therapy.

88. The pharmaceutical composition of any one of claims 75 to 87, wherein the subject is a human.

89. A method of treating or preventing a disease in a subject comprising administering to a subject at least one active agent, wherein the active agent is at least one selected from:
(i) an antibody of any one of claims 1 to 60;
(ii) a conjugate of claim 62 or 63;
(iii) a multimer of claim 64 or 65;
(iv) a nucleic acid of claim 66 or 67;
(v) a vector of any one of claims 68 to 72;
(vi) a host cell of claim 73; and/or
(vii) a virus of claim 74.

90. The method of claim 89, wherein a pharmaceutical composition of any one of claims 75 to 77 is administered to the subject.

91. The method of claim 89 or 90, wherein the subject has a diseased organ or tissue characterized by cells expressing PD-L1 and/or being characterized by association of PD-L1 with their surface.

92. The method of any one of claims 89 to 91, wherein the disease is cancer growth and/or cancer metastasis.

93. The method of claim 92, wherein the cancer is selected from the group consisting of melanoma, lung cancer, renal cell carcinoma, bladder cancer, breast cancer, gastric and gastroesophageal junction cancers, pancreatic adenocarcinoma, ovarian cancer and lymphomas.

94. The method of any one of claims 89 to 93, wherein the active agent or the pharmaceutical composition is administered into the cardiovascular system.

95. The method of claim 94, wherein the active agent or the pharmaceutical composition is administered by intravenous or intraarterial administration such as administration into a peripheral vein.

96. The method of any one of claims 89 to 95, wherein the active agent or the pharmaceutical composition are specifically delivered to, accumulate in and/or are retained in a target organ or tissue.

97. The method of any one of claims 89 to 96, wherein the vector, the host cell or the virus releases the nucleic acid at the target organ or tissue and/or enters cells at the target organ or tissue.

98. The method of claim 97, wherein the antibody is expressed in cells of the target organ or tissue.

99. The method of any one of claims 89 to 98, wherein the treatment is a monotherapy or a combination therapy.

100. The method of claim 99, wherein the combinatorial treatment is at least one treatment selected from the group consisting chemotherapy, molecular-targeted therapy, radiation therapy, and other forms of immune therapy.

101. The method of any one of claims 89 to 100, wherein the subject is a human.

102. A kit for qualitative or quantitative detection of PD-1 in a sample, wherein the kit comprises an antibody of any one of claim 1 to 60 or a conjugate of claim 62 or 63 or a multimer of claim 64 or 65.

103. Use of an antibody of any one of claims 1 to 60 or of a conjugate of claim 62 or 63 or of a multimer of claim 64 or 65 or of a kit of claim 102 in a method of determining the presence or quantity of PD-1 expressed in a sample, the method comprising the steps of:
(i) contacting a sample with the antibody or the conjugate or the multimer, and
(ii) detecting the formation of and/or determining the quantity of a complex between the antibody or the conjugate or the multimer and PD-1.
